(19) United States
(12) Patent Application Publication
(10) Pub. No.: US 2023/0081390 A1
(43) Pub. Date: Mar. 16, 2023

(54) INHIBITORS OF RAF KINASES

(71) Applicant: Kinnate Biopharma Inc., San Diego, CA (US)

(72) Inventors: Stephen W. KALDOR, San Diego, CA (US); Toufike KANOUNI, Rancho Santa Fe, CA (US); Eric A. MURPHY, San Marcos, CA (US); Lee D. ARNOLD, Rancho Santa Fe, CA (US); John TYHONAS, San Diego, CA (US)

(21) Appl. No.: 17/441,403

(22) PCT Filed: Mar. 20, 2020
(86) PCT No.: PCT/US2020/024009

§ 371 Date: Sep. 21, 2021

Related U.S. Application Data
(60) Provisional application No. 62/822,733, filed on Mar. 22, 2019.

(51) Int. Cl. C07D 401/12; C07D 413/12; C07D 401/14; C07D 491/107; C07D 405/14; C07D 213/74; C07D 413/14; C07D 417/12

(52) U.S. Cl. CPC C07D 401/12; C07D 413/12; C07D 401/14; C07D 417/12; C07D 405/14; C07D 213/74; C07D 413/14; C07D 491/107

(57) ABSTRACT

Provided herein are inhibitors of receptor tyrosine kinase effector, RAF, pharmaceutical compositions comprising said compounds, and methods for using said compounds for the treatment of diseases.

### CROSS-REFERENCE TO RELATED APPLICATION

This application claims benefit of U.S. Patent Application No. 62/822,733, filed on Mar. 22, 2019, which is hereby incorporated by reference in its entirety.

### BACKGROUND

RAF kinase functions in the Ras-Raf-MEK-ERK mitogen activated protein kinase (MAPK) pathway (also known as MAPK/ERK pathway) by phosphorylating and activating MEK. By altering the levels and activities of transcription factors, MAPK leads to altered transcription of genes that are important for the cell cycle. Deregulation of MAPK activity occurs frequently in tumors. Accordingly, therapies that target RAF kinase activity are desired for use in the treatment of cancer and other disorders characterized by aberrant MAPK/ERK pathway signaling.

### BRIEF SUMMARY OF THE INVENTION

Provided herein are inhibitors of the receptor tyrosine kinase effector Raf (RAF), pharmaceutical compositions comprising said compounds, and methods for using said compounds for the treatment of diseases.

One embodiment provides a compound, or pharmaceutically acceptable salt or solvate thereof, having the structure of Formula (I):

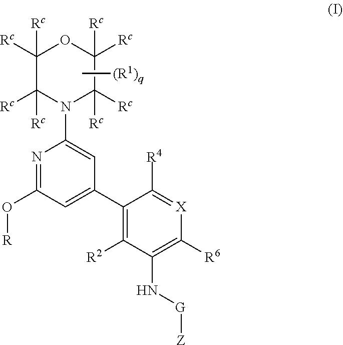

wherein,
G is C═O or SO₂;
R is C1-C8 optionally substituted alkyl, —(C1-C8 optionally substituted alkylene)-OPO(OH)₂, —(C1-C8 optionally substituted alkylene)-S(O)NHMe, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C₁-C₃ optionally substituted alkyl, and q is 0, 1, or 2; or optionally, if q is 2, then two R¹ groups join to form a fused ring;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b)

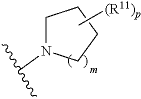

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c)

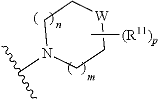

wherein m is 0, 1, 2, or 3; p is 0, 1, 2, 3, or 4; W is O, S, S(O), SO₂, NH or N(optionally substituted C1-C6 alkyl); and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d)

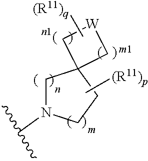

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e)

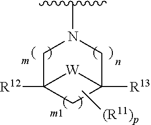

(wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—, —O—CH₂—, or —CH₂—O—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f)

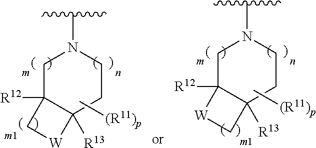

wherein m is 0, 1, or 2; n is 0, 1, or 2; m1 is 0, 1, or 2; p is 0, 1, or 2;
W is O, S, S(O), SO₂, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g)

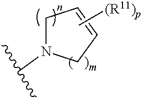

wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups to ether form an oxo;
(h)

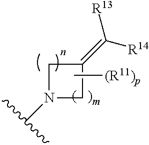

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl;
(i)

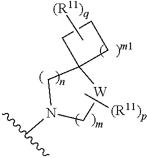

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

One embodiment provides a compound, or pharmaceutically acceptable salt or solvate thereof, having the structure of Formula (II):

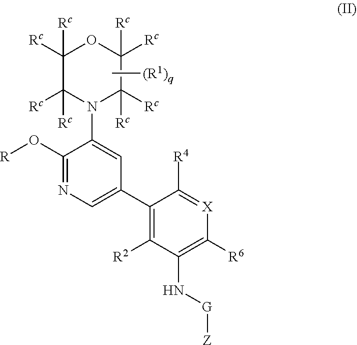

wherein,
G is C═O or SO₂;
R is C1-C8 optionally substituted alkyl, —(C1-C8 optionally substituted alkylene)-OPO(OH)₂, —(C1-C8 optionally substituted alkylene)-S(O)NHMe, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2; or optionally, if q is 2, then two R¹ groups join to form a fused ring;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
   R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b)

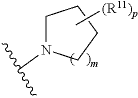

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c)

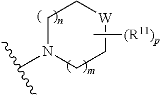

wherein m is 0, 1, 2, or 3; p is 0, 1, 2, 3, or 4; W is O, S, S(O), SO₂, NH or N(optionally substituted C1-C6 alkyl); and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d)

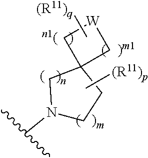

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e)

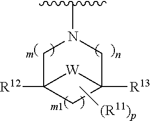

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—, —O—CH₂—, or —CH₂—O—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f)

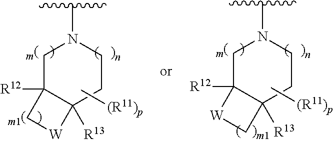

wherein m is 0, 1, or 2; n is 0, 1, or 2; m1 is 0, 1, or 2; p is 0, 1, or 2; W is O, S, S(O), SO₂, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g)

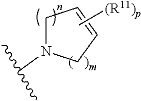

wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h)

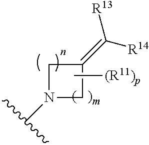

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl;
(i)

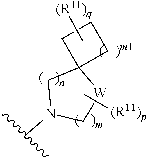

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

One embodiment provides a pharmaceutical composition comprising a compound of Formula (II), or pharmaceutically acceptable salt or solvate thereof, and at least one pharmaceutically acceptable excipient.

One embodiment provides a method of treating a disease or disorder in a patient in need thereof comprising administering to the patient a compound of Formula (II), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

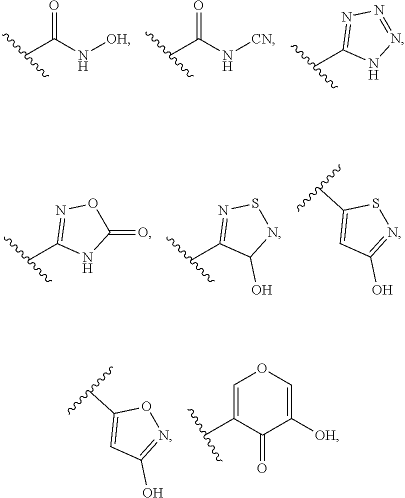

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R-heteroaryl, where R is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

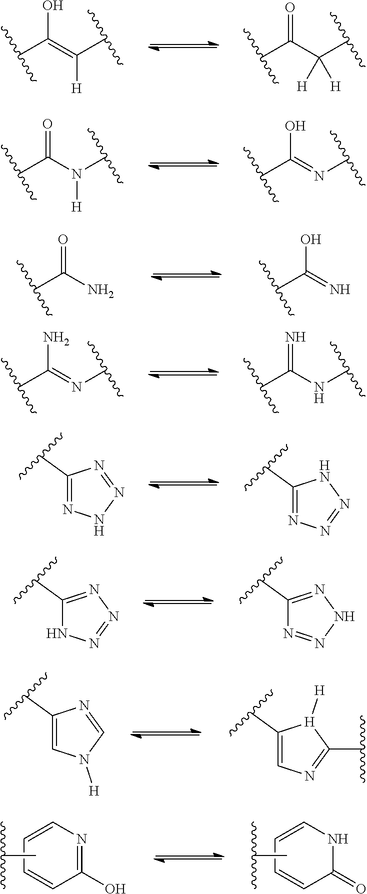

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

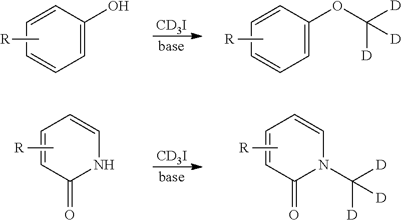

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

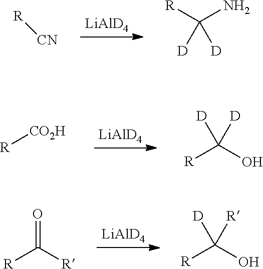

Deuterium gas and palladium catalyst are employed to reduce unsaturated carbon-carbon linkages and to perform a reductive substitution of aryl carbon-halogen bonds as illustrated, by way of example only, in the reaction schemes below.

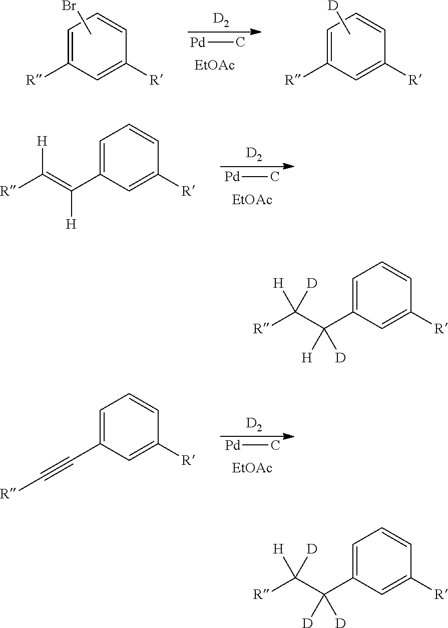

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heteroaromatic RAF inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### The RAF Family of Kinases

The RAF kinases are a family of serine/thronine protein kinases constitute core components of the RAS-RAF-MEK-ERK mitogen activated protein kinase (MAPK) signalling cascade (also known as the MAPK/ERK pathway), a pathway that mediates signals from cell surface receptors to the nucleus to regulate cell growth, differentiation and survival. The RAF proteins are related to retroviral oncogenes and are structurally conserved from metazoans to mammals, as is the MAPK/ERK pathway. Their dysregulation leads to uncontrolled cellular proliferation, survival and dedifferentiation. Consequently, RAF kinases are altered or inappropriately activated in a majority of cancers.

The MAPK/ERK signalling pathway is a network of proteins in the cell that communicates a signal from a receptor on the surface of the cell to the DNA in the nucleus of the cell. The signal starts when a signaling molecule binds to the receptor on the cell surface and ends when the DNA in the nucleus expresses a protein and produces some change in the cell, such as cell division. The pathway includes many proteins, which communicate by adding phosphate groups to a neighboring protein, which acts as a molecular “on” or “off” switch, and overall the pathway can be divided into 3 steps: (i) Ras activation, (ii) a kinase signal transduction cascade, and (iii) regulation of translation and transcription. Briefly, an extracellular mitogen or a signaling molecule binds to the membrane receptor. This allows Ras (a small GTPase) to swap its GDP for a GTP and become active. Activated Ras activates the protein kinase activity of RAF kinase. RAF kinase phosphorylates and activates MEK (MEK1 and MEK2). MEK then phosphorylates and activates a MAPK (also known as ERK). MAPK activation regulates activities of several transcription factors and also alters the translation of mRNA to proteins. By altering the levels and activities of transcription factors, MAPK leads to altered transcription of genes that are important for the cell cycle.

There are three known mammalian RAF isoforms: C-RAF (also known as RAF-1, or c-RAF-1), B-RAF, and A-RAF. All RAF kinases share a common modular structure consisting of 3 conserved regions (CR1, CR2, and CR3) with distinct functions. CR1 contains (i) a Ras-binding domain (RBD), which is necessary for the interaction with Ras and with membrane phospholipids required for membrane recruitment, and (ii) a cysteine-rich domain (CRD), which is a secondary Ras-binding site and also necessary for the interaction of CR1 with the kinase domain for RAF autoinhibition. CR2 contains important inhibitory phosphorylation sites participating in the negative regulation of Ras binding and RAF activation. CR3 features the kinase domain, including the activation segment, whose phosphorylation is crucial for kinase activation.

Functionally, the RAF structure can be split into a regulatory N-terminal region, containing the RBD, which is critical for activation as well as inhibitory phosphorylation sites, and a catalytic C-terminal region, which includes phosphorylation sites necessary for the kinase activation. The regulatory domain restrains the activity of the kinase domain, and its removal results in constitutive oncogenic activation. However, the activity of the isolated C-RAF kinase domain is subjected to further regulation and can be stimulated by phorbol esters, v-Src, and phosphorylation.

The common and key step in the activation of all 3 RAF kinase isoforms is membrane recruitment by a Ras family protein. The RAF kinases are located in the cytosol in their inactive state when bound to 14-3-3 proteins. In the presence of active Ras, they translocate to the plasma membrane. Membrane translocation triggers further activation events, such as the binding of PP2A to dephosphorylate the inhibitory pS259 site in RAF-1 (and presumably the corresponding sites in A-RAF and B-RAF) and the co-localization with the kinases responsible for the multiple activating phosphorylations. The sequences forming the binding interface are well conserved in the RAF as well as Ras family indicating that several members of the Ras family have the ability to bind RAF kinases. H-Ras, N-Ras, and K-Ras stimulate all 3 RAF isoforms and are the only Ras proteins that activate B-RAF. In contrast, A-RAF is also activated by R-Ras3, while C-RAF responds weakly to R-Ras3, Rit, and TC21 as well. But, all RAF kinases share MEK1/2 kinases as substrates. MEK1/2 in turn activate ERK1/2, and this pathway regulates many cellular functions such as cell proliferation, differentiation, migration, or apoptosis.

### C-RAF

C-RAF was first to be identified and is a ubiquitously expressed isoform. In humans, C-RAF is encoded by the RAF1 gene. C-RAF also has a known splice variant preferentially expressed in the muscle and brain. C-RAF plays a critical role in mediating the cellular effects of growth factor signals. In the inactive state, C-RAF exists in a closed conformation in which the N-terminal regulatory region folds over and occludes the catalytic region. This conformation is stabilized by a 14-3-3 dimer binding to an N-terminal site, phospho-S259 (pS259), and a C-terminal site, pS621. Dephosphorylation of pS259 at the cell membrane by specific phosphatases (PP2A, PP1) releases 14-3-3 from its N-terminal binding site in C-RAF, thereby allowing conformational changes to occur that unmask the RBD and CRD domains in the CR1 region to enable Ras binding and membrane recruitment.

### B-RAF

B-RAF is encoded in humans by the BRAF gene, also known as proto-oncogene B-RAF and v-RAF murine sarcoma viral oncogene homolog B. Alternative splicing gives rise to multiple B-RAF isoforms which are differentially expressed in various tissues. Whereas activation of A-RAF and C-RAF requires both phosphorylation and dephosphorylation of certain residues, as well as binding to other proteins, B-RAF becomes activated immediately upon translocation to the plasma membrane. B-RAF exhibits higher basal kinase activity than A-RAF and C-RAF. B-RAF requires Ras and 14-3-3 binding for its activation, and is inhibited or activated by PKA depending on the levels of 14-3-3 expression, which need to be high for permitting activation. B-RAF activity is also regulated by splicing. B-RAF isoforms containing exon 8b are more phosphorylated on the inhibitory S365 site, leading to an increased interaction with 14-3-3 and strengthening the inhibitory interaction between N-terminal regulatory domain and kinase domain, altogether resulting in lower kinase activity.

### A-RAF

Serine/threonine-protein kinase A-RAF or A-RAF is an enzyme encoded by the ARAF gene in humans. There are 2 known splice isoforms of A-RAF-DA-RAF1 and D-RAF2. They lack the kinase domain and act as dominant inhibitory mutants of Ras and ARF GTPases. DA-RAF1 is a positive regulator of myogenic differentiation by mediating the inhibition of the ERK pathway required for differentiation. There are several ways A-RAF is different from the other RAF kinases. A-RAF is the only steroid hormone-regulated Raf isoform. In addition, the A-RAF protein has amino acid substitutions in a negatively charged region upstream of the kinase domain (N-region), which contributes to its low basal activity. A-RAF is also only weakly activated by oncogenic H-Ras and Src and also displays low kinase activity towards MEK (the lowest kinase activity towards MEK proteins in the Raf kinase family). In addition to phosphorylating MEK, A-RAF also inhibits MST2, a tumor suppressor and pro-apoptotic kinase not found in the MAPK pathway. By inhibiting MST2, A-RAF prevents apoptosis from occurring. However, this inhibition is only occurs when the splice factor heterogenous nuclear ribonucleoprotein H (hnRNP H) maintains the expression of a full-length A-RAF protein. Tumorous cells often overexpress hnRNP H which leads to full-length expression of A-Raf which then inhibits apoptosis, allowing cancerous cells that should be destroyed to stay alive. A-RAF also binds to pyruvate kinase M2 (PKM2), again outside the MAPK pathway. PKM2 is an isozyme of pyruvate kinase that is responsible for the Warburg effect in cancer cells. A-RAF upregulates the activity of PKM2 by promoting a conformational change in PKM2. This causes PKM2 to transition from its low-activity dimeric form to a highly active tetrameric form. This causes more glucose carbons to be converted to pyruvate and lactate, producing energy for the cell, linking A-Raf to energy metabolism regulation and cell transformation, both of which are very important in tumorigenesis.

### RAF Kinase Inhibitors

Aberrant activation of the MAPK/ERK pathway is frequently found in various cancers and is a target for cancer therapeutics. In particular, B-RAF has emerged as one of the most attractive molecular targets for cancer therapeutics because somatic mutations of B-RAF have frequently been found in human tumors. Approximately 20% of all cancer samples tested to date harbor mutations in B-RAF. B-RAF-V600E, a missense mutation in the kinase domain generated by the substitution of glutamic acid with valine at position 600 is the most common B-RAF mutation. C-RAF is mutated in ˜1% of the various tumor types tested and the rate of mutations in A-RAF is even lower. B-RAF and C-RAF form both homo- and heterodimers as part of their activation mechanism and A-RAF stabilizes the B-RAF:C-RAF complexes to sustain signaling efficiency. Also, it is C-RAF, not B-RAF, that transmits signals from oncogenic RAS to MEK. Therefore, in different contexts, each of the RAF isoforms act as a potential therapeutic target.

Sorafenib was the first RAF inhibitor to enter clinical trials. Sorafenib is a broad specificity drug that inhibits additional kinases, including vascular endothelial growth factor receptor family (VEGFR-2 and VEGFR-3), platelet-derived growth factor receptor family (PDGFR-b and KIT) and FLT3. Clinical trials showed no correlation between the clinical responses with B-RAF mutation status, indicating it is a poor inhibitor of B-RAF. This led to the development of a new generation of B-RAF inhibitors, including, but not limited to vemurafenib, SB-590885, and dabrafenib (GSK2118436). Although the initial results of the clinical studies in B-RAF-mutant melanoma were encouraging, as clinical testing began in other B-RAF-mutated cancers (such as thyroid and colorectal cancers) it became apparent that tumors of different cell types harboring B-RAF mutations responded differently to selective B-RAF inhibition. Moreover, the existence of both primary and secondary resistance to RAF inhibition poses as one of the greatest challenges to the progress of RAF kinase inhibitor therapy. The mechanisms of resistance fall into two broad categories. Intrinsic/primary resistance is displayed by approximately 50% of patients. The other 50% of the patients initially respond (>30% tumor shrinkage) to RAF inhibitor but subsequently develop progressive disease associated with acquired/secondary resistance to RAF inhibitor. These two categories are not mutually exclusive because nearly all responders have remaining disease and, thus, may display intrinsic resistance. The determinants of primary RAF inhibitor resistance seem to vary with tumor type, with alteration in RTK signaling also being involved. Potential mechanisms of secondary B-RAF inhibitor resistance include, but are not limited to, reactivation of ERK1/2 pathways, upregulation of RTK signaling, the upregulation of receptor tyrosine kinases, mutations in RAS, and upregulation of COT. B-Raf alternative splicing and amplification of B-RAF-V600E have also been implicated in ˜30 and 20% of patients, respectively. Moreover, RAF kinase inhibitors cause paradoxical activation of the MAPK pathway, which, in some instances, leads to the development of secondary RAS mutation-driven malignancies. As such, there is a need in the field for new RAF kinase inhibitors that overcome the existing pitfalls and challenges posed by the current inhibitors.

### Heteroaromatic RAF Inhibitory Compounds

In one aspect, provided herein is a heteroaromatic RAF inhibitory compound.

One embodiment provides a compound, or pharmaceutically acceptable salt or solvate thereof, having the structure of Formula (I):

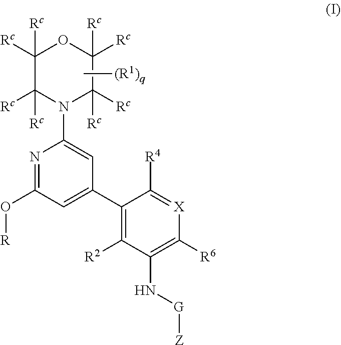

wherein,
G is C═O or SO₂;
R is C1-C8 optionally substituted alkyl, —(C1-C8 optionally substituted alkylene)-OPO(OH)₂, —(C1-C8 optionally substituted alkylene)-S(O)NHMe, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2; or optionally, if q is 2, then two R¹ groups join to form a fused ring;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
   R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b)

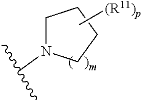

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c)

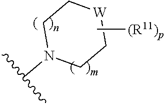

wherein m is 0, 1, 2, or 3; p is 0, 1, 2, 3, or 4; W is O, S, S(O), SO₂, NH or N(optionally substituted C1-C6 alkyl); and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d)

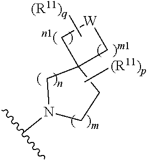

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e)

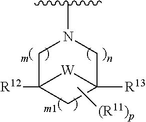

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—, —O—CH₂—, or —CH₂—O—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f)

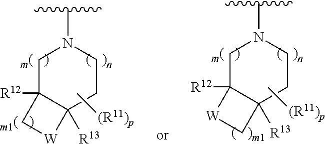

wherein m is 0, 1, or 2; n is 0, 1, or 2; m1 is 0, 1, or 2; p is 0, 1, or 2;
W is O, S, S(O), SO₂, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g)

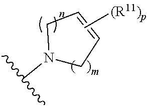

wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h)

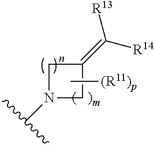

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl;
(i)

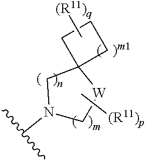

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

One embodiment provides a compound, or pharmaceutically acceptable salt or solvate thereof, having the structure of Formula (II):

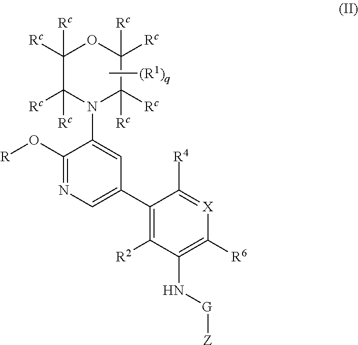

wherein,
G is C═O or SO₂;
R is C1-C8 optionally substituted alkyl, —(C1-C8 optionally substituted alkylene)-OPO(OH)₂, —(C1-C8 optionally substituted alkylene)-S(O)NHMe, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2; or optionally, if q is 2, then two R¹ groups join to form a fused ring;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
   R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b)

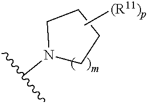

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c)

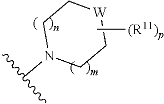

wherein m is 0, 1, 2, or 3; p is 0, 1, 2, 3, or 4; W is O, S, S(O), SO₂, NH or N(optionally substituted C1-C6 alkyl); and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d)

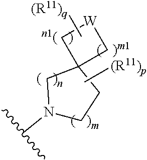

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e)

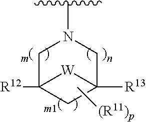

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—, —O—CH₂—, or —CH₂—O—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f)

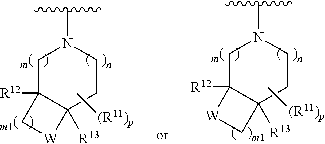

wherein m is 0, 1, or 2; n is 0, 1, or 2; m1 is 0, 1, or 2; p is 0, 1, or 2; W is O, S, S(O), SO₂, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g)

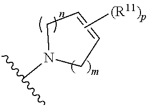

wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h)

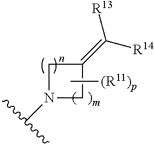

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl;
(i)

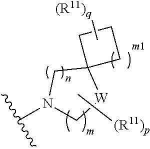

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

One embodiment provides the compound of Formula (I) or (II), or pharmaceutically acceptable salt or solvate thereof, wherein G is C═O.

One embodiment provides the compound of Formula (I) or (II), or pharmaceutically acceptable salt or solvate thereof, wherein R^{c} is hydrogen. Another embodiment provides the compound of Formula (I) or (II), or pharmaceutically acceptable salt or solvate thereof, wherein R^{c} is deuterium.

One embodiment provides the compound of Formula (I) or (II), or pharmaceutically acceptable salt or solvate thereof, wherein R² is hydrogen or deuterium. One embodiment provides the compound of Formula (I) or (II), or pharmaceutically acceptable salt or solvate thereof, wherein R² is F.

One embodiment provides the compound of Formula (I) or (II), or pharmaceutically acceptable salt or solvate thereof, wherein R⁶ is hydrogen or deuterium. One embodiment provides the compound of Formula (I) or (II), or pharmaceutically acceptable salt or solvate thereof, wherein R⁶ is F.

One embodiment provides the compound of Formula (I) or (II), or pharmaceutically acceptable salt or solvate thereof, wherein X is N. One embodiment provides the compound of Formula (I) or (II), or pharmaceutically acceptable salt or solvate thereof, wherein X is C—H or C-D. One embodiment provides the compound of Formula (I) or (II), or pharmaceutically acceptable salt or solvate thereof, wherein X is C—F.

One embodiment provides the compound of Formula (I) or (II), or pharmaceutically acceptable salt or solvate thereof, wherein R¹ is optionally substituted C1 alkyl. One embodiment provides the compound of Formula (I) or (II), or pharmaceutically acceptable salt or solvate thereof, wherein q is 0. One embodiment provides the compound of Formula (I) or (II), or pharmaceutically acceptable salt or solvate thereof, wherein q is 1. One embodiment provides the compound of Formula (I) or (II), or pharmaceutically acceptable salt or solvate thereof, wherein R¹ is CH₃, q is 1, and R¹ is positioned to provide a 3-methylmorpholino.

One embodiment provides the compound of Formula (I) or (II), or pharmaceutically acceptable salt or solvate thereof, wherein R is C1-C8 optionally substituted alkyl, C3-C6 optionally substituted cycloalkyl, C4-C6 optionally substituted cycloalkylalkyl, C3-C6 optionally substituted heterocyclyl, or C3-C6 optionally substituted heterocyclylalkyl.

One embodiment provides the compound of Formula (I) or (II), or pharmaceutically acceptable salt or solvate thereof, wherein R is C1-C8 optionally substituted alkyl. Another embodiment provides the compound of Formula (I) or (II), or pharmaceutically acceptable salt or solvate thereof, wherein the C1-C8 optionally substituted alkyl is a C2 optionally substituted alkyl.

One embodiment provides the compound of Formula (I) or (II), or pharmaceutically acceptable salt or solvate thereof, wherein R is —(C1-C8 optionally substituted alkylene)-OPO(OH)₂. Another embodiment provides the compound of Formula (I) or (II), or pharmaceutically acceptable salt or solvate thereof, wherein the —(C1-C8 optionally substituted alkylene)-OPO(OH)₂ is a C2 optionally substituted alkylene.

One embodiment provides the compound of Formula (I) or (II), or pharmaceutically acceptable salt or solvate thereof, wherein R is C3-C6 optionally substituted cycloalkyl. One embodiment provides the compound of Formula (I) or (II), or pharmaceutically acceptable salt or solvate thereof, wherein R is C4-C6 optionally substituted cycloalkylalkyl. One embodiment provides the compound of Formula (I) or (II), or pharmaceutically acceptable salt or solvate thereof, wherein R is C3-C6 optionally substituted heterocyclyl. One embodiment provides the compound of Formula (I) or (II), or pharmaceutically acceptable salt or solvate thereof, wherein R is C3-C6 optionally substituted heterocyclylalkyl.

One embodiment provides the compound of Formula (I) or (II), or pharmaceutically acceptable salt or solvate thereof, wherein R⁴ is halogen. One embodiment provides the compound of Formula (I) or (II), or pharmaceutically acceptable salt or solvate thereof, wherein R⁴ is optionally substituted C1-C3 alkyl. One embodiment provides the compound of Formula (I) or (II), or pharmaceutically acceptable salt or solvate thereof, wherein R⁴ is methyl.

One embodiment provides the compound of Formula (I) or (II), or pharmaceutically acceptable salt or solvate thereof, wherein Z is —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl. Another embodiment provides the compound of Formula (I) or (II), or pharmaceutically acceptable salt or solvate thereof, wherein R^{a} is H. Another embodiment provides the compound of Formula (I) or (II), or pharmaceutically acceptable salt or solvate thereof, wherein R^{a} is optionally substituted alkyl. Another embodiment provides the compound of Formula (I) or (II), or pharmaceutically acceptable salt or solvate thereof, wherein R^{b} is optionally substituted alkyl.

One embodiment provides the compound of Formula (I) or (II), or pharmaceutically acceptable salt or solvate thereof, wherein Z is

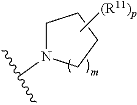

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo. Another embodiment provides the compound of Formula (I) or (II), or pharmaceutically acceptable salt or solvate thereof, wherein m is 0. Another embodiment provides the compound of Formula (I) or (II), or pharmaceutically acceptable salt or solvate thereof, wherein m is 1. Another embodiment provides the compound of Formula (I) or (II), or pharmaceutically acceptable salt or solvate thereof, wherein m is 2. Another embodiment provides the compound of Formula (I) or (II), or pharmaceutically acceptable salt or solvate thereof, wherein m is 3. Another embodiment provides the compound of Formula (I) or (II), or pharmaceutically acceptable salt or solvate thereof, wherein p is 0. Another embodiment provides the compound of Formula (I) or (II), or pharmaceutically acceptable salt or solvate thereof, wherein p is 1. Another embodiment provides the compound of Formula (I) or (II), or pharmaceutically acceptable salt or solvate thereof, wherein p is 2. Another embodiment provides the compound of Formula (I) or (II), or pharmaceutically acceptable salt or solvate thereof, wherein p is 1. Another embodiment provides the compound of Formula (I) or (II), or pharmaceutically acceptable salt or solvate thereof, wherein R¹¹ is optionally substituted C1-C6 alkyl, or optionally substituted C3-C6 cycloalkyl. Another embodiment provides the compound of Formula (I) or (II) or pharmaceutically acceptable salt or solvate thereof, wherein the optionally substituted C1-C6 alkyl, or optionally substituted C3-C6 cycloalkyl is substituted with at least a halogen.

One embodiment provides the compound of Formula (I) or (II), or pharmaceutically acceptable salt or solvate thereof, wherein Z is

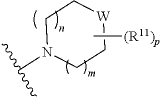

wherein m is 0, 1, 2, or 3; p is 0, 1, 2, 3, or 4;
W is O, S, S(O), SO₂, NH or N(optionally substituted C1-C6 alkyl); and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo. Another embodiment provides the compound of Formula (I) or (II), or pharmaceutically acceptable salt or solvate thereof, wherein W is O. Another embodiment provides the compound of Formula (I) or (II), or pharmaceutically acceptable salt or solvate thereof, wherein W is S. Another embodiment provides the compound of Formula (I) or (II), or pharmaceutically acceptable salt or solvate thereof, wherein m is 1, and n is 1. Another embodiment provides the compound of Formula (I) or (II), or pharmaceutically acceptable salt or solvate thereof, wherein m is 1, and n is 2. Another embodiment provides the compound of Formula (I) or (II), or pharmaceutically acceptable salt or solvate thereof, wherein R¹¹ is optionally substituted C1-C6 alkyl, or optionally substituted C3-C6 cycloalkyl. Another embodiment provides the compound of Formula (I) or (II), or pharmaceutically acceptable salt or solvate thereof, wherein the optionally substituted C1-C6 alkyl, or optionally substituted C3-C6 cycloalkyl is substituted with at least a halogen.

One embodiment provides the compound of Formula (I) or (II), or pharmaceutically acceptable salt or solvate thereof, wherein Z is

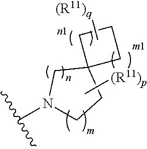

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo. Another embodiment provides the compound of Formula (I) or (II), or pharmaceutically acceptable salt or solvate thereof, wherein m is 1, and n is 1. Another embodiment provides the compound of Formula (I) or (II), or pharmaceutically acceptable salt or solvate thereof, wherein m is 0, and n is 2. Another embodiment provides the compound of Formula (I) or (II), or pharmaceutically acceptable salt or solvate thereof, wherein m1 is 0, and n1 is 2. Another embodiment provides the compound of Formula (I) or (II), or pharmaceutically acceptable salt or solvate thereof, wherein m1 is 1, and n1 is 1. Another embodiment provides the compound of Formula (I) or (II), or pharmaceutically acceptable salt or solvate thereof, wherein W is O. Another embodiment provides the compound of Formula (I) or (II), or pharmaceutically acceptable salt or solvate thereof, wherein W is CH₂. Another embodiment provides the compound of Formula (I) or (II), or pharmaceutically acceptable salt or solvate thereof, wherein W is CHR¹¹. Another embodiment provides the compound of Formula (I) or (II), or pharmaceutically acceptable salt or solvate thereof, wherein W is C(R¹¹)₂. Another embodiment provides the compound of Formula (I) or (II), or pharmaceutically acceptable salt or solvate thereof, wherein R¹¹ is halogen and q is 1.

One embodiment provides the compound of Formula (I) or (II), or pharmaceutically acceptable salt or solvate thereof, wherein Z is

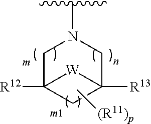

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl. Another embodiment provides the compound of Formula (I) or (II), or pharmaceutically acceptable salt or solvate thereof, wherein m is 0, n is 1, and m1 is 1; and W is —O—CH₂—, or —CH₂—O—.

One embodiment provides the compound of Formula (I) or (II), or pharmaceutically acceptable salt or solvate thereof, wherein Z is

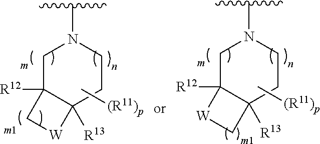

wherein m is 0, 1, or 2; n is 0, 1, or 2; m1 is 0, 1, or 2; p is 0, 1, or 2; W is O, S, S(O), SO₂, NH or N(optionally substituted C1-C6 alkyl), CH₂, CHR¹¹, or C(R¹¹)₂; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl. Another embodiment provides the compound of Formula (I) or (II), or pharmaceutically acceptable salt or solvate thereof, wherein W is O. Another embodiment provides the compound of Formula (I) or (II), or pharmaceutically acceptable salt or solvate thereof, wherein W is CH₂, or CHR¹¹. Another embodiment provides the compound of Formula (I) or (II), or pharmaceutically acceptable salt or solvate thereof, wherein m1 is 0. Another embodiment provides the compound of Formula (I) or (II), or pharmaceutically acceptable salt or solvate thereof, wherein m1 is 1. Another embodiment provides the compound of Formula (I) or (II), or pharmaceutically acceptable salt or solvate thereof, wherein m is 1 and n is 1. Another embodiment provides the compound of Formula (I) or (II), or pharmaceutically acceptable salt or solvate thereof, wherein m is 1 and n is 0. Another embodiment provides the compound of Formula (I) or (II), or pharmaceutically acceptable salt or solvate thereof, wherein m is 0 and n is 1.

One embodiment provides the compound of Formula (I) or (II), or pharmaceutically acceptable salt or solvate thereof, wherein Z is

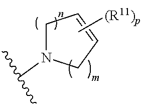

wherein m is 0, 1, 2, or 3; n is 0, 1, 2, or 3 provided both m and n are not both 0; p is 0, 1, 2, 3, or 4; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl. Another embodiment provides the compound of Formula (I) or (II), or pharmaceutically acceptable salt or solvate thereof, wherein m is 1, and n is 1. Another embodiment provides the compound of Formula (I) or (II), or pharmaceutically acceptable salt or solvate thereof, wherein m is 1, and n is 2. Another embodiment provides the compound of Formula (I) or (II), or pharmaceutically acceptable salt or solvate thereof, wherein p is 1. Another embodiment provides the compound of Formula (I) or (II), or pharmaceutically acceptable salt or solvate thereof, wherein p is 2. Another embodiment provides the compound of Formula (I) or (II), or pharmaceutically acceptable salt or solvate thereof, wherein at least one R¹¹ is attached to an alkene carbon. Another embodiment provides the compound of Formula (I) or (II), or pharmaceutically acceptable salt or solvate thereof, wherein at least one R¹¹ is not attached to an alkene carbon. Another embodiment provides the compound of Formula (I) or (II), or pharmaceutically acceptable salt or solvate thereof, wherein R¹¹ is optionally substituted C1-C6 alkyl, or optionally substituted C3-C6 cycloalkyl. Another embodiment provides the compound of Formula (I) or (II), or pharmaceutically acceptable salt or solvate thereof, wherein p is 0.

One embodiment provides the compound of Formula (I) or (II), or pharmaceutically acceptable salt or solvate thereof, wherein Z is

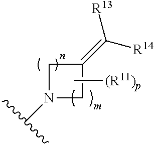

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl. Another embodiment provides the compound of Formula (I) or (II), or pharmaceutically acceptable salt or solvate thereof, wherein m is 2, and n is 1. Another embodiment provides the compound of Formula (I) or (II), or pharmaceutically acceptable salt or solvate thereof, wherein p is 0. Another embodiment provides the compound of Formula (I) or (II), or pharmaceutically acceptable salt or solvate thereof, wherein p is 1. Another embodiment provides the compound of Formula (I) or (II), or pharmaceutically acceptable salt or solvate thereof, wherein one of R¹³ or R¹⁴ is not hydrogen. Another embodiment provides the compound of Formula (I) or (II), or pharmaceutically acceptable salt or solvate thereof, wherein one of R¹³ or R¹⁴ is optionally substituted C1-C6 alkyl. Another embodiment provides the compound of Formula (I) or (II), or pharmaceutically acceptable salt or solvate thereof, wherein R¹³ is optionally substituted C1-C6 alkyl. Another embodiment provides the compound of Formula (I) or (II), or pharmaceutically acceptable salt or solvate thereof, wherein R¹⁴ is optionally substituted C1-C6 alkyl.

One embodiment provides the compound of Formula (I) or (II), or pharmaceutically acceptable salt or solvate thereof, wherein Z is

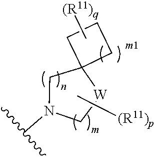

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo. Another embodiment provides the compound of Formula (I) or (II), or pharmaceutically acceptable salt or solvate thereof, wherein W is O. Another embodiment provides the compound of Formula (I) or (II), or pharmaceutically acceptable salt or solvate thereof, wherein m is 2, and n is 1. Another embodiment provides the compound of Formula (I) or (II), or pharmaceutically acceptable salt or solvate thereof, wherein m1 is 1 or 2. Another embodiment provides the compound of Formula (I) or (II), or pharmaceutically acceptable salt or solvate thereof, wherein p is 0 or 1, and q is 0 or 1.

One embodiment provides a compound, or pharmaceutically acceptable salt or solvate thereof, of Formula (I) having the structure of Formula (Ia):

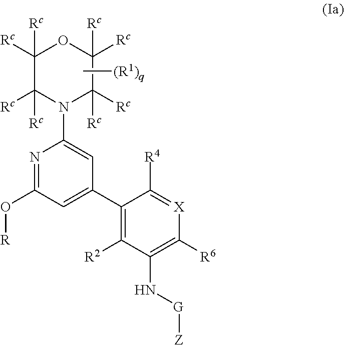

wherein,
G is C═O or SO₂;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
   R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b)

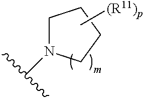

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c)

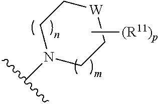

wherein m is 0, 1, 2, or 3; p is 0, 1, 2, 3, or 4; W is O, S, S(O), SO₂, NH or N(optionally substituted C1-C6 alkyl); and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d)

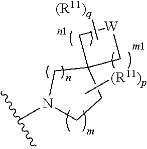

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2;
W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e)

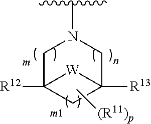

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f)

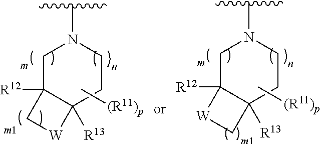

wherein m is 0, 1, or 2; n is 0, 1, or 2; m1 is 0, 1, or 2; p is 0, 1, or 2;
W is O, S, S(O), S02, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g)

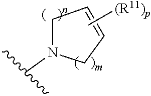

wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h)

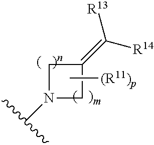

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or
(i)

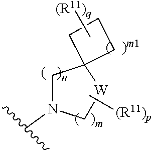

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

One embodiment provides a compound, or pharmaceutically acceptable salt or solvate thereof, of Formula (II) having the structure of Formula (IIa):

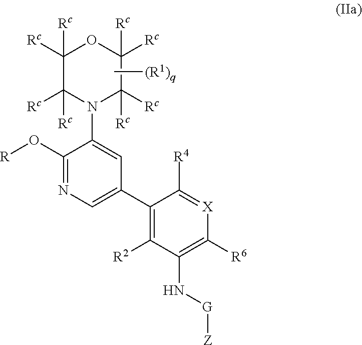

wherein,
G is C═O or SO₂;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
   R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b)

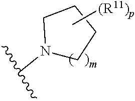

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c)

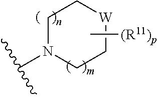

wherein m is 0, 1, 2, or 3; p is 0, 1, 2, 3, or 4; W is O, S, S(O), S02, NH or N(optionally substituted C1-C6 alkyl); and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d)

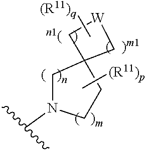

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e)

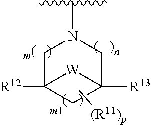

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f)

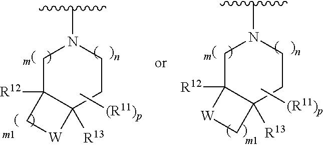

wherein m is 0, 1, or 2; n is 0, 1, or 2; m1 is 0, 1, or 2; p is 0, 1, or 2;
W is O, S, S(O), SO₂, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g)

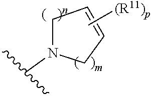

wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h)

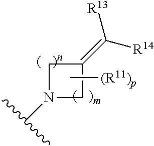

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or
(i)

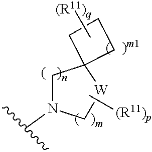

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

One embodiment provides the compound of Formula (Ia) or (IIa), or pharmaceutically acceptable salt or solvate thereof, wherein G is C═O.

One embodiment provides the compound of Formula (Ia) or (IIa), or pharmaceutically acceptable salt or solvate thereof, wherein R^{c} is hydrogen. Another embodiment provides the compound of Formula (Ia) or (IIa), or pharmaceutically acceptable salt or solvate thereof, wherein R^{c} is deuterium.

One embodiment provides the compound of Formula (Ia) or (IIa), or pharmaceutically acceptable salt or solvate thereof, wherein R² is hydrogen or deuterium. One embodiment provides the compound of Formula (Ia) or (IIa), or pharmaceutically acceptable salt or solvate thereof, wherein R² is F.

One embodiment provides the compound of Formula (Ia) or (IIa), or pharmaceutically acceptable salt or solvate thereof, wherein R⁶ is hydrogen or deuterium. One embodiment provides the compound of Formula (Ia) or (IIa), or pharmaceutically acceptable salt or solvate thereof, wherein R⁶ is F.

One embodiment provides the compound of Formula (Ia) or (IIa), or pharmaceutically acceptable salt or solvate thereof, wherein X is N. One embodiment provides the compound of Formula (Ia) or (IIa), or pharmaceutically acceptable salt or solvate thereof, wherein X is C—H or C-D. One embodiment provides the compound of Formula (Ia) or (IIa), or pharmaceutically acceptable salt or solvate thereof, wherein X is C—F.

One embodiment provides the compound of Formula (Ia) or (IIa), or pharmaceutically acceptable salt or solvate thereof, wherein R¹ is optionally substituted C1 alkyl. One embodiment provides the compound of Formula (Ia) or (IIa), or pharmaceutically acceptable salt or solvate thereof, wherein q is 0. One embodiment provides the compound of Formula (Ia) or (IIa), or pharmaceutically acceptable salt or solvate thereof, wherein q is 1. One embodiment provides the compound of Formula (Ia) or (IIa), or pharmaceutically acceptable salt or solvate thereof, wherein R¹ is CH₃, q is 1, and R¹ is positioned to provide a 3-methylmorpholino.

One embodiment provides the compound of Formula (Ia) or (IIa), or pharmaceutically acceptable salt or solvate thereof, wherein R is C1-C8 optionally substituted alkyl, C3-C6 optionally substituted cycloalkyl, C4-C6 optionally substituted cycloalkylalkyl, C3-C6 optionally substituted heterocyclyl, or C3-C6 optionally substituted heterocyclylalkyl.

One embodiment provides the compound of Formula (Ia) or (IIa), or pharmaceutically acceptable salt or solvate thereof, wherein R is C1-C8 optionally substituted alkyl. Another embodiment provides the compound of Formula (Ia) or (IIa), or pharmaceutically acceptable salt or solvate thereof, wherein the C1-C8 optionally substituted alkyl is a C2 optionally substituted alkyl.

One embodiment provides the compound of Formula (Ia) or (IIa), or pharmaceutically acceptable salt or solvate thereof, wherein R is —(C1-C8 optionally substituted alkylene)-OPO(OH)₂. Another embodiment provides the compound of Formula (Ia) or (IIa), or pharmaceutically acceptable salt or solvate thereof, wherein the —(C1-C8 optionally substituted alkylene)-OPO(OH)₂ is a C2 optionally substituted alkylene.

One embodiment provides the compound of Formula (Ia) or (IIa), or pharmaceutically acceptable salt or solvate thereof, wherein R is C3-C6 optionally substituted cycloalkyl. One embodiment provides the compound of Formula (Ia) or (IIa), or pharmaceutically acceptable salt or solvate thereof, wherein R is C4-C6 optionally substituted cycloalkylalkyl. One embodiment provides the compound of Formula (Ia) or (IIa), or pharmaceutically acceptable salt or solvate thereof, wherein R is C3-C6 optionally substituted heterocyclyl. One embodiment provides the compound of Formula (Ia) or (IIa), or pharmaceutically acceptable salt or solvate thereof, wherein R is C3-C6 optionally substituted heterocyclylalkyl.

One embodiment provides the compound of Formula (Ia) or (IIa), or pharmaceutically acceptable salt or solvate thereof, wherein R⁴ is halogen. One embodiment provides the compound of Formula (Ia) or (IIa), or pharmaceutically acceptable salt or solvate thereof, wherein R⁴ is optionally substituted C1-C3 alkyl. One embodiment provides the compound of Formula (Ia) or (IIa), or pharmaceutically acceptable salt or solvate thereof, wherein R⁴ is methyl.

One embodiment provides the compound of Formula (Ia) or (IIa), or pharmaceutically acceptable salt or solvate thereof, wherein Z is —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl. Another embodiment provides the compound of Formula (Ia) or (IIa), or pharmaceutically acceptable salt or solvate thereof, wherein R^{a} is H. Another embodiment provides the compound of Formula (Ia) or (IIa), or pharmaceutically acceptable salt or solvate thereof, wherein R^{a} is optionally substituted alkyl. Another embodiment provides the compound of Formula (Ia) or (IIa), or pharmaceutically acceptable salt or solvate thereof, wherein R^{b} is optionally substituted alkyl.

One embodiment provides the compound of Formula (Ia) or (IIa), or pharmaceutically acceptable salt or solvate thereof, wherein Z is

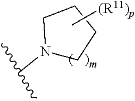

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo. Another embodiment provides the compound of Formula (Ia) or (IIa), or pharmaceutically acceptable salt or solvate thereof, wherein m is 0. Another embodiment provides the compound of Formula (Ia) or (IIa), or pharmaceutically acceptable salt or solvate thereof, wherein m is 1. Another embodiment provides the compound of Formula (Ia) or (IIa), or pharmaceutically acceptable salt or solvate thereof, wherein m is 2. Another embodiment provides the compound of Formula (Ia) or (IIa), or pharmaceutically acceptable salt or solvate thereof, wherein m is 3. Another embodiment provides the compound of Formula (Ia) or (IIa), or pharmaceutically acceptable salt or solvate thereof, wherein p is 0. Another embodiment provides the compound of Formula (Ia) or (IIa), or pharmaceutically acceptable salt or solvate thereof, wherein p is 1. Another embodiment provides the compound of Formula (Ia) or (IIa), or pharmaceutically acceptable salt or solvate thereof, wherein p is 2. Another embodiment provides the compound of Formula (Ia) or (IIa), or pharmaceutically acceptable salt or solvate thereof, wherein p is 1. Another embodiment provides the compound of Formula (Ia) or (IIa), or pharmaceutically acceptable salt or solvate thereof, wherein R¹¹ is optionally substituted C1-C6 alkyl, or optionally substituted C3-C6 cycloalkyl. Another embodiment provides the compound of Formula (Ia) or (IIa) or pharmaceutically acceptable salt or solvate thereof, wherein the optionally substituted C1-C6 alkyl, or optionally substituted C3-C6 cycloalkyl is substituted with at least a halogen.

One embodiment provides the compound of Formula (Ia) or (IIa), or pharmaceutically acceptable salt or solvate thereof, wherein Z is

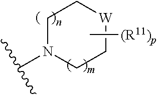

wherein m is 0, 1, 2, or 3; p is 0, 1, 2, 3, or 4; W is O, S, S(O), SO₂, NH or N(optionally substituted C1-C6 alkyl); and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo. Another embodiment provides the compound of Formula (Ia) or (IIa), or pharmaceutically acceptable salt or solvate thereof, wherein W is O. Another embodiment provides the compound of Formula (Ia) or (IIa), or pharmaceutically acceptable salt or solvate thereof, wherein W is S. Another embodiment provides the compound of Formula (Ia) or (IIa), or pharmaceutically acceptable salt or solvate thereof, wherein m is 1, and n is 1. Another embodiment provides the compound of Formula (Ia) or (IIa), or pharmaceutically acceptable salt or solvate thereof, wherein m is 1, and n is 2. Another embodiment provides the compound of Formula (Ia) or (IIa), or pharmaceutically acceptable salt or solvate thereof, wherein R¹¹ is optionally substituted C1-C6 alkyl, or optionally substituted C3-C6 cycloalkyl. Another embodiment provides the compound of Formula (Ia) or (IIa), or pharmaceutically acceptable salt or solvate thereof, wherein the optionally substituted C1-C6 alkyl, or optionally substituted C3-C6 cycloalkyl is substituted with at least a halogen.

One embodiment provides the compound of Formula (Ia) or (IIa), or pharmaceutically acceptable salt or solvate thereof, wherein Z is

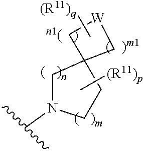

wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo. Another embodiment provides the compound of Formula (Ia) or (IIa), or pharmaceutically acceptable salt or solvate thereof, wherein m is 1, and n is 1. Another embodiment provides the compound of Formula (Ia) or (IIa), or pharmaceutically acceptable salt or solvate thereof, wherein m is 0, and n is 2. Another embodiment provides the compound of Formula (Ia) or (IIa), or pharmaceutically acceptable salt or solvate thereof, wherein m1 is 0, and n1 is 2. Another embodiment provides the compound of Formula (Ia) or (IIa), or pharmaceutically acceptable salt or solvate thereof, wherein m1 is 1, and n1 is 1. Another embodiment provides the compound of Formula (Ia) or (IIa), or pharmaceutically acceptable salt or solvate thereof, wherein W is O. Another embodiment provides the compound of Formula (Ia) or (IIa), or pharmaceutically acceptable salt or solvate thereof, wherein W is CH₂. Another embodiment provides the compound of Formula (Ia) or (IIa), or pharmaceutically acceptable salt or solvate thereof, wherein W is CHR¹¹. Another embodiment provides the compound of Formula (Ia) or (IIa), or pharmaceutically acceptable salt or solvate thereof, wherein W is C(R¹¹)₂. Another embodiment provides the compound of Formula (Ia) or (IIa), or pharmaceutically acceptable salt or solvate thereof, wherein R¹¹ is halogen and q is 1.

One embodiment provides the compound of Formula (Ia) or (IIa), or pharmaceutically acceptable salt or solvate thereof, wherein Z is

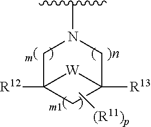

wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl. Another embodiment provides the compound of Formula (Ia) or (IIa), or pharmaceutically acceptable salt or solvate thereof, wherein m is 0, n is 1, and m1 is 1; and W is —O—CH₂—, or —CH₂—O—.

One embodiment provides the compound of Formula (Ia) or (IIa), or pharmaceutically acceptable salt or solvate thereof, wherein Z is

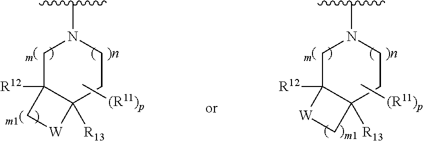

wherein m is 0, 1, or 2; n is 0, 1, or 2; m1 is 0, 1, or 2; p is 0, 1, or 2; W is O, S, S(O), SO₂, NH or N(optionally substituted C1-C6 alkyl), CH₂, CHR¹¹, or C(R¹¹)₂; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl. Another embodiment provides the compound of Formula (Ia) or (IIa), or pharmaceutically acceptable salt or solvate thereof, wherein W is O. Another embodiment provides the compound of Formula (Ia) or (IIa), or pharmaceutically acceptable salt or solvate thereof, wherein W is CH₂, or CHR¹¹. Another embodiment provides the compound of Formula (Ia) or (IIa), or pharmaceutically acceptable salt or solvate thereof, wherein m1 is 0. Another embodiment provides the compound of Formula (Ia) or (IIa), or pharmaceutically acceptable salt or solvate thereof, wherein m1 is 1. Another embodiment provides the compound of Formula (Ia) or (IIa), or pharmaceutically acceptable salt or solvate thereof, wherein m is 1 and n is 1. Another embodiment provides the compound of Formula (Ia) or (IIa), or pharmaceutically acceptable salt or solvate thereof, wherein m is 1 and n is 0. Another embodiment provides the compound of Formula (Ia) or (IIa), or pharmaceutically acceptable salt or solvate thereof, wherein m is 0 and n is 1.

One embodiment provides the compound of Formula (Ia) or (IIa), or pharmaceutically acceptable salt or solvate thereof, wherein Z is

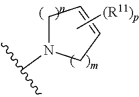

wherein m is 0, 1, 2, or 3; n is 0, 1, 2, or 3 provided both m and n are not both 0; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl. Another embodiment provides the compound of Formula (Ia) or (IIa), or pharmaceutically acceptable salt or solvate thereof, wherein m is 1, and n is 1. Another embodiment provides the compound of Formula (Ia) or (IIa), or pharmaceutically acceptable salt or solvate thereof, wherein m is 1, and n is 2. Another embodiment provides the compound of Formula (Ia) or (IIa), or pharmaceutically acceptable salt or solvate thereof, wherein p is 1. Another embodiment provides the compound of Formula (Ia) or (IIa), or pharmaceutically acceptable salt or solvate thereof, wherein p is 2. Another embodiment provides the compound of Formula (Ia) or (IIa), or pharmaceutically acceptable salt or solvate thereof, wherein at least one R¹¹ is attached to an alkene carbon. Another embodiment provides the compound of Formula (Ia) or (IIa), or pharmaceutically acceptable salt or solvate thereof, wherein at least one R¹¹ is not attached to an alkene carbon. Another embodiment provides the compound of Formula (Ia) or (IIa), or pharmaceutically acceptable salt or solvate thereof, wherein R¹¹ is optionally substituted C1-C6 alkyl, or optionally substituted C3-C6 cycloalkyl. Another embodiment provides the compound of Formula (Ia) or (IIa), or pharmaceutically acceptable salt or solvate thereof, wherein p is 0.

Another embodiment provides the compound of Formula (Ia) or (IIa), or pharmaceutically acceptable salt or solvate thereof, wherein Z is

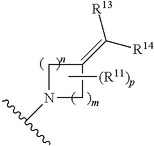

wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl. Another embodiment provides the compound of Formula (Ia) or (IIa), or pharmaceutically acceptable salt or solvate thereof, wherein m is 2, and n is 1. Another embodiment provides the compound of Formula (Ia) or (IIa), or pharmaceutically acceptable salt or solvate thereof, wherein p is 0. Another embodiment provides the compound of Formula (Ia) or (IIa), or pharmaceutically acceptable salt or solvate thereof, wherein p is 1. Another embodiment provides the compound of Formula (Ia) or (IIa), or pharmaceutically acceptable salt or solvate thereof, wherein one of R¹³ or R¹⁴ is not hydrogen. Another embodiment provides the compound of Formula (Ia) or (IIa), or pharmaceutically acceptable salt or solvate thereof, wherein one of R¹³ or R¹⁴ is optionally substituted C1-C6 alkyl. Another embodiment provides the compound of Formula (Ia) or (IIa), or pharmaceutically acceptable salt or solvate thereof, wherein R¹³ is optionally substituted C1-C6 alkyl. Another embodiment provides the compound of Formula (Ia) or (IIa), or pharmaceutically acceptable salt or solvate thereof, wherein R¹⁴ is optionally substituted C1-C6 alkyl.

One embodiment provides the compound of Formula (Ia) or (IIa), or pharmaceutically acceptable salt or solvate thereof, wherein Z is

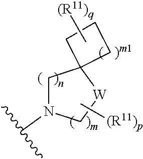

wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo. Another embodiment provides the compound of Formula (Ia) or (IIa), or pharmaceutically acceptable salt or solvate thereof, wherein W is O. Another embodiment provides the compound of Formula (Ia) or (IIa), or pharmaceutically acceptable salt or solvate thereof, wherein m is 2, and n is 1. Another embodiment provides the compound of Formula (Ia) or (IIa), or pharmaceutically acceptable salt or solvate thereof, wherein m1 is 1 or 2. Another embodiment provides the compound of Formula (Ia) or (IIa), or pharmaceutically acceptable salt or solvate thereof, wherein p is 0 or 1, and q is 0 or 1.

One embodiment provides a compound, or pharmaceutically acceptable salt or solvate thereof, having the structure of Formula (III):

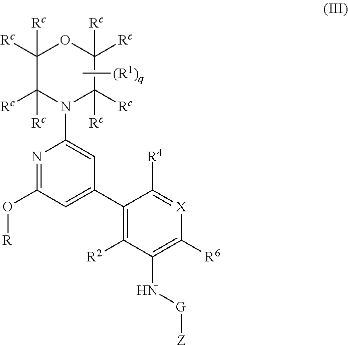

wherein,
G is C═O or SO₂;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a)

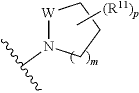

wherein m is 0, 1, 2, or 3; p is 0, 1, 2, 3, or 4; and W is O, NH or N(optionally substituted C1-C6 alkyl); each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(b)

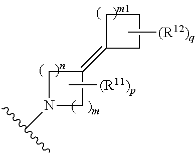

wherein m is 1, 2, or 3; n is 1, 2, or 3; m1 is 0, 1, 2, or 3; p is 0, 1, or 2; q is 0, 1, or 2; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo; and each R¹² is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹² groups together form an oxo.

One embodiment provides a compound, or pharmaceutically acceptable salt or solvate thereof, having the structure of Formula (IV):

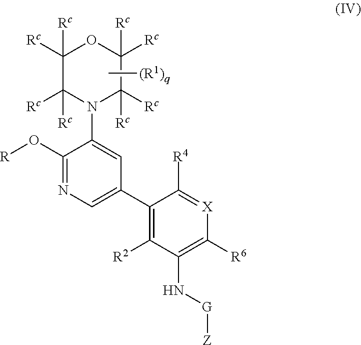

wherein,
G is C═O or SO₂;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a)

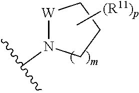

wherein m is 0, 1, 2, or 3; p is 0, 1, 2, 3, or 4; and W is O, NH or N(optionally substituted C1-C6 alkyl); each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(b)

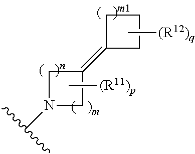

wherein m is 1, 2, or 3; n is 1, 2, or 3; m1 is 0, 1, 2, or 3; p is 0, 1, or 2; q is 0, 1, or 2; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo; and each R¹² is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹² groups together form an oxo.

One embodiment provides the compound of Formula (III) or (IV), or pharmaceutically acceptable salt or solvate thereof, wherein G is C═O.

One embodiment provides the compound of Formula (III) or (IV), or pharmaceutically acceptable salt or solvate thereof, wherein R^{c} is hydrogen. Another embodiment provides the compound of Formula (III) or (IV), or pharmaceutically acceptable salt or solvate thereof, wherein R^{c} is deuterium.

One embodiment provides the compound of Formula (III) or (IV), or pharmaceutically acceptable salt or solvate thereof, wherein R² is hydrogen or deuterium. One embodiment provides the compound of Formula (III) or (IV), or pharmaceutically acceptable salt or solvate thereof, wherein R² is F.

One embodiment provides the compound of Formula (III) or (IV), or pharmaceutically acceptable salt or solvate thereof, wherein R⁶ is hydrogen or deuterium. One embodiment provides the compound of Formula (III) or (IV), or pharmaceutically acceptable salt or solvate thereof, wherein R⁶ is F.

One embodiment provides the compound of Formula (III) or (IV), or pharmaceutically acceptable salt or solvate thereof, wherein X is N. One embodiment provides the compound of Formula (III) or (IV), or pharmaceutically acceptable salt or solvate thereof, wherein X is C—H or C-D. One embodiment provides the compound of Formula (III) or (IV), or pharmaceutically acceptable salt or solvate thereof, wherein X is C—F.

One embodiment provides the compound of Formula (III) or (IV), or pharmaceutically acceptable salt or solvate thereof, wherein R¹ is optionally substituted C1 alkyl. One embodiment provides the compound of Formula (III) or (IV), or pharmaceutically acceptable salt or solvate thereof, wherein q is 0. One embodiment provides the compound of Formula (III) or (IV), or pharmaceutically acceptable salt or solvate thereof, wherein q is 1. One embodiment provides the compound of Formula (III) or (IV), or pharmaceutically acceptable salt or solvate thereof, wherein R¹ is CH₃, q is 1, and R¹ is positioned to provide a 3-methylmorpholino.

One embodiment provides the compound of Formula (III) or (IV), or pharmaceutically acceptable salt or solvate thereof, wherein R is C1-C8 optionally substituted alkyl, C3-C6 optionally substituted cycloalkyl, C4-C6 optionally substituted cycloalkylalkyl, C3-C6 optionally substituted heterocyclyl, or C3-C6 optionally substituted heterocyclylalkyl.

One embodiment provides the compound of Formula (III) or (IV), or pharmaceutically acceptable salt or solvate thereof, wherein R is C1-C8 optionally substituted alkyl. Another embodiment provides the compound of Formula (III) or (IV), or pharmaceutically acceptable salt or solvate thereof, wherein the C1-C8 optionally substituted alkyl is a C2 optionally substituted alkyl.

One embodiment provides the compound of Formula (III) or (IV), or pharmaceutically acceptable salt or solvate thereof, wherein R is —(C1-C8 optionally substituted alkylene)-OPO(OH)₂. Another embodiment provides the compound of Formula (III) or (IV), or pharmaceutically acceptable salt or solvate thereof, wherein the —(C1-C8 optionally substituted alkylene)-OPO(OH)₂ is a C2 optionally substituted alkylene.

One embodiment provides the compound of Formula (III) or (IV), or pharmaceutically acceptable salt or solvate thereof, wherein R is C3-C6 optionally substituted cycloalkyl. One embodiment provides the compound of Formula (III) or (IV), or pharmaceutically acceptable salt or solvate thereof, wherein R is C4-C6 optionally substituted cycloalkylalkyl. One embodiment provides the compound of Formula (III) or (IV), or pharmaceutically acceptable salt or solvate thereof, wherein R is C3-C6 optionally substituted heterocyclyl. One embodiment provides the compound of Formula (III) or (IV), or pharmaceutically acceptable salt or solvate thereof, wherein R is C3-C6 optionally substituted heterocyclylalkyl.

One embodiment provides the compound of Formula (III) or (IV), or pharmaceutically acceptable salt or solvate thereof, wherein R⁴ is halogen. One embodiment provides the compound of Formula (III) or (IV), or pharmaceutically acceptable salt or solvate thereof, wherein R⁴ is optionally substituted C1-C3 alkyl. One embodiment provides the compound of Formula (III) or (IV), or pharmaceutically acceptable salt or solvate thereof, wherein R⁴ is methyl.

One embodiment provides the compound of Formula (III) or (IV), or pharmaceutically acceptable salt or solvate thereof, wherein Z is

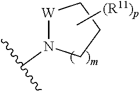

wherein m is 0, 1, 2, or 3; p is 0, 1, 2, 3, or 4; and W is O, NH or N(optionally substituted C1-C6 alkyl); each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo. Another embodiment provides the compound of Formula (III) or (IV), or pharmaceutically acceptable salt or solvate thereof, wherein m is 1 or 2. Another embodiment provides the compound of Formula (III) or (IV), or pharmaceutically acceptable salt or solvate thereof, wherein m is 1. Another embodiment provides the compound of Formula (III) or (IV), or pharmaceutically acceptable salt or solvate thereof, wherein W is O. Another embodiment provides the compound of Formula (III) or (IV), or pharmaceutically acceptable salt or solvate thereof, wherein W is NH. Another embodiment provides the compound of Formula (III) or (IV), or pharmaceutically acceptable salt or solvate thereof, wherein W is N(optionally substituted C1-C6 alkyl). Another embodiment provides the compound of Formula (III) or (IV), or pharmaceutically acceptable salt or solvate thereof, wherein p is 0. Another embodiment provides the compound of Formula (III) or (IV), or pharmaceutically acceptable salt or solvate thereof, wherein p is 1. Another embodiment provides the compound of Formula (III) or (IV), or pharmaceutically acceptable salt or solvate thereof, wherein two R¹¹ groups together form an oxo.

One embodiment provides the compound of Formula (III) or (IV), or pharmaceutically acceptable salt or solvate thereof, wherein Z is

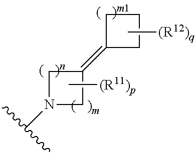

wherein m is 1, 2, or 3; n is 1, 2, or 3; m1 is 0, 1, 2, or 3; p is 0, 1, or 2; q is 0, 1, or 2; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo; and each R¹² is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹² groups together form an oxo. Another embodiment provides the compound of Formula (III) or (IV), or pharmaceutically acceptable salt or solvate thereof, wherein m is 1 and n is 2. Another embodiment provides the compound of Formula (III) or (IV), or pharmaceutically acceptable salt or solvate thereof, wherein m1 is 0, 1, or 2. Another embodiment provides the compound of Formula (III) or (IV), or pharmaceutically acceptable salt or solvate thereof, wherein m is 1, n is 2, and m1 is 0, 1, or 2. Another embodiment provides the compound of Formula (III) or (IV), or pharmaceutically acceptable salt or solvate thereof, wherein m is 1, n is 2, and m1 is 0. Another embodiment provides the compound of Formula (III) or (IV), or pharmaceutically acceptable salt or solvate thereof, wherein p is 0 or 1. Another embodiment provides the compound of Formula (III) or (IV), or pharmaceutically acceptable salt or solvate thereof, wherein q is 0 or 1.

One embodiment provides a compound, or pharmaceutically acceptable salt or solvate thereof, having the structure of Formula (V):

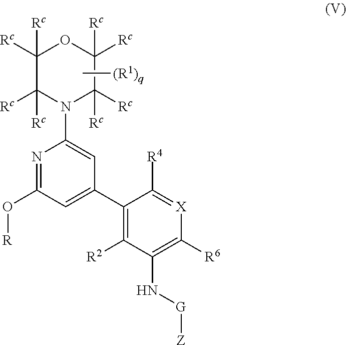

wherein,
G is C═O or SO₂;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is an optionally substituted N-linked pyrrole, optionally substituted-NH-pyrazole, or optionally substituted —N(optionally substituted C1-C6 alkyl)-pyrazole.

One embodiment provides a compound, or pharmaceutically acceptable salt or solvate thereof, having the structure of Formula (VI):

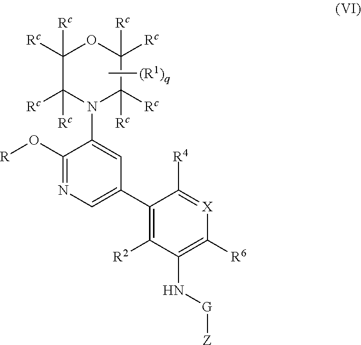

wherein,
G is C═O or SO₂;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is an optionally substituted N-linked pyrrole, optionally substituted-NH-pyrazole, or optionally substituted —N(optionally substituted C1-C6 alkyl)-pyrazole.

One embodiment provides the compound of Formula (V) or (VI), or pharmaceutically acceptable salt or solvate thereof, wherein G is C═O.

One embodiment provides the compound of Formula (V) or (VI), or pharmaceutically acceptable salt or solvate thereof, wherein R^{c} is hydrogen. Another embodiment provides the compound of Formula (V) or (VI), or pharmaceutically acceptable salt or solvate thereof, wherein R^{c} is deuterium.

One embodiment provides the compound of Formula (V) or (VI), or pharmaceutically acceptable salt or solvate thereof, wherein R² is hydrogen or deuterium. One embodiment provides the compound of Formula (V) or (VI), or pharmaceutically acceptable salt or solvate thereof, wherein R² is F.

One embodiment provides the compound of Formula (V) or (VI), or pharmaceutically acceptable salt or solvate thereof, wherein R⁶ is hydrogen or deuterium. One embodiment provides the compound of Formula (V) or (VI), or pharmaceutically acceptable salt or solvate thereof, wherein R⁶ is F.

One embodiment provides the compound of Formula (V) or (VI), or pharmaceutically acceptable salt or solvate thereof, wherein X is N. One embodiment provides the compound of Formula (V) or (VI), or pharmaceutically acceptable salt or solvate thereof, wherein X is C—H or C-D. One embodiment provides the compound of Formula (V) or (VI), or pharmaceutically acceptable salt or solvate thereof, wherein X is C—F.

One embodiment provides the compound of Formula (V) or (VI), or pharmaceutically acceptable salt or solvate thereof, wherein R¹ is optionally substituted C1 alkyl. One embodiment provides the compound of Formula (V) or (VI), or pharmaceutically acceptable salt or solvate thereof, wherein q is 0. One embodiment provides the compound of Formula (V) or (VI), or pharmaceutically acceptable salt or solvate thereof, wherein q is 1. One embodiment provides the compound of Formula (V) or (VI), or pharmaceutically acceptable salt or solvate thereof, wherein R¹ is CH₃, q is 1, and R¹ is positioned to provide a 3-methylmorpholino.

One embodiment provides the compound of Formula (V) or (VI), or pharmaceutically acceptable salt or solvate thereof, wherein R is C1-C8 optionally substituted alkyl, C3-C6 optionally substituted cycloalkyl, C4-C6 optionally substituted cycloalkylalkyl, C3-C6 optionally substituted heterocyclyl, or C3-C6 optionally substituted heterocyclylalkyl.

One embodiment provides the compound of Formula (V) or (VI), or pharmaceutically acceptable salt or solvate thereof, wherein R is C1-C8 optionally substituted alkyl. Another embodiment provides the compound of Formula (V) or (VI), or pharmaceutically acceptable salt or solvate thereof, wherein the C1-C8 optionally substituted alkyl is a C2 optionally substituted alkyl.

One embodiment provides the compound of Formula (V) or (VI), or pharmaceutically acceptable salt or solvate thereof, wherein R is —(C1-C8 optionally substituted alkylene)-OPO(OH)₂. Another embodiment provides the compound of Formula (V) or (VI), or pharmaceutically acceptable salt or solvate thereof, wherein the —(C1-C8 optionally substituted alkylene)-OPO(OH)₂ is a C2 optionally substituted alkylene.

One embodiment provides the compound of Formula (V) or (VI), or pharmaceutically acceptable salt or solvate thereof, wherein R is C3-C6 optionally substituted cycloalkyl. One embodiment provides the compound of Formula (V) or (VI), or pharmaceutically acceptable salt or solvate thereof, wherein R is C4-C6 optionally substituted cycloalkylalkyl. One embodiment provides the compound of Formula (V) or (VI), or pharmaceutically acceptable salt or solvate thereof, wherein R is C3-C6 optionally substituted heterocyclyl. One embodiment provides the compound of Formula (V) or (VI), or pharmaceutically acceptable salt or solvate thereof, wherein R is C3-C6 optionally substituted heterocyclylalkyl.

One embodiment provides the compound of Formula (V) or (VI), or pharmaceutically acceptable salt or solvate thereof, wherein R⁴ is halogen. One embodiment provides the compound of Formula (V) or (VI), or pharmaceutically acceptable salt or solvate thereof, wherein R⁴ is optionally substituted C1-C3 alkyl. One embodiment provides the compound of Formula (V) or (VI), or pharmaceutically acceptable salt or solvate thereof, wherein R⁴ is methyl.

One embodiment provides the compound of Formula (V) or (VI), or pharmaceutically acceptable salt or solvate thereof, wherein Z is an optionally substituted N-linked pyrrole.

One embodiment provides the compound of Formula (V) or (VI), or pharmaceutically acceptable salt or solvate thereof, wherein Z is an optionally substituted —NH-pyrazole.

One embodiment provides the compound of Formula (V) or (VI), or pharmaceutically acceptable salt or solvate thereof, wherein Z is an optionally substituted —N(optionally substituted C1-C6 alkyl)-pyrazole.

In some embodiments, the heteroaromatic RAF kinase inhibitory compound as described herein has a structure provided in Table 1.

TABLE 1

| Synthetic | | |
|---|---|---|
| Chemistry | | |
| Example | Compound Structure | Compound Name |
| 1 | 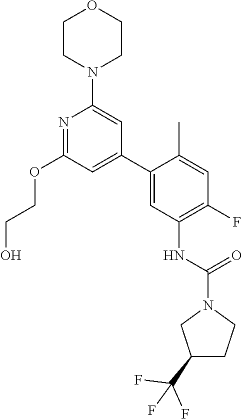 [SMILES: Cc1cc(F)c(NC(=O)N2CC[C@@H](C(F)(F)F)C2)cc1-c1cc(OCCO)nc(N2CCOCC2)c1] | (R)-N-(2-fluoro-5-(2-(2- hydroxyethoxy)-6-morpholinopyridin- 4-yl)-4-methylphenyl)-3- (trifluoromethyl)pyrrolidine-1- carboxamide |
| 2 | 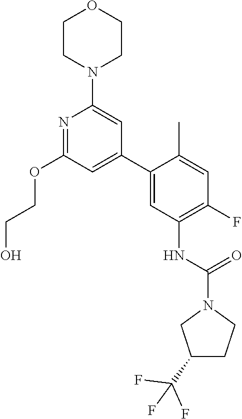 [SMILES: Cc1cc(F)c(NC(=O)N2CC[C@H](C(F)(F)F)C2)cc1-c1cc(OCCO)nc(N2CCOCC2)c1] | (S)-N-(2-fluoro-5-(2-(2- hydroxyethoxy)-6-morpholinopyridin- 4-yl)-4-methylphenyl)-3- (trifluoromethyl)pyrrolidine-1- carboxamide |
| 3 | 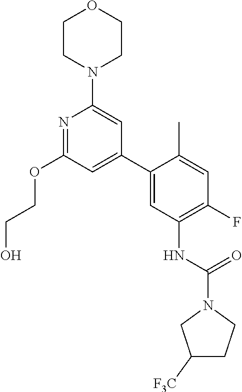 [SMILES: Cc1cc(F)c(NC(=O)N2CCC(C(F)(F)F)C2)cc1-c1cc(OCCO)nc(N2CCOCC2)c1] | (RS)-N-(2-fluoro-5-(2-(2- hydroxyethoxy)-6-morpholinopyridin- 4-yl)-4-methylphenyl)-3- (trifluoromethyl)pyrrolidine-1- carboxamide |
| 4 | 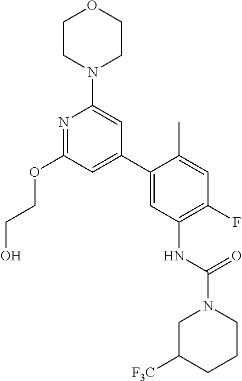 [SMILES: Cc1cc(F)c(NC(=O)N2CCCC(C(F)(F)F)C2)cc1-c1cc(OCCO)nc(N2CCOCC2)c1] | (RS)-N-(2-fluoro-5-(2-(2- hydroxyethoxy)-6-morpholinopyridin- 4 -yl)-4-methylphenyl)-3- (trifluoromethyl)piperidine-1- carboxamide |
| 5 | 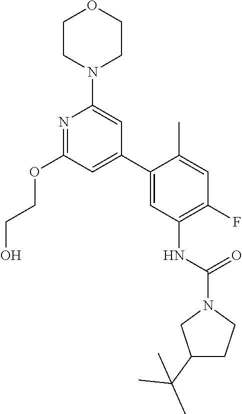 [SMILES: Cc1cc(F)c(NC(=O)N2CCC(C(C)(C)C)C2)cc1-c1cc(OCCO)nc(N2CCOCC2)c1] | (RS)-3-(tert-butyl)-N-(2-fluoro-5-(2- (2-hydroxyethoxy)-6- morpholinopyridin-4-yl)-4- methylphenyl)pyrrolidine-1- carboxamide |
| 6 | 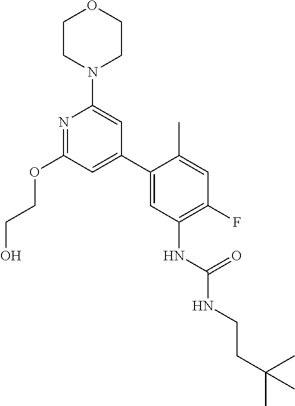 [SMILES: Cc1cc(F)c(NC(=O)NCCC(C)(C)C)cc1-c1cc(OCCO)nc(N2CCOCC2)c1] | 1-(3,3-dimethylbutyl)-3-(2-fluoro-5-(2- (2-hydroxyethoxy)-6- morpholinopyridin-4-yl)-4- methylphenyl)urea |
| 7 | 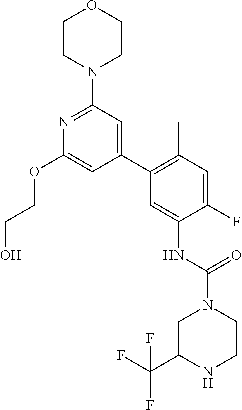 [SMILES: Cc1cc(F)c(NC(=O)N2CCNC(C(F)(F)F)C2)cc1-c1cc(OCCO)nc(N2CCOCC2)c1] | (RS)-N-(2-fluoro-5-(2-(2- hydroxyethoxy)-6-morpholinopyridin- 4-yl)-4-methylphenyl)-3- (trifluoromethyl)piperazine-1- carboxamide |
| 8 | 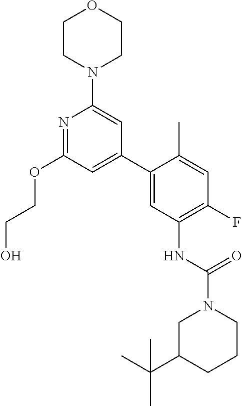 [SMILES: Cc1cc(F)c(NC(=O)N2CCCC(C(C)(C)C)C2)cc1-c1cc(OCCO)nc(N2CCOCC2)c1] | (RS)-3-(tert-butyl)-N-(2-fluoro-5-(2- (2-hydroxyethoxy)-6- morpholinopyridin-4-yl)-4- methylphenyl)piperidine-1- carboxamide |
| 9 | 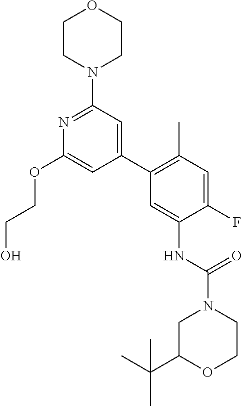 [SMILES: Cc1cc(F)c(NC(=O)N2CCOC(C(C)(C)C)C2)cc1-c1cc(OCCO)nc(N2CCOCC2)c1] | (RS)-2-(tert-butyl)-N-(2-fluoro-5-(2- (2-hydroxyethoxy)-6- morpholinopyridin-4-yl)-4- methylphenyl)morpholine-4- carboxamide |
| 10 | 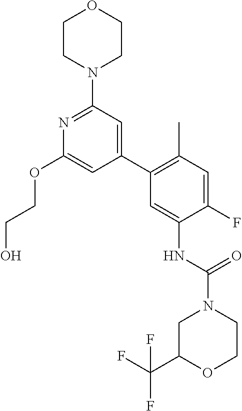 [SMILES: Cc1cc(F)c(NC(=O)N2CCOC(C(F)(F)F)C2)cc1-c1cc(OCCO)nc(N2CCOCC2)c1] | (RS)-N-(2-fluoro-5-(2-(2- hydroxyethoxy)-6-morpholinopyridin- 4-yl)-4-methylphenyl)-2- (trifluoromethyl)morpholine-4- carboxamide |
| 11 | 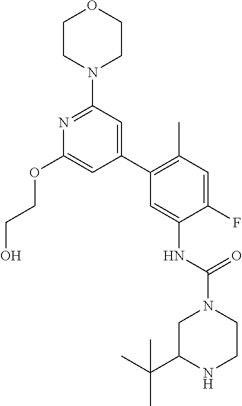 [SMILES: Cc1cc(F)c(NC(=O)N2CCNC(C(C)(C)C)C2)cc1-c1cc(OCCO)nc(N2CCOCC2)c1] | (RS)-3-(tert-butyl)-N-(2-fluoro-5-(2- (2-hydroxyethoxy)-6- morpholinopyridin-4-yl)-4- methylphenyl)piperazine-1- carboxamide |
| 12 and 13 | 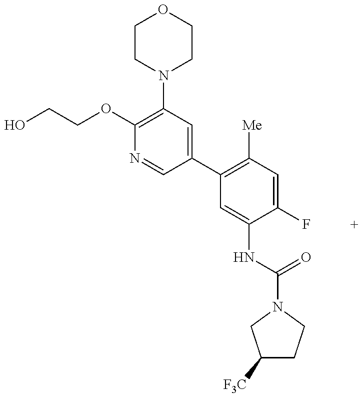 [SMILES: Cc1cc(F)c(NC(=O)N2CC[C@@H](C(F)(F)F)C2)cc1-c1cnc(OCCO)c(N2CCOCC2)c1] | (3R)-N-([2-fluoro-5-[6-(2- hydroxyethoxy)-5-(morpholin-4- yl)pyridin-3-yl]-4-methylphenyl]-3- (trifluoromethyl)pyrrolidine-1- carboxamide and (3S)-N-[2-fluoro-5-[6-(2- hydroxyethoxy)-5-(morpholin-4- yl)pyridin-3-yl]-4-methylphenyl]-3- (trifluoromethyl)pyrrolidine-1- carboxamide |
| | 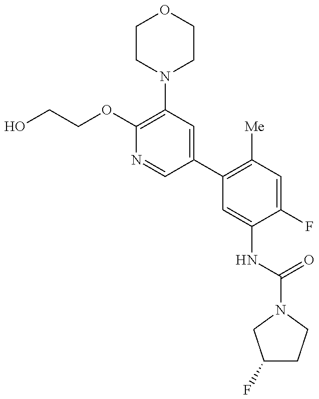 [SMILES: Cc1cc(F)c(NC(=O)N2CC[C@H](F)C2)cc1-c1cnc(OCCO)c(N2CCOCC2)c1] | |
| 14 and 15 | 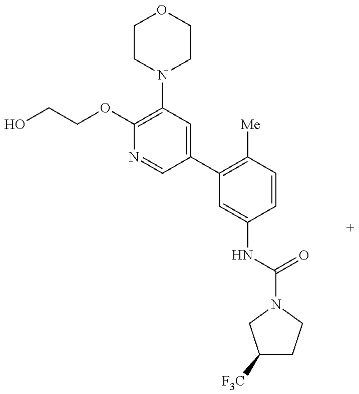 [SMILES: Cc1ccc(NC(=O)N2CC[C@@H](C(F)(F)F)C2)cc1-c1cnc(OCCO)c(N2CCOCC2)c1] | (3R)-N-[3-[6-(2-hydroxyethoxy)-5- (morpholin-4-yl)pyridin-3-yl]-4- methylphenyl]-3- (trifluoromethyl)pyrrolidine-1- carboxamide and (3S)-N-[3-[6-(2-hydroxyethoxy)-5- (morpholin-4-yl)pyridin-3-yl]-4- methylphenyl]-3- (trifluoromethyl)pyrrolidine-1- carboxamide |
| | 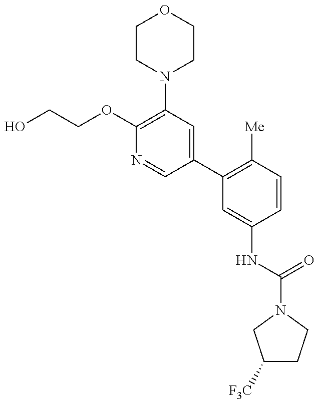 [SMILES: Cc1ccc(NC(=O)N2CC[C@H](C(F)(F)F)C2)cc1-c1cnc(OCCO)c(N2CCOCC2)c1] | |
| 16 and 17 | 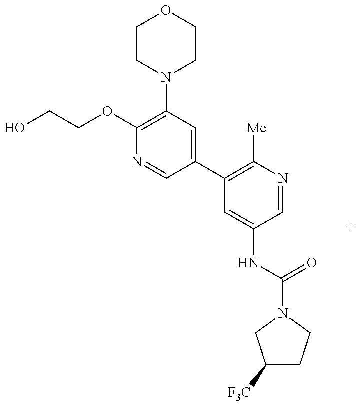 [SMILES: Cc1ncc(NC(=O)N2CC[C@@H](C(F)(F)F)C2)cc1-c1cnc(OCCO)c(N2CCOCC2)c1] | (3R)-N-[6′-(2-hydroxyethoxy)-2- methyl-5′-(morpholin-4-yl)-[3,3′- bipyridin]-5-yl]-3- (trifluoromethyl)pyrrolidine-1- carboxamide and (3S)-N-[6′-(2-hydroxyethoxy)-2- methyl-5′-(morpholin-4-yl)-[3,3′- bipyridin]-5-yl]-3- (trifluoromethyl)pyrrolidine-1- carboxamide |
| | 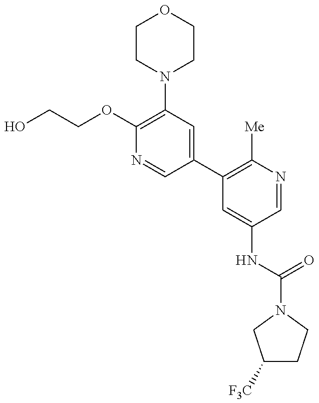 [SMILES: Cc1ncc(NC(=O)N2CC[C@H](C(F)(F)F)C2)cc1-c1cnc(OCCO)c(N2CCOCC2)c1] | |
| 18 | 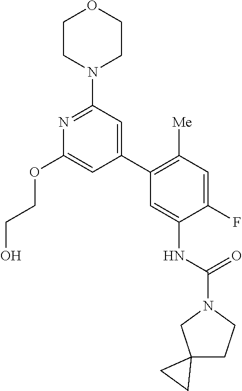 [SMILES: Cc1cc(F)c(NC(=O)N2CCC3(CC3)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-5-azaspiro[2.4]heptane- 5-carboxamide |
| 19 | 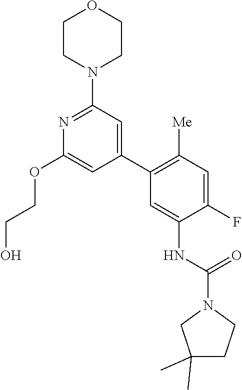 [SMILES: Cc1cc(F)c(NC(=O)N2CCC(C)(C)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3,3- dimethylpyrrolidine-1-carboxamide |
| 20 | 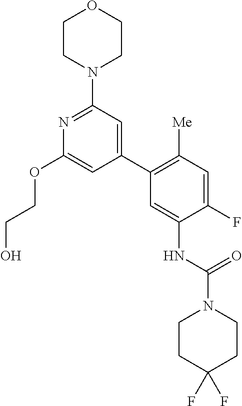 [SMILES: Cc1cc(F)c(NC(=O)N2CCC(F)(F)CC2)cc1-c1cc(OCCO)nc(N2CCOCC2)c1] | 4,4-difluoro-N-[2-fluoro-5-[2-(2- hydroxyethoxy)-6-(morpholin-4- yl)pyridin-4-yl]-4- methylphenyl]piperidine-1- carboxamide |
| 21 | 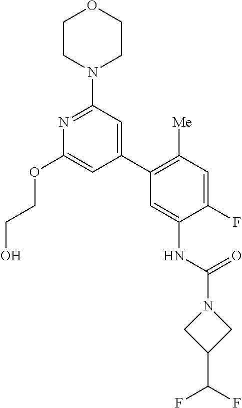 [SMILES: Cc1cc(F)c(NC(=O)N2CC(C(F)F)C2)cc1-c1cc(OCCO)nc(N2CCOCC2)c1] | 3-(difluoromethyl)-N-[2-fluoro-5-[2- (2-hydroxyethoxy)-6-(morpholin-4- yl)pyridin-4-yl]-4- methylphenyl]azetidine-1- carboxamide |
| 22 | 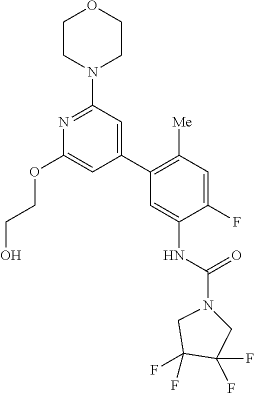 [SMILES: Cc1cc(F)c(NC(=O)N2CC(F)(F)C(F)(F)C2)cc1-c1cc(OCCO)nc(N2CCOCC2)c1] | 3,3,4,4-tetrafluoro-N-[2-fluoro-5-[2- (2-hydroxyethoxy)-6-(morpholin-4- yl)pyridin-4-yl]-4- methylphenyl]pyrrolidine-1- carboxamide |
| 23 | 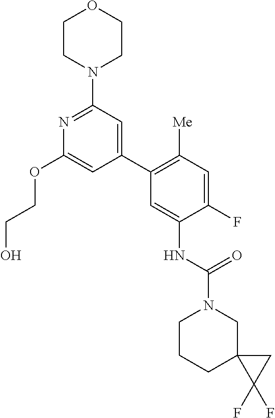 [SMILES: Cc1cc(F)c(NC(=O)N2CCCC3(C2)CC3(F)F)cc1-c1cc(OCCO)nc(N2CCOCC2)c1] | 3,3,4,4-tetrafluoro-N-[2-fluoro-5-[2- (2-hydroxyethoxy)-6-(morpholin-4- yl)pyridin-4-yl]-4- methylphenyl]pyrrolidine-1- carboxamide |
| 24 | 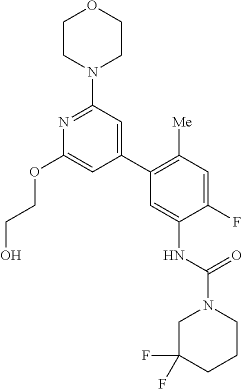 [SMILES: Cc1cc(F)c(NC(=O)N2CCCC(F)(F)C2)cc1-c1cc(OCCO)nc(N2CCOCC2)c1] | 3,3-difluroo-N-(2-fluoro-5-(2-(2- hydroxyethoxy)-6-morpholinopyridin- 4-yl)-4-methylphenyl)piperidine-1- carboxamide |
| 25 | 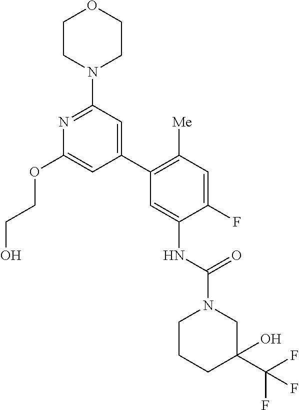 [SMILES: Cc1cc(F)c(NC(=O)N2CCCC(O)(C(F)(F)F)C2)cc1-c1cc(OCCO)nc(N2CCOCC2)c1] | N-(2-fluoro-5-(2-(2-hydroxyethoxy)-6- morpholinopyridin-4-yl)-4- methylphenyl)-3-hydroxy-3- (trifluoromethyl)piperidine-1- carboxamide |
| 26 | 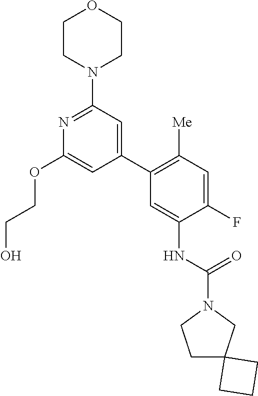 [SMILES: Cc1cc(F)c(NC(=O)N2CCC3(CCC3)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-6-azaspiro[3.4]octane- 6-carboxamide |
| 27 | 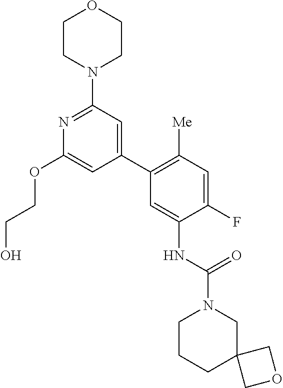 [SMILES: Cc1cc(F)c(NC(=O)N2CCCC3(COC3)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-2-oxa-6- azaspiro[3.5]nonane-6-carboxamide |
| 28 | 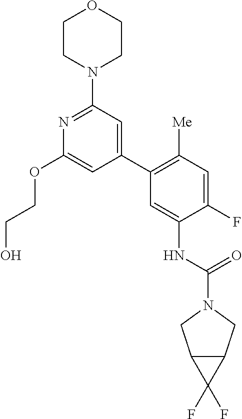 [SMILES: Cc1cc(F)c(NC(=O)N2CC3C(C2)C3(F)F)cc1-c1cc(OCCO)nc(N2CCOCC2)c1] | 6,6-difluoro-N-[2-fluoro-5-[2-(2- hydroxyethoxy)-6-(morpholin-4- yl)pyridin-4-yl]-4-mehtylphenyl]-3- azabicyclo[3.1.0]hexane-3- carboxamide |
| 29 and 30 | 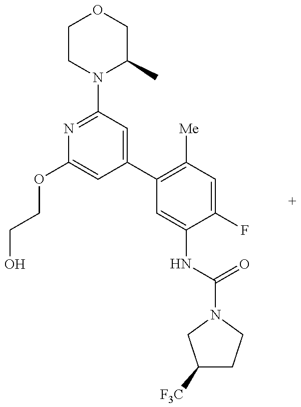 [SMILES: Cc1cc(F)c(NC(=O)N2CC[C@@H](C(F)(F)F)C2)cc1-c1cc(OCCO)nc(N2CCOC[C@H]2C)c1] | (3R)-N-[2-fluoro-5-[2-(2- hydroxyethoxy)-6-[(3R)-3- methylmorpholin-4-yl]pyridin-4-yl]-4- methylphenyl]-3-(trifluoromethyl) pyrrolidine-1-carboxamide and (3S)-N-[2-fluoro-5-[2-(2- hydroxyethoxy)-6-[(3R)-3- methylmorpholin-4-yl]pyridin-4-yl]-4- methylphenyl]-3- (trifluoromethyl)pyrrolidine-1- carboxamide |
| | 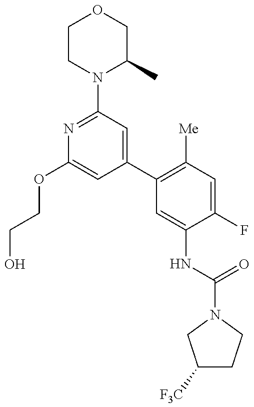 [SMILES: Cc1cc(F)c(NC(=O)N2CC[C@H](C(F)(F)F)C2)cc1-c1cc(OCCO)nc(N2CCOC[C@H]2C)c1] | |
| 31 and 32 | 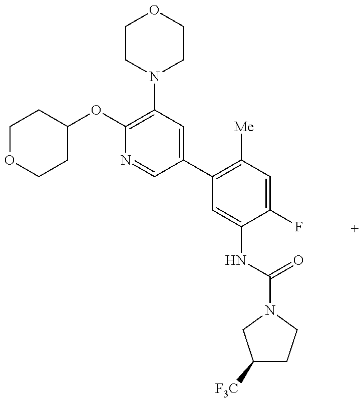 [SMILES: Cc1cc(F)c(NC(=O)N2CC[C@@H](C(F)(F)F)C2)cc1-c1cnc(OC2CCOCC2)c(N2CCOCC2)c1] | (3R)-N-[2-fluoro-4-methyl-5-[5- (morpholin-4-yl)-6-(oxan-4- yloxy)pyridin-3-yl]phenyl]-3- (trifluoromethyl)pyrrolidine-1- carboxamide and (3S)-N-[2-fluoro-4-methyl-5-[5- (morpholin-4-yl)-6-(oxan-4- yloxy)pyridin-3-yl]phenyl]-3- (trifluoromethyl)pyrrolidine-1- carboxamide |
| | 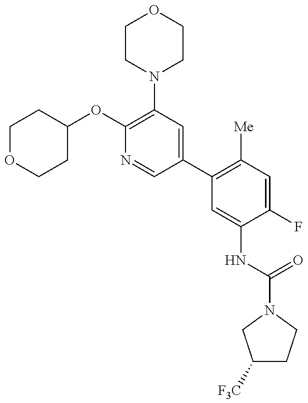 [SMILES: Cc1cc(F)c(NC(=O)N2CC[C@H](C(F)(F)F)C2)cc1-c1cnc(OC2CCOCC2)c(N2CCOCC2)c1] | |
| 33 and 34 | 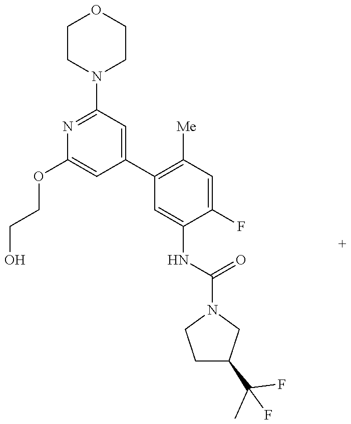 [SMILES: Cc1cc(F)c(NC(=O)N2CC[C@H](C(C)(F)F)C2)cc1-c1cc(OCCO)nc(N2CCOCC2)c1] | (3R)-3-(1,1-difluoroethyl)-N-[2-fluoro- 5-[2-(2-hydroxyethoxy)-6-(morpholin- 4-yl)pyridin-4-yl]-4- methylphenyl]pyrrolidine-1- carboxamide and (3S)-3-(1,1-difluoroethyl)-N-[2-fluoro- 5-[2-(2-hydroxyethoxy)-6-(morpholin- 4-yl)pyridin-4-yl]-4- methylphenyl]pyrrolidine-1- carboxamide |
| | 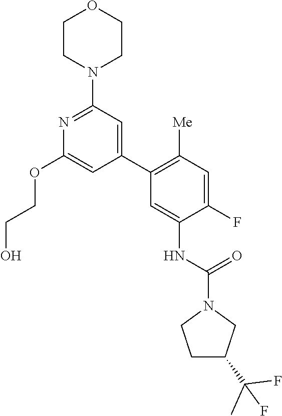 [SMILES: Cc1cc(F)c(NC(=O)N2CC[C@@H](C(C)(F)F)C2)cc1-c1cc(OCCO)nc(N2CCOCC2)c1] | |
| 35 | 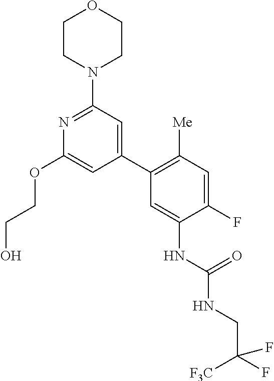 [SMILES: Cc1cc(F)c(NC(=O)NCC(F)(F)C(F)(F)F)cc1-c1cc(OCCO)nc(N2CCOCC2)c1] | 1-[2-fluoro-5-[2-(2-hydroxyethoxy)-6- (morpholin-4-yl)pyridin-4-yl]-4- methylphenyl]-3-(2,2,3,3,3- pentafluoropropyl)urea |
| 36 and 37 | 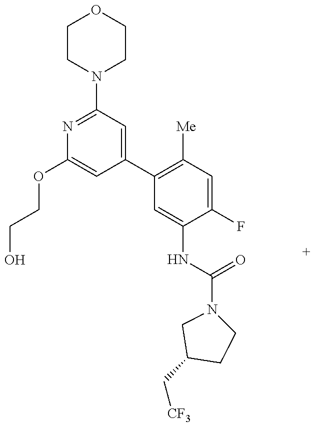 [SMILES: Cc1cc(F)c(NC(=O)N2CC[C@H](CC(F)(F)F)C2)cc1-c1cc(OCCO)nc(N2CCOCC2)c1] | (3R)-N-[2-fluoro-5-[2-(2- hydroxyethoxy)-6-(morpholin-4- yl)pyridin-4-yl]-4-methylphenyl]-3- (2,2,2-trifluoroethyl)pyrrolidine-1- carboxamide and (3R)-N-[2-fluoro-5-[2-(2- hydroxyethoxy)-6-(morpholin-4- yl)pyridin-4-yl]-4-methylphenyl]-3- (2,2,2-trifluoroethyl)pyrrolidine-1- carboxamide |
| | 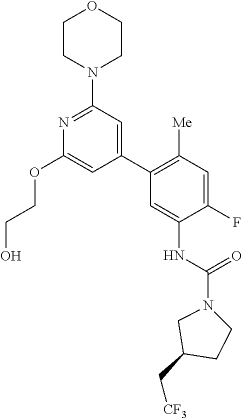 [SMILES: Cc1cc(F)c(NC(=O)N2CC[C@@H](CC(F)(F)F)C2)cc1-c1cc(OCCO)nc(N2CCOCC2)c1] | |
| 38 | 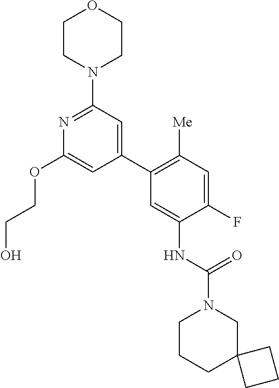 [SMILES: Cc1cc(F)c(NC(=O)N2CCCC3(CCC3)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-6-azaspiro[3.5]nonane- 6-carboxamide |
| 39 | 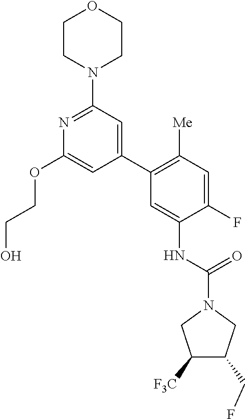 [SMILES: Cc1cc(F)c(NC(=O)N2C[C@H](CF)[C@@H](C(F)(F)F)C2)cc1-c1cc(OCCO)nc(N2CCOCC2)c1] | (trans)-N-[2-fluoro-5-[2-(2- hydroxyethoxy)-6-(morpholin-4- yl)pyridin-4-yl]-4-methylphenyl]-3- (fluoromethyl)-4-(trifluoromethyl) pyrrolidine-1-carboxamide |
| 40 | 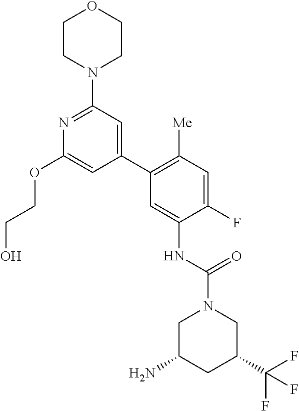 [SMILES: Cc1cc(F)c(NC(=O)N2C[C@@H](N)C[C@@H](C(F)(F)F)C2)cc1-c1cc(OCCO)nc(N2CCOCC2)c1] | (3S,5R)-3-amino-N-[2-fluoro-5-[2-(2- hydroxyethoxy)-6-(morpholin-4- yl)pyridin-4-yl]-4-methylphenyl]-5- (trifluoromethyl)piperidine-1- carboxamide |
| 41 | 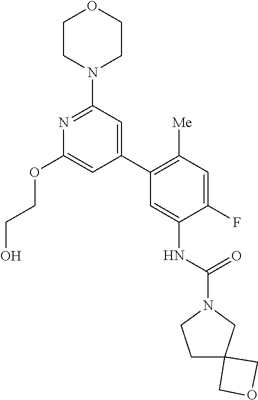 [SMILES: Cc1cc(F)c(NC(=O)N2CCC3(COC3)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-2-oxa-6- azaspiro[3.4]octane-6-carboxamide |
| 42 | 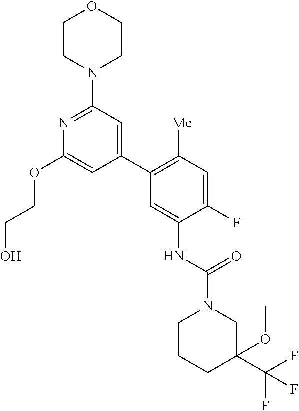 [SMILES: COC1(C(F)(F)F)CCCN(C(=O)Nc2cc(-c3cc(OCCO)nc(N4CCOCC4)c3)c(C)cc2F)C1] | N-[2-fluoro-5-[2-(2-hydroxyethoxy)-6- (morpholin-4-yl)pyridin-4-yl]-4- methylphenyl]-3-methoxy-3- (trifluoromethyl)piperidine-1- carboxamide |
| 43 | 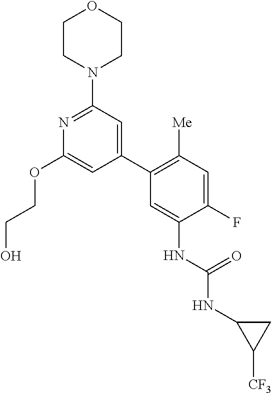 [SMILES: Cc1cc(F)c(NC(=O)NC2CC2C(F)(F)F)cc1-c1cc(OCCO)nc(N2CCOCC2)c1] | 1-[2-fluoro-5-[2-(2-hydroxyethoxy)-6- (morpholin-4-yl)pyridin-4-yl]-4- methylphenyl]-3-[2-(trifluoromethyl) cyclopropyl]urea |
| 44 | 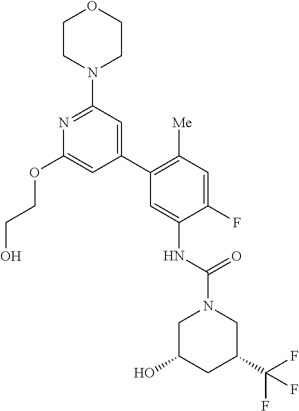 [SMILES: Cc1cc(F)c(NC(=O)N2C[C@@H](O)C[C@@H](C(F)(F)F)C2)cc1-c1cc(OCCO)nc(N2CCOCC2)c1] | (3S,5R)-N-[2-fluoro-5-[2-(2- hydroxyethoxy)-6-(morpholin-4- yl)pyridin-4-yl]-4-methylphenyl]-3- hydroxy-5-(trifluoromethyl) piperidine-1-carboxamide |
| 45 | 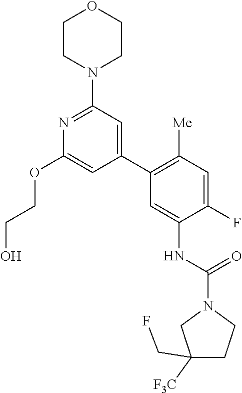 [SMILES: Cc1cc(F)c(NC(=O)N2CCC(CF)(C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(fluoromethyl)-3- (trifluoromethyl)pyrrolidine-1- carboxamide |
| 46 | 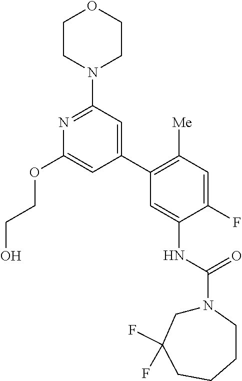 [SMILES: Cc1cc(F)c(NC(=O)N2CCCCC(F)(F)C2)cc1-c1cc(OCCO)nc(N2CCOCC2)c1] | 3,3-difluoro-N-[2-fluoro-5-[2-(2- hydroxyethoxy)-6-(morpholin-4- yl)pyridin-4-yl]-4- methylphenyl]azepane-1-carboxamide |
| 47 and 48 | 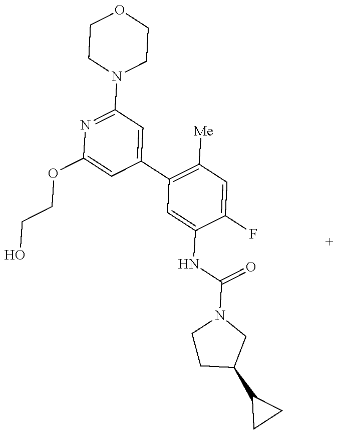 [SMILES: Cc1cc(F)c(NC(=O)N2CC[C@H](C3CC3)C2)cc1-c1cc(OCCO)nc(N2CCOCC2)c1] | (3R)-3-cyclopropyl-N-[2-fluoro-5-[2- (2-hydroxyethoxy)-6-(morpholin-4- yl)pyridin-4-yl]-4- methylphenyl]pyrrolidine-1- carboxamide and (3S)-3-cyclopropyl-N-[2-fluoro-5-[2- (2-hydroxyethoxy)-6-(morpholin-4- yl)pyridin-4-yl]-4- methylphenyl]pyrrolidine-1- carboxamide |
| | 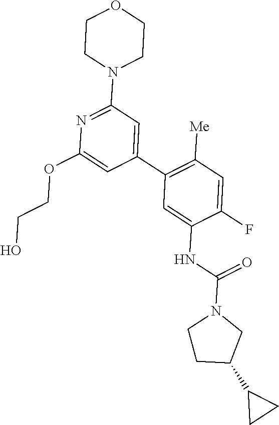 [SMILES: Cc1cc(F)c(NC(=O)N2CC[C@@H](C3CC3)C2)cc1-c1cc(OCCO)nc(N2CCOCC2)c1] | |
| 49 | 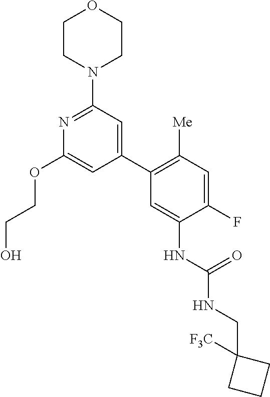 [SMILES: Cc1cc(F)c(NC(=O)NCC2(C(F)(F)F)CCC2)cc1-c1cc(OCCO)nc(N2CCOCC2)c1] | 1-[2-fluoro-5-[2-(2-hydroxyethoxy)-6- (morpholin-4-yl)pyridin-4-yl]-4- methylphenyl]-3-[[1-(trifluoromethyl) cyclobutyl]methyl]urea |
| 50 and 51 | 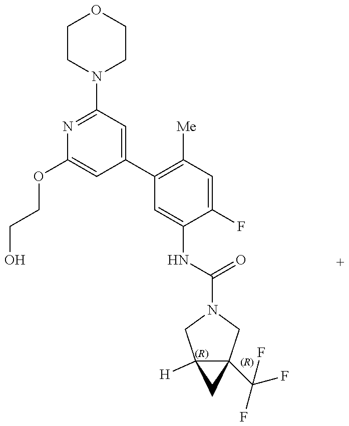 | (1R,5R)-N-(2-fluoro-5-(2-(2- hydroxyethoxy)-6-morpholinopyridin- 4-yl)-4-methylphenyl)-1- (trifluoromethyl)-3- azabicyclo[3.1.0]hexane-3- carboxamide and (1S,5S)-N-(2-fluoro-5-(2-(2- hydroxyethoxy)-6-morpholinopyrridin- 4-yl)-4-methylphenyl)-1- (trifluoromethyl)-3- azabicyclo[3.1.0]hexane-3- carboxamide |
| | 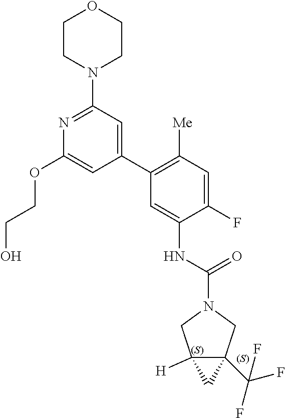 | |
| 52 | 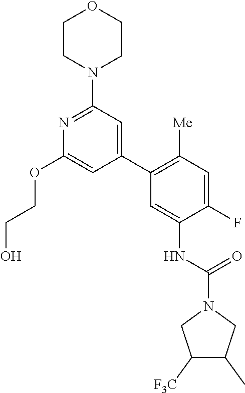 [SMILES: Cc1cc(F)c(NC(=O)N2CC(C)C(C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-methyl-4- (trifluoromethyl)pyrrolidine-1- carboxamide |
| 53 | 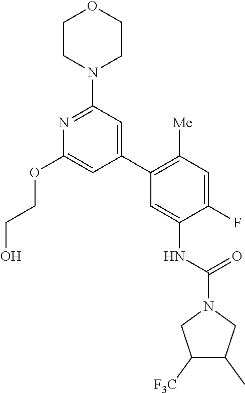 [SMILES: Cc1cc(F)c(NC(=O)N2CC(C)C(C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-methyl-4- (trifluoromethyl)pyrrolidine-1- carboxamide |
| 54 | 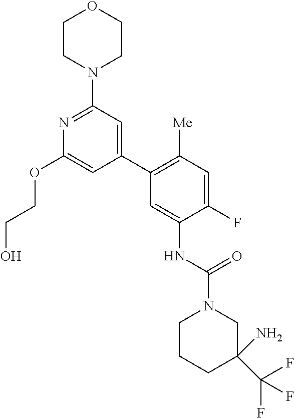 [SMILES: Cc1cc(F)c(NC(=O)N2CCCC(N)(C(F)(F)F)C2)cc1-c1cc(OCCO)nc(N2CCOCC2)c1] | 3-amino-N-[2-fluoro-5-[2-(2- hydroxyethoxy)-6-(morpholin-4- yl)pyridin-4-yl]-4-methylphenyl]-3- (trifluoromethyl)piperiidne-1- carboxamide |
| 55 | 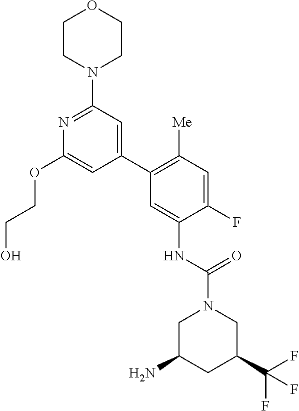 [SMILES: Cc1cc(F)c(NC(=O)N2C[C@H](N)C[C@H](C(F)(F)F)C2)cc1-c1cc(OCCO)nc(N2CCOCC2)c1] | (3R,5S)-3-amino-N-[2-fluoro-5-[2-(2- hydroxyethoxy)-6-(morpholin-4- yl)pyridin-4-yl]-4-methylphenyl]-5- (trifluoromethyl)piperidine-1- carboxamide |
| 56 | 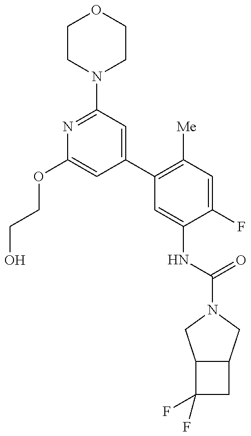 [SMILES: Cc1cc(F)c(NC(=O)N2CC3CC(F)(F)C3C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(fluoromethyl)-3- (trifluoromethyl)pyrrolidine-1- carboxamide |
| 57 | 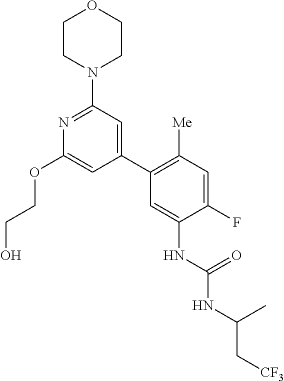 [SMILES: Cc1cc(F)c(NC(=O)NC(C)CC(F)(F)F)cc1-c1cc(OCCO)nc(N2CCOCC2)c1] | 1-[2-fluoro-5-[2-(2-hydroxyethoxy)-6- (morpholin-4-yl)pyridin-4-yl]-4- methylphenyl]-3-(4,4,4-trifluorobutan- 2-yl)urea |
| 58 and 59 | 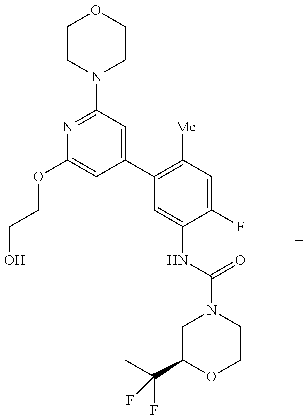 [SMILES: Cc1cc(F)c(NC(=O)N2CCO[C@@H](C(C)(F)F)C2)cc1-c1cc(OCCO)nc(N2CCOCC2)c1] | (2R)-2-(1,1-difluoroethyl)-N-[2-fluoro- 5-[2-(2-hydroxyethoxy)-6-(morpholin- 4-yl)pyridin-4-yl]-4- methylphenyl]morpholine-4- carboxamide and (2R)-2-(1,1-difluoroethyl)-N-[2-fluoro- 5-[2-(2-hydroxyethoxy)-6-(morpholin- 4-yl)pyridin-4-yl]-4- methylphenyl]morpholine-4- carboxamide |
| | 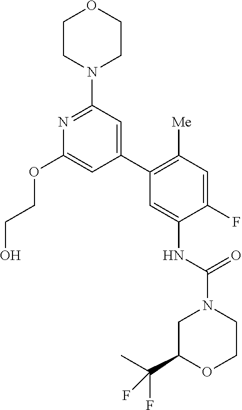 [SMILES: Cc1cc(F)c(NC(=O)N2CCO[C@@H](C(C)(F)F)C2)cc1-c1cc(OCCO)nc(N2CCOCC2)c1] | |
| 60 | 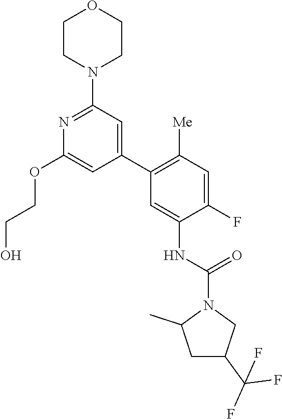 [SMILES: Cc1cc(F)c(NC(=O)N2CC(C(F)(F)F)CC2C)cc1-c1cc(OCCO)nc(N2CCOCC2)c1] | (cis)-N-[2-fluoro-5-[2-(2- hydroxyethoxy)-6-(morpholin-4- yl)pyridin-4-yl]-4-methylphenyl]-2- methyl-4-(trifluoromethyl)pyrrolidine- 1-carboxamide |
| 61 | 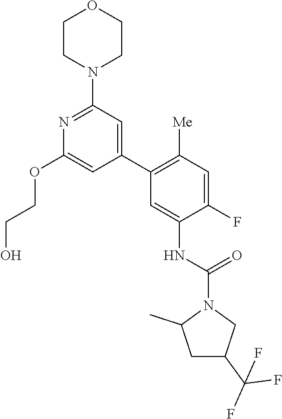 [SMILES: Cc1cc(F)c(NC(=O)N2CC(C(F)(F)F)CC2C)cc1-c1cc(OCCO)nc(N2CCOCC2)c1] | (cis)-N-[2-fluoro-5-[2-(2- hydroxyethoxy)-6-(morpholin-4- yl)pyridin-4-yl]-4-methylphenyl]-2- methyl-4-(trifluoromethyl)pyrrolidine- 1-carboxamide |
| 62 and 63 | 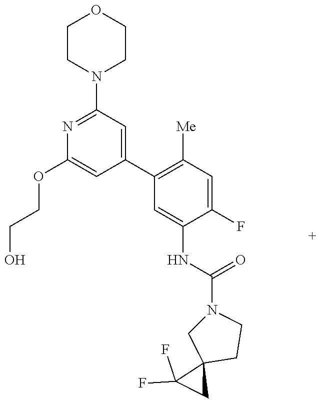 [SMILES: Cc1cc(F)c(NC(=O)N2CC[C@]3(C2)CC3(F)F)cc1-c1cc(OCCO)nc(N2CCOCC2)c1] | (3R)-1,1-difluoro-N-[2-fluoro-5-[2-(2- hydroxyethoxy)-6-(morpholin-4- yl)pyridin-4-yl]-4-methylphenyl]-5- azaspiro[2.4]heptane-5-carboxamide and (3S)-1,1-difluoro-N-[2-fluoro-5-[2-(2- hydroxyethoxy)-6-(morpholin-4- yl)pyridin-4-yl]-4-methylphenyl]-5- azaspiro[2.4]heptane-5-carboxamide |
| | 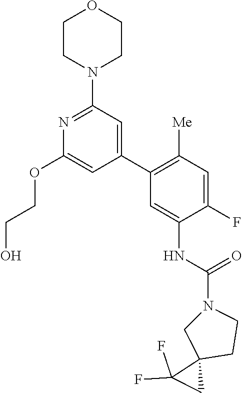 [SMILES: Cc1cc(F)c(NC(=O)N2CC[C@@]3(C2)CC3(F)F)cc1-c1cc(OCCO)nc(N2CCOCC2)c1] | |
| 64 | 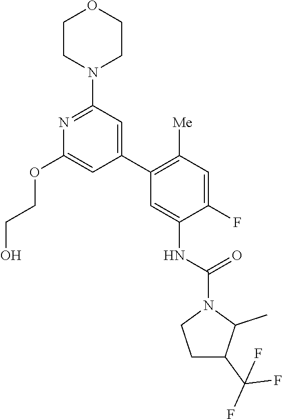 [SMILES: Cc1cc(F)c(NC(=O)N2CCC(C(F)(F)F)C2C)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-2-methyl-3- (trifluoromethyl)pyrrolidine-1- carboxamide |
| 65 | 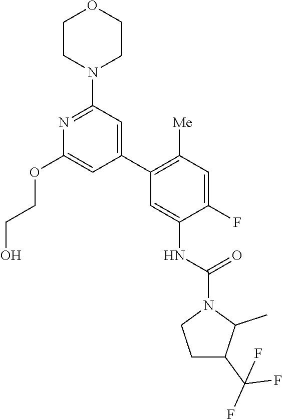 [SMILES: Cc1cc(F)c(NC(=O)N2CCC(C(F)(F)F)C2C)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-2-methyl-3- (trifluoromethyl)pyrrolidine-1- carboxamide |
| 66 | 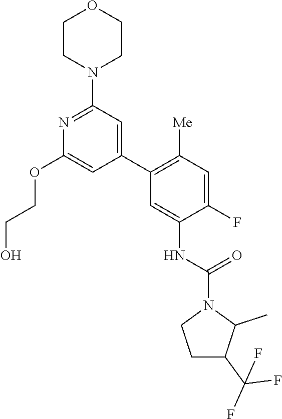 [SMILES: Cc1cc(F)c(NC(=O)N2CCC(C(F)(F)F)C2C)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-2-methyl-3- (trifluoromethyl)pyrrolidine-1- carboxamide |
| 67 | 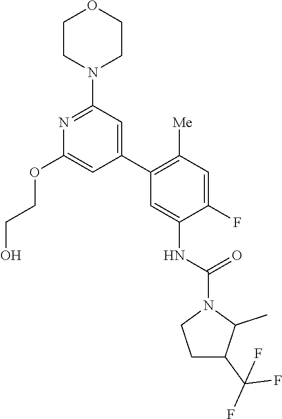 [SMILES: Cc1cc(F)c(NC(=O)N2CCC(C(F)(F)F)C2C)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-2-methyl-3- (trifluoromethyl)pyrrolidine-1- carboxamide |
| 68 | 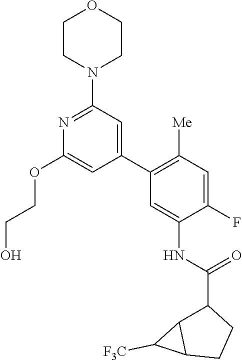 [SMILES: Cc1cc(F)c(NC(=O)C2CCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-6-(trifluoromethyl)-2- azabicyclo[3.1.0]hexane-2- carboxamide |
| 69 | 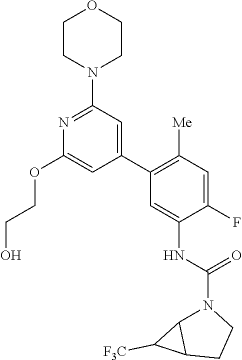 [SMILES: Cc1cc(F)c(NC(=O)N2CCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-6-(trifluoromethyl)-2- azabicyclo[3.1.0]hexane-2- carboxamide |
| 70 | 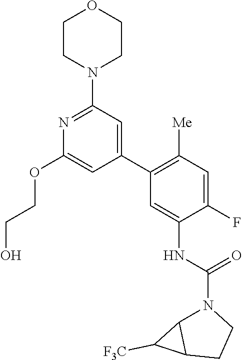 [SMILES: Cc1cc(F)c(NC(=O)N2CCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-6-(trifluoromethyl)-2- azabicyclo[3.1.0]hexane-2- carboxamide |
| 71 | 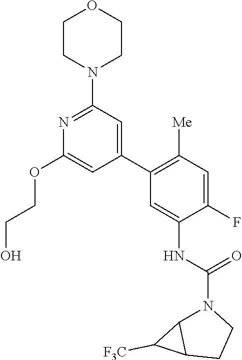 [SMILES: Cc1cc(F)c(NC(=O)N2CCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-6-(trifluoromethyl)-2- azabicyclo[3.1.0]hexane-2- carboxamide |
| 72 | 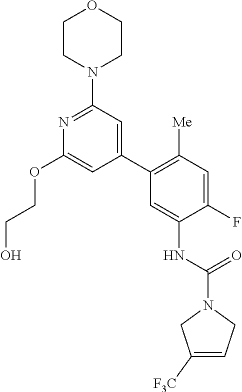 [SMILES: Cc1cc(F)c(NC(=O)N2CC=C(C(F)(F)F)C2)cc1-c1cc(OCCO)nc(N2CCOCC2)c1] | N-[2-fluoro-5-[2-(2-hydroxyethoxy)-6- (morpholin-4-yl)pyriidn-4-yl]-4- methylphenyl]-3-(trifluoromethyl)-2,5- dihydropyrrole-1-carboxamide |
| 73 | 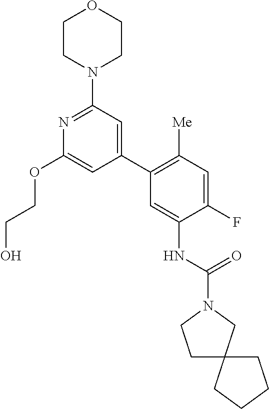 [SMILES: Cc1cc(F)c(NC(=O)N2CCC3(CCCC3)C2)cc1-c1cc(OCCO)nc(N2CCOCC2)c1] | N-(2-fluoro-5-(2-(2-hydroxyethoxy)-6- morpholinopyridin-4-yl)-4- methylphenyl)-2-azaspiro[4.4]nonane- 2-carboxamide |
| 74 and 75 | 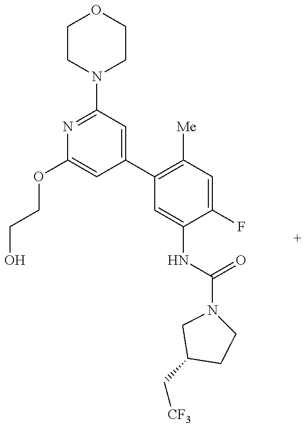 [SMILES: Cc1cc(F)c(NC(=O)N2CC[C@H](CC(F)(F)F)C2)cc1-c1cc(OCCO)nc(N2CCOCC2)c1] | (3R)-N-[2-fluoro-5-[2-(2- hydroxyethoxy)-6-(morpholin-4- yl)pyridin-4-yl]-4-methylphenyl]-3- (2,2,2-trifluoroethyl)pyrrolidine-1- carboxamide and (3S)-N-[2-fluoro-5-[2-(2- hydroxyethoxy)-6-(morpholin-4- yl)pyridin-4-yl]-4-methylphenyl]-3- (2,2,2-trifluoroethyl)pyrrolidine-1- carboxamide |
| | 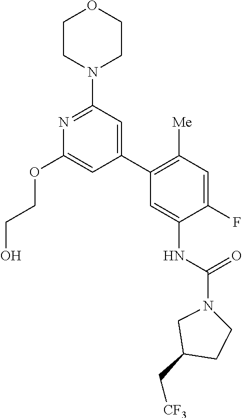 [SMILES: Cc1cc(F)c(NC(=O)N2CC[C@@H](CC(F)(F)F)C2)cc1-c1cc(OCCO)nc(N2CCOCC2)c1] | |
| 76 | 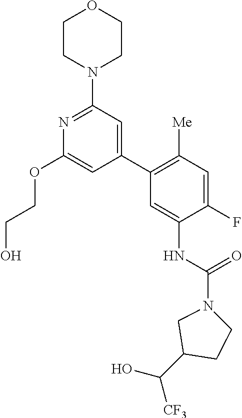 [SMILES: Cc1cc(F)c(NC(=O)N2CCC(C(O)C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(2,2,2-trifluoro-1- hydroxyethyl)pyrrolidine-1- carboxamide |
| 77 and 78 | 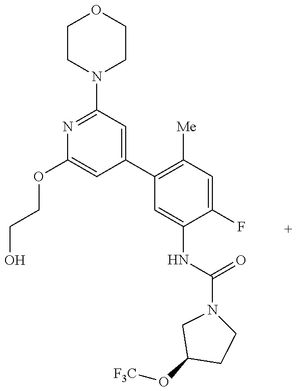 [SMILES: Cc1cc(F)c(NC(=O)N2CC[C@@H](OC(F)(F)F)C2)cc1-c1cc(OCCO)nc(N2CCOCC2)c1] | (3R)-N-[2-fluoro-5-[2-(2- hydroxyethoxy)-6-(morpholin-4- yl)pyridin-4-yl]-4-methylphenyl]-3- (trifluoromethoxy)pyrrolidine-1- carboxamide and (3S)-N-[2-fluoro-5-[2-(2- hydroxyethoxy)-6-(morpholin-4- yl)pyridin-4-yl]-4-methylphenyl]-3- (trifluoromethoxy)pyrrolidine-1- carboxamide |
| | 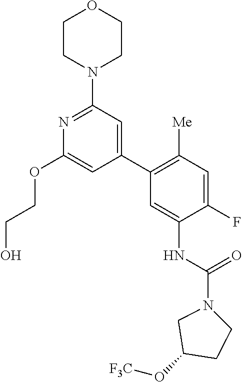 [SMILES: Cc1cc(F)c(NC(=O)N2CC[C@H](OC(F)(F)F)C2)cc1-c1cc(OCCO)nc(N2CCOCC2)c1] | |
| 79 | 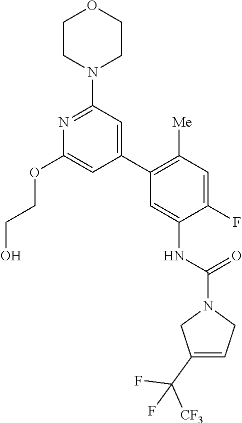 [SMILES: Cc1cc(F)c(NC(=O)N2CC=C(C(F)(F)C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(1,1,2,2,2- pentafluoroethyl)-2,5-dihydropyrrole- 1-carboxamide |
| 80 | 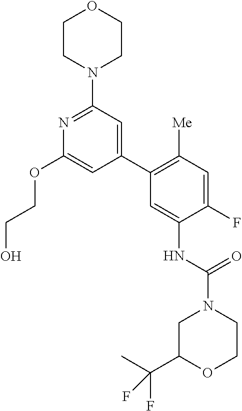 [SMILES: Cc1cc(F)c(NC(=O)N2CCOC(C(C)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(trifluoromethyl)-5,6- dihydro-2H-pyridine-1-carboxamide |
| 81 | 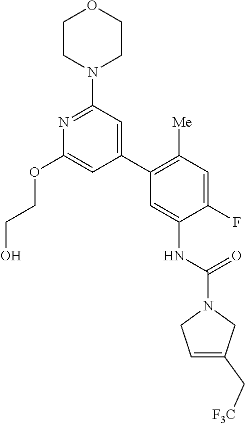 [SMILES: Cc1cc(F)c(NC(=O)N2CC=C(CC(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(2,2,2-trifluoroethyl)- 2,5-dihydropyrrole-1-carboxamide |
| 82 | 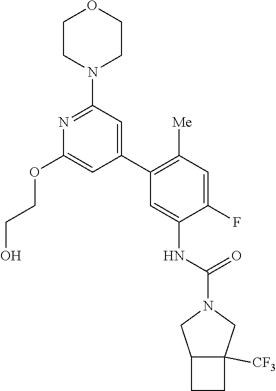 [SMILES: Cc1cc(F)c(NC(=O)N2CC3CCC3(C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-1-(trifluoromethyl)-3- azabicyclo[3.2.0]heptane-3- carboxamide |
| 83 | 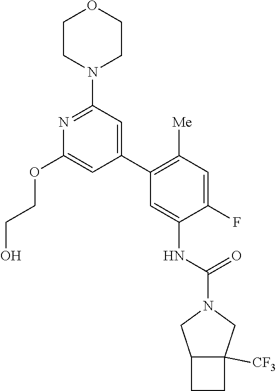 [SMILES: Cc1cc(F)c(NC(=O)N2CC3CCC3(C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-1-(trifluoromethyl)-3- azabicyclo[3.2.0]heptane-3- carboxamide |
| 84 and 85 | 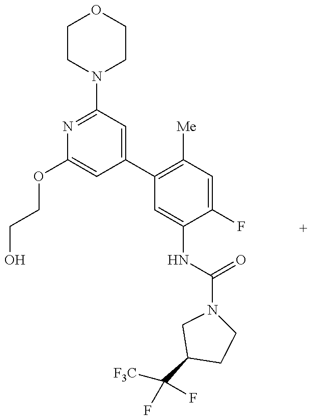 [SMILES: Cc1cc(F)c(NC(=O)N2CC[C@@H](C(F)(F)C(F)(F)F)C2)cc1-c1cc(OCCO)nc(N2CCOCC2)c1] | (3R)-N-[2-fluoro-5-[2-(2- hydroxyethoxy)-6-(morpholin-4- yl)pyridin-4-yl]-4-methylphenyl]-3- (1,1,2,2,2- pentafluoroethyl)pyrrolidine-1- carboxamide and (3S)-N-[2-fluoro-5-[2-(2- hydroxyethoxy)-6-(morpholin-4- yl)pyridin-4-yl]-4-methylphenyl]-3- (1,1,2,2,2- pentafluoroethyl)pyrrolidine-1- carboxamide |
| | 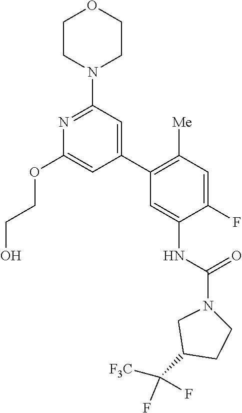 | |
| 86 | 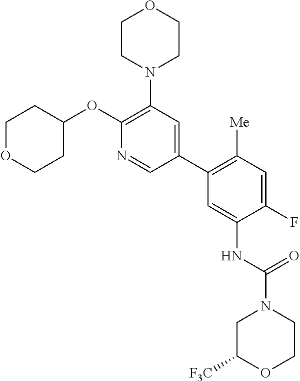 [SMILES: Cc1cc(F)c(NC(=O)N2CCO[C@H](C(F)(F)F)C2)cc1-c1cnc(OC2CCOCC2)c(N2CCOCC2)c1] | (2S)-N-[2-fluoro-4-methyl-5-[5- (morpholin-4-yl)-6-(morpholin-4- yloxy)pyridin-3-yl]phenyl]-2- (trifluoromethyl)morpholine-4- carboxamide |
| 87 | 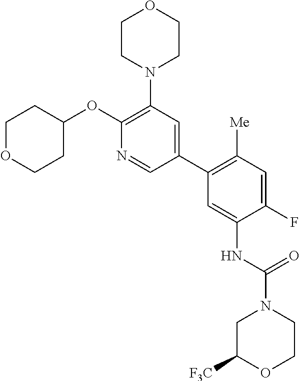 [SMILES: Cc1cc(F)c(NC(=O)N2CCO[C@@H](C(F)(F)F)C2)cc1-c1cnc(OC2CCOCC2)c(N2CCOCC2)c1] | (2R)-N-[2-fluoro-4-methyl-5-[5- (morpholin-4-yl)-6-(oxan-4- yloxy)pyridin-3-yl]phenyl]-2- (trifluoromethyl)morpholine-4- carboxamide |
| 88 | 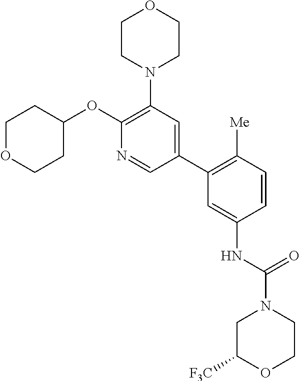 [SMILES: Cc1ccc(NC(=O)N2CCO[C@H](C(F)(F)F)C2)cc1-c1cnc(OC2CCOCC2)c(N2CCOCC2)c1] | (2S)-N-[4-methyl-3-[5-(morpholin-4- yl)-6-(morpholin-4-yloxy)pyridin-3- yl]phenyl]-2- (trifluoromethyl)morpholine-4- carboxamide |
| 89 | 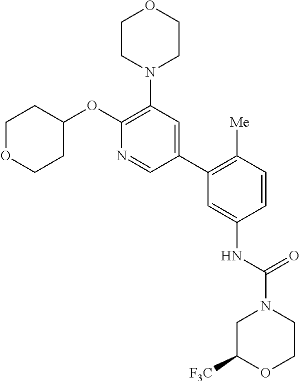 [SMILES: Cc1ccc(NC(=O)N2CCO[C@@H](C(F)(F)F)C2)cc1-c1cnc(OC2CCOCC2)c(N2CCOCC2)c1] | (2R)-N-[4-methyl-3-[5-(morpholin-4- yl)-6-(morpholin-4-yloxy)pyridin-3- yl]phenyl]-2- (trifluoromethyl)morpholine-4- carboxamide |
| 90 | 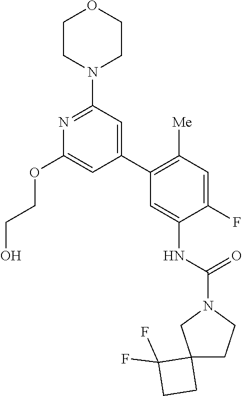 [SMILES: Cc1cc(F)c(NC(=O)N2CCC3(CCC3(F)F)C2)cc1-c1cc(OCCO)nc(N2CCOCC2)c1] | 1,1-difluoro-N-[2-fluoro-5-[2-(2- hydroxyethoxy)-6-(morpholin-4- yl)pyridin-4-yl]-4-methylphenyl]-6- azaspiro[3.4]octane-6-carboxamide |
| 91 | 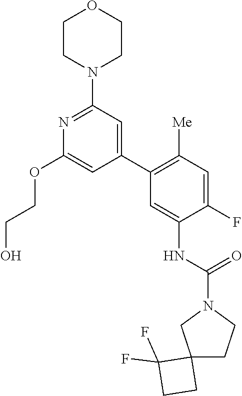 [SMILES: Cc1cc(F)c(NC(=O)N2CCC3(CCC3(F)F)C2)cc1-c1cc(OCCO)nc(N2CCOCC2)c1] | 1,1-difluoro-N-[2-fluoro-5-[2-(2- hydroxyethoxy)-6-(morpholin-4- yl)pyridin-4-yl]-4-methylphenyl]-6- azaspiro[3.4]octane-6-carboxamide |
| 92 | 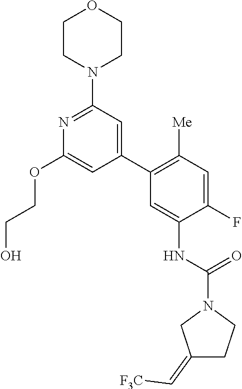 [SMILES: Cc1cc(F)c(NC(=O)N2CC/C(=C/C(F)(F)F)C2)cc1-c1cc(OCCO)nc(N2CCOCC2)c1] | (Z)-N-(2-fluoro-5-(2-(2- hydroxyethoxy)-6-morpholinopyridin- 4-yl)-4-methylphenyl)-3-(2,2,2- trifluoroethylidene)pyrrolidine-1- carboxamide |
| 93 | 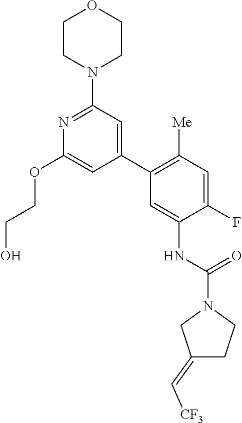 [SMILES: Cc1cc(F)c(NC(=O)N2CC/C(=C\C(F)(F)F)C2)cc1-c1cc(OCCO)nc(N2CCOCC2)c1] | (E)-N-(2-fluoro-5-(2-(2- hydroxyethoxy)-6-morpholinopyridin- 4-yl)-4-methylphenyl)-3-(2,2,2- trifluoroethylidene)pyrrolidine-1- carboxamide |
| 94 | 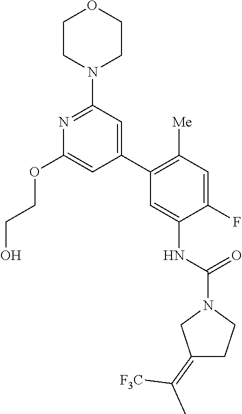 [SMILES: C/C(=C1\CCN(C(=O)Nc2cc(-c3cc(OCCO)nc(N4CCOCC4)c3)c(C)cc2F)C1)C(F)(F)F] | (3Z)-N-[2-fluoro-5-[2-(2- hydroxyethoxy)-6-(morpholin-4- yl)pyridin-4-yl]-4-methylphenyl]-3- (1,1,1-trifluoropropan-2- ylidene)pyrrolidine-1-carboxamide |
| 95 | 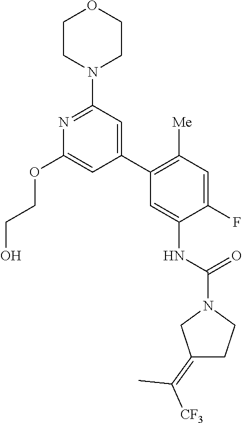 [SMILES: C/C(=C1/CCN(C(=O)Nc2cc(-c3cc(OCCO)nc(N4CCOCC4)c3)c(C)cc2F)C1)C(F)(F)F] | (3E)-N-[2-fluoro-5-[2-(2- hydroxyethoxy)-6-(morpholin-4- yl)pyridin-4-yl]-4-methylphenyl]-3- (1,1,1-trifluoropropan-2- ylidene)pyrrolidine-1-carboxamide |
| 96 | 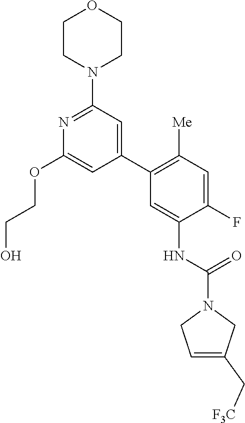 [SMILES: Cc1cc(F)c(NC(=O)N2CC=C(CC(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(2,2,2-trifluoroethyl)- 2,5-dihydropyrrole-1-carboxamide |
| 97 | 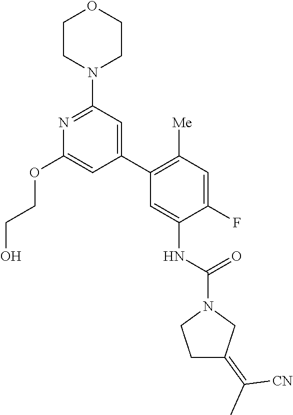 [SMILES: C/C(C#N)=C1\CCN(C(=O)Nc2cc(-c3cc(OCCO)nc(N4CCOCC4)c3)c(C)cc2F)C1] | (3E)-3-(1-cyanoethylidene)-N-[2- fluoro-5-[2-(2-hydroxyethoxy)-6- (morpholin-4-yl)pyridin-4-yl]-4- methylphenyl]pyrrolidine-1- carboxamide |
| 98 | 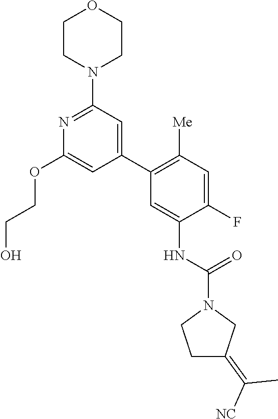 [SMILES: C/C(C#N)=C1/CCN(C(=O)Nc2cc(-c3cc(OCCO)nc(N4CCOCC4)c3)c(C)cc2F)C1] | (3E)-3-(1-cyanoethylidene)-N-[2- fluoro-5-[2-(2-hydroxyethoxy)-6- (morpholin-4-yl)pyridin-4-yl]-4- methylphenyl]pyrrolidine-1- carboxamide |
| 99 | 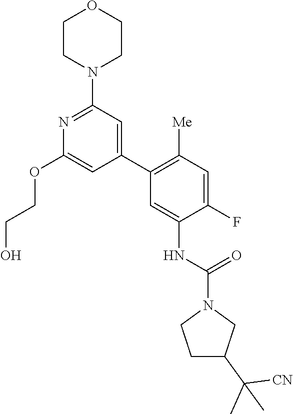 [SMILES: Cc1cc(F)c(NC(=O)N2CCC(C(C)(C)C#N)C2)cc1-c1cc(OCCO)nc(N2CCOCC2)c1] | 3-(1-cyano-1-methylethyl)-N-[2- fluoro-5-[2-(2-hydroxyethoxy)-6- (morpholin-4-yl)pyridin-4-yl]-4- methylphenyl]pyrrolidine-1- carboxamide |
| 100 | 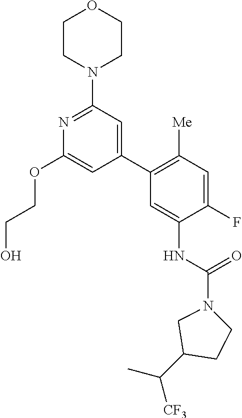 [SMILES: Cc1cc(F)c(NC(=O)N2CCC(C(C)C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(1,1,1- trifluoropropan-2-yl)pyrrolidine-1- carboxamide |
| 101 | 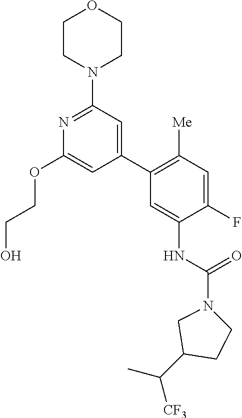 [SMILES: Cc1cc(F)c(NC(=O)N2CCC(C(C)C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(1,1,1- trifluoropropan-2-yl)pyrrolidine-1- carboxamide |
| 102 | 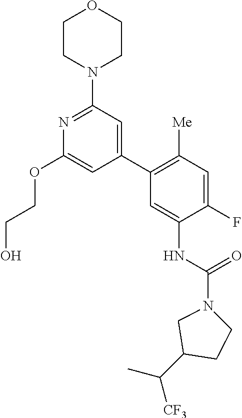 [SMILES: Cc1cc(F)c(NC(=O)N2CCC(C(C)C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(1,1,1- trifluoropropan-2-yl)pyrrolidine-1- carboxamide |
| 103 | 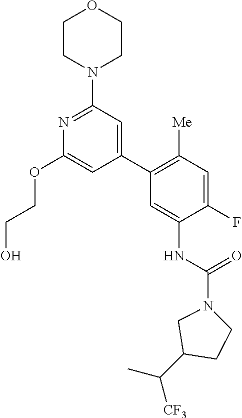 [SMILES: Cc1cc(F)c(NC(=O)N2CCC(C(C)C(F)(F)F)C2)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-3-(1,1,1- trifluoropropan-2-yl)pyrrolidine-1- carboxamide |
| 104 | 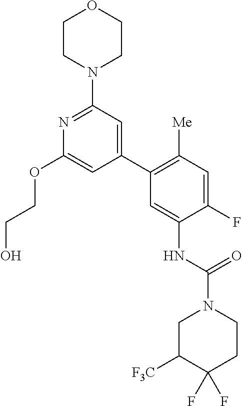 [SMILES: Cc1cc(F)c(NC(=O)N2CCC(F)(F)C(C(F)(F)F)C2)cc1-c1cc(OCCO)nc(N2CCOCC2)c1] | 4,4-difluoro-N-[2-fluoro-5-[2-(2- hydroxyethoxy)-6-(morpholin-4- yl)pyridin-4-yl]-4-methylphenyl]-3- (trifluoromethyl)piperidine-1- carboxamide |
| 105 and 106 | 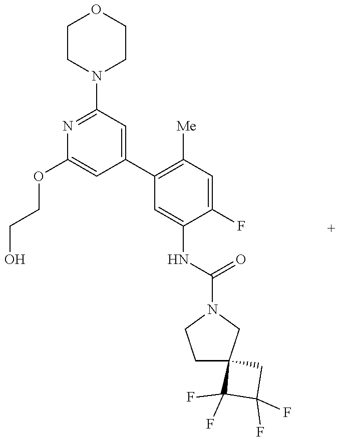 [SMILES: Cc1cc(F)c(NC(=O)N2CC[C@]3(C2)CC(F)(F)C3(F)F)cc1-c1cc(OCCO)nc(N2CCOCC2)c1] | (4R)-1,1,2,2-tetrafluoro-N-[2-fluoro-5- [2-(2-hydroxyethoxy)-6-(morpholin-4- yl)pyridin-4-yl]-4-methylphenyl]-6- azaspiro[3.4]octane-6-carboxamide and (4S)-1,1,2,2-tetrafluoro-N-[2-fluoro-5- [2-(2-hydroxyethoxy)-6-(morpholin-4- yl)pyridin-4-yl]-4-methylphenyl]-6- azaspiro[3.4]octane-6-carboxamide |
| | 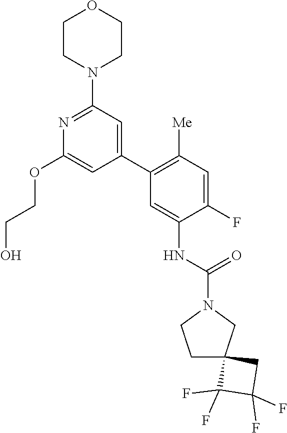 [SMILES: Cc1cc(F)c(NC(=O)N2CC[C@@]3(C2)CC(F)(F)C3(F)F)cc1-c1cc(OCCO)nc(N2CCOCC2)c1] | |
| 107 | 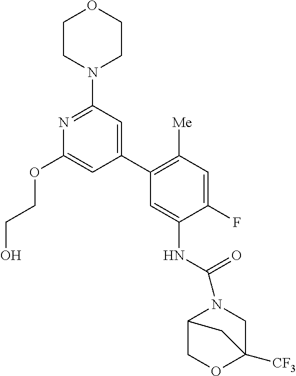 [SMILES: Cc1cc(F)c(NC(=O)N2CC3(C(F)(F)F)CC2CO3)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-1-(trifluoromethyl)-2- oxa-5-azabicyclo[2.2.1]heptane-5- carboxamide |
| 108 | 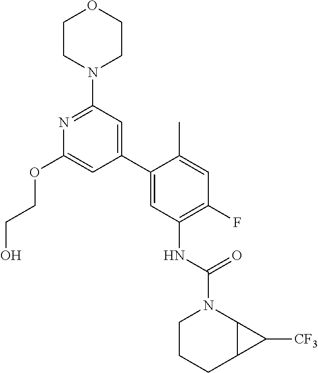 [SMILES: Cc1cc(F)c(NC(=O)N2CCCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-7-(trifluoromethyl)-2- azabicyclo[4.1.0]heptane-2- carboxamide |
| 109 | 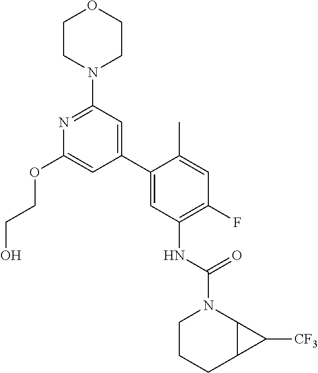 [SMILES: Cc1cc(F)c(NC(=O)N2CCCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-7-(trifluoromethyl)-2- azabicyclo[4.1.0]heptane-2- carboxamide |
| 110 | 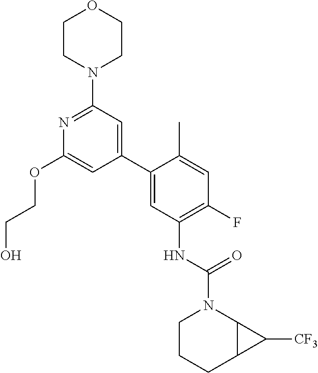 [SMILES: Cc1cc(F)c(NC(=O)N2CCCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-7-(trifluoromethyl)-2- azabicyclo[4.1.0]heptane-2- carboxamide |
| 111 | 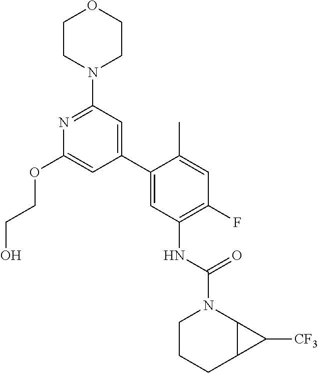 [SMILES: Cc1cc(F)c(NC(=O)N2CCCC3C2C3C(F)(F)F)cc1-c1cc(OCCO)nc(N2CCOCC2)c1] | N-[2-fluoro-5-[2-(2-hydroxyethoxy)-6- (morpholin-4-yl)pyridin-4-yl]-4- methylphenyl]-7-(trifluoromethyl)-2- aabicyclo[4.1.0]heptane-2- carboxamide |
| 112 | 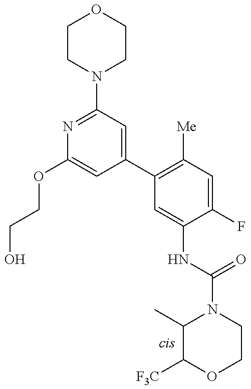 [SMILES: Cc1cc(F)c(NC(=O)N2CCOC(C(F)(F)F)C2C)cc1-c1cc(OCCO)nc(N2CCOCC2)c1] | (2R,3R)-N-(2-fluoro-5-(2-(2- hydroxyethoxy)-6-morpholinopyridin- 4-yl)-4-methylphenyl)-3-methyl-2- (trifluoromethyl)morpholine-4- carboxamide |
| 113 | 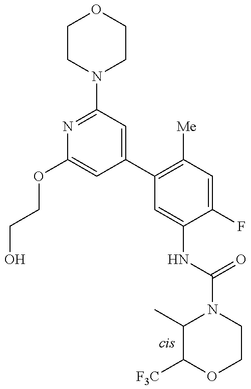 [SMILES: Cc1cc(F)c(NC(=O)N2CCOC(C(F)(F)F)C2C)cc1-c1cc(OCCO)nc(N2CCOCC2)c1] | (2S,3S)-N-(2-fluoro-5-(2-(2- hydroxyethoxy)-6-morpholinopyridin- 4-yl)-4-methylphenyl)-3-methyl-2- (trifluoromethyl)morpholine-4- carboxamide |
| 114 | 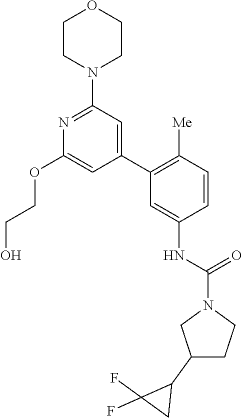 [SMILES: Cc1ccc(NC(=O)N2CCC(C3CC3(F)F)C2)cc1-c1cc(OCCO)nc(N2CCOCC2)c1] | 3-(2,2-difluorocyclopropyl)-N-[3-[2- (2-hydroxyethoxy)-6-(morpholin-4- yl)pyridin-4-yl]-4- methylphenyl]pyrrolidine-1- carboxamide |
| 115 and 116 | 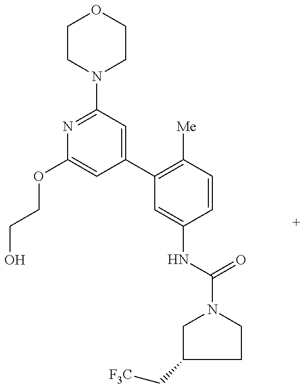 [SMILES: Cc1ccc(NC(=O)N2CC[C@H](CC(F)(F)F)C2)cc1-c1cc(OCCO)nc(N2CCOCC2)c1] | (3R)-N-[3-[2-(2-hydroxyethoxy)-6- (morpholin-4-yl)pyridin-4-yl]-4- methylphenyl]-3-(2,2,2- trifluoroethyl)pyrrolidine-1- carboxamide and (3S)-N-[3-[2-(2-hydroxyethoxy)-6- (morpholin-4-yl)pyridin-4-yl]-4- methylphenyl]-3-(2,2,2- trifluoroethyl)pyrrolidine-1- carboxamide |
| | 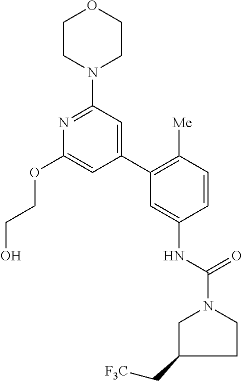 [SMILES: Cc1ccc(NC(=O)N2CC[C@@H](CC(F)(F)F)C2)cc1-c1cc(OCCO)nc(N2CCOCC2)c1] | |
| 117 and 118 | 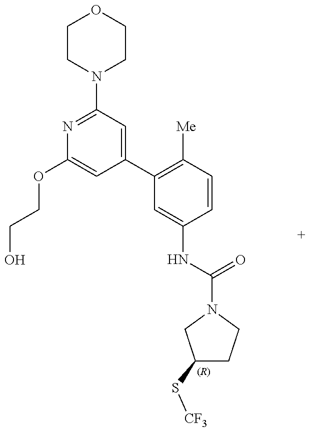 | (3R)-N-[3-[2-(2-hydroxyethoxy)-6- (morpholin-4-yl)pyridin-4-yl]-4- methylphenyl]-3- [(trifluoromethyl)sulfanyl]pyrrolidine- 1-carboxamide and (3S)-N-[3-[2-(2-hydroxyethoxy)-6- (morpholin-4-yl)pyridin-4-yl]-4- methylphenyl]-3- [(trifluoromethyl)sulfanyl]pyrrolidine- 1-carboxamide |
| | 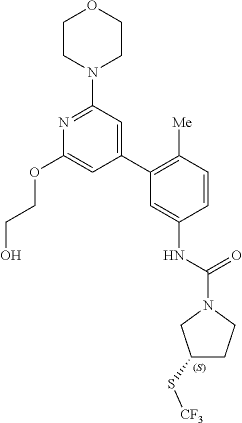 | |
| 119 and 20 | 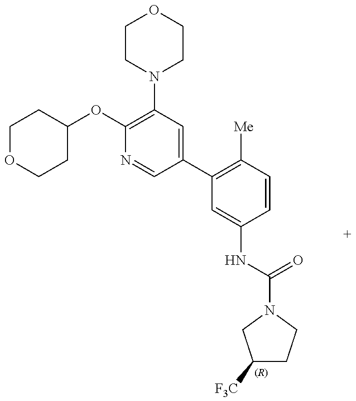 | (3R)-N-[4-methyl-3-[5-(morpholin-4- yl)-6-(oxan-4-yloxy)pyridin-3- yl]phenyl]-3- (trifluoromethyl)pyrrolidine-1- carboxamide and (3S)-N-[4-methyl-3-[5-(morpholin-4- yl)-6-(oxan-4-yloxy)pyridin-3- yl]phenyl]-3- (trifluoromethyl)pyrrolidine-1- carboxamide |
| | 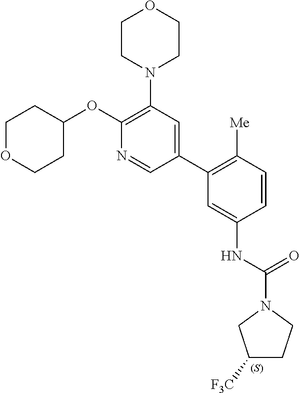 | |
| 121 | 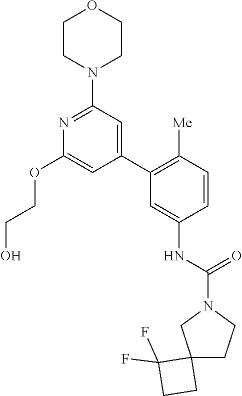 [SMILES: Cc1ccc(NC(=O)N2CCC3(CCC3(F)F)C2)cc1-c1cc(OCCO)nc(N2CCOCC2)c1] | 1,1-difluoro-N-[3-[2-(2- hydroxyethoxy)-6-(morpholin-4- yl)pyridin-4-yl]-4-methylphenyl]-6- azaspiro[3.4]octane-6-carboxamide |
| 122 | 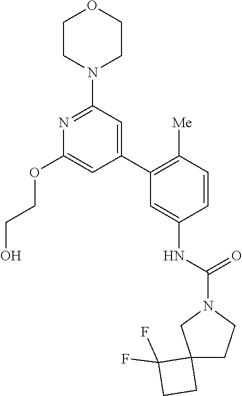 [SMILES: Cc1ccc(NC(=O)N2CCC3(CCC3(F)F)C2)cc1-c1cc(OCCO)nc(N2CCOCC2)c1] | 1,1-difluoro-N-[3-[2-(2- hydroxyethoxy)-6-(morpholin-4- yl)pyridin-4-yl]-4-methylphenyl]-6- azaspiro[3.4]octane-6-carboxamide |
| 123 | 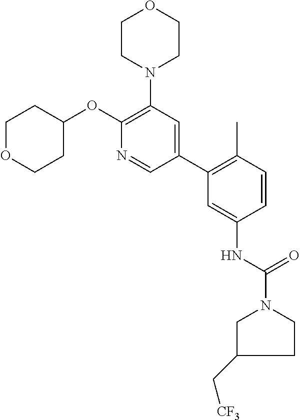 [SMILES: Cc1ccc(NC(=O)N2CCC(CC(F)(F)F)C2)cc1-c1cnc(OC2CCOCC2)c(N2CCOCC2)c1] | N-[4-methyl-3-[5-(morpholin-4-yl)-6- (oxan-4-yloxy)pyridin-3-yl]phenyl]-3- (2,2,2-trifluoroethyl)pyrrolidine-1- carboxamide |
| 124 and 125 | 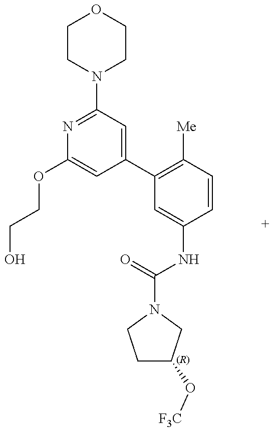 | (3R)-N-[3-[2-(2-hydroxyethoxy)-6- (morpholin-4-yl)pyridin-4-yl]-4- methylphenyl]-3- (trifluoromethoxy)pyrrolidine-1- carboxamide and (3S)-N-[3-[2-(2-hydroxyethoxy)-6- (morpholin-4-yl)pyridin-4-yl]-4- methylphenyl]-3- (trifluoromethoxy)pyrrolidine-1- carboxamide |
| | 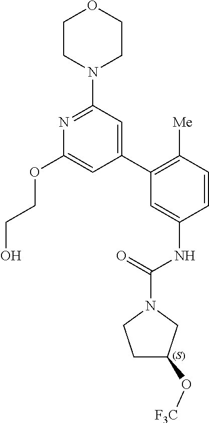 | |
| 125 | 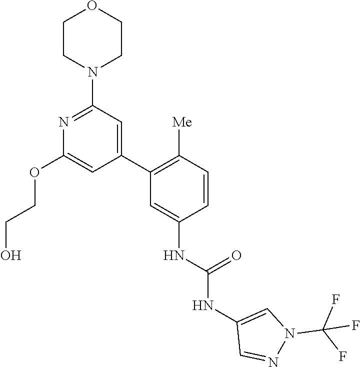 [SMILES: Cc1ccc(NC(=O)Nc2cnn(C(F)(F)F)c2)cc1-c1cc(OCCO)nc(N2CCOCC2)c1] | 1-[3-[2-(2-hydroxyethoxy)-6- (morpholin-4-yl)pyridin-4-yl]-4- methylphenyl]-3-[1- (trifluoromethyl)pyrazol-4-yl]urea |
| 126 | 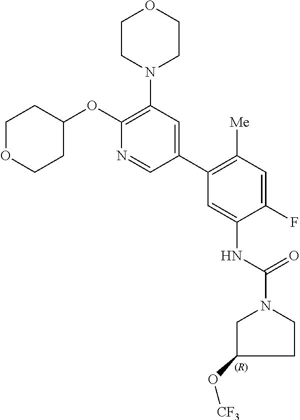 | (3R)-N-[2-fluoro-4-methyl-5-[5- (morpholin-4-yl)-6-(oxan-4- yloxy)pyridin-3-yl]phenyl]-3- (trifluoromethoxy)pyrrolidine-1- carboxamide |
| 127 | 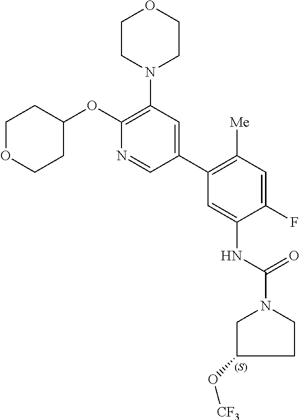 | (3S)-N-[2-fluoro-4-methyl-5-[5- (morpholin-4-yl)-6-(oxan-4- yloxy)pyridin-3-yl]phenyl]-3- (trifluoromethoxy)pyrrolidine-1- carboxamide |
| 128 | 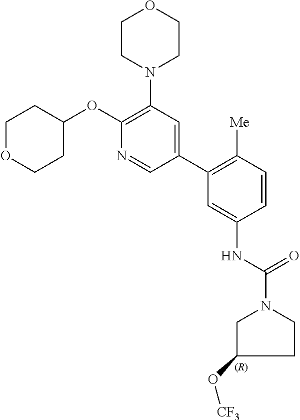 | (3R)-N-[4-methyl-3-[5-(morpholin-4- yl)-6-(oxan-4-yloxy)pyridin-3- yl]phenyl]-3- (trifluoromethoxy)pyrrolidine-1- carboxamide |
| 129 | 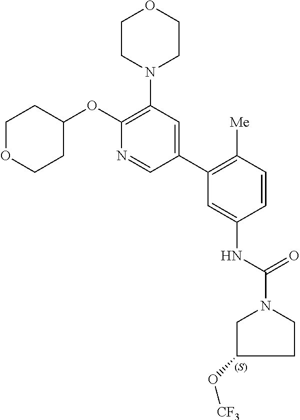 | (3S)-N-[4-methyl-3-[5-(morpholin-4- yl)-6-(oxan-4-yloxy)pyridin-3- yl]phenyl]-3- (trifluoromethoxy)pyrrolidine-1- carboxamide |
| 130 | 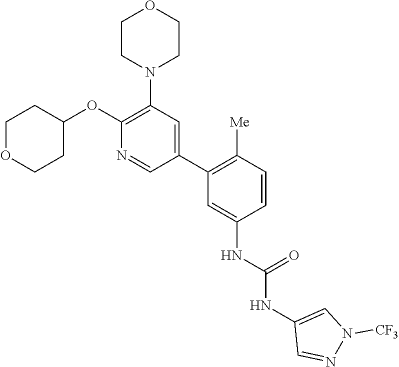 [SMILES: Cc1ccc(NC(=O)Nc2cnn(C(F)(F)F)c2)cc1-c1cnc(OC2CCOCC2)c(N2CCOCC2)c1] | 1-[4-methyl-3-[5-(morpholin-4-yl)-6- (oxan-4-yloxy)pyridin-3-yl]phenyl]-3- [1-(trifluoromethyl)pyrazol-4-yl]urea |
| 131 | 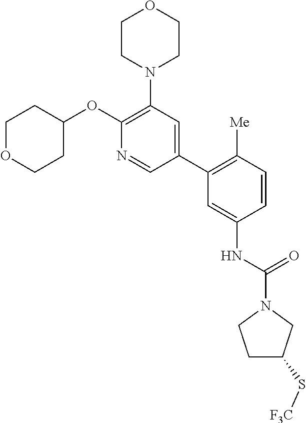 [SMILES: Cc1ccc(NC(=O)N2CC[C@@H](SC(F)(F)F)C2)cc1-c1cnc(OC2CCOCC2)c(N2CCOCC2)c1] | (3R)-N-[4-methyl-3-[5-(morpholin-4- yl)-6-(oxan-4-yloxy)pyridin-3- yl]phenyl]-3- [(trifluoromethyl)sulfanyl]pyrrolidine- 1-carboxamide |
| 132 | 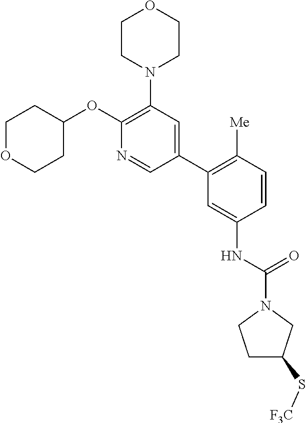 [SMILES: Cc1ccc(NC(=O)N2CC[C@H](SC(F)(F)F)C2)cc1-c1cnc(OC2CCOCC2)c(N2CCOCC2)c1] | (3S)-N-[4-methyl-3-[5-(morpholin-4- yl)-6-(oxan-4-yloxy)pyridin-3- yl]phenyl]-3- [(trifluoromethyl)sulfanyl]pyrrolidine- 1-carboxamide |
| 133 | 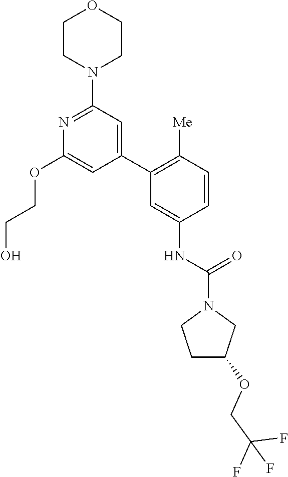 [SMILES: Cc1ccc(NC(=O)N2CC[C@@H](OCC(F)(F)F)C2)cc1-c1cc(OCCO)nc(N2CCOCC2)c1] | (3R)-N-[3-[2-(2-hydroxyethoxy)-6- (morpholin-4-yl)pyridin-4-yl]-4- methylphenyl]-3-(2,2,2- trifluoroethoxy)pyrrolidine-1- carboxamide |
| 134 | 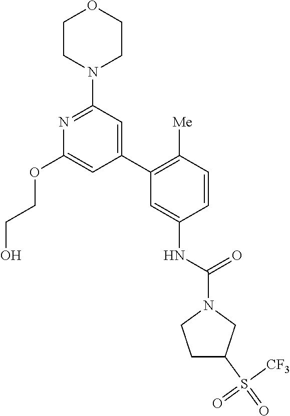 [SMILES: Cc1ccc(NC(=O)N2CCC(S(=O)(=O)C(F)(F)F)C2)cc1-c1cc(OCCO)nc(N2CCOCC2)c1] | N-[3-[2-(2-hydroxyethoxy)-6- (morpholin-4-yl)pyridin-4-yl]-4- methylphenyl]-3- trifluoromethanesulfonylpyrrolidine-1- carboxamide |
| 135 | 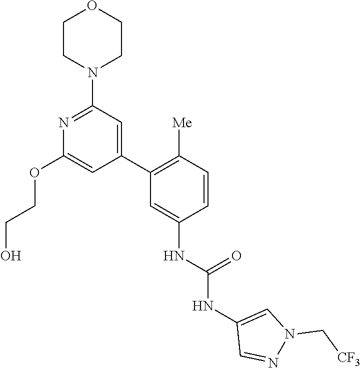 [SMILES: Cc1ccc(NC(=O)Nc2cnn(CC(F)(F)F)c2)cc1-c1cc(OCCO)nc(N2CCOCC2)c1] | 1-[3-[2-(2-hydroxyethoxy)-6- (morpholin-4-yl)pyridin-4-yl]-4- methylphenyl]-3-[1-(2,2,2- trifluoroethyl)pyrazol-4-yl]urea |
| 136 | 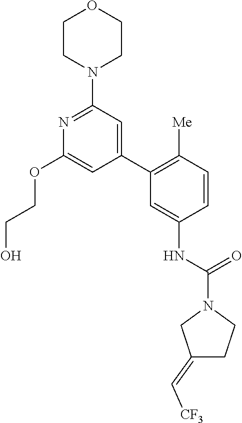 [SMILES: Cc1ccc(NC(=O)N2CC/C(=C\C(F)(F)F)C2)cc1-c1cc(OCCO)nc(N2CCOCC2)c1] | (3E)-N-[3-[2-(2-hydroxyethoxy)-6- (morpholin-4-yl)pyridin-4-yl]-4- methylphenyl]-3-(2,2,2- trifluoroethylidene)pyrrolidine-1- carboxamide |
| 137 | 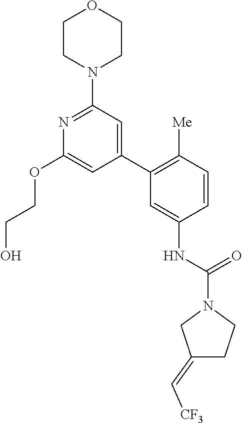 [SMILES: Cc1ccc(NC(=O)N2CC/C(=C\C(F)(F)F)C2)cc1-c1cc(OCCO)nc(N2CCOCC2)c1] | (3S)-N-[3-[2-(2-hydroxyethoxy)-6- (morpholin-4-yl)pyridin-4-yl]-4- methylphenyl]-3-(2,2,2- trifluoroethoxy)pyrrolidine-1- carboxamide |
| 138 | 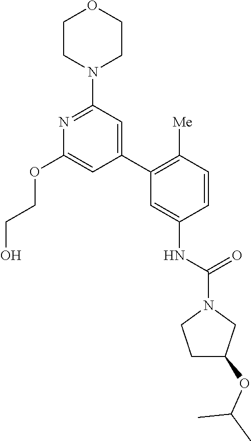 [SMILES: Cc1ccc(NC(=O)N2CC[C@H](OC(C)C)C2)cc1-c1cc(OCCO)nc(N2CCOCC2)c1] | (3S)-N-[3-[2-(2-hydroxyethoxy)-6- (morpholin-4-yl)pyridin-4-yl]-4- methylphenyl]-3- isopropoxypyrrolidine-1-carboxamide |
| 139 | 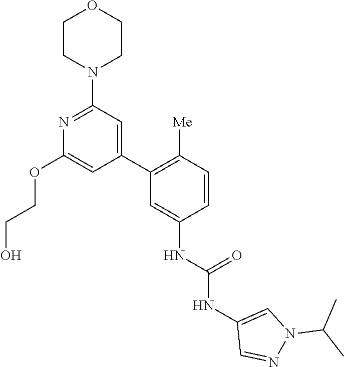 [SMILES: Cc1ccc(NC(=O)Nc2cnn(C(C)C)c2)cc1-c1cc(OCCO)nc(N2CCOCC2)c1] | 1-[3-[2-(2-hydroxyethoxy)-6- (morpholin-4-yl)pyridin-4-yl]-4- methylphenyl]-3-(1-isopropylpyrazol- 4-yl)urea |
| 140 | 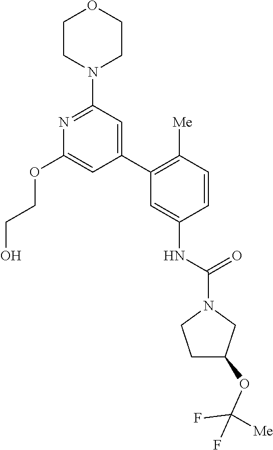 [SMILES: Cc1ccc(NC(=O)N2CC[C@H](OC(C)(F)F)C2)cc1-c1cc(OCCO)nc(N2CCOCC2)c1] | (3S)-3-(1,1-difluoroethoxy)-N-[3-[2- (2-hydroxyethoxy)-6-(morpholin-4- yl)pyridin-4-yl]-4- methylphenyl]pyrrolidine-1- carboxamide |
| 141 | 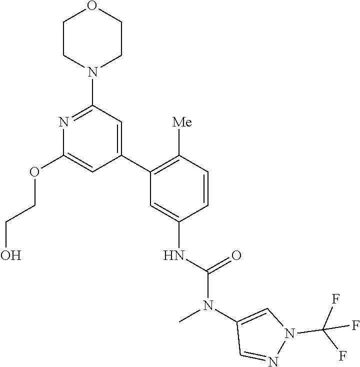 [SMILES: Cc1ccc(NC(=O)N(C)c2cnn(C(F)(F)F)c2)cc1-c1cc(OCCO)nc(N2CCOCC2)c1] | 1-[3-[2-(2-hydroxyethoxy)-6- (morpholin-4-yl)pyridin-4-yl]-4- methylphenyl]-3-methyl-3-[1- (trifluoromethyl)pyrazol-4-yl]urea |
| 142 | 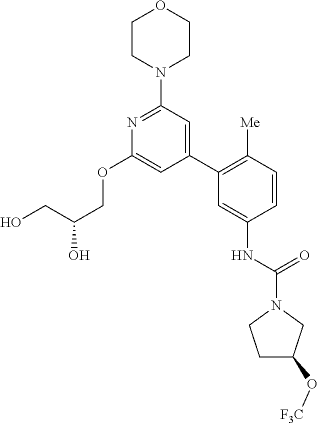 [SMILES: Cc1ccc(NC(=O)N2CC[C@H](OC(F)(F)F)C2)cc1-c1cc(OC[C@H](O)CO)nc(N2CCOCC2)c1] | (3S)-N-(3-[2-[(2R)-2,3- dihydroxypropoxy]-6-(morpholin-4- yl)pyridin-4-yl]-4-methylphenyl)-3- (trifluoromethoxy)pyrrolidine-1- carboxamide |
| 143 | 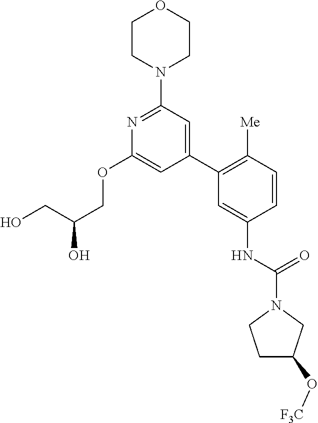 [SMILES: Cc1ccc(NC(=O)N2CC[C@H](OC(F)(F)F)C2)cc1-c1cc(OC[C@@H](O)CO)nc(N2CCOCC2)c1] | (3S)-N-(3-[2-[(2S)-2,3- dihydroxypropoxy]-6-(morpholin-4- yl)pyridin-4-yl]-4-methylphenyl)-3- (trifluoromethoxy)pyrrolidine-1- carboxamide |
| 144 | 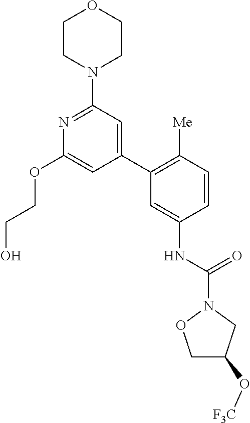 [SMILES: Cc1ccc(NC(=O)N2C[C@@H](OC(F)(F)F)CO2)cc1-c1cc(OCCO)nc(N2CCOCC2)c1] | (4R)-N-[3-[2-(2-hydroxyethoxy)-6- (morpholin-4-yl)pyridin-4-yl]-4- methylphenyl]-4-(trifluoromethoxy)- 1,2-oxazolidine-2-carboxamide |
| 145 | 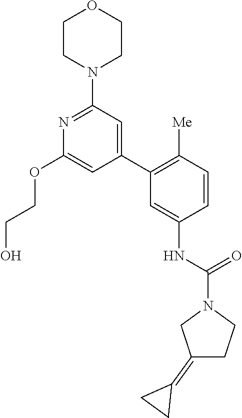 [SMILES: Cc1ccc(NC(=O)N2CCC(=C3CC3)C2)cc1-c1cc(OCCO)nc(N2CCOCC2)c1] | 3-cyclopropylidene-N-[3-[2-(2- hydroxyethoxy)-6-(morpholin-4- yl)pyridin-4-yl]-4- methylphenyl]pyrrolidine-1- carboxamide |
| 146 | 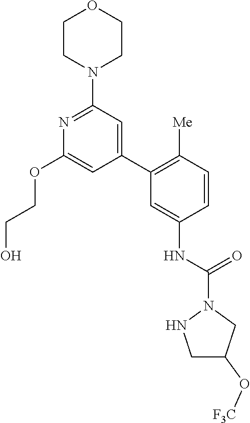 [SMILES: Cc1ccc(NC(=O)N2CC(OC(F)(F)F)CN2)cc1-c1cc(OCCO)nc(N2CCOCC2)c1] | N-[3-[2-(2-hydroxyethoxy)-6- (morpholin-4-yl)pyridin-4-yl]-4- methylphenyl]-4-(trifluoromethoxy) pyrazolidine-1-carboxamide |
| 147 | 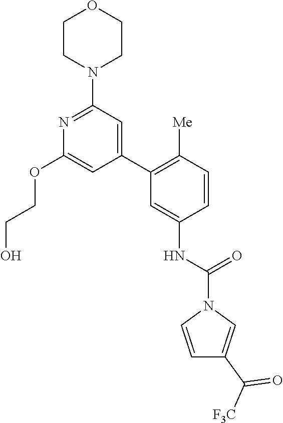 [SMILES: Cc1ccc(NC(=O)n2ccc(C(=O)C(F)(F)F)c2)cc1-c1cc(OCCO)nc(N2CCOCC2)c1] | N-[3-[2-(2-hydroxyethoxy)-6- (morpholin-4-yl)pyridin-4-yl]-4- methylphenyl]-3-(2,2,2- trifluoroacetyl)pyrrole-1-carboxamide |
| 148 | 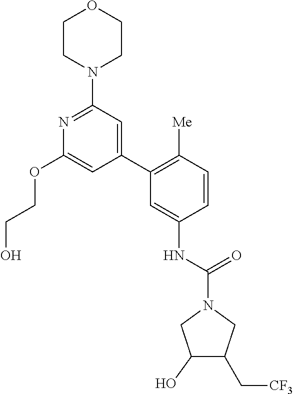 [SMILES: Cc1ccc(NC(=O)N2CC(O)C(CC(F)(F)F)C2)cc1-c1cc(OCCO)nc(N2CCOCC2)c1] | 3-hydroxy-N-[3-[2-(2- hydroxyethoxy)-6-(morpholin-4- yl)pyridin-4-yl]-4-methylphenyl]- 4- (2,2,2-trifluoroethyl)pyrrolidine-1- carboxamide |
| 149 | 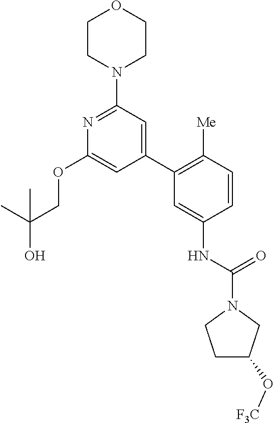 [SMILES: Cc1ccc(NC(=O)N2CC[C@@H](OC(F)(F)F)C2)cc1-c1cc(OCC(C)(C)O)nc(N2CCOCC2)c1] | (3R)-N-[3-[2-(2-hydroxy-2- methylpropoxy)-6-(morpholin-4- yl)pyridin-4-yl]-4-methylphenyl]-3- (trifluoromethoxy)pyrrolidine-1- carboxamide |
| 150 | 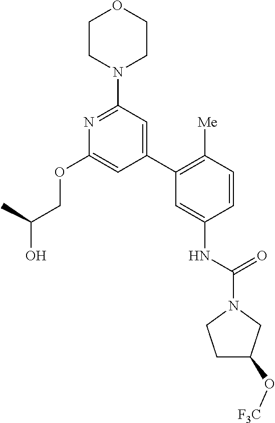 [SMILES: Cc1ccc(NC(=O)N2CC[C@H](OC(F)(F)F)C2)cc1-c1cc(OC[C@H](C)O)nc(N2CCOCC2)c1] | (3S)-N-(3-[2-[(2S)-2- hydroxypropoxy]-6-(morpholin-4- yl)pyridin-4-yl]-4-methylphenyl)-3- (trifluoromethoxy)pyrrolidine-- carboxamide |
| 151 | 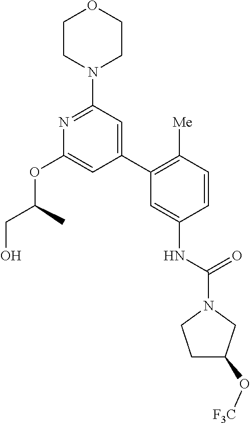 [SMILES: Cc1ccc(NC(=O)N2CC[C@H](OC(F)(F)F)C2)cc1-c1cc(O[C@@H](C)CO)nc(N2CCOCC2)c1] | (3S)-N-[3-(2-[[(2S)-1-hydroxypropan- 2-yl]oxy]-6-(morpholin-4-yl)pyridin- 4-yl)-4-methylphenyl]-3- (trifluoromethoxy)pyrrolidine-1- carboxamide |
| 152 | 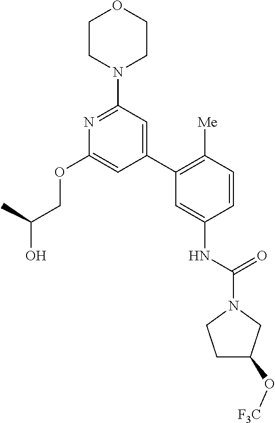 [SMILES: Cc1ccc(NC(=O)N2CC[C@H](OC(F)(F)F)C2)cc1-c1cc(OC[C@H](C)O)nc(N2CCOCC2)c1] | (3S)-N-(3-[2-[(2R)-2- hydroxypropoxy]-6-(morpholin-4- yl)pyridin-4-yl]-4-methylphenyl)-3- (trifluoromethoxy)pyrrolidine-1- carboxamide |
| 153 | 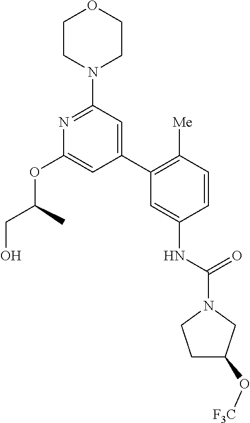 [SMILES: Cc1ccc(NC(=O)N2CC[C@H](OC(F)(F)F)C2)cc1-c1cc(O[C@@H](C)CO)nc(N2CCOCC2)c1] | (3S)-N-[3-(2-[[(2R)-1-hydroxypropan- 2-yl]oxy]-6-(morpholin-4-yl)pyridin- 4-yl)-4-methylphenyl]-3- (trifluoromethoxy)pyrrolidine-1- carboxamide |
| 154 | 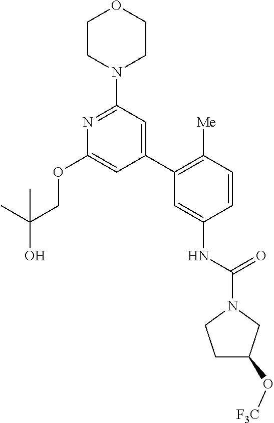 [SMILES: Cc1ccc(NC(=O)N2CC[C@H](OC(F)(F)F)C2)cc1-c1cc(OCC(C)(C)O)nc(N2CCOCC2)c1] | (3S)-N-[3-[2-(2-hydroxy-2- methylpropoxy)-6-(morpholin-4- yl)pyridin-4-yl]-4-methylphenyl]-3- (trifluoromethoxy)pyrrolidine-1- carboxamide |
| 155 | 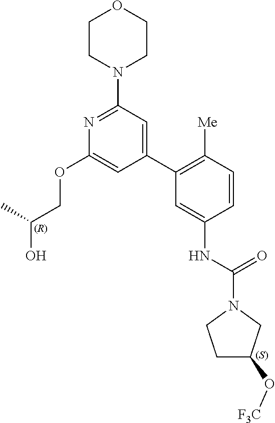 | (3S)-N-(3-[2-[(2R)-2- hydroxypropoxy]-6-(morpholin-4- yl)pyridin-4-yl]-4-methylphenyl)-3- (trifluoromethoxy)pyrrolidine-1- carboxamide |
| 156 | 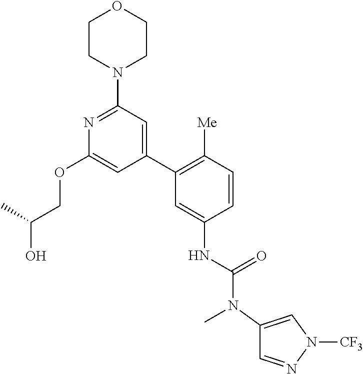 [SMILES: Cc1ccc(NC(=O)N(C)c2cnn(C(F)(F)F)c2)cc1-c1cc(OC[C@@H](C)O)nc(N2CCOCC2)c1] | 1-(3-[2-[(2R)-2-hydroxypropoxy]-6- (morpholin-4-yl)pyridin-4-yl]-4- methylphenyl)-3-methyl-3-[1- (trifluoromethyl)pyrazol-4-yl]urea |
| 157 and 158 | 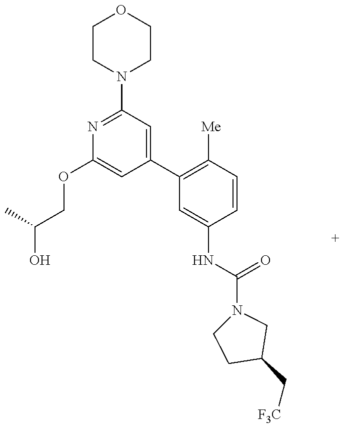 [SMILES: Cc1ccc(NC(=O)N2CC[C@H](CC(F)(F)F)C2)cc1-c1cc(OC[C@@H](C)O)nc(N2CCOCC2)c1] | (R)-N-(3-(2-((R)-2-hydroxypropoxy)- 6-morpholinopyridin-4-yl)-4- methylphenyl)-3-(2,2,2- trifluoroethyl)pyrrolidine-1- carboxamide and (S)-N-(3-(2-((R)-2-hydroxypropoxy)- 6-morpholinopyrridin-4-yl)-4- methylphenyl)-3-(2,2,2- trifluoroethyl)pyrrolidine-1- carboxamide |
| | 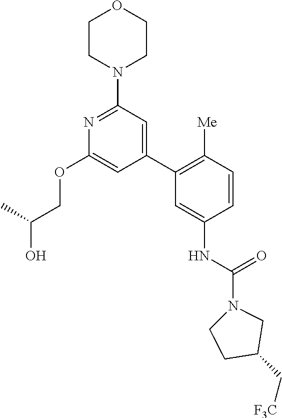 [SMILES: Cc1ccc(NC(=O)N2CC[C@@H](CC(F)(F)F)C2)cc1-c1cc(OC[C@@H](C)O)nc(N2CCOCC2)c1] | |
| 159 and 160 | 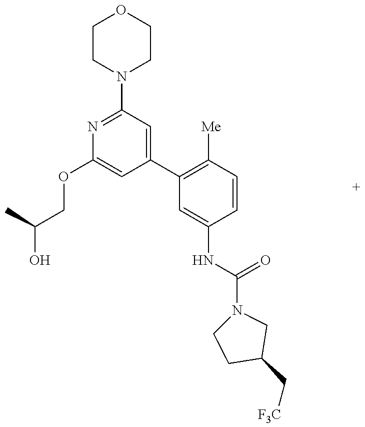 [SMILES: Cc1ccc(NC(=O)N2CC[C@H](CC(F)(F)F)C2)cc1-c1cc(OC[C@H](C)O)nc(N2CCOCC2)c1] | (R)-N-(3-(2-((S)-2-hydroxypropoxy)- 6-morpholinopyrridin-4-yl)-4- methylphenyl)-3-(2,2,2- trifluoroethyl)pyrrolidine-1- carboxamide and (S)-N-(3-(2-((S)-2-hydroxypropoxy)- 6-morpholinopyridin-4-yl)-4- methylphenyl)-3-(2,2,2- trifluoroethyl)pyrrolidine-1- carboxamide |
| | 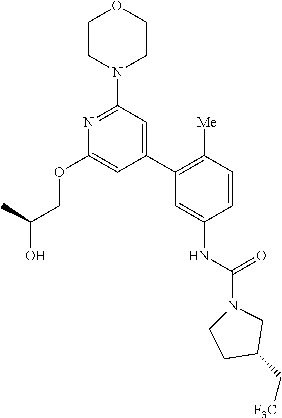 [SMILES: Cc1ccc(NC(=O)N2CC[C@@H](CC(F)(F)F)C2)cc1-c1cc(OC[C@H](C)O)nc(N2CCOCC2)c1] | |
| 161 and 162 | 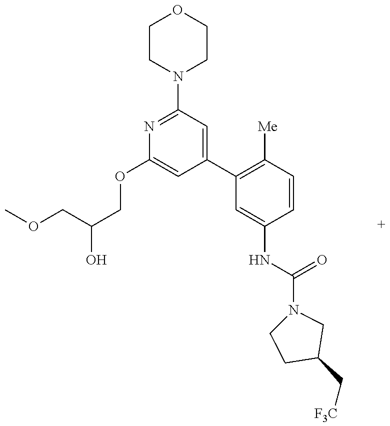 [SMILES: COCC(O)COc1cc(-c2cc(NC(=O)N3CC[C@H](CC(F)(F)F)C3)ccc2C)cc(N2CCOCC2)n1] | (3R)-N-[3-[2-(2-hydroxy-3- methoxypropoxy)-6-(morpholin-4- yl)pyridin-4-yl]-4-methylphenyl]-3- (2,2,2-trifluoroethyl)pyrrolidine-1- carboxamide and (3S)-N-[3-[2-(2-hydroxy-3- methoxypropoxy)-6-(morpholin-4- yl)pyridin-4-yl]-4-methylphenyl]-3- (2,2,2-trifluoroethyl)pyrrolidine-1- carboxamide |
| | 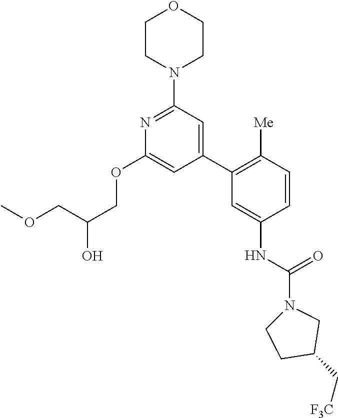 [SMILES: COCC(O)COc1cc(-c2cc(NC(=O)N3CC[C@@H](CC(F)(F)F)C3)ccc2C)cc(N2CCOCC2)n1] | |
| 163, 164, 165 and 166 | 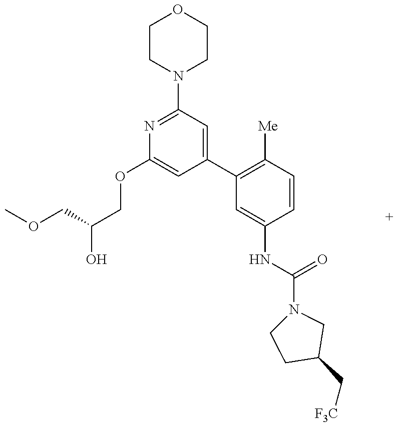 [SMILES: COC[C@@H](O)COc1cc(-c2cc(NC(=O)N3CC[C@H](CC(F)(F)F)C3)ccc2C)cc(N2CCOCC2)n1] | (3R)-N-(3-[2-[(2R)-2-hydroxy-3- methoxypropoxy]-6-(morpholin-4- yl)pyridin-4-yl]-4-methylphenyl)-3- (2,2,2-trifluoroethyl)pyrrolidine-1- carboxamide and (3R)-N-(3-[2-[(2S)-2-hydroxy-3- methoxypropoxy]-6-(morpholin-4- yl)pyridin-4-yl]-4-methylphenyl)-3- (2,2,2-trifluoroethyl)pyrrolidine-1- carboxamide and |
| | 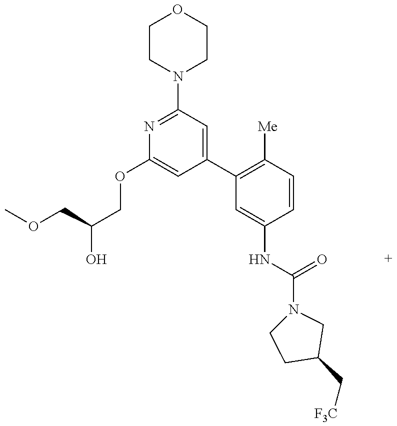 [SMILES: COC[C@H](O)COc1cc(-c2cc(NC(=O)N3CC[C@H](CC(F)(F)F)C3)ccc2C)cc(N2CCOCC2)n1] | |
| | 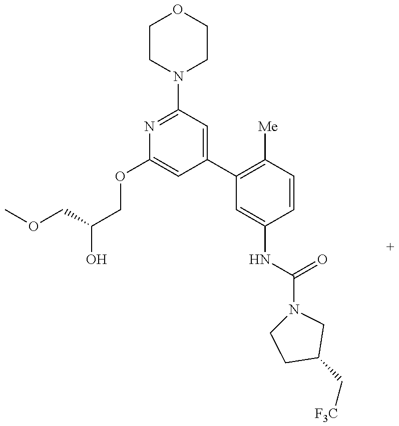 [SMILES: COC[C@@H](O)COc1cc(-c2cc(NC(=O)N3CC[C@@H](CC(F)(F)F)C3)ccc2C)cc(N2CCOCC2)n1] | (3S)-N-(3-[2-[(2R)-2-hydroxy-3- methoxypropoxy]-6-(morpholin-4- yl)pyridin-4-yl]-4-methylphenyl)-3- (2,2,2-trifluoroethyl)pyrrolidine-1- carboxamide and (3S)-N-(3-[2-[(2S)-2-hydroxy-3- methoxypropoxy]-6-(morpholin-4- yl)pyridin-4-yl]-4-methylphenyl)-3- (2,2,2-trifluoroethyl)pyrrolidine-1- carboxamide |
| | 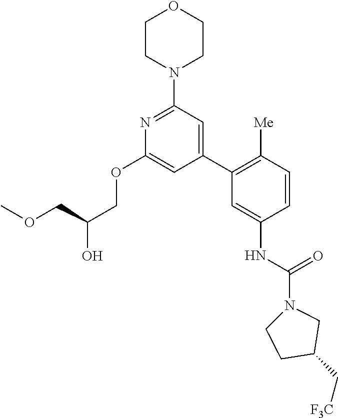 [SMILES: COC[C@H](O)COc1cc(-c2cc(NC(=O)N3CC[C@@H](CC(F)(F)F)C3)ccc2C)cc(N2CCOCC2)n1] | |
| 167 | 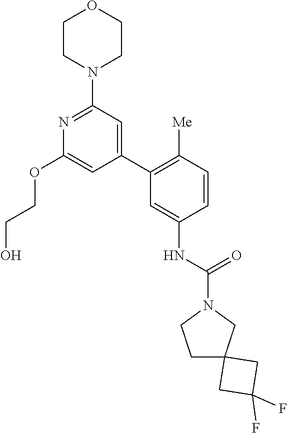 [SMILES: Cc1ccc(NC(=O)N2CCC3(C2)CC(F)(F)C3)cc1-c1cc(OCCO)nc(N2CCOCC2)c1] | 2,2-difluoro-N-(3-(2-(2- hydroxyethoxy)-6-morpholinopyridin- 4-yl)-4-methylphenyl)-6- azaspiro[3.4]octane-6-carboxamide |
| 168 | 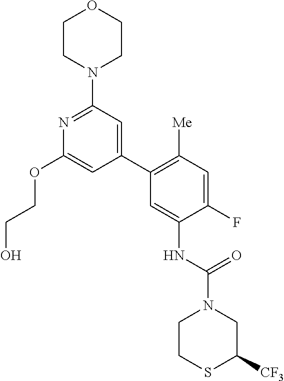 | (S)-N-(2-fluoro-5-(2-(2- hydroxyethoxy)-6-morpholinopyridin- 4-yl)-4-methylphenyl)-2- (trifluoromethyl)thiomorpholine-4- carboxamide |
| 169 | 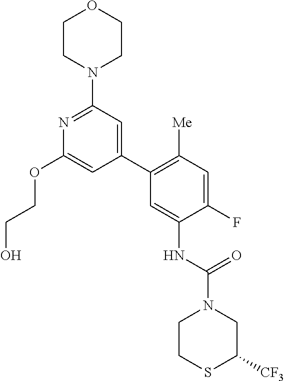 | (R)-N-(2-fluoro-5-(2-(2- hydroxyethoxy)-6-morpholinopyridin- 4-yl)-4-methylphenyl)-2- (trifluoromethyl)thiomorpholine-4- carboxamide |
| 170 | 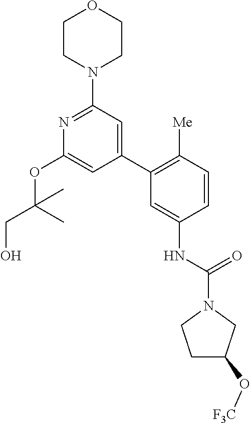 [SMILES: Cc1ccc(NC(=O)N2CC[C@H](OC(F)(F)F)C2)cc1-c1cc(OC(C)(C)CO)nc(N2CCOCC2)c1] | (3S)-N-(3-[2-[(1-hydroxy-2- methylpropan-2-yl)oxy]-6-(morpholin- 4-yl)pyridin-4-yl]-4-methylphenyl)-3- (trifluoromethoxy)pyrrolidine-1- carboxamide |
| 171 | 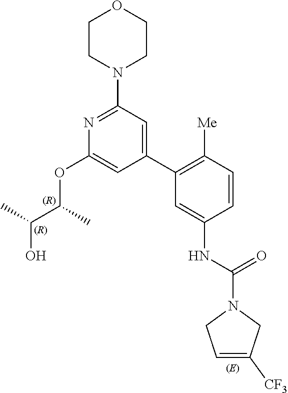 | N-(3-(2-(((2R,3R)-3-hydroxybutan-2- yl)oxy)-6-morpholinopyridin-4-yl)-4- methylphenyl)-3-(trifluoromethyl)-2,5- dihydro-1H-pyrrole-1-carboxamide |
| 172 | 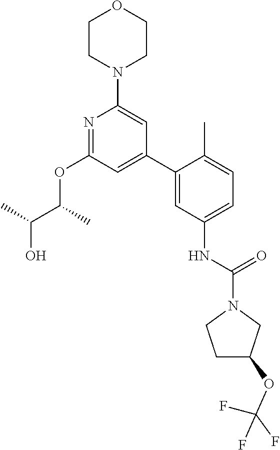 [SMILES: Cc1ccc(NC(=O)N2CC[C@H](OC(F)(F)F)C2)cc1-c1cc(O[C@H](C)[C@@H](C)O)nc(N2CCOCC2)c1] | (S)-N-(3-(2-(((2R,3R)-3- hydroxybutan-2-yl)oxy)-6- morpholinopyridin-4-yl)-4- methylphenyl)-3- (trifluoromethoxy)pyrrolidine-1- carboxamide |
| 173 | 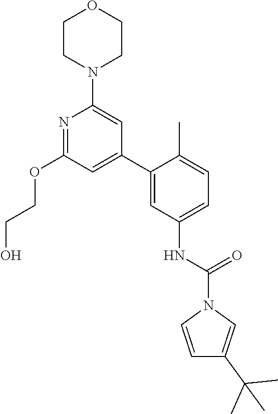 [SMILES: Cc1ccc(NC(=O)n2ccc(C(C)(C)C)c2)cc1-c1cc(OCCO)nc(N2CCOCC2)c1] | 3-(tert-butyl)-N-(3-(2-(2- hydroxyethoxy)-6-morpholinopyridin- 4-yl)-4-methylphenyl)-1H-pyrrole-1- carboxamide |
| 174 | 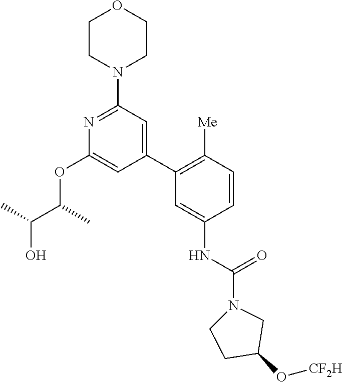 [SMILES: Cc1ccc(NC(=O)N2CC[C@H](OC(F)F)C2)cc1-c1cc(O[C@H](C)[C@@H](C)O)nc(N2CCOCC2)c1] | (S)-3-(difluoromethoxy)-N-(3-(2- (((2R,3R)-3-hydroxybutan-2-yl)oxy)- 6-morpholinopyridin-4-yl)-4- methylphenyl)pyrrolidine-1- carboxamide |
| 175 | 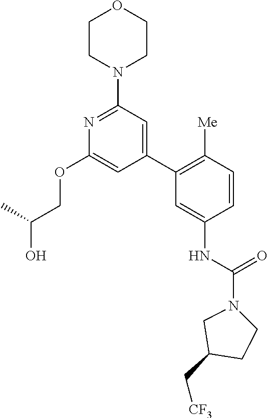 [SMILES: Cc1ccc(NC(=O)N2CC[C@@H](CC(F)(F)F)C2)cc1-c1cc(OC[C@@H](C)O)nc(N2CCOCC2)c1] | (S)-N-(3-(2-((R)-2-hydroxypropoxy)- 6-morpholinopyridin-4-yl)-4- methylphenyl)-3-(2,2,2- trifluoroethyl)pyrrolidine-1- carboxamide |
| 176 | 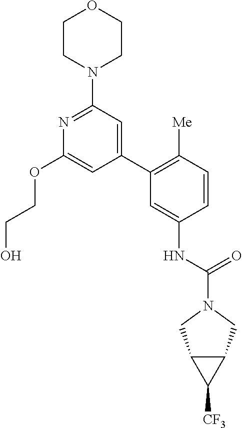 [SMILES: Cc1ccc(NC(=O)N2C[C@@H]3[C@H](C2)[C@H]3C(F)(F)F)cc1-c1cc(OCCO)nc(N2CCOCC2)c1] | (1R,5S,6r)-N-(3-(2-(2- hydroxyethoxy)-6-morpholinopyridin- 4-yl)-4-methylphenyl)-6- (trifluoromethyl)-3- azabicyclo[3.1.0]hexane-3- carboxamide |
| 177 and 178 | 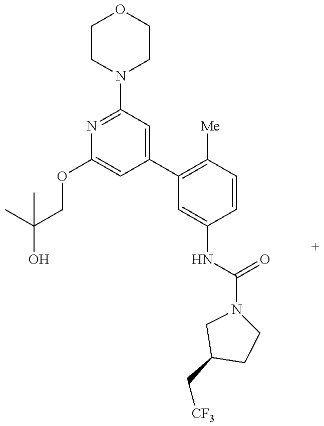 [SMILES: Cc1ccc(NC(=O)N2CC[C@@H](CC(F)(F)F)C2)cc1-c1cc(OCC(C)(C)O)nc(N2CCOCC2)c1] | (S)-N-(3-(2-(2-hydroxy-2- methylpropoxy)-6-morpholinopyridin- 4-yl)-4-methylphenyl)-3-(2,2,2- trifluoroethyl)pyrrolidine-1- carboxamide and (R)-N-(3-(2-(2-hydroxy-2- mehtylpropoxy)-6-morpholinopyridin- 4-yl)-4-methylphenyl)-3-(2,2,2- trifluoroethyl)pyrrolidine-1- carboxamide |
| | 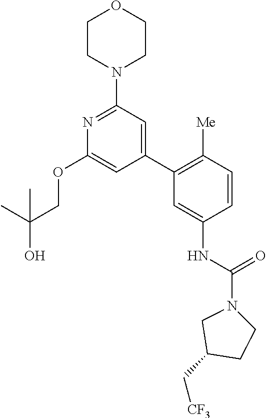 [SMILES: Cc1ccc(NC(=O)N2CC[C@H](CC(F)(F)F)C2)cc1-c1cc(OCC(C)(C)O)nc(N2CCOCC2)c1] | |
| 179 | 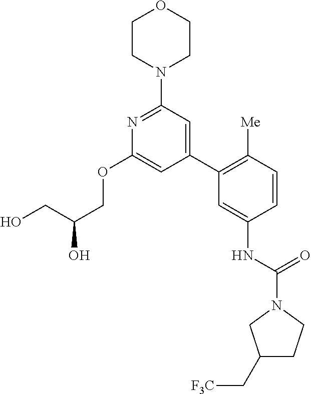 [SMILES: Cc1ccc(NC(=O)N2CCC(CC(F)(F)F)C2)cc1-c1cc(OC[C@@H](O)CO)nc(N2CCOCC2)c1] | N-(3-(2-((S)-2,3-dihydroxypropoxy)-6- morpholinopyridin-4-yl)-4- methylphenyl)-3-(2,2,2- trifluoroethyl)pyrrolidine-1- carboxamide |
| 180 | 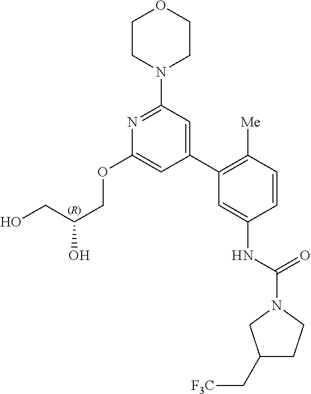 | N-(3-(2-((R)-2,3-dihydroxypropoxy)- 6-morpholinopyridin-4-yl)-4- methylphenyl)-3-(2,2,2- trifluoroethyl)pyrrolidine-1- carboxamide |
| 181, 182, 183 and 184 | 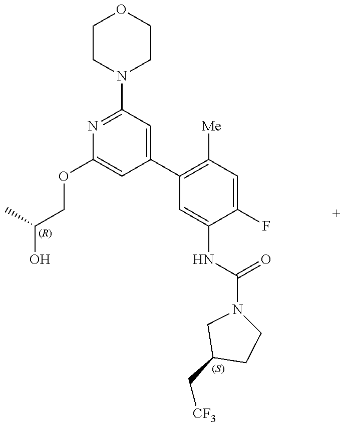 | (S)-N-(2-fluoro-5-(2-((R)-2- hydroxypropoxy)-6- morpholinopyridin-4-yl)-4- methylphenyl)-3-(2,2,2- trifluoroethyl)pyrrolidine-1- carboxamide and (S)-N-(2-fluoro-5-(2-(((R)-1- hydroxypropan-2-yl)oxy)-6- morpholinopyridin-4-yl)-4- methylphenyl)-3-(2,2,2- trifluoroethyl)pyrrolidine-1- carboxamide and |
| | 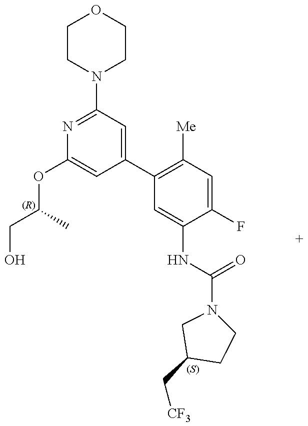 | |
| | 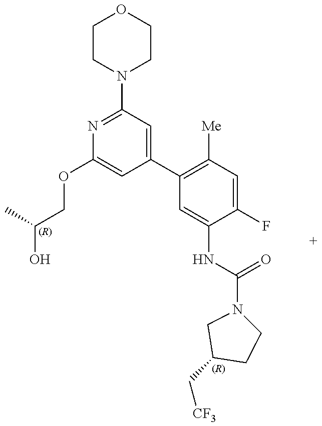 | (R)-N-(2-fluoro-5-(2-((R)-2- hydroxypropoxy)-6- morpholinopyridin-4-yl)-4- methylphenyl)-3-(2,2,2- trifluoroethyl)pyrrolidine-1- carboxamide and (R)-N-(2-fluoro-5-(2-(((R)-1- hydroxypropan-2-yl)oxy)-6- morpholinopyridin-4-yl)-4- methylphenyl)-3-(2,2,2- trifluoroethyl)pyrrolidine-1- carboxamide |
| | 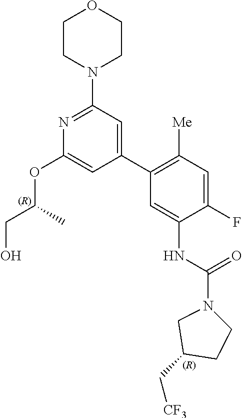 | |
| 185 and 186 | 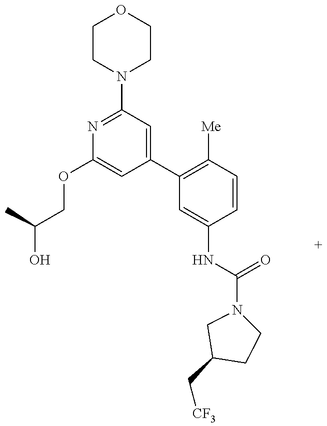 [SMILES: Cc1ccc(NC(=O)N2CC[C@@H](CC(F)(F)F)C2)cc1-c1cc(OC[C@H](C)O)nc(N2CCOCC2)c1] | (S)-N-(3-(2-((S)-2-hydroxypropoxy)- 6-morpholinopyridin-4-yl)-4- methylphenyl)-3-(2,2,2- trifluoroethyl)pyrrolidine-1- carboxamide and (R)-N-(3-(2-((S)-2-hydroxypropoxy)- 6-morpholinopyridin-4-yl)-4- methylphenyl)-3-(2,2,2- trifluoroethyl)pyrrolidine-1- carboxamide |
| | 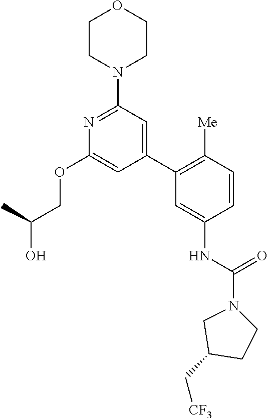 [SMILES: Cc1ccc(NC(=O)N2CC[C@H](CC(F)(F)F)C2)cc1-c1cc(OC[C@H](C)O)nc(N2CCOCC2)c1] | |
| 187 | 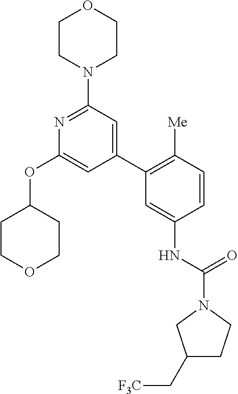 [SMILES: Cc1ccc(NC(=O)N2CCC(CC(F)(F)F)C2)cc1-c1cc(OC2CCOCC2)nc(N2CCOCC2)c1] | N-(4-methyl-3-(2-morpholino-6- ((tetrahydro-2H-pyran-4- yl)oxy)pyridin-4-yl)phenyl)-3-(2,2,2- trifluoroethyl)pyrrolidine-1- carboxamide |
| 188 | 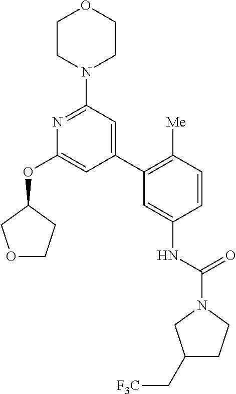 [SMILES: Cc1ccc(NC(=O)N2CCC(CC(F)(F)F)C2)cc1-c1cc(O[C@H]2CCOC2)nc(N2CCOCC2)c1] | N-(4-methyl-3-(2-morpholino-6-(((S)- tetrahydrofuran-3-yl)oxy)pyridin-4- yl)phenyl)-3-(2,2,2- trifluoroethyl)pyrrolidine-1- carboxamide |
| 189 | 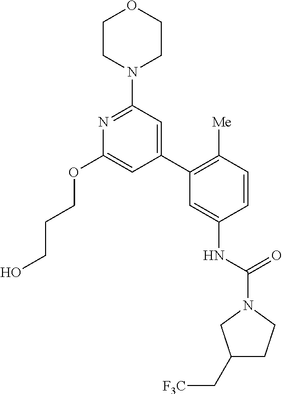 [SMILES: Cc1ccc(NC(=O)N2CCC(CC(F)(F)F)C2)cc1-c1cc(OCCCO)nc(N2CCOCC2)c1] | N-(3-(2-(3-hydroxypropoxy)-6- morpholinopyridin-4-yl)-4- methylphenyl)-3-(2,2,2- trifluoroethyl)pyrrolidine-1- carboxamide |
| 190 | 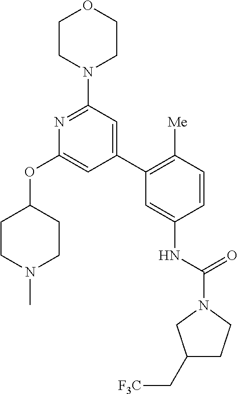 [SMILES: Cc1ccc(NC(=O)N2CCC(CC(F)(F)F)C2)cc1-c1cc(OC2CCN(C)CC2)nc(N2CCOCC2)c1] | N-(4-methyl-3-(2-((1-methylpiperidin- 4-yl)oxy)-6-morpholinopyridin-4- yl)phenyl)-3-(2,2,2- trifluoroethyl)pyrrolidine-1- carboxamide |
| 191 and 192 | 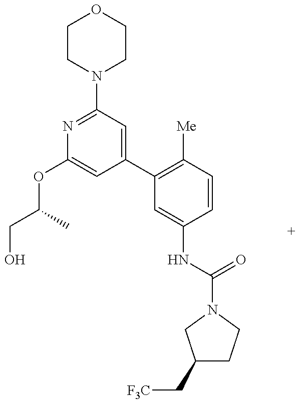 [SMILES: Cc1ccc(NC(=O)N2CC[C@@H](CC(F)(F)F)C2)cc1-c1cc(O[C@H](C)CO)nc(N2CCOCC2)c1] | (S)-N-(3-(2-(((R)-1-hydroxypropaon-2- yl)oxy)-6-morpholinopyridin-4-yl)-4- methylphenyl)-3-(2,2,2- trifluoroethyl)pyrrolidine-1- carboxamide and (R)-N-(3-(2-(((R)-1-hydroxypropan-2- yl)oxy)-6-morpholinopyridin-4-yl)-4- methylphenyl)-3-(2,2,2- trifluoroethyl)pyrrolidine-1- carboxamide |
| | 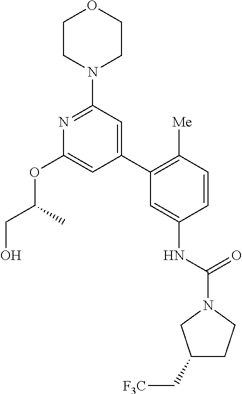 [SMILES: Cc1ccc(NC(=O)N2CC[C@H](CC(F)(F)F)C2)cc1-c1cc(O[C@H](C)CO)nc(N2CCOCC2)c1] | |
| 193 and 194 | 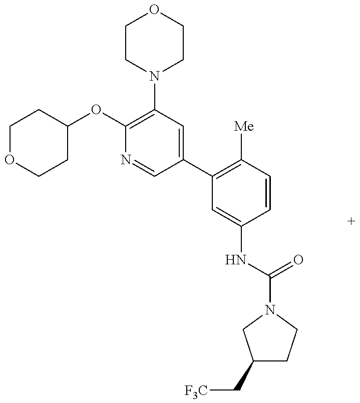 [SMILES: Cc1ccc(NC(=O)N2CC[C@@H](CC(F)(F)F)C2)cc1-c1cnc(OC2CCOCC2)c(N2CCOCC2)c1] | (S)-N-(4-methyl-3-(5-morpholino-6- ((tetrahydro-2H-pyran-4- yl)oxy)pyridin-3-yl)phenyl)-3-(2,2,2- trifluoroethyl)pyrrolidine-1- carboxamide and (R)-N-(4-methyl-3-(5-morpholino-6- ((tetrahydro-2H-pyran-4- yl)oxy)pyridin-3-yl)phenyl)-3-(2,2,2- trifluoroethyl)pyrrolidine-1- carboxamide |
| | 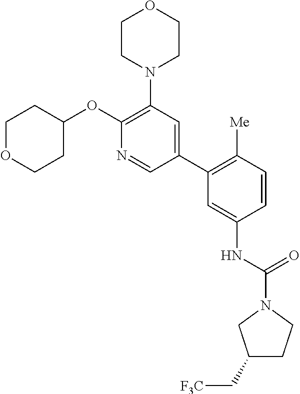 [SMILES: Cc1ccc(NC(=O)N2CC[C@H](CC(F)(F)F)C2)cc1-c1cnc(OC2CCOCC2)c(N2CCOCC2)c1] | |
| 195 | 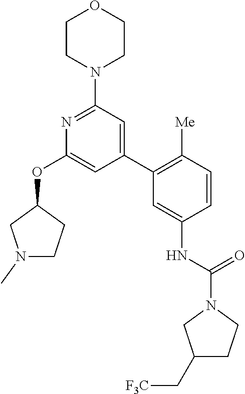 [SMILES: Cc1ccc(NC(=O)N2CCC(CC(F)(F)F)C2)cc1-c1cc(O[C@H]2CCN(C)C2)nc(N2CCOCC2)c1] | N-(4-methyl-3-(2-(((S)-1- methylpyrrolidin-3-yl)oxy)-6- morpholinopyridin-4-yl)phenyl)-3- (2,2,2-trifluoroethyl)pyrrolidine-1- carboxamide |
| 196 | 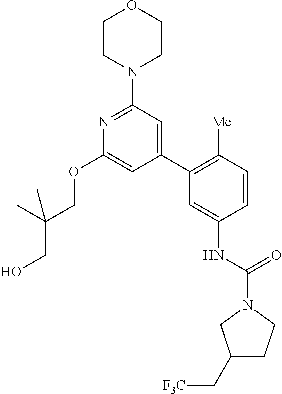 [SMILES: Cc1ccc(NC(=O)N2CCC(CC(F)(F)F)C2)cc1-c1cc(OCC(C)(C)CO)nc(N2CCOCC2)c1] | N-(3-(2-(3-hydroxy-2,2- dimethylpropoxy)-6- morpholinopyridin-4-yl)-4- methylphenyl)-3-(2,2,2- trifluoroethyl)pyrrolidine-1- carboxamide |
| 197 and 198 | 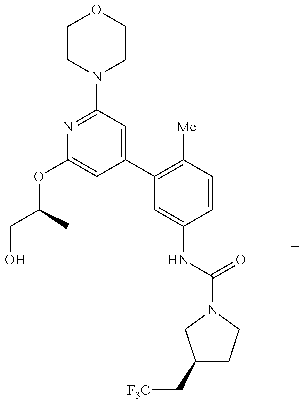 [SMILES: Cc1ccc(NC(=O)N2CC[C@@H](CC(F)(F)F)C2)cc1-c1cc(O[C@@H](C)CO)nc(N2CCOCC2)c1] | (S)-N-(3-(2-(((S)-1-hydroxypropan-2- yl)oxy)-6-morpholinopyridin-4-yl)-4- methylphenyl)-3-(2,2,2- trifluoroethyl)pyrrolidine-1- carboxamide and (R)-N-(3-(2-(((S)-1-hydroxypropan-2- yl)oxy)-6-morpholinopyridin-4-yl)-4- methylphenyl)-3-(2,2,2- trifluoroethyl)pyrrolidine-1- carboxamide |
| | 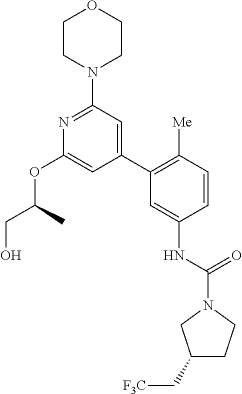 [SMILES: Cc1ccc(NC(=O)N2CC[C@H](CC(F)(F)F)C2)cc1-c1cc(O[C@@H](C)CO)nc(N2CCOCC2)c1] | |
| 199 | 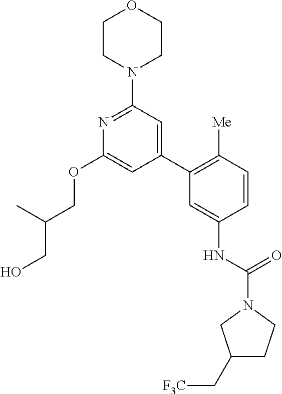 [SMILES: Cc1ccc(NC(=O)N2CCC(CC(F)(F)F)C2)cc1-c1cc(OCC(C)CO)nc(N2CCOCC2)c1] | N-(3-(2-(3-hydroxy-2- methylpropoxy)-6-morpholinopyridin- 4-yl)-4-methylphenyl)-3-(2,2,2- trifluoroethyl)pyrrolidine-1- carboxamide |
| 200 | 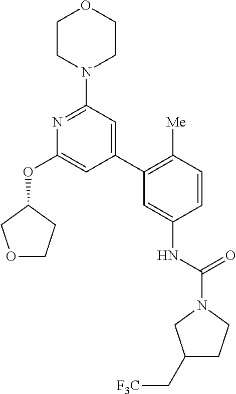 [SMILES: Cc1ccc(NC(=O)N2CCC(CC(F)(F)F)C2)cc1-c1cc(O[C@@H]2CCOC2)nc(N2CCOCC2)c1] | N-(4-methyl-3-(2-morpholino-6-(((R)- tetrahydrofuran-3-yl)oxy)pyridin-4- yl)phenyl)-3-(2,2,2- trifluoroethyl)pyrrolidine-1- carboxamide |
| 201 | 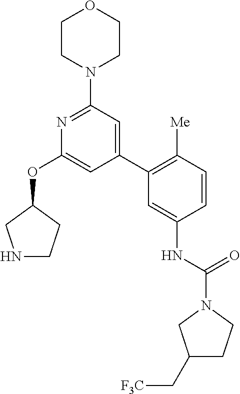 [SMILES: Cc1ccc(NC(=O)N2CCC(CC(F)(F)F)C2)cc1-c1cc(O[C@H]2CCNC2)nc(N2CCOCC2)c1] | N-(4-methyl-3-(2-morpholino-6-((S)- pyrrolidin-3-yloxy)pyridin-4- yl)phenyl)-3-(2,2,2,- trifluoroethyl)pyrrolidine-1- carboxamide |
| 202 | 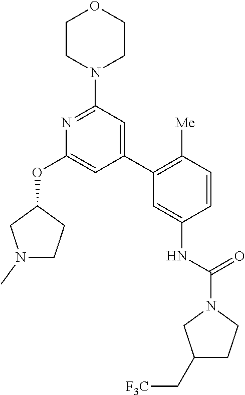 [SMILES: Cc1ccc(NC(=O)N2CCC(CC(F)(F)F)C2)cc1-c1cc(O[C@@H]2CCN(C)C2)nc(N2CCOCC2)c1] | N-(4-methyl-3-(2-(((R)-1- methylpyrrolidin-3-yl)oxy)-6- morpholinopyridin-4-yl)phenyl)-3- (2,2,2-trifluoroethyl)pyrrolidine-1- carboxamide |
| 203 | 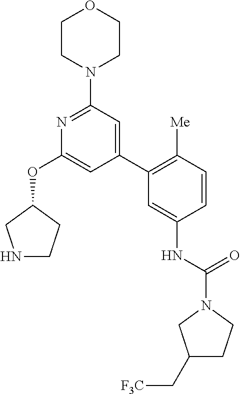 [SMILES: Cc1ccc(NC(=O)N2CCC(CC(F)(F)F)C2)cc1-c1cc(O[C@@H]2CCNC2)nc(N2CCOCC2)c1] | N-(4-methyl-3-(2-morpholino-6-((R)- pyrrolidin-3-yloxy)pyridin-4- yl)phenyl)-3-(2,2,2- trifluoroethyl)pyrrolidine-1- carboxamide |
| 204 | 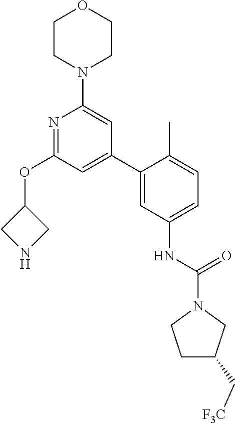 [SMILES: Cc1ccc(NC(=O)N2CC[C@@H](CC(F)(F)F)C2)cc1-c1cc(OC2CNC2)nc(N2CCOCC2)c1] | (S)-N-(3-(2-(azetidin-3-yloxy)-6- morpholinopyridin-4-yl)-4- methylphenyl)-3-(2,2,2- trifluoroethyl)pyrrolidine-1- carboxamide |
| 205 | 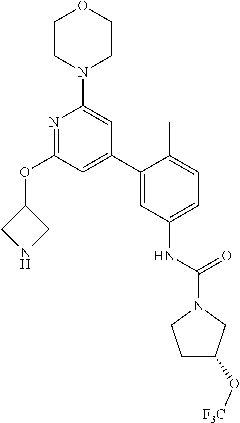 [SMILES: Cc1ccc(NC(=O)N2CC[C@@H](OC(F)(F)F)C2)cc1-c1cc(OC2CNC2)nc(N2CCOCC2)c1] | (R)-N-(3-(2-(azetidin-3-yloxy)-6- morpholinopyridin-4-yl)-4- methylphenyl)-3- (trifluoromethoxy)pyrrolidine-1- carboxamide |
| 206 | 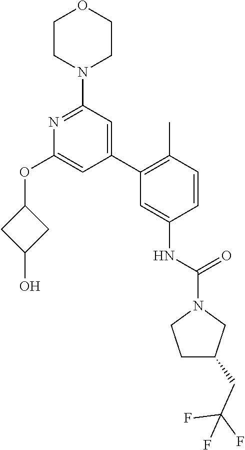 [SMILES: Cc1ccc(NC(=O)N2CC[C@@H](CC(F)(F)F)C2)cc1-c1cc(OC2CC(O)C2)nc(N2CCOCC2)c1] | (3S)-N-[3-[2-(3-hydroxycyclobutoxy)- 6-(morpholin-4-yl)pyridin-4-yl]-4- methylphenyl]-3-(2,2,2- trifluoroethyl)pyrrolidine-1- carboxamide |
| 207, 208, 209 and 210 | 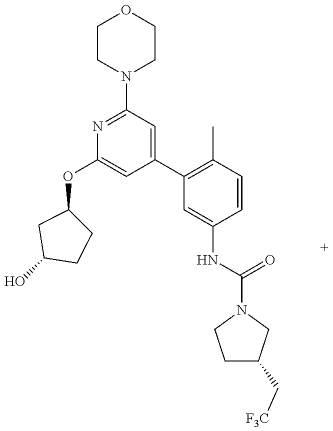 [SMILES: Cc1ccc(NC(=O)N2CC[C@@H](CC(F)(F)F)C2)cc1-c1cc(O[C@H]2CC[C@H](O)C2)nc(N2CCOCC2)c1] | (3S)-N-[3-(2-[[(1S,3S)-3- hydroxycyclopentyl]oxy]-6- (morpholin-4-yl)pyridin-4-yl)-4- methylphenyl]-3-(2,2,2- trifluoroethyl)pyrrolidine-1- carboxamide and (3S)-N-[3-(2-[[(1R,3R)-3- hydroxycyclopentyl]oxy]-6- (morpholin-4-yl)pyridin-4-yl)-4- methylphenyl]-3-(2,2,2- trifluoroethyl)pyrrolidine-1- carboxamide and |
| | 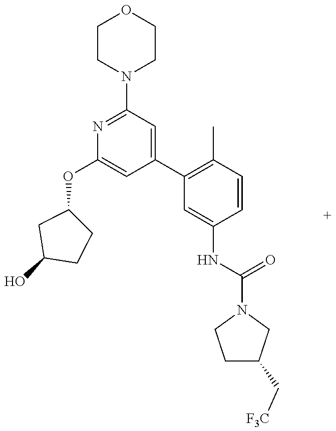 [SMILES: Cc1ccc(NC(=O)N2CC[C@@H](CC(F)(F)F)C2)cc1-c1cc(O[C@@H]2CC[C@@H](O)C2)nc(N2CCOCC2)c1] | |
| | 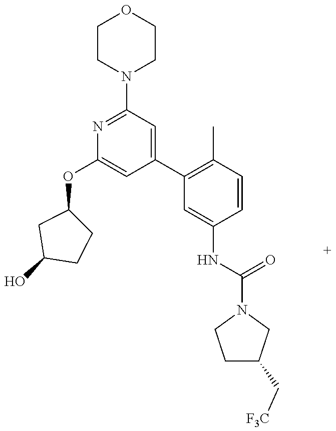 [SMILES: Cc1ccc(NC(=O)N2CC[C@@H](CC(F)(F)F)C2)cc1-c1cc(O[C@H]2CC[C@@H](O)C2)nc(N2CCOCC2)c1] | (3S)-N-[3-(2-[[(1S,3R)-3- hydroxycyclopentyl]oxy]-6- (morpholin-4-yl)pyridin-4-yl)-4- methylphenyl]-3-(2,2,2- trifluoroethyl)pyrrolidine-1- carboxamide and (3S)-N-[3-(2-[[(1R,3S)-3- hydroxycyclopentyl]oxy]-6- (morpholin-4-yl)pyridin-4-yl)-4- methylphenyl]-3-(2,2,2- trifluoroethyl)pyrrolidine-1- carboxamide |
| | 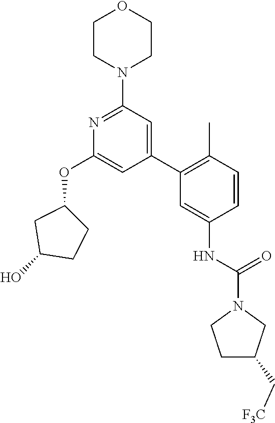 [SMILES: Cc1ccc(NC(=O)N2CC[C@@H](CC(F)(F)F)C2)cc1-c1cc(O[C@@H]2CC[C@H](O)C2)nc(N2CCOCC2)c1] | |
| 211 | 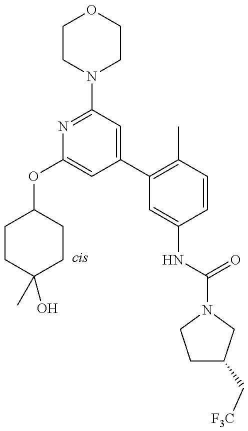 [SMILES: Cc1ccc(NC(=O)N2CC[C@@H](CC(F)(F)F)C2)cc1-c1cc(OC2CCC(C)(O)CC2)nc(N2CCOCC2)c1] | (3S)-N-(3-[2-[(4-hydroxy-4- methylcyclohexyl)oxy]-6-(morpholin- 4-yl)pyridin-4-yl]-4-methylphenyl)-3- (2,2,2-trifluoroethyl)pyrrolidine-1- carboxamide (cis) |
| 212 | 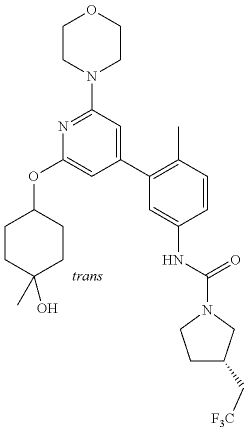 [SMILES: Cc1ccc(NC(=O)N2CC[C@@H](CC(F)(F)F)C2)cc1-c1cc(OC2CCC(C)(O)CC2)nc(N2CCOCC2)c1] | (3S)-N-(3-[2-[(4-hydroxy-4- methylcyclohexyl)oxy]-6-(morpholin- 4-yl)pyridin-4-yl]-4-methylphenyl)-3- (2,2,2-trifluoroethyl)pyrrolidine-1- carboxamide (trans) |
| 213 | 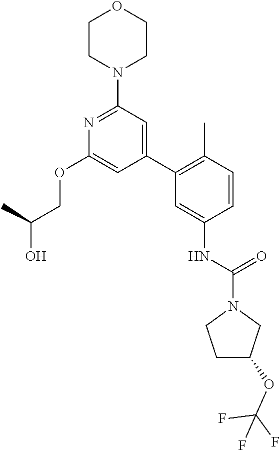 [SMILES: Cc1ccc(NC(=O)N2CC[C@@H](OC(F)(F)F)C2)cc1-c1cc(OC[C@H](C)O)nc(N2CCOCC2)c1] | (3R)-N-(3-[2-[(2S)-2- hydroxypropoxy]-6-(morpholin-4- yl)pyridin-4-yl]-4-methylphenyl)-3- (trifluoromethoxy)pyrrolidine-1- carboxamide |
| 214 | 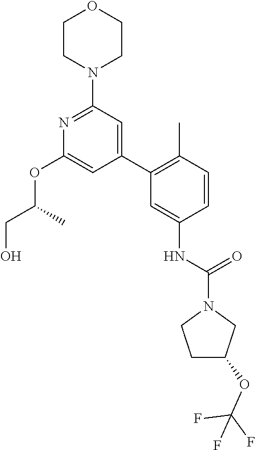 [SMILES: Cc1ccc(NC(=O)N2CC[C@@H](OC(F)(F)F)C2)cc1-c1cc(O[C@H](C)CO)nc(N2CCOCC2)c1] | (3R)-N-[3-(2-[[(2R)-1-hydroxypropan- 2-yl]oxy]-6-(morpholin-4-yl)pyridin-4- yl)-4-methylphenyl]-3- (trifluoromethoxy)pyrrolidine-1- carboxamide |
| 215 | 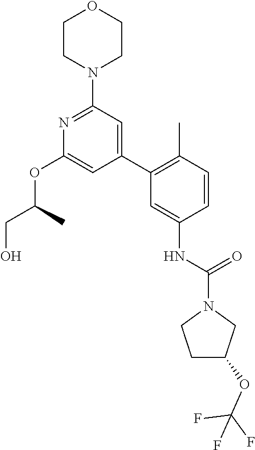 [SMILES: Cc1ccc(NC(=O)N2CC[C@@H](OC(F)(F)F)C2)cc1-c1cc(O[C@@H](C)CO)nc(N2CCOCC2)c1] | (3R)-N-[3-(2-[[(2S)-1-hydroxypropan- 2-yl]oxy]-6-(morpholin-4-yl)pyridin-4- yl)-4-methylphenyl]-3- (trifluoromethoxy)pyrrolidine-1- carboxamide |
| 216 | 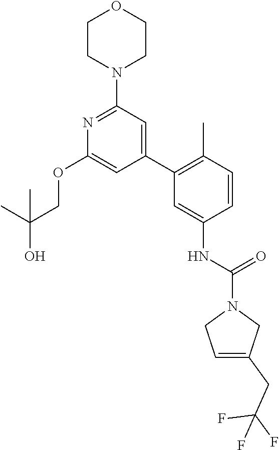 [SMILES: Cc1ccc(NC(=O)N2CC=C(CC(F)(F)F)C2)cc1-c1cc(OCC(C)(C)O)nc(N2CCOCC2)c1] | N-[3-[2-(2-hydroxy-2-methylpropoxy)- 6-(morpholin-4-yl)pyridin-4-yl]-4- methylphenyl]-3-(2,2,2-trifluoroethyl)- 2,5-dihydropyrrole-1-carboxamide |
| 217 | 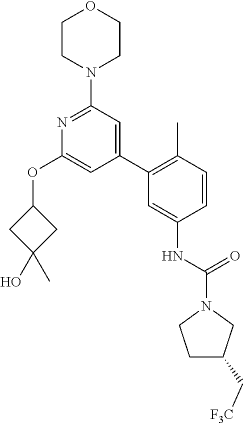 [SMILES: Cc1ccc(NC(=O)N2CC[C@@H](CC(F)(F)F)C2)cc1-c1cc(OC2CC(C)(O)C2)nc(N2CCOCC2)c1] | (3S)-N-[3-[2-(3-hydroxy-3- methylcyclobutoxy)-6-(morpholin-4- yl)pyridin-4-yl]-4-methylphenyl]-3- (2,2,2-trifluoroethyl)pyrrolidine-1- carboxamide |
| 218 | 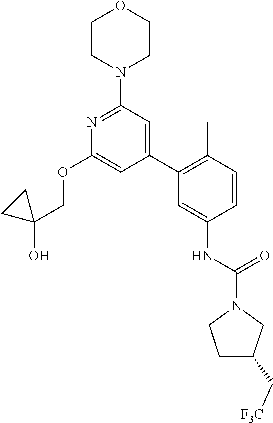 [SMILES: Cc1ccc(NC(=O)N2CC[C@@H](CC(F)(F)F)C2)cc1-c1cc(OCC2(O)CC2)nc(N2CCOCC2)c1] | (3S)-N-(3-[2-[(1- hydroxycyclopropyl)methoxy]-6- (morpholin-4-yl)pyridin-4-yl]-4- methylphenyl)-3-(2,2,2- trifluoroethyl)pyrrolidine-1- carboxamide |
| 219 | 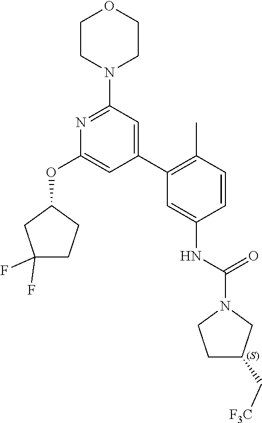 | (3S)-N-[3-(2-[[(1R)-3,3- difluorocyclopentyl]oxy]-6- (morpholin-4-yl)pyridin-4-yl)-4- methylphenyl]-3-(2,2,2- trifluoroethyl)pyrrolidine-1- carboxamide |
| 220 | 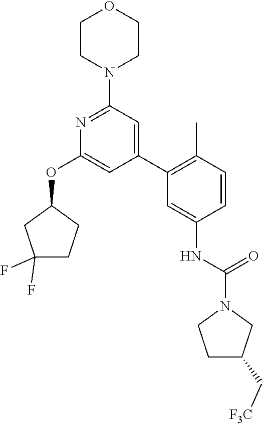 [SMILES: Cc1ccc(NC(=O)N2CC[C@@H](CC(F)(F)F)C2)cc1-c1cc(O[C@H]2CCC(F)(F)C2)nc(N2CCOCC2)c1] | (3S)-N-[3-(2-[[(1S)-3,3- difluorocyclopentyl]oxy]-6- (morpholin-4-yl)pyridin-4-yl)-4- methylphenyl]-3-(2,2,2- trifluoroethyl)pyrrolidine-1- carboxamide |
| 221 | 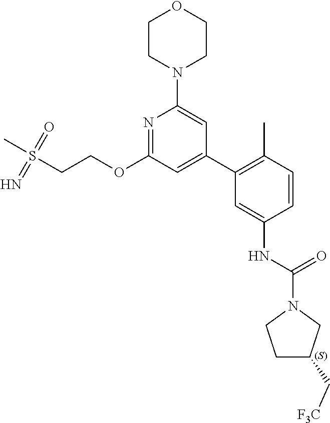 | (3S)-N-[3-(2-[2-[imino(methyl)oxo-λ⁶- sulfanyl]ethoxy]-6-(morpholin-4- yl)pyridin-4-yl)-4-methylphenyl]-3- (2,2,2-trifluoroethyl)pyrrolidine-1- carboxamide |
| 222 | 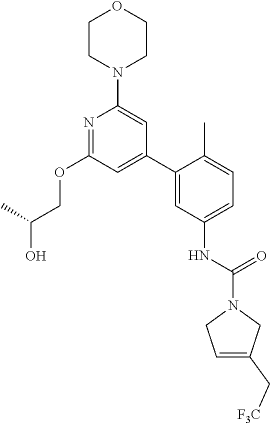 [SMILES: Cc1ccc(NC(=O)N2CC=C(CC(F)(F)F)C2)cc1-c1cc(OC[C@@H](C)O)nc(N2CCOCC2)c1] | (R)-N-(3-(2-(2-hydroxypropoxy)-6- morpholinopyridin-4-yl)-4- methylphenyl)-3-(2,2,2-trifluoroethyl)- 2,5-dihydro-1H-pyrrole-1-carboxamide |
| 223 | 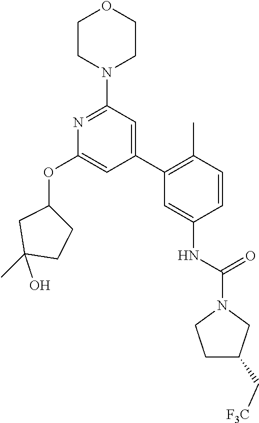 [SMILES: Cc1ccc(NC(=O)N2CC[C@@H](CC(F)(F)F)C2)cc1-c1cc(OC2CCC(C)(O)C2)nc(N2CCOCC2)c1] | (3S)-N-(3-[2-[(3-hydroxy-3- methylcyclopentyl)oxy]-6-(morpholin- 4-yl)pyridin-4-yl]-4-methylphenyl)-3- (2,2,2-trifluoroethyl)pyrrolidine-1- carboxamide |
| 224 | 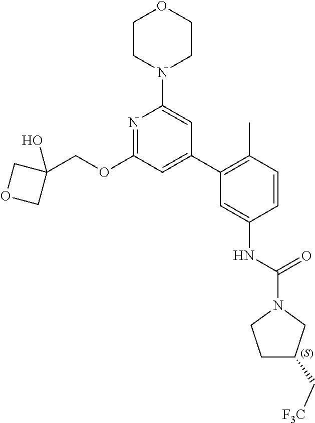 | (3S)-N-(3-[2-[(3-hydroxyoxetan-3- yl)methoxy]-6-(morpholin-4- yl)pyridin-4-yl]-4-methylphenyl)-3- (2,2,2-trifluoroethyl)pyrrolidine-1- carboxamide |
| 225 | 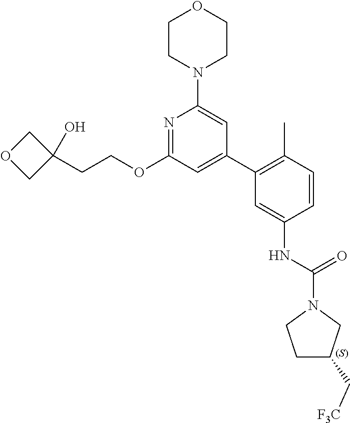 | (3S)-N-(3-[2-[2-(3-hydroxyoxetan-3- yl)ethoxy]-6-(morpholin-4-yl)pyridin- 4-yl]-4-methylphenyl)-3-(2,2,2- trifluoroethyl)pyrrolidine-1- carboxamide |
| 226 | 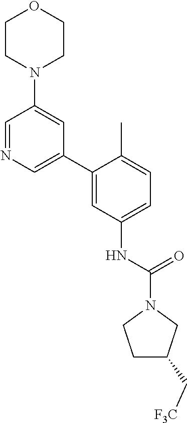 [SMILES: Cc1ccc(NC(=O)N2CC[C@@H](CC(F)(F)F)C2)cc1-c1cncc(N2CCOCC2)c1] | (3S)-N-[4-methyl-3-[5-(morpholin-4- yl)pyridin-3-yl]phenyl]-3-(2,2,2- trifluoroethyl)pyrrolidine-1- carboxamide |
| 227 | 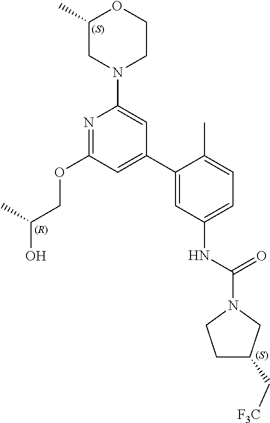 | (3S)-N-(3-[2-[(2R)-2- hydroxypropoxy]-6-[(2S)-2- methylmorpholin-4-yl]pyridin-4-yl]-4- methylphenyl)-3-(2,2,2- trifluoroethyl)pyrrolidine-1- carboxamide |
| 228 | 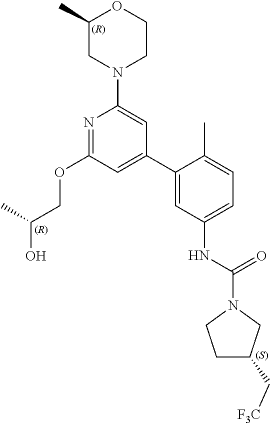 | (S)-N-(3-(2-((R)-2-hydroxypropoxy)-6- ((R)-2-methylmorpholino)pyridin-4- yl)-4-methylphenyl)-3-(2,2,2- trifluoroethyl)pyrrolidine-1- carboxamide |
| 229 | 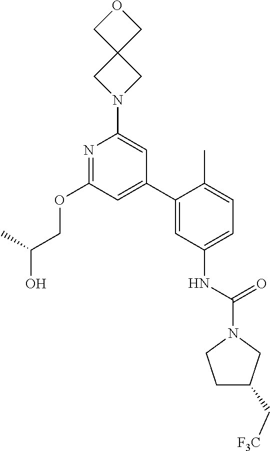 [SMILES: Cc1ccc(NC(=O)N2CC[C@@H](CC(F)(F)F)C2)cc1-c1cc(OC[C@@H](C)O)nc(N2CC3(COC3)C2)c1] | (3S)-N-(3-[2-[(2R)-2- hydroxypropoxy]-6-[2-oxa-6- azaspiro[3.3]heptan-6-yl]pyridin-4-yl]- 4-methylphenyl)-3-(2,2,2- trifluoroethyl)pyrrolidine-1- carboxamide |
| 230 | 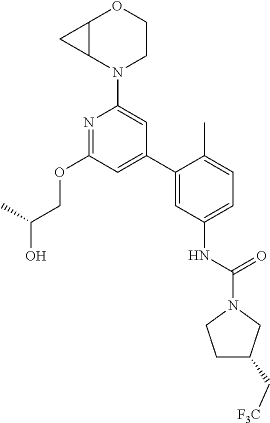 [SMILES: Cc1ccc(NC(=O)N2CC[C@@H](CC(F)(F)F)C2)cc1-c1cc(OC[C@@H](C)O)nc(N2CCOC3CC32)c1] | (3S)-N-(3-(2-(2-oxa-5- azabicyclo[4.1.0]heptan-5-yl)-6-((R)- 2-hydroxypropoxy)pyridin-4-yl)-4- methylphenyl)-3-(2,2,2- trifluoroethyl)pyrrolidine-1- carboxamide |
| 231 | 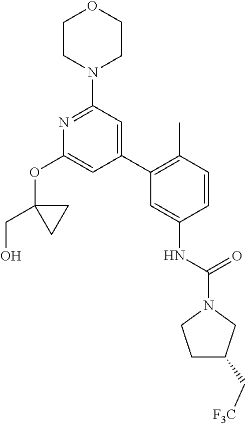 [SMILES: Cc1ccc(NC(=O)N2CC[C@@H](CC(F)(F)F)C2)cc1-c1cc(OC2(CO)CC2)nc(N2CCOCC2)c1] | (S)-N-(3-(2-(1- (hydroxymethyl)cyclopropoxy)-6- morpholinopyridin-4-yl)-4- methylphenyl)-3-(2,2,2- trifluoroethyl)pyrrolidine-1- carboxamide |
| 232 | 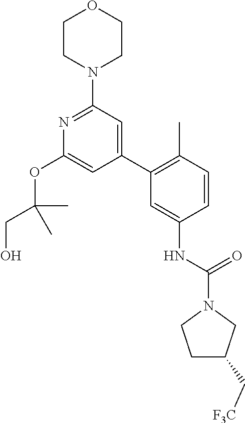 [SMILES: Cc1ccc(NC(=O)N2CC[C@@H](CC(F)(F)F)C2)cc1-c1cc(OC(C)(C)CO)nc(N2CCOCC2)c1] | (S)-N-(3-(2-((1-hydroxy-2- methylpropan-2-yl)oxy)-6- morpholinopyridin-4-yl)-4- methylphenyl)-3-(2,2,2- trifluoroethyl)pyrrolidine-1- carboxamide |
| 233 | 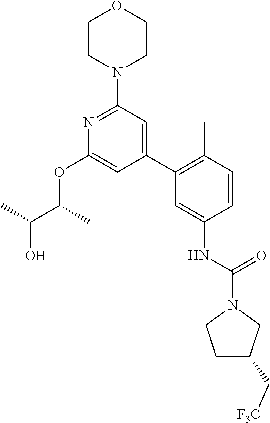 [SMILES: Cc1ccc(NC(=O)N2CC[C@@H](CC(F)(F)F)C2)cc1-c1cc(O[C@H](C)[C@@H](C)O)nc(N2CCOCC2)c1] | (S)-N-(3-(2-(((2R,3R)-3- hydroxybutan-2-yl)oxy)-6- morpholinopyridin-4-yl)-4- methylphenyl)-3-(2,2,2- trifluoroethyl)pyrrolidine-1- carboxamide |

In some embodiments, the heteroaromatic RAF kinase inhibitory compound as described herein has a structure provided below.

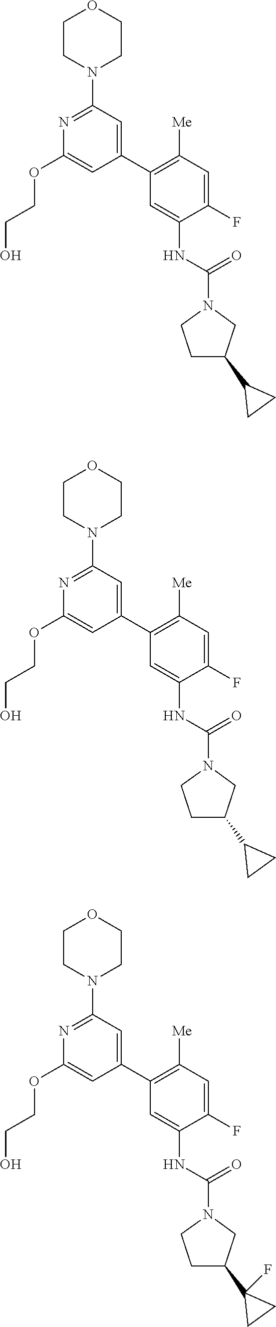 [SMILES: Cc1cc(F)c(NC(=O)N2CC[C@H](C3CC3)C2)cc1-c1cc(OCCO)nc(N2CCOCC2)c1 ; Cc1cc(F)c(NC(=O)N2CC[C@@H](C3CC3)C2)cc1-c1cc(OCCO)nc(N2CCOCC2)c1 ; Cc1cc(F)c(NC(=O)N2CC[C@H](C3(F)CC3)C2)cc1-c1cc(OCCO)nc(N2CCOCC2)c1]

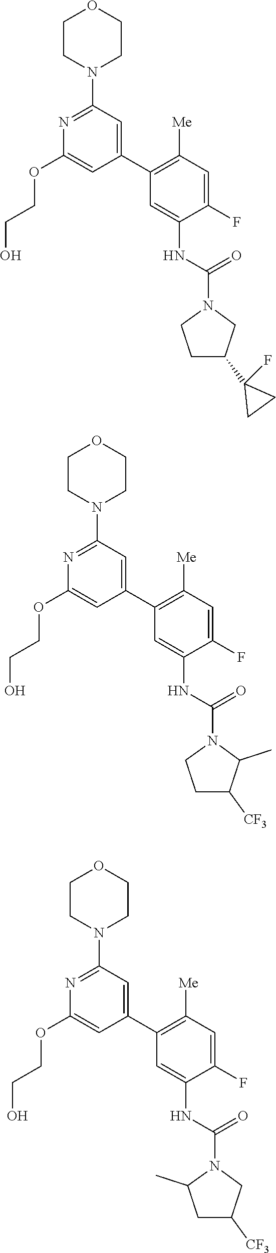 [SMILES: Cc1cc(F)c(NC(=O)N2CC[C@@H](C3(F)CC3)C2)cc1-c1cc(OCCO)nc(N2CCOCC2)c1 ; Cc1cc(F)c(NC(=O)N2CCC(C(F)(F)F)C2C)cc1-c1cc(OCCO)nc(N2CCOCC2)c1 ; Cc1cc(F)c(NC(=O)N2CC(C(F)(F)F)CC2C)cc1-c1cc(OCCO)nc(N2CCOCC2)c1]

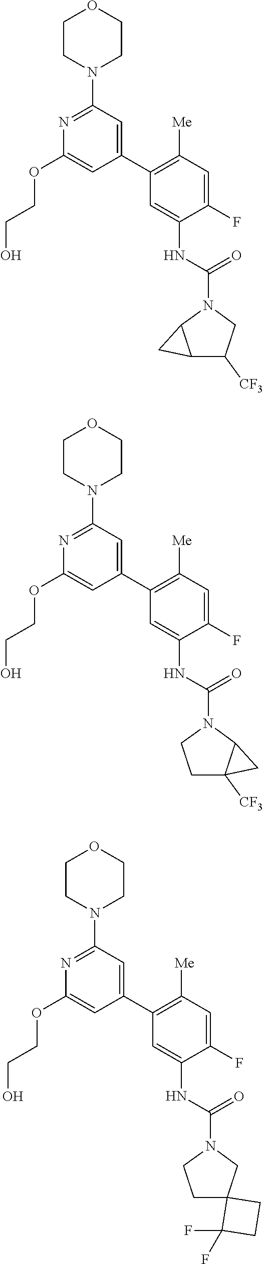 [SMILES: Cc1cc(F)c(NC(=O)N2CC(C(F)(F)F)C3CC32)cc1-c1cc(OCCO)nc(N2CCOCC2)c1 ; Cc1cc(F)c(NC(=O)N2CCC3(C(F)(F)F)CC23)cc1-c1cc(OCCO)nc(N2CCOCC2)c1 ; Cc1cc(F)c(NC(=O)N2CCC3(CCC3(F)F)C2)cc1-c1cc(OCCO)nc(N2CCOCC2)c1]

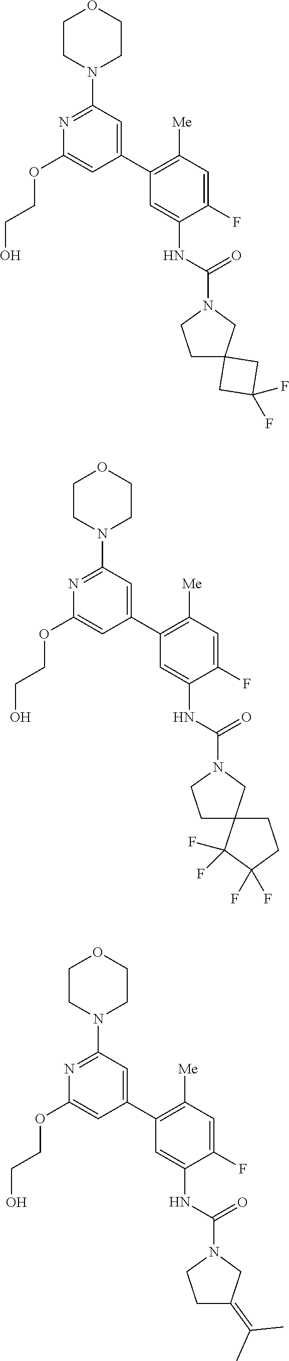 [SMILES: Cc1cc(F)c(NC(=O)N2CCC3(C2)CC(F)(F)C3)cc1-c1cc(OCCO)nc(N2CCOCC2)c1 ; Cc1cc(F)c(NC(=O)N2CCC3(CCC(F)(F)C3(F)F)C2)cc1-c1cc(OCCO)nc(N2CCOCC2)c1 ; CC(C)=C1CCN(C(=O)Nc2cc(-c3cc(OCCO)nc(N4CCOCC4)c3)c(C)cc2F)C1]

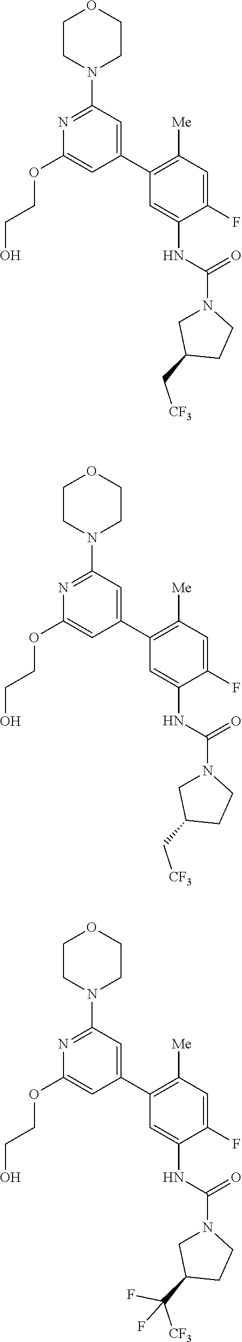 [SMILES: Cc1cc(F)c(NC(=O)N2CC[C@@H](CC(F)(F)F)C2)cc1-c1cc(OCCO)nc(N2CCOCC2)c1 ; Cc1cc(F)c(NC(=O)N2CC[C@H](CC(F)(F)F)C2)cc1-c1cc(OCCO)nc(N2CCOCC2)c1 ; Cc1cc(F)c(NC(=O)N2CC[C@@H](C(F)(F)C(F)(F)F)C2)cc1-c1cc(OCCO)nc(N2CCOCC2)c1]

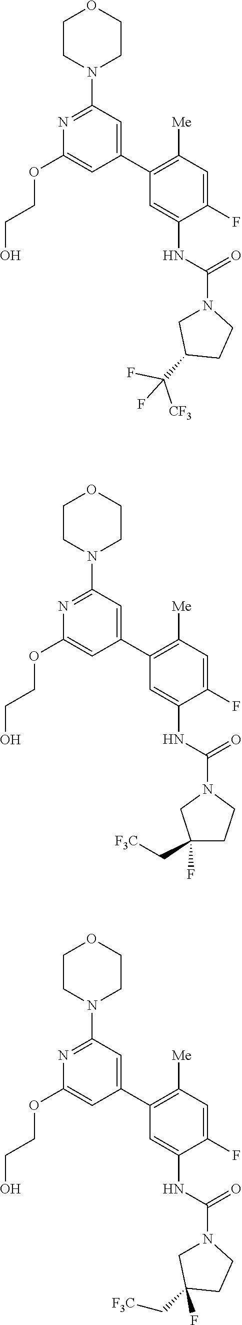 [SMILES: Cc1cc(F)c(NC(=O)N2CC[C@H](C(F)(F)C(F)(F)F)C2)cc1-c1cc(OCCO)nc(N2CCOCC2)c1 ; Cc1cc(F)c(NC(=O)N2CC[C@@](F)(CC(F)(F)F)C2)cc1-c1cc(OCCO)nc(N2CCOCC2)c1 ; Cc1cc(F)c(NC(=O)N2CC[C@](F)(CC(F)(F)F)C2)cc1-c1cc(OCCO)nc(N2CCOCC2)c1]

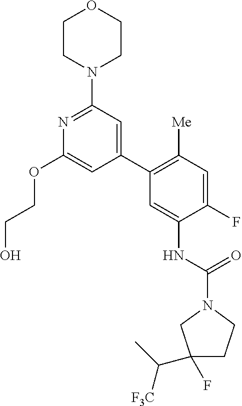 [SMILES: Cc1cc(F)c(NC(=O)N2CCC(F)(C(C)C(F)(F)F)C2)cc1-c1cc(OCCO)nc(N2CCOCC2)c1]

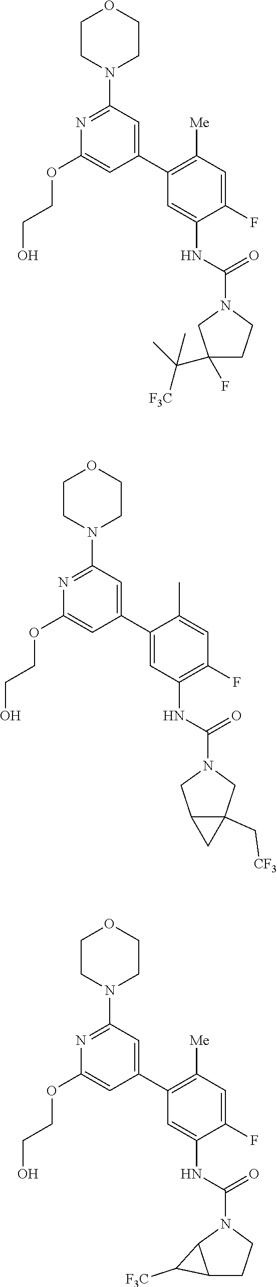 [SMILES: Cc1cc(F)c(NC(=O)N2CCC(F)(C(C)(C)C(F)(F)F)C2)cc1-c1cc(OCCO)nc(N2CCOCC2)c1 ; Cc1cc(F)c(NC(=O)N2CC3CC3(CC(F)(F)F)C2)cc1-c1cc(OCCO)nc(N2CCOCC2)c1 ; Cc1cc(F)c(NC(=O)N2CCC3C2C3C(F)(F)F)cc1-c1cc(OCCO)nc(N2CCOCC2)c1]

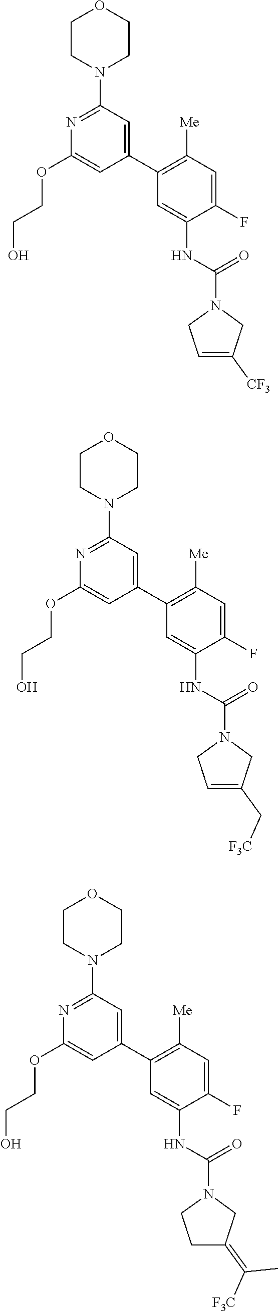 [SMILES: Cc1cc(F)c(NC(=O)N2CC=C(C(F)(F)F)C2)cc1-c1cc(OCCO)nc(N2CCOCC2)c1 ; Cc1cc(F)c(NC(=O)N2CC=C(CC(F)(F)F)C2)cc1-c1cc(OCCO)nc(N2CCOCC2)c1 ; C/C(=C1/CCN(C(=O)Nc2cc(-c3cc(OCCO)nc(N4CCOCC4)c3)c(C)cc2F)C1)C(F)(F)F]

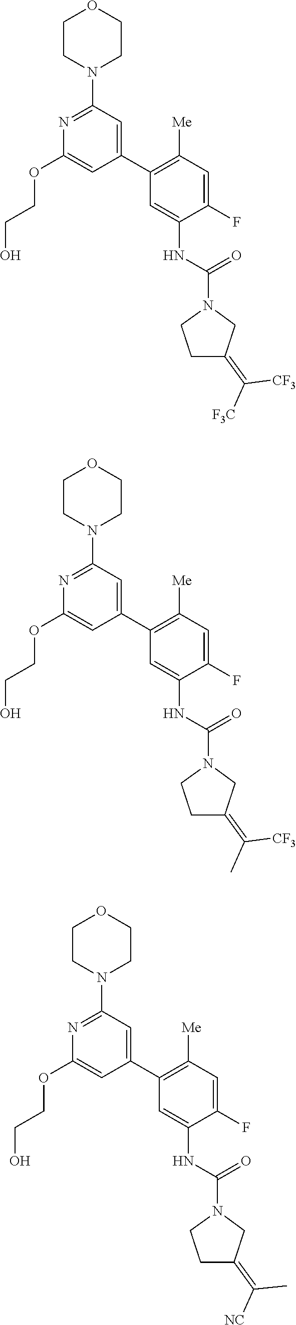 [SMILES: Cc1cc(F)c(NC(=O)N2CCC(=C(C(F)(F)F)C(F)(F)F)C2)cc1-c1cc(OCCO)nc(N2CCOCC2)c1 ; C/C(=C1\CCN(C(=O)Nc2cc(-c3cc(OCCO)nc(N4CCOCC4)c3)c(C)cc2F)C1)C(F)(F)F ; C/C(C#N)=C1/CCN(C(=O)Nc2cc(-c3cc(OCCO)nc(N4CCOCC4)c3)c(C)cc2F)C1]

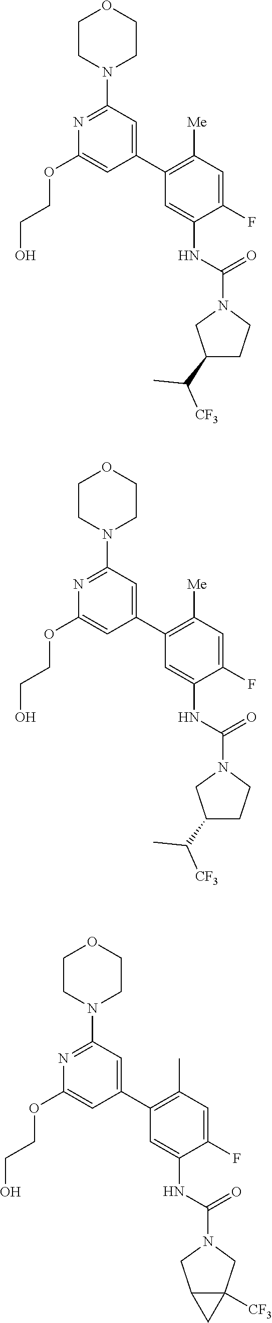 [SMILES: Cc1cc(F)c(NC(=O)N2CC[C@@H](C(C)C(F)(F)F)C2)cc1-c1cc(OCCO)nc(N2CCOCC2)c1 ; Cc1cc(F)c(NC(=O)N2CC[C@H](C(C)C(F)(F)F)C2)cc1-c1cc(OCCO)nc(N2CCOCC2)c1 ; Cc1cc(F)c(NC(=O)N2CC3CC3(C(F)(F)F)C2)cc1-c1cc(OCCO)nc(N2CCOCC2)c1]

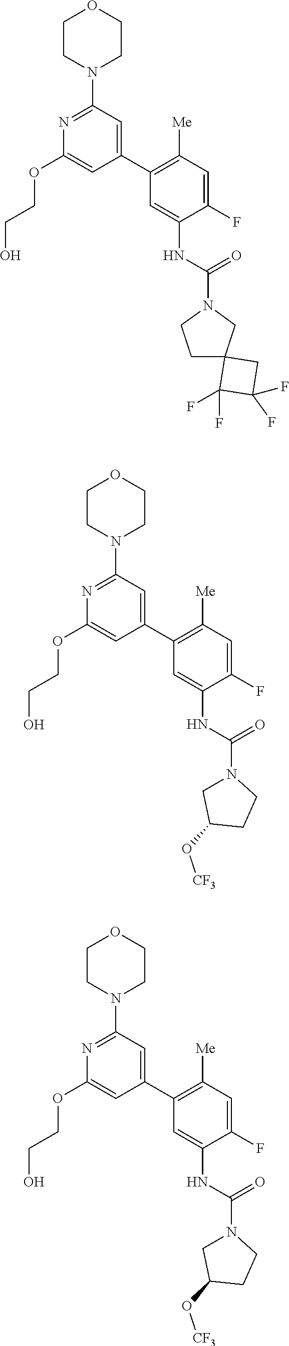 [SMILES: Cc1cc(F)c(NC(=O)N2CCC3(C2)CC(F)(F)C3(F)F)cc1-c1cc(OCCO)nc(N2CCOCC2)c1 ; Cc1cc(F)c(NC(=O)N2CC[C@H](OC(F)(F)F)C2)cc1-c1cc(OCCO)nc(N2CCOCC2)c1 ; Cc1cc(F)c(NC(=O)N2CC[C@@H](OC(F)(F)F)C2)cc1-c1cc(OCCO)nc(N2CCOCC2)c1]

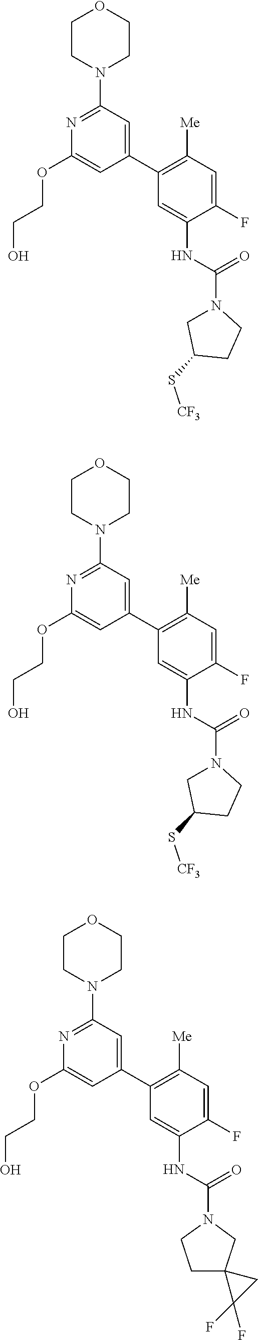 [SMILES: Cc1cc(F)c(NC(=O)N2CC[C@H](SC(F)(F)F)C2)cc1-c1cc(OCCO)nc(N2CCOCC2)c1 ; Cc1cc(F)c(NC(=O)N2CC[C@@H](SC(F)(F)F)C2)cc1-c1cc(OCCO)nc(N2CCOCC2)c1 ; Cc1cc(F)c(NC(=O)N2CCC3(C2)CC3(F)F)cc1-c1cc(OCCO)nc(N2CCOCC2)c1]

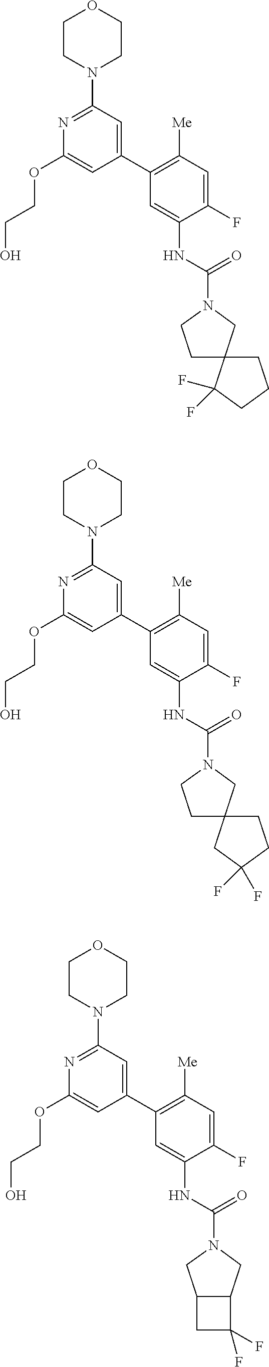 [SMILES: Cc1cc(F)c(NC(=O)N2CCC3(CCCC3(F)F)C2)cc1-c1cc(OCCO)nc(N2CCOCC2)c1 ; Cc1cc(F)c(NC(=O)N2CCC3(CCC(F)(F)C3)C2)cc1-c1cc(OCCO)nc(N2CCOCC2)c1 ; Cc1cc(F)c(NC(=O)N2CC3CC(F)(F)C3C2)cc1-c1cc(OCCO)nc(N2CCOCC2)c1]

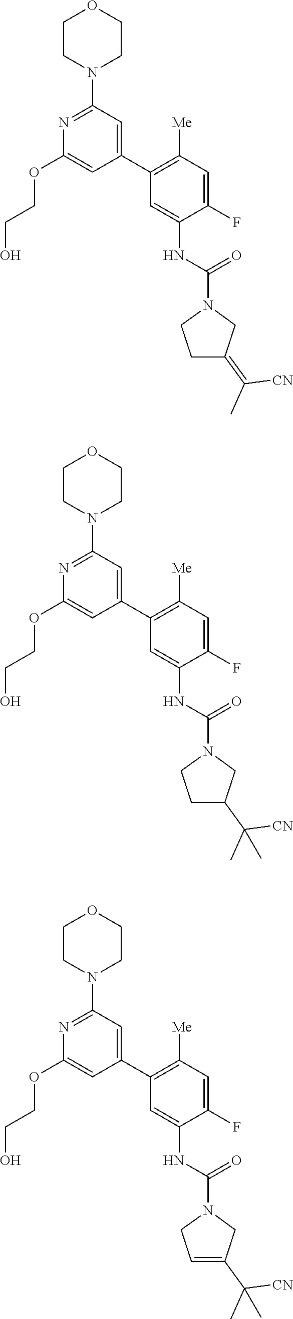 [SMILES: C/C(C#N)=C1\CCN(C(=O)Nc2cc(-c3cc(OCCO)nc(N4CCOCC4)c3)c(C)cc2F)C1 ; Cc1cc(F)c(NC(=O)N2CCC(C(C)(C)C#N)C2)cc1-c1cc(OCCO)nc(N2CCOCC2)c1 ; Cc1cc(F)c(NC(=O)N2CC=C(C(C)(C)C#N)C2)cc1-c1cc(OCCO)nc(N2CCOCC2)c1]

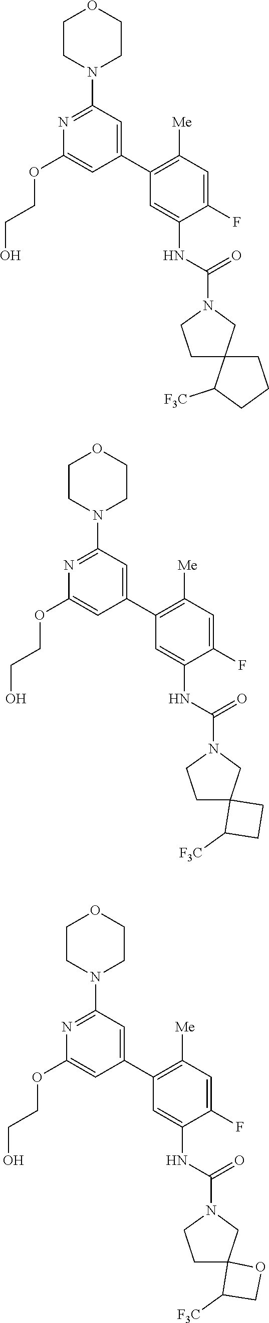 [SMILES: Cc1cc(F)c(NC(=O)N2CCC3(CCCC3C(F)(F)F)C2)cc1-c1cc(OCCO)nc(N2CCOCC2)c1 ; Cc1cc(F)c(NC(=O)N2CCC3(CCC3C(F)(F)F)C2)cc1-c1cc(OCCO)nc(N2CCOCC2)c1 ; Cc1cc(F)c(NC(=O)N2CCC3(C2)OCC3C(F)(F)F)cc1-c1cc(OCCO)nc(N2CCOCC2)c1]

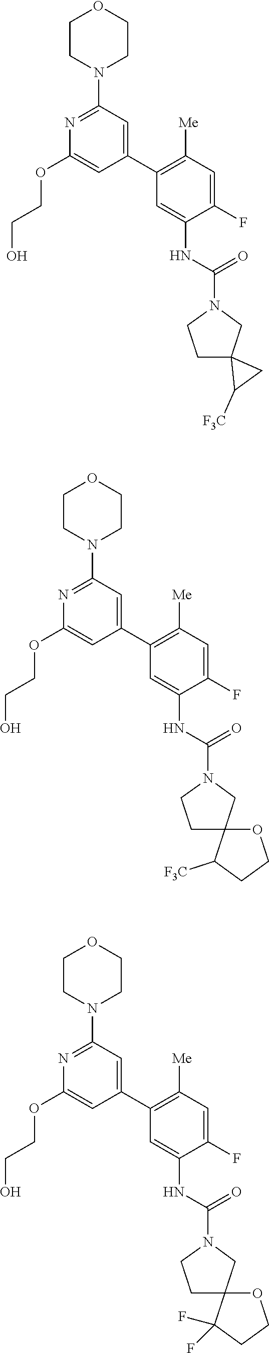 [SMILES: Cc1cc(F)c(NC(=O)N2CCC3(CC3C(F)(F)F)C2)cc1-c1cc(OCCO)nc(N2CCOCC2)c1 ; Cc1cc(F)c(NC(=O)N2CCC3(C2)OCCC3C(F)(F)F)cc1-c1cc(OCCO)nc(N2CCOCC2)c1 ; Cc1cc(F)c(NC(=O)N2CCC3(C2)OCCC3(F)F)cc1-c1cc(OCCO)nc(N2CCOCC2)c1]

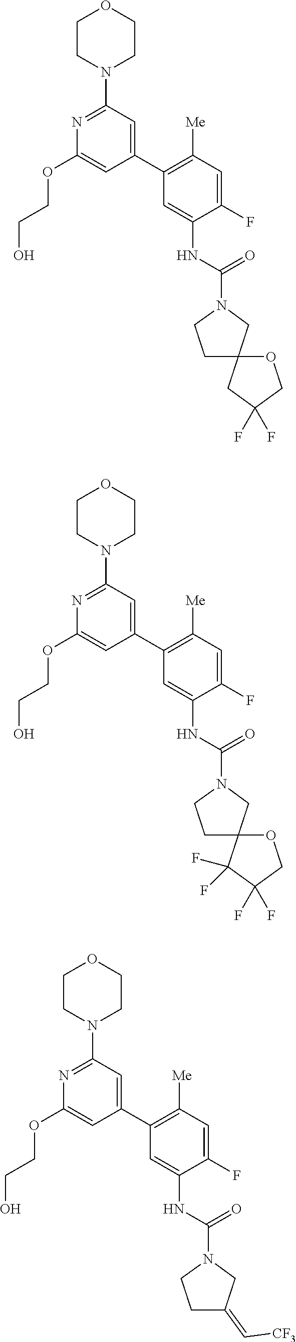 [SMILES: Cc1cc(F)c(NC(=O)N2CCC3(C2)CC(F)(F)CO3)cc1-c1cc(OCCO)nc(N2CCOCC2)c1 ; Cc1cc(F)c(NC(=O)N2CCC3(C2)OCC(F)(F)C3(F)F)cc1-c1cc(OCCO)nc(N2CCOCC2)c1 ; Cc1cc(F)c(NC(=O)N2CC/C(=C/C(F)(F)F)C2)cc1-c1cc(OCCO)nc(N2CCOCC2)c1]

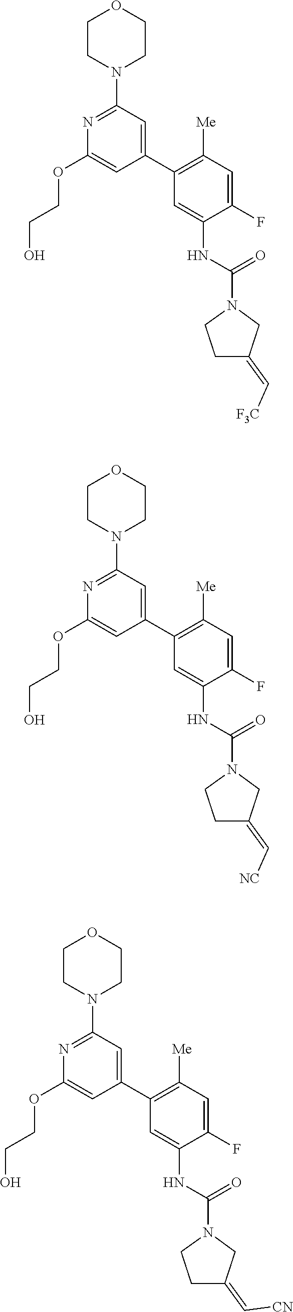 [SMILES: Cc1cc(F)c(NC(=O)N2CC/C(=C\C(F)(F)F)C2)cc1-c1cc(OCCO)nc(N2CCOCC2)c1 ; Cc1cc(F)c(NC(=O)N2CC/C(=C\C#N)C2)cc1-c1cc(OCCO)nc(N2CCOCC2)c1 ; Cc1cc(F)c(NC(=O)N2CC/C(=C/C#N)C2)cc1-c1cc(OCCO)nc(N2CCOCC2)c1]

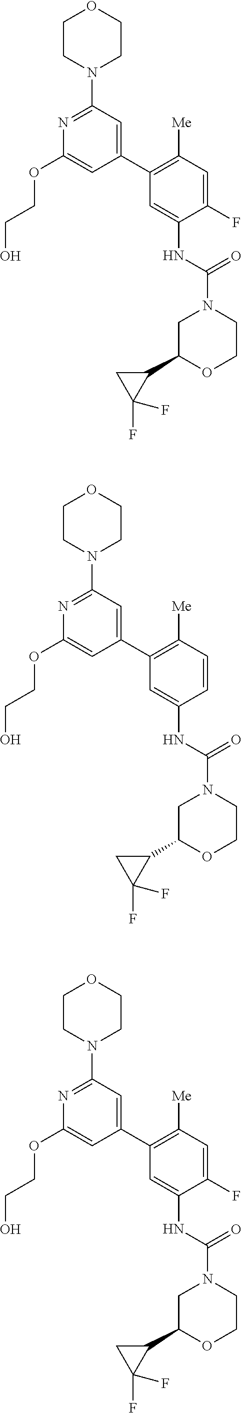 [SMILES: Cc1cc(F)c(NC(=O)N2CCO[C@@H](C3CC3(F)F)C2)cc1-c1cc(OCCO)nc(N2CCOCC2)c1 ; Cc1ccc(NC(=O)N2CCO[C@H](C3CC3(F)F)C2)cc1-c1cc(OCCO)nc(N2CCOCC2)c1 ; Cc1cc(F)c(NC(=O)N2CCO[C@@H](C3CC3(F)F)C2)cc1-c1cc(OCCO)nc(N2CCOCC2)c1]

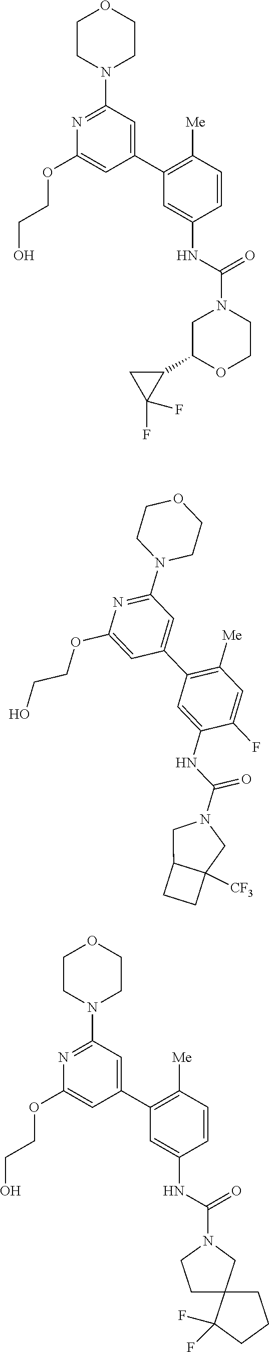 [SMILES: Cc1ccc(NC(=O)N2CCO[C@H](C3CC3(F)F)C2)cc1-c1cc(OCCO)nc(N2CCOCC2)c1 ; Cc1cc(F)c(NC(=O)N2CC3CCC3(C(F)(F)F)C2)cc1-c1cc(OCCO)nc(N2CCOCC2)c1 ; Cc1ccc(NC(=O)N2CCC3(CCCC3(F)F)C2)cc1-c1cc(OCCO)nc(N2CCOCC2)c1]

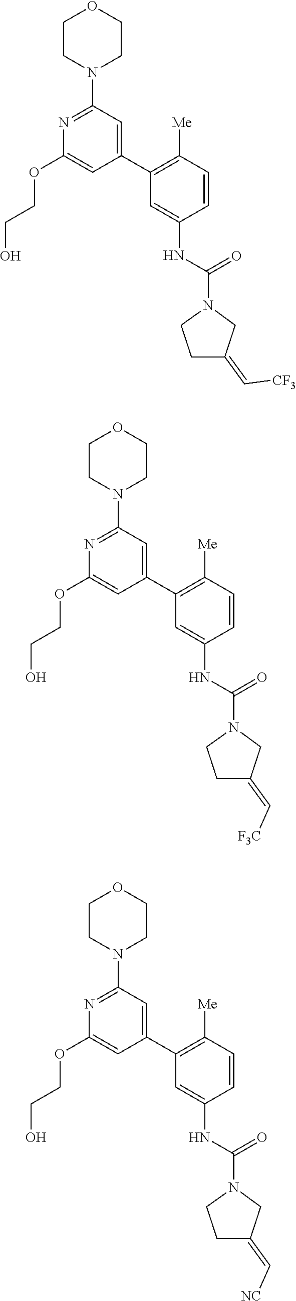 [SMILES: Cc1ccc(NC(=O)N2CC/C(=C/C(F)(F)F)C2)cc1-c1cc(OCCO)nc(N2CCOCC2)c1 ; Cc1ccc(NC(=O)N2CC/C(=C\C(F)(F)F)C2)cc1-c1cc(OCCO)nc(N2CCOCC2)c1 ; Cc1ccc(NC(=O)N2CC/C(=C\C#N)C2)cc1-c1cc(OCCO)nc(N2CCOCC2)c1]

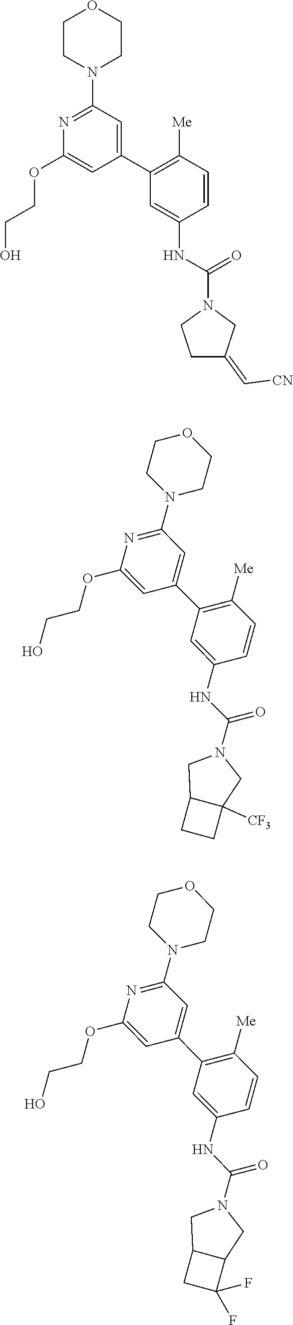 [SMILES: Cc1ccc(NC(=O)N2CC/C(=C/C#N)C2)cc1-c1cc(OCCO)nc(N2CCOCC2)c1 ; Cc1ccc(NC(=O)N2CC3CCC3(C(F)(F)F)C2)cc1-c1cc(OCCO)nc(N2CCOCC2)c1 ; Cc1ccc(NC(=O)N2CC3CC(F)(F)C3C2)cc1-c1cc(OCCO)nc(N2CCOCC2)c1]

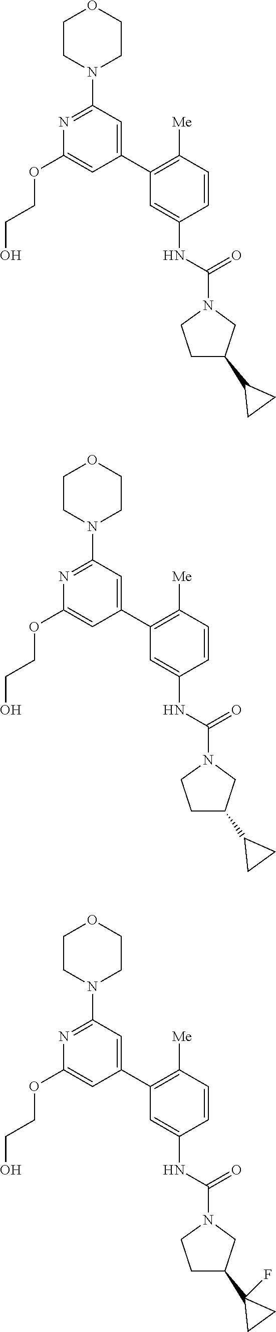 [SMILES: Cc1ccc(NC(=O)N2CC[C@H](C3CC3)C2)cc1-c1cc(OCCO)nc(N2CCOCC2)c1 ; Cc1ccc(NC(=O)N2CC[C@@H](C3CC3)C2)cc1-c1cc(OCCO)nc(N2CCOCC2)c1 ; Cc1ccc(NC(=O)N2CC[C@H](C3(F)CC3)C2)cc1-c1cc(OCCO)nc(N2CCOCC2)c1]

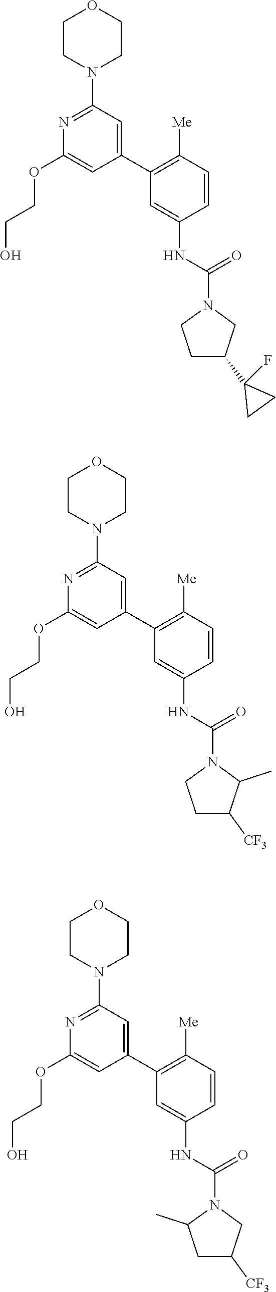 [SMILES: Cc1ccc(NC(=O)N2CC[C@@H](C3(F)CC3)C2)cc1-c1cc(OCCO)nc(N2CCOCC2)c1 ; Cc1ccc(NC(=O)N2CCC(C(F)(F)F)C2C)cc1-c1cc(OCCO)nc(N2CCOCC2)c1 ; Cc1ccc(NC(=O)N2CC(C(F)(F)F)CC2C)cc1-c1cc(OCCO)nc(N2CCOCC2)c1]

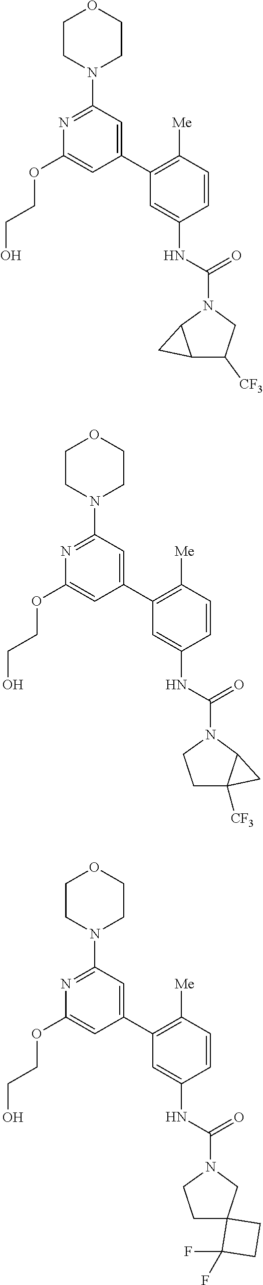 [SMILES: Cc1ccc(NC(=O)N2CC(C(F)(F)F)C3CC32)cc1-c1cc(OCCO)nc(N2CCOCC2)c1 ; Cc1ccc(NC(=O)N2CCC3(C(F)(F)F)CC23)cc1-c1cc(OCCO)nc(N2CCOCC2)c1 ; Cc1ccc(NC(=O)N2CCC3(CCC3(F)F)C2)cc1-c1cc(OCCO)nc(N2CCOCC2)c1]

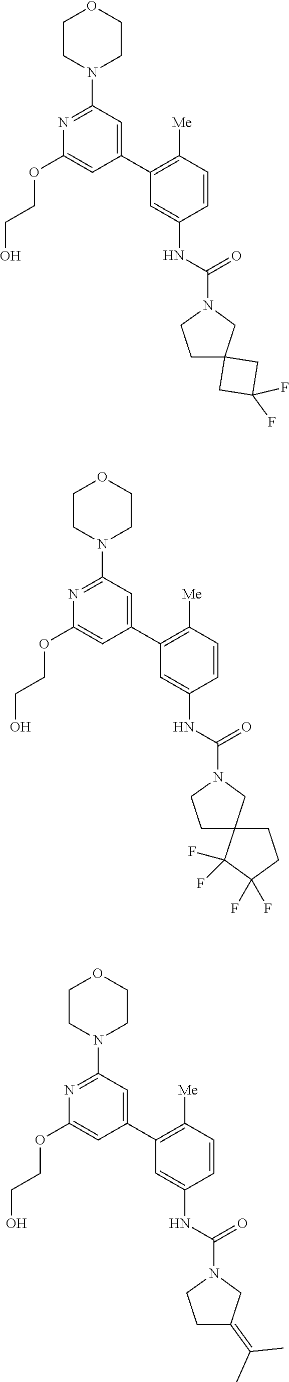 [SMILES: Cc1ccc(NC(=O)N2CCC3(C2)CC(F)(F)C3)cc1-c1cc(OCCO)nc(N2CCOCC2)c1 ; Cc1ccc(NC(=O)N2CCC3(CCC(F)(F)C3(F)F)C2)cc1-c1cc(OCCO)nc(N2CCOCC2)c1 ; CC(C)=C1CCN(C(=O)Nc2ccc(C)c(-c3cc(OCCO)nc(N4CCOCC4)c3)c2)C1]

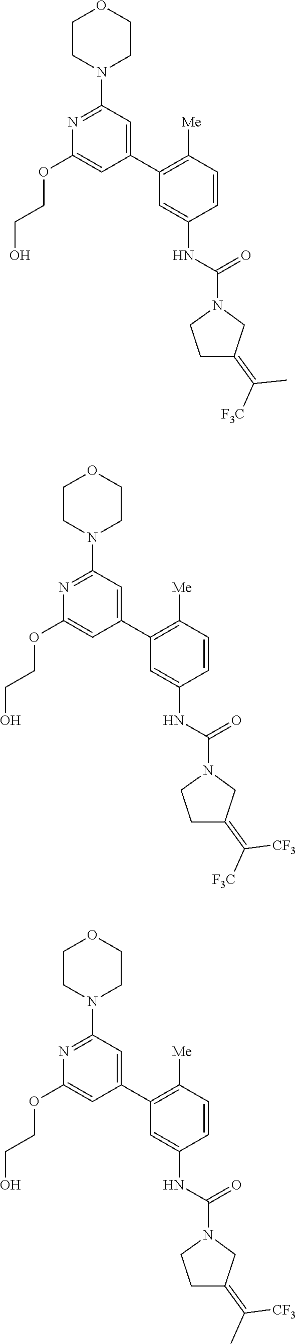 [SMILES: C/C(=C1/CCN(C(=O)Nc2ccc(C)c(-c3cc(OCCO)nc(N4CCOCC4)c3)c2)C1)C(F)(F)F ; Cc1ccc(NC(=O)N2CCC(=C(C(F)(F)F)C(F)(F)F)C2)cc1-c1cc(OCCO)nc(N2CCOCC2)c1 ; C/C(=C1\CCN(C(=O)Nc2ccc(C)c(-c3cc(OCCO)nc(N4CCOCC4)c3)c2)C1)C(F)(F)F]

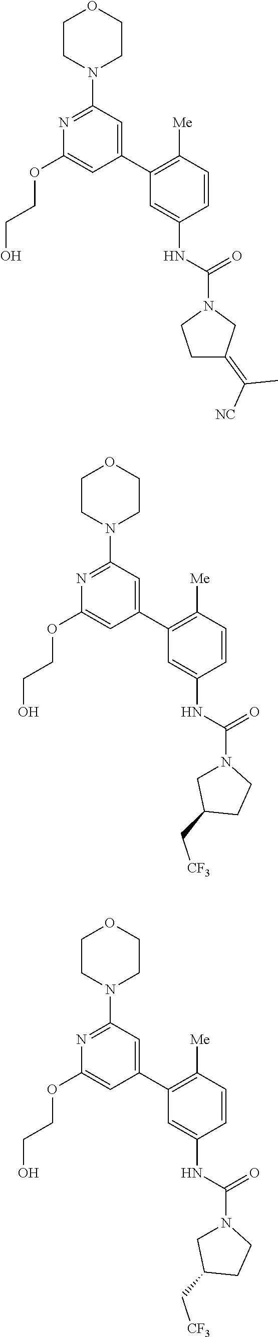 [SMILES: C/C(C#N)=C1/CCN(C(=O)Nc2ccc(C)c(-c3cc(OCCO)nc(N4CCOCC4)c3)c2)C1 ; Cc1ccc(NC(=O)N2CC[C@@H](CC(F)(F)F)C2)cc1-c1cc(OCCO)nc(N2CCOCC2)c1 ; Cc1ccc(NC(=O)N2CC[C@H](CC(F)(F)F)C2)cc1-c1cc(OCCO)nc(N2CCOCC2)c1]

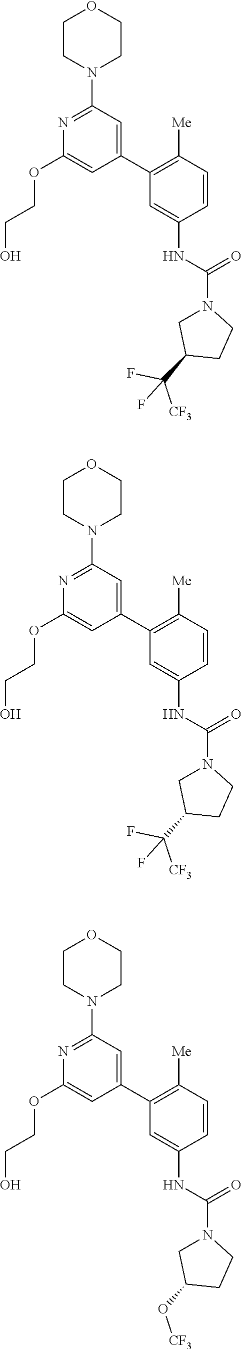 [SMILES: Cc1ccc(NC(=O)N2CC[C@@H](C(F)(F)C(F)(F)F)C2)cc1-c1cc(OCCO)nc(N2CCOCC2)c1 ; Cc1ccc(NC(=O)N2CC[C@H](C(F)(F)C(F)(F)F)C2)cc1-c1cc(OCCO)nc(N2CCOCC2)c1 ; Cc1ccc(NC(=O)N2CC[C@H](OC(F)(F)F)C2)cc1-c1cc(OCCO)nc(N2CCOCC2)c1]

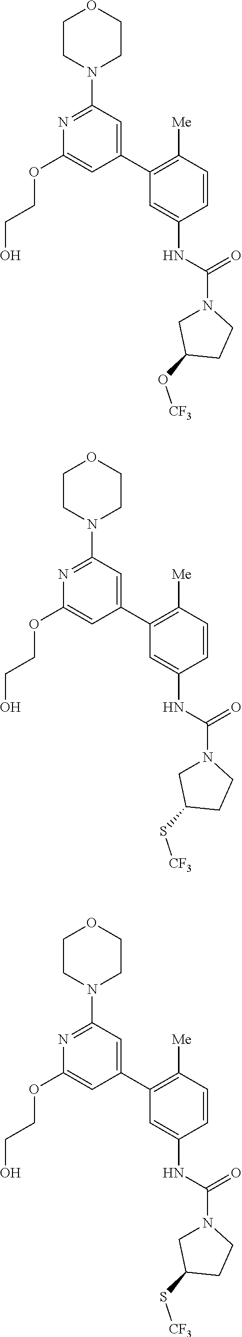 [SMILES: Cc1ccc(NC(=O)N2CC[C@@H](OC(F)(F)F)C2)cc1-c1cc(OCCO)nc(N2CCOCC2)c1 ; Cc1ccc(NC(=O)N2CC[C@H](SC(F)(F)F)C2)cc1-c1cc(OCCO)nc(N2CCOCC2)c1 ; Cc1ccc(NC(=O)N2CC[C@@H](SC(F)(F)F)C2)cc1-c1cc(OCCO)nc(N2CCOCC2)c1]

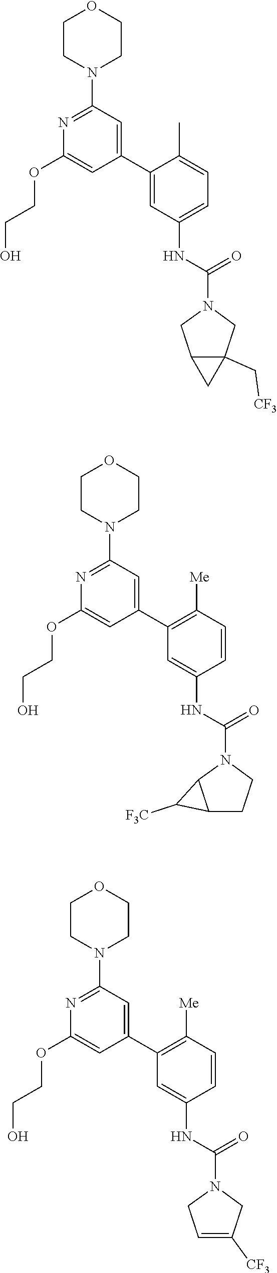 [SMILES: Cc1ccc(NC(=O)N2CC3CC3(CC(F)(F)F)C2)cc1-c1cc(OCCO)nc(N2CCOCC2)c1 ; Cc1ccc(NC(=O)N2CCC3C2C3C(F)(F)F)cc1-c1cc(OCCO)nc(N2CCOCC2)c1 ; Cc1ccc(NC(=O)N2CC=C(C(F)(F)F)C2)cc1-c1cc(OCCO)nc(N2CCOCC2)c1]

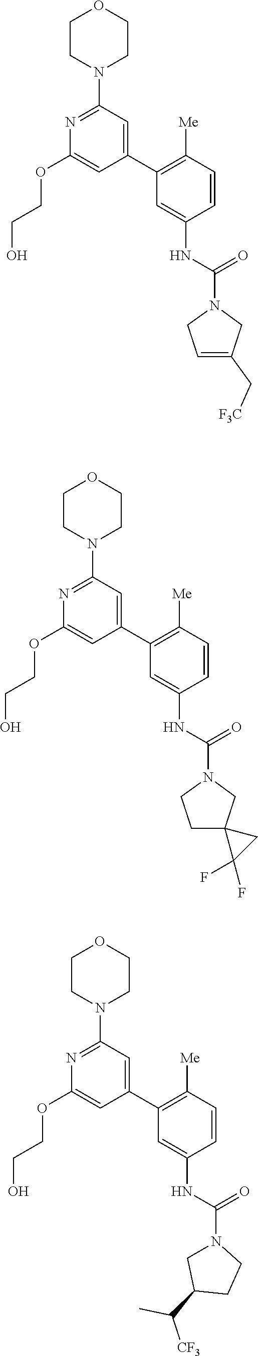 [SMILES: Cc1ccc(NC(=O)N2CC=C(CC(F)(F)F)C2)cc1-c1cc(OCCO)nc(N2CCOCC2)c1 ; Cc1ccc(NC(=O)N2CCC3(C2)CC3(F)F)cc1-c1cc(OCCO)nc(N2CCOCC2)c1 ; Cc1ccc(NC(=O)N2CC[C@@H](C(C)C(F)(F)F)C2)cc1-c1cc(OCCO)nc(N2CCOCC2)c1]

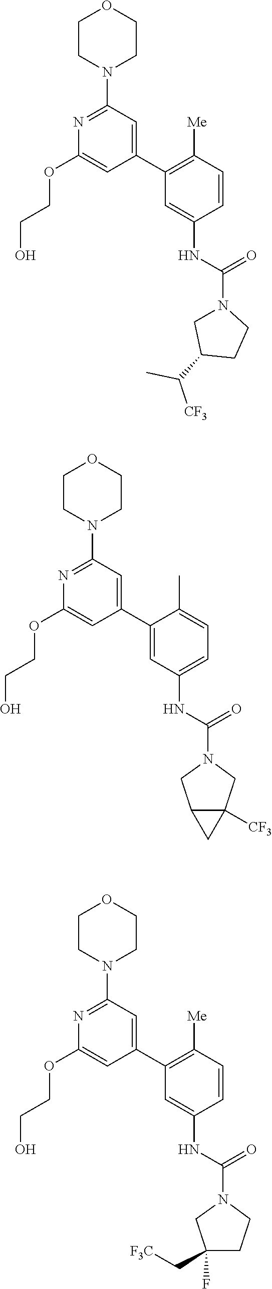 [SMILES: Cc1ccc(NC(=O)N2CC[C@H](C(C)C(F)(F)F)C2)cc1-c1cc(OCCO)nc(N2CCOCC2)c1 ; Cc1ccc(NC(=O)N2CC3CC3(C(F)(F)F)C2)cc1-c1cc(OCCO)nc(N2CCOCC2)c1 ; Cc1ccc(NC(=O)N2CC[C@@](F)(CC(F)(F)F)C2)cc1-c1cc(OCCO)nc(N2CCOCC2)c1]

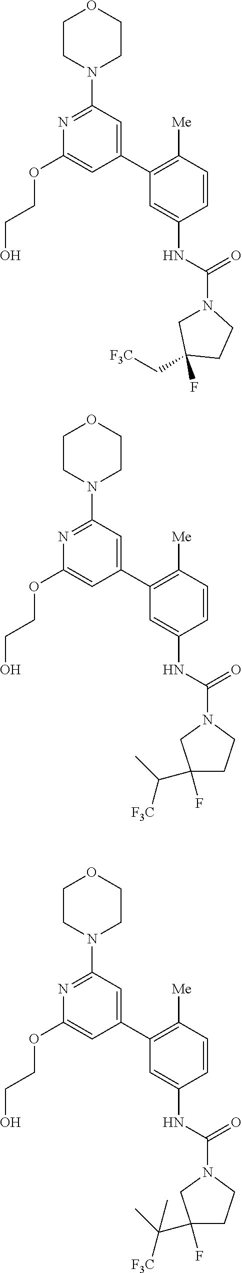 [SMILES: Cc1ccc(NC(=O)N2CC[C@](F)(CC(F)(F)F)C2)cc1-c1cc(OCCO)nc(N2CCOCC2)c1 ; Cc1ccc(NC(=O)N2CCC(F)(C(C)C(F)(F)F)C2)cc1-c1cc(OCCO)nc(N2CCOCC2)c1 ; Cc1ccc(NC(=O)N2CCC(F)(C(C)(C)C(F)(F)F)C2)cc1-c1cc(OCCO)nc(N2CCOCC2)c1]

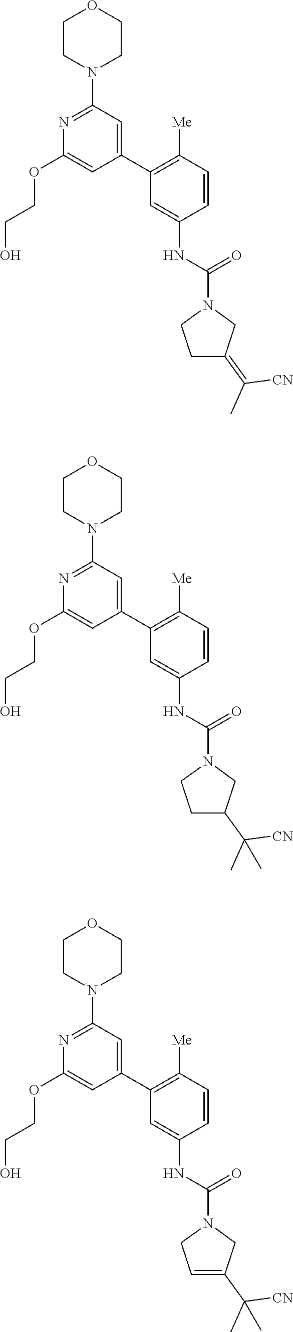 [SMILES: C/C(C#N)=C1\CCN(C(=O)Nc2ccc(C)c(-c3cc(OCCO)nc(N4CCOCC4)c3)c2)C1 ; Cc1ccc(NC(=O)N2CCC(C(C)(C)C#N)C2)cc1-c1cc(OCCO)nc(N2CCOCC2)c1 ; Cc1ccc(NC(=O)N2CC=C(C(C)(C)C#N)C2)cc1-c1cc(OCCO)nc(N2CCOCC2)c1]

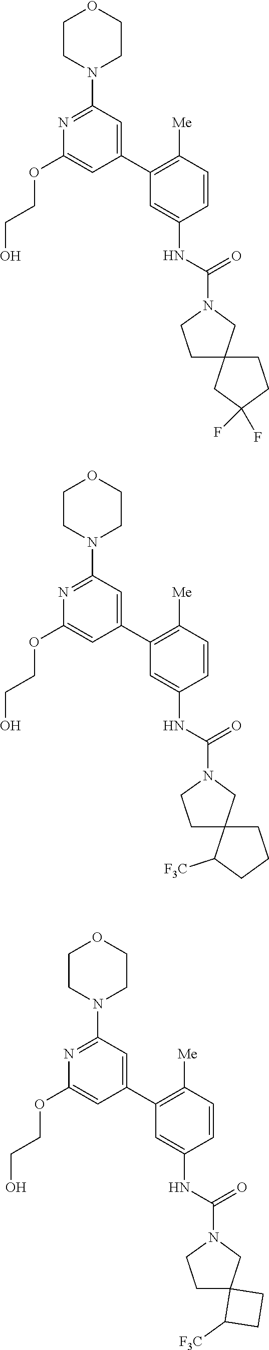 [SMILES: Cc1ccc(NC(=O)N2CCC3(CCC(F)(F)C3)C2)cc1-c1cc(OCCO)nc(N2CCOCC2)c1 ; Cc1ccc(NC(=O)N2CCC3(CCCC3C(F)(F)F)C2)cc1-c1cc(OCCO)nc(N2CCOCC2)c1 ; Cc1ccc(NC(=O)N2CCC3(CCC3C(F)(F)F)C2)cc1-c1cc(OCCO)nc(N2CCOCC2)c1]

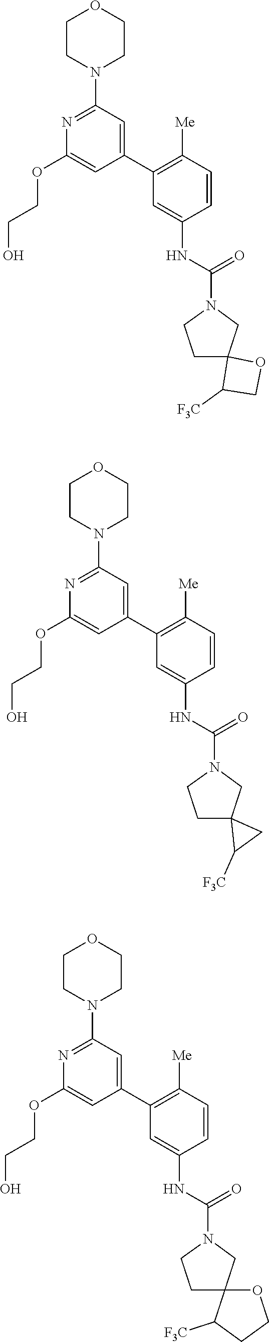 [SMILES: Cc1ccc(NC(=O)N2CCC3(C2)OCC3C(F)(F)F)cc1-c1cc(OCCO)nc(N2CCOCC2)c1 ; Cc1ccc(NC(=O)N2CCC3(CC3C(F)(F)F)C2)cc1-c1cc(OCCO)nc(N2CCOCC2)c1 ; Cc1ccc(NC(=O)N2CCC3(C2)OCCC3C(F)(F)F)cc1-c1cc(OCCO)nc(N2CCOCC2)c1]

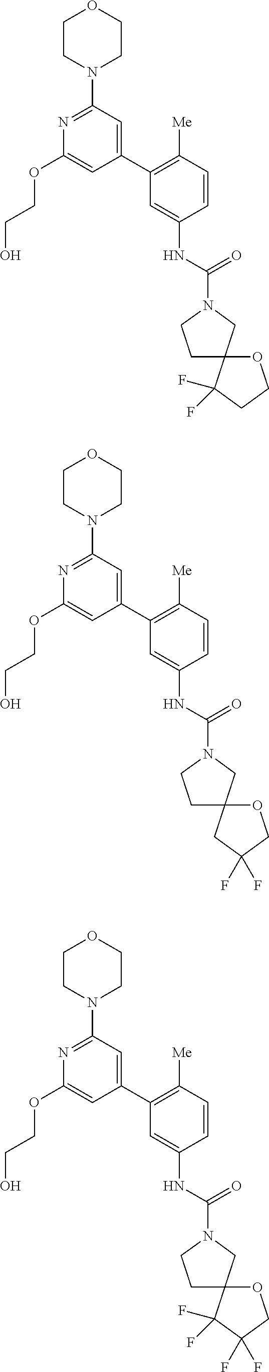 [SMILES: Cc1ccc(NC(=O)N2CCC3(C2)OCCC3(F)F)cc1-c1cc(OCCO)nc(N2CCOCC2)c1 ; Cc1ccc(NC(=O)N2CCC3(C2)CC(F)(F)CO3)cc1-c1cc(OCCO)nc(N2CCOCC2)c1 ; Cc1ccc(NC(=O)N2CCC3(C2)OCC(F)(F)C3(F)F)cc1-c1cc(OCCO)nc(N2CCOCC2)c1]

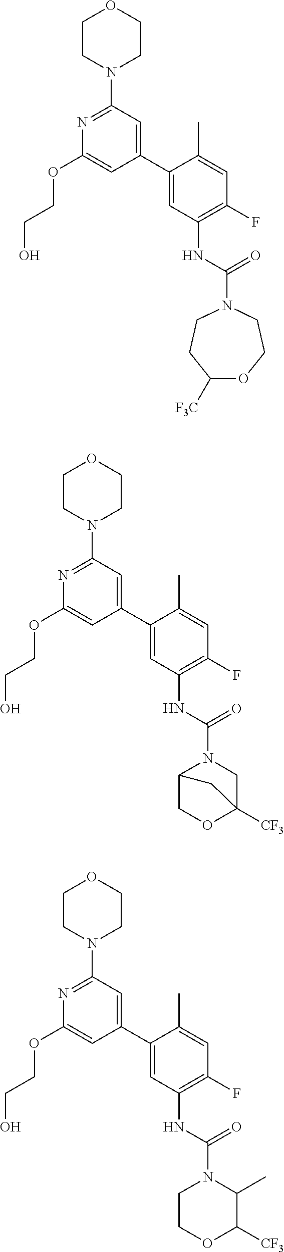 [SMILES: Cc1cc(F)c(NC(=O)N2CCOC(C(F)(F)F)CC2)cc1-c1cc(OCCO)nc(N2CCOCC2)c1 ; Cc1cc(F)c(NC(=O)N2CC3(C(F)(F)F)CC2CO3)cc1-c1cc(OCCO)nc(N2CCOCC2)c1 ; Cc1cc(F)c(NC(=O)N2CCOC(C(F)(F)F)C2C)cc1-c1cc(OCCO)nc(N2CCOCC2)c1]

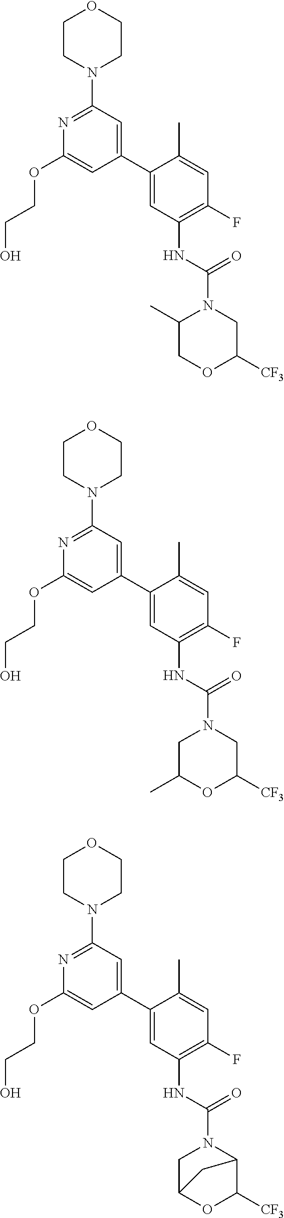 [SMILES: Cc1cc(F)c(NC(=O)N2CC(C(F)(F)F)OCC2C)cc1-c1cc(OCCO)nc(N2CCOCC2)c1 ; Cc1cc(F)c(NC(=O)N2CC(C)OC(C(F)(F)F)C2)cc1-c1cc(OCCO)nc(N2CCOCC2)c1 ; Cc1cc(F)c(NC(=O)N2CC3CC2C(C(F)(F)F)O3)cc1-c1cc(OCCO)nc(N2CCOCC2)c1]

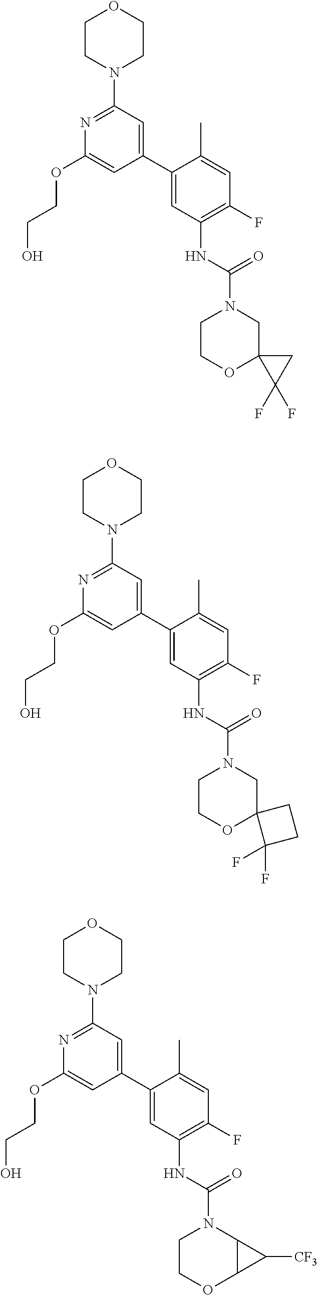 [SMILES: Cc1cc(F)c(NC(=O)N2CCOC3(C2)CC3(F)F)cc1-c1cc(OCCO)nc(N2CCOCC2)c1 ; Cc1cc(F)c(NC(=O)N2CCOC3(CCC3(F)F)C2)cc1-c1cc(OCCO)nc(N2CCOCC2)c1 ; Cc1cc(F)c(NC(=O)N2CCOC3C2C3C(F)(F)F)cc1-c1cc(OCCO)nc(N2CCOCC2)c1]

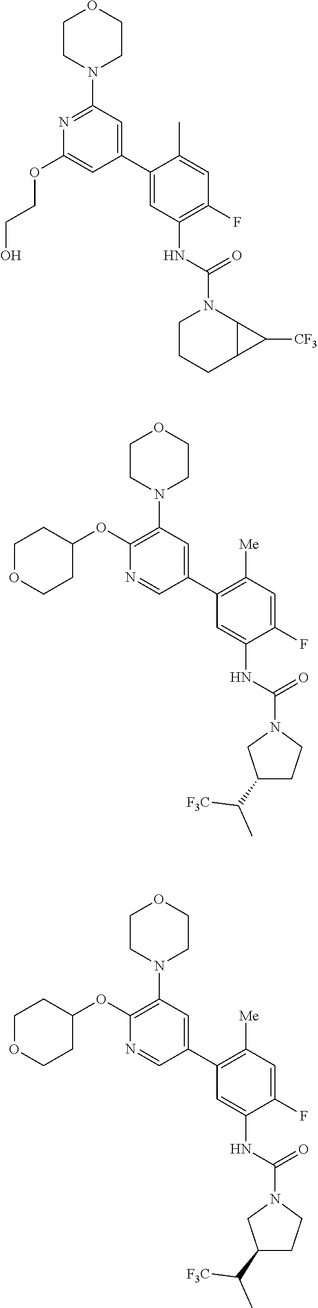 [SMILES: Cc1cc(F)c(NC(=O)N2CCCC3C2C3C(F)(F)F)cc1-c1cc(OCCO)nc(N2CCOCC2)c1 ; Cc1cc(F)c(NC(=O)N2CC[C@H](C(C)C(F)(F)F)C2)cc1-c1cnc(OC2CCOCC2)c(N2CCOCC2)c1 ; Cc1cc(F)c(NC(=O)N2CC[C@@H](C(C)C(F)(F)F)C2)cc1-c1cnc(OC2CCOCC2)c(N2CCOCC2)c1]

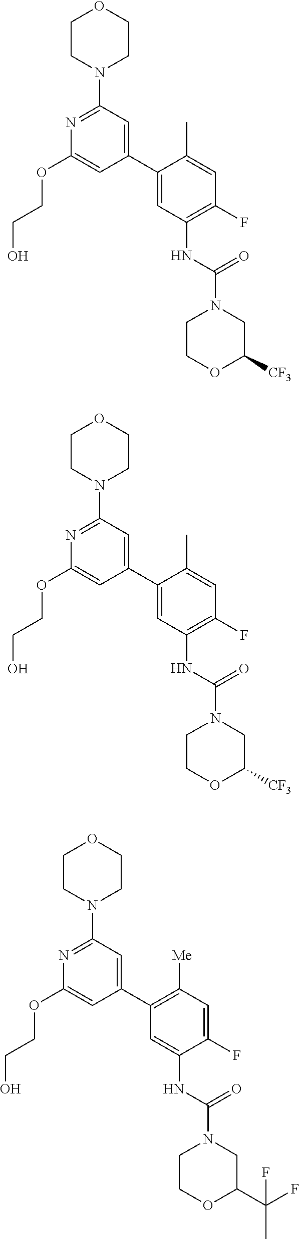 [SMILES: Cc1cc(F)c(NC(=O)N2CCO[C@H](C(F)(F)F)C2)cc1-c1cc(OCCO)nc(N2CCOCC2)c1 ; Cc1cc(F)c(NC(=O)N2CCO[C@@H](C(F)(F)F)C2)cc1-c1cc(OCCO)nc(N2CCOCC2)c1 ; Cc1cc(F)c(NC(=O)N2CCOC(C(C)(F)F)C2)cc1-c1cc(OCCO)nc(N2CCOCC2)c1]

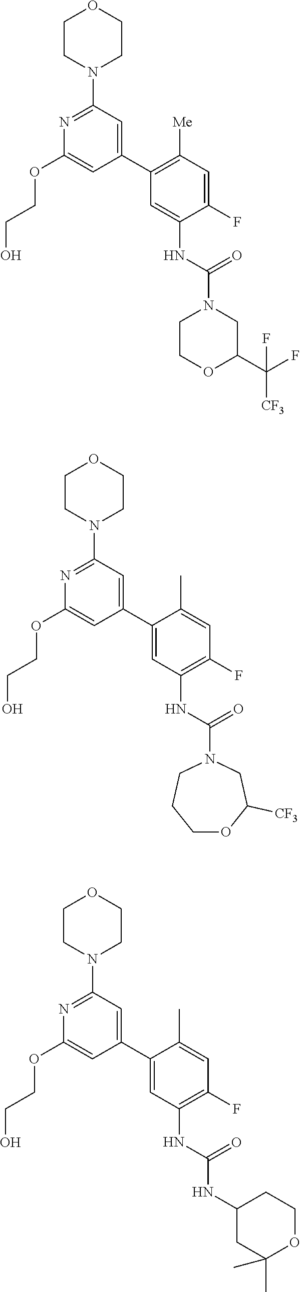 [SMILES: Cc1cc(F)c(NC(=O)N2CCOC(C(F)(F)C(F)(F)F)C2)cc1-c1cc(OCCO)nc(N2CCOCC2)c1 ; Cc1cc(F)c(NC(=O)N2CCCOC(C(F)(F)F)C2)cc1-c1cc(OCCO)nc(N2CCOCC2)c1 ; Cc1cc(F)c(NC(=O)NC2CCOC(C)(C)C2)cc1-c1cc(OCCO)nc(N2CCOCC2)c1]

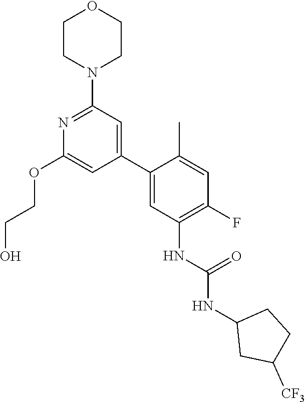 [SMILES: Cc1cc(F)c(NC(=O)NC2CCC(C(F)(F)F)C2)cc1-c1cc(OCCO)nc(N2CCOCC2)c1]

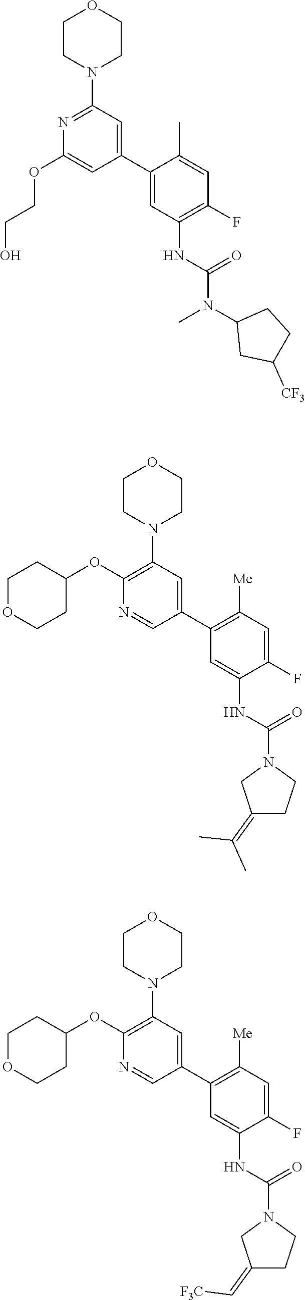 [SMILES: Cc1cc(F)c(NC(=O)N(C)C2CCC(C(F)(F)F)C2)cc1-c1cc(OCCO)nc(N2CCOCC2)c1 ; CC(C)=C1CCN(C(=O)Nc2cc(-c3cnc(OC4CCOCC4)c(N4CCOCC4)c3)c(C)cc2F)C1 ; Cc1cc(F)c(NC(=O)N2CC/C(=C/C(F)(F)F)C2)cc1-c1cnc(OC2CCOCC2)c(N2CCOCC2)c1]

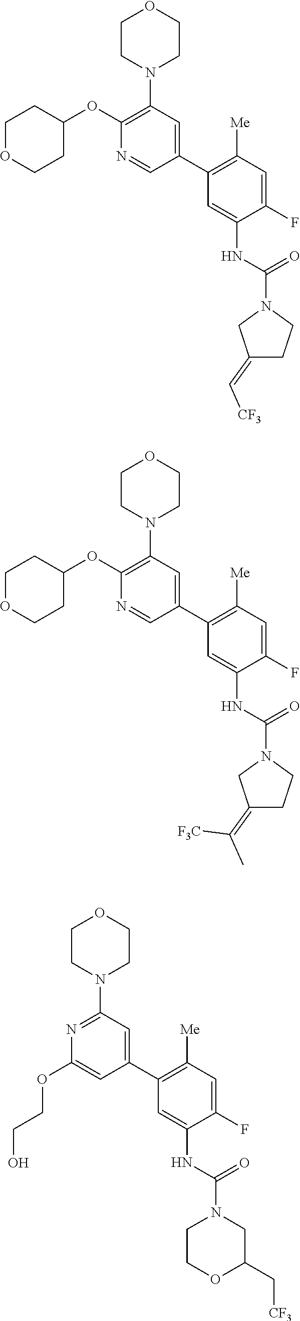 [SMILES: Cc1cc(F)c(NC(=O)N2CC/C(=C\C(F)(F)F)C2)cc1-c1cnc(OC2CCOCC2)c(N2CCOCC2)c1 ; C/C(=C1\CCN(C(=O)Nc2cc(-c3cnc(OC4CCOCC4)c(N4CCOCC4)c3)c(C)cc2F)C1)C(F)(F)F ; Cc1cc(F)c(NC(=O)N2CCOC(CC(F)(F)F)C2)cc1-c1cc(OCCO)nc(N2CCOCC2)c1]

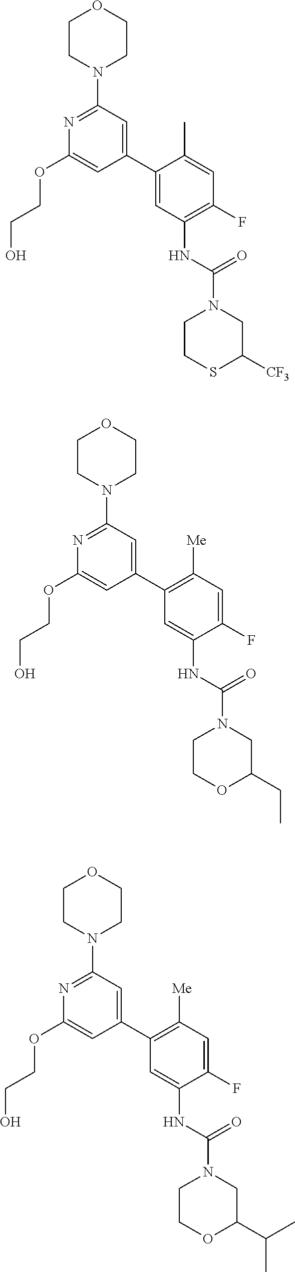 [SMILES: Cc1cc(F)c(NC(=O)N2CCSC(C(F)(F)F)C2)cc1-c1cc(OCCO)nc(N2CCOCC2)c1 ; CCC1CN(C(=O)Nc2cc(-c3cc(OCCO)nc(N4CCOCC4)c3)c(C)cc2F)CCO1 ; Cc1cc(F)c(NC(=O)N2CCOC(C(C)C)C2)cc1-c1cc(OCCO)nc(N2CCOCC2)c1]

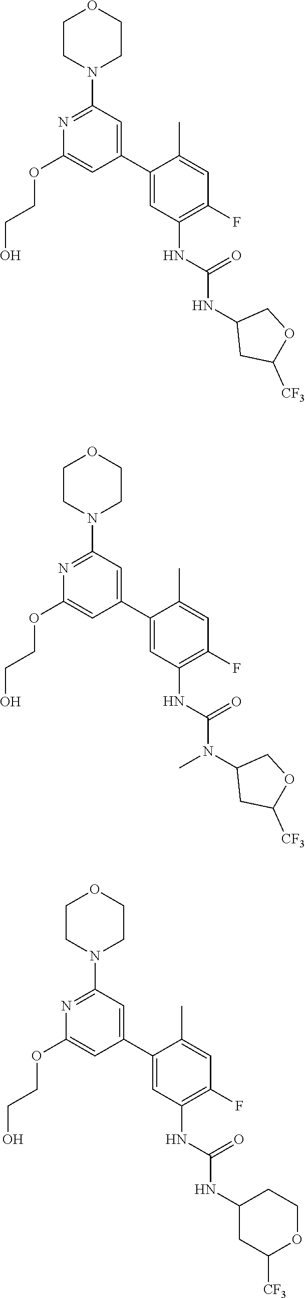 [SMILES: Cc1cc(F)c(NC(=O)NC2COC(C(F)(F)F)C2)cc1-c1cc(OCCO)nc(N2CCOCC2)c1 ; Cc1cc(F)c(NC(=O)N(C)C2COC(C(F)(F)F)C2)cc1-c1cc(OCCO)nc(N2CCOCC2)c1 ; Cc1cc(F)c(NC(=O)NC2CCOC(C(F)(F)F)C2)cc1-c1cc(OCCO)nc(N2CCOCC2)c1]

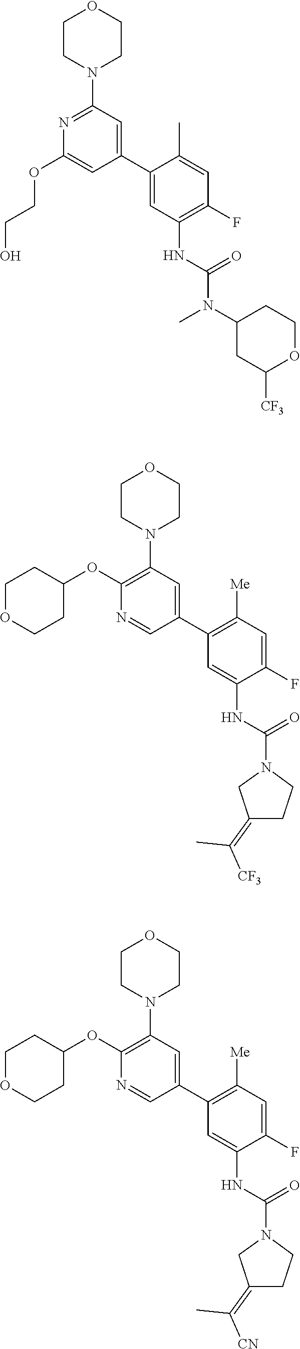 [SMILES: Cc1cc(F)c(NC(=O)N(C)C2CCOC(C(F)(F)F)C2)cc1-c1cc(OCCO)nc(N2CCOCC2)c1 ; C/C(=C1/CCN(C(=O)Nc2cc(-c3cnc(OC4CCOCC4)c(N4CCOCC4)c3)c(C)cc2F)C1)C(F)(F)F ; C/C(C#N)=C1/CCN(C(=O)Nc2cc(-c3cnc(OC4CCOCC4)c(N4CCOCC4)c3)c(C)cc2F)C1]

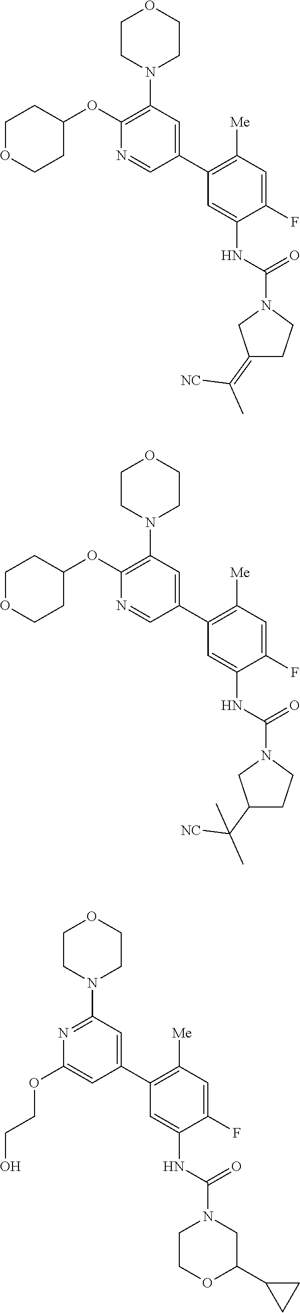 [SMILES: C/C(C#N)=C1\CCN(C(=O)Nc2cc(-c3cnc(OC4CCOCC4)c(N4CCOCC4)c3)c(C)cc2F)C1 ; Cc1cc(F)c(NC(=O)N2CCC(C(C)(C)C#N)C2)cc1-c1cnc(OC2CCOCC2)c(N2CCOCC2)c1 ; Cc1cc(F)c(NC(=O)N2CCOC(C3CC3)C2)cc1-c1cc(OCCO)nc(N2CCOCC2)c1]

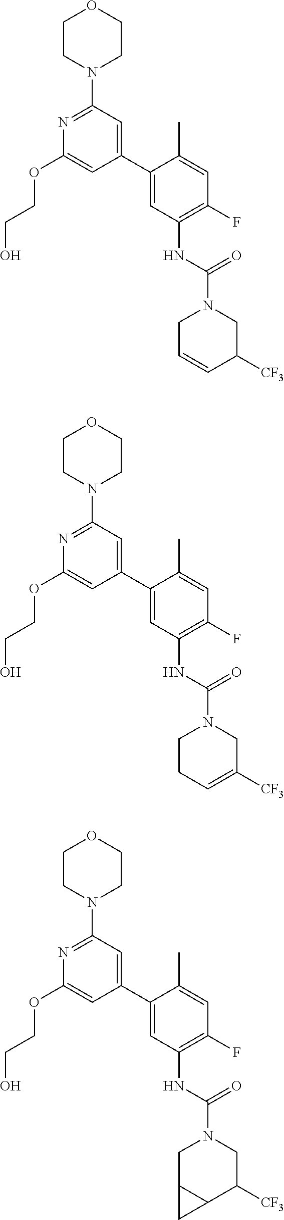 [SMILES: Cc1cc(F)c(NC(=O)N2CC=CC(C(F)(F)F)C2)cc1-c1cc(OCCO)nc(N2CCOCC2)c1 ; Cc1cc(F)c(NC(=O)N2CCC=C(C(F)(F)F)C2)cc1-c1cc(OCCO)nc(N2CCOCC2)c1 ; Cc1cc(F)c(NC(=O)N2CC3CC3C(C(F)(F)F)C2)cc1-c1cc(OCCO)nc(N2CCOCC2)c1]

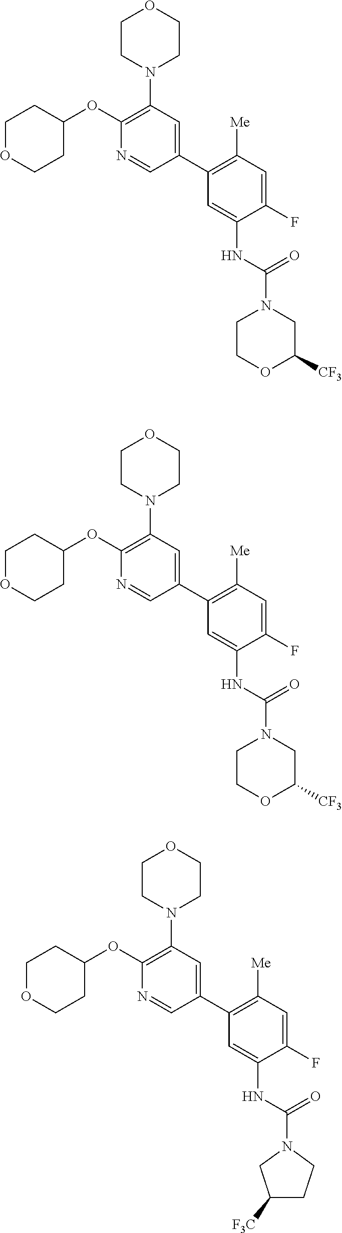 [SMILES: Cc1cc(F)c(NC(=O)N2CCO[C@H](C(F)(F)F)C2)cc1-c1cnc(OC2CCOCC2)c(N2CCOCC2)c1 ; Cc1cc(F)c(NC(=O)N2CCO[C@@H](C(F)(F)F)C2)cc1-c1cnc(OC2CCOCC2)c(N2CCOCC2)c1 ; Cc1cc(F)c(NC(=O)N2CC[C@@H](C(F)(F)F)C2)cc1-c1cnc(OC2CCOCC2)c(N2CCOCC2)c1]

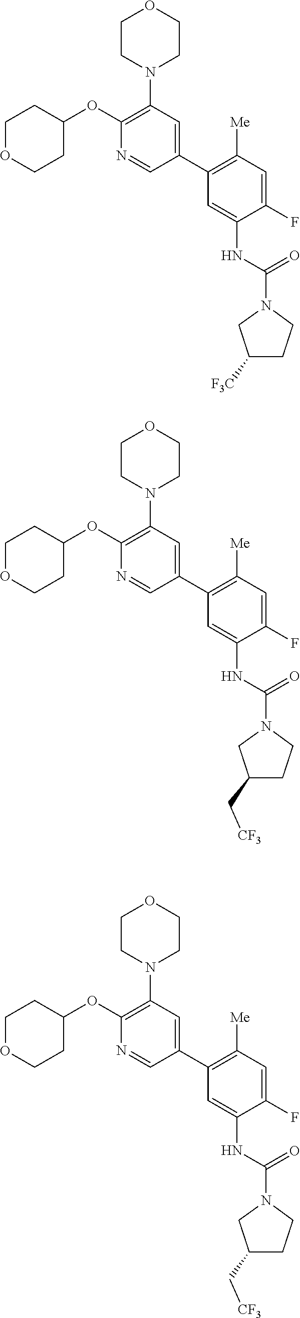 [SMILES: Cc1cc(F)c(NC(=O)N2CC[C@H](C(F)(F)F)C2)cc1-c1cnc(OC2CCOCC2)c(N2CCOCC2)c1 ; Cc1cc(F)c(NC(=O)N2CC[C@@H](CC(F)(F)F)C2)cc1-c1cnc(OC2CCOCC2)c(N2CCOCC2)c1 ; Cc1cc(F)c(NC(=O)N2CC[C@H](CC(F)(F)F)C2)cc1-c1cnc(OC2CCOCC2)c(N2CCOCC2)c1]

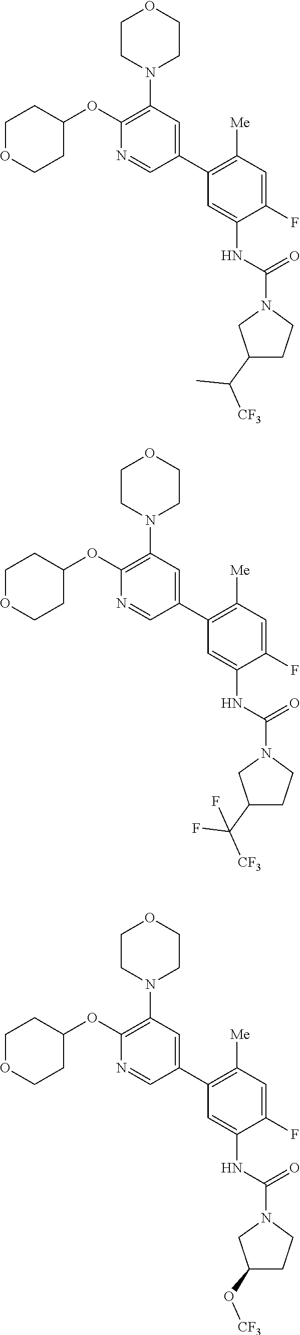 [SMILES: Cc1cc(F)c(NC(=O)N2CCC(C(C)C(F)(F)F)C2)cc1-c1cnc(OC2CCOCC2)c(N2CCOCC2)c1 ; Cc1cc(F)c(NC(=O)N2CCC(C(F)(F)C(F)(F)F)C2)cc1-c1cnc(OC2CCOCC2)c(N2CCOCC2)c1 ; Cc1cc(F)c(NC(=O)N2CC[C@@H](OC(F)(F)F)C2)cc1-c1cnc(OC2CCOCC2)c(N2CCOCC2)c1]

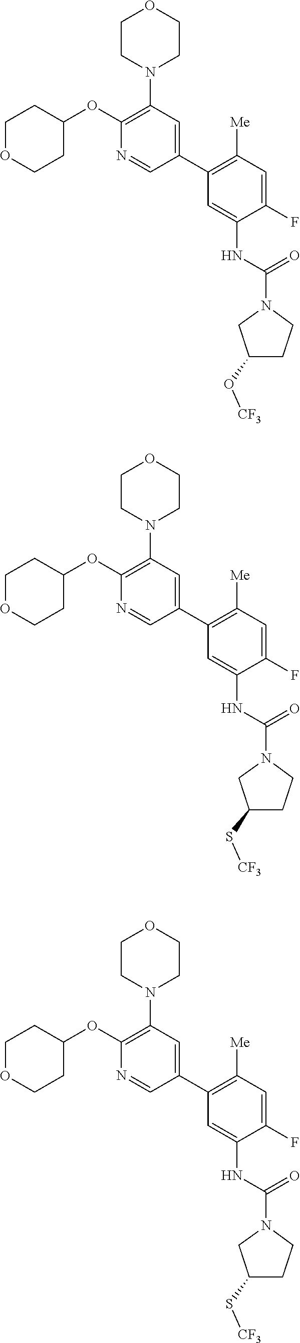 [SMILES: Cc1cc(F)c(NC(=O)N2CC[C@H](OC(F)(F)F)C2)cc1-c1cnc(OC2CCOCC2)c(N2CCOCC2)c1 ; Cc1cc(F)c(NC(=O)N2CC[C@@H](SC(F)(F)F)C2)cc1-c1cnc(OC2CCOCC2)c(N2CCOCC2)c1 ; Cc1cc(F)c(NC(=O)N2CC[C@H](SC(F)(F)F)C2)cc1-c1cnc(OC2CCOCC2)c(N2CCOCC2)c1]

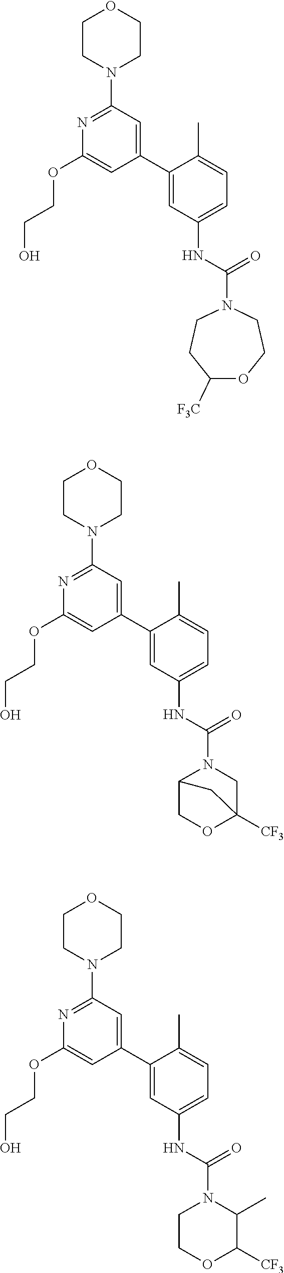 [SMILES: Cc1ccc(NC(=O)N2CCOC(C(F)(F)F)CC2)cc1-c1cc(OCCO)nc(N2CCOCC2)c1 ; Cc1ccc(NC(=O)N2CC3(C(F)(F)F)CC2CO3)cc1-c1cc(OCCO)nc(N2CCOCC2)c1 ; Cc1ccc(NC(=O)N2CCOC(C(F)(F)F)C2C)cc1-c1cc(OCCO)nc(N2CCOCC2)c1]

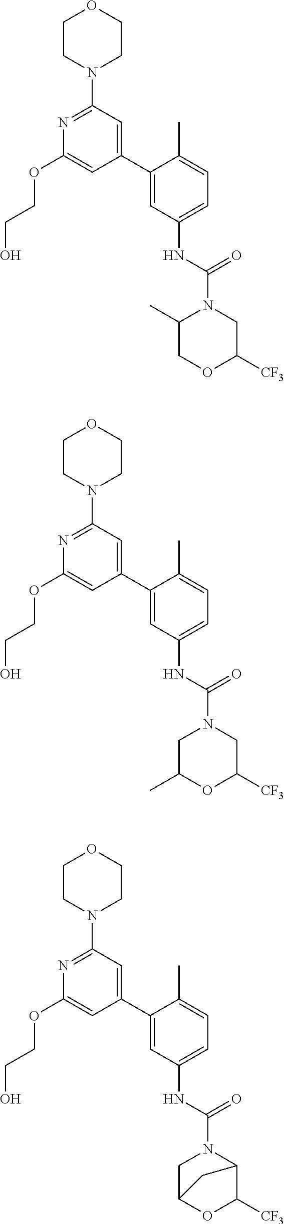 [SMILES: Cc1ccc(NC(=O)N2CC(C(F)(F)F)OCC2C)cc1-c1cc(OCCO)nc(N2CCOCC2)c1 ; Cc1ccc(NC(=O)N2CC(C)OC(C(F)(F)F)C2)cc1-c1cc(OCCO)nc(N2CCOCC2)c1 ; Cc1ccc(NC(=O)N2CC3CC2C(C(F)(F)F)O3)cc1-c1cc(OCCO)nc(N2CCOCC2)c1]

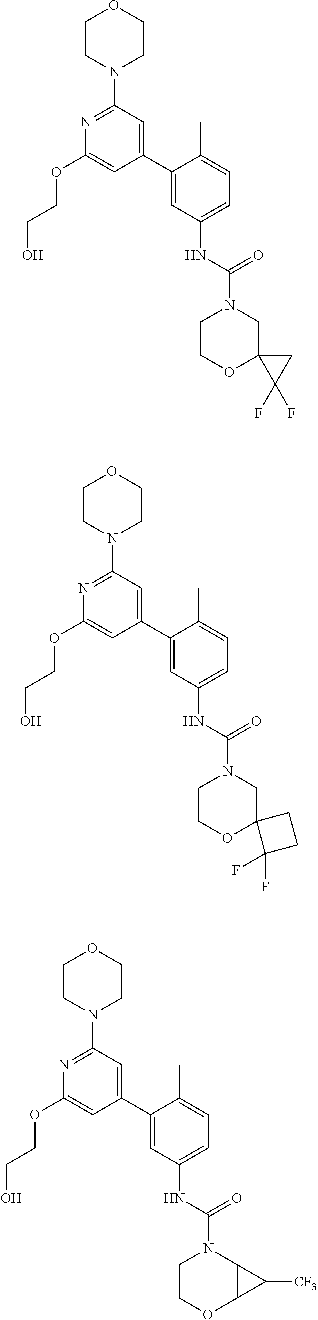 [SMILES: Cc1ccc(NC(=O)N2CCOC3(C2)CC3(F)F)cc1-c1cc(OCCO)nc(N2CCOCC2)c1 ; Cc1ccc(NC(=O)N2CCOC3(CCC3(F)F)C2)cc1-c1cc(OCCO)nc(N2CCOCC2)c1 ; Cc1ccc(NC(=O)N2CCOC3C2C3C(F)(F)F)cc1-c1cc(OCCO)nc(N2CCOCC2)c1]

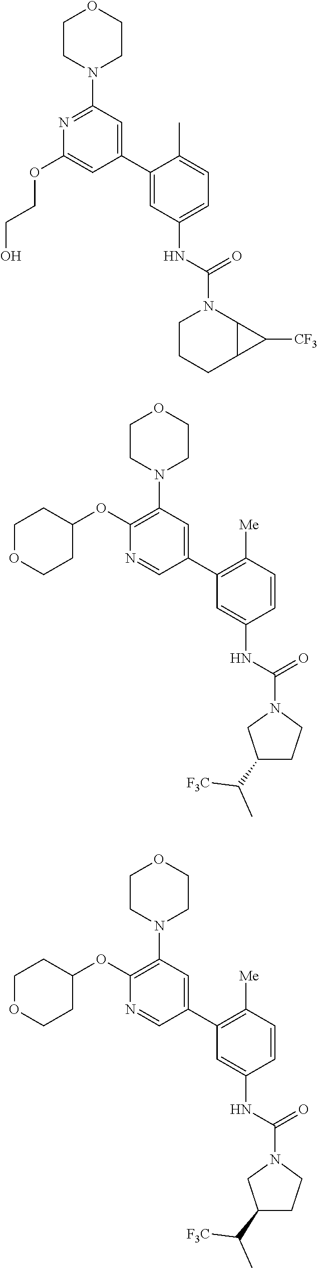 [SMILES: Cc1ccc(NC(=O)N2CCCC3C2C3C(F)(F)F)cc1-c1cc(OCCO)nc(N2CCOCC2)c1 ; Cc1ccc(NC(=O)N2CC[C@H](C(C)C(F)(F)F)C2)cc1-c1cnc(OC2CCOCC2)c(N2CCOCC2)c1 ; Cc1ccc(NC(=O)N2CC[C@@H](C(C)C(F)(F)F)C2)cc1-c1cnc(OC2CCOCC2)c(N2CCOCC2)c1]

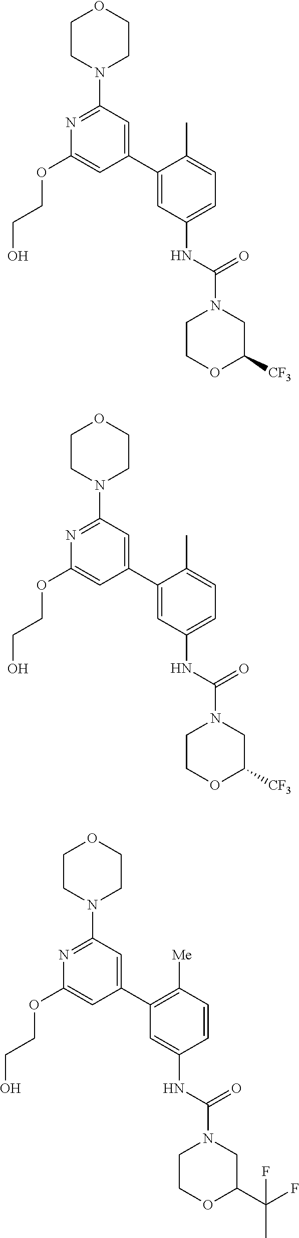 [SMILES: Cc1ccc(NC(=O)N2CCO[C@H](C(F)(F)F)C2)cc1-c1cc(OCCO)nc(N2CCOCC2)c1 ; Cc1ccc(NC(=O)N2CCO[C@@H](C(F)(F)F)C2)cc1-c1cc(OCCO)nc(N2CCOCC2)c1 ; Cc1ccc(NC(=O)N2CCOC(C(C)(F)F)C2)cc1-c1cc(OCCO)nc(N2CCOCC2)c1]

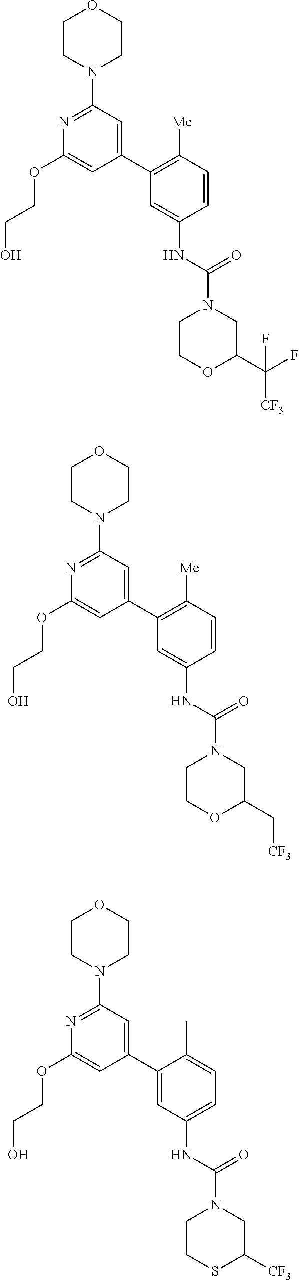 [SMILES: Cc1ccc(NC(=O)N2CCOC(C(F)(F)C(F)(F)F)C2)cc1-c1cc(OCCO)nc(N2CCOCC2)c1 ; Cc1ccc(NC(=O)N2CCOC(CC(F)(F)F)C2)cc1-c1cc(OCCO)nc(N2CCOCC2)c1 ; Cc1ccc(NC(=O)N2CCSC(C(F)(F)F)C2)cc1-c1cc(OCCO)nc(N2CCOCC2)c1]

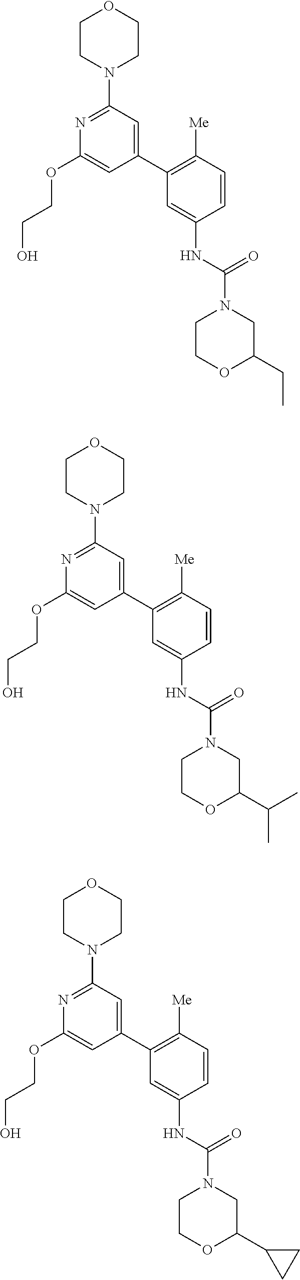 [SMILES: CCC1CN(C(=O)Nc2ccc(C)c(-c3cc(OCCO)nc(N4CCOCC4)c3)c2)CCO1 ; Cc1ccc(NC(=O)N2CCOC(C(C)C)C2)cc1-c1cc(OCCO)nc(N2CCOCC2)c1 ; Cc1ccc(NC(=O)N2CCOC(C3CC3)C2)cc1-c1cc(OCCO)nc(N2CCOCC2)c1]

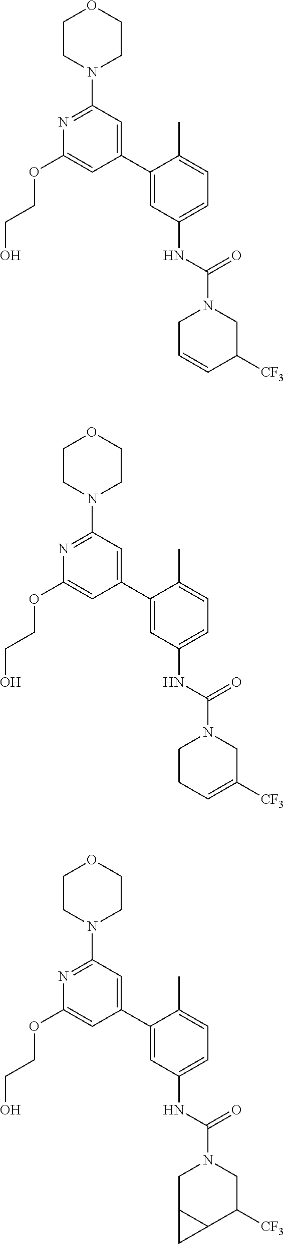 [SMILES: Cc1ccc(NC(=O)N2CC=CC(C(F)(F)F)C2)cc1-c1cc(OCCO)nc(N2CCOCC2)c1 ; Cc1ccc(NC(=O)N2CCC=C(C(F)(F)F)C2)cc1-c1cc(OCCO)nc(N2CCOCC2)c1 ; Cc1ccc(NC(=O)N2CC3CC3C(C(F)(F)F)C2)cc1-c1cc(OCCO)nc(N2CCOCC2)c1]

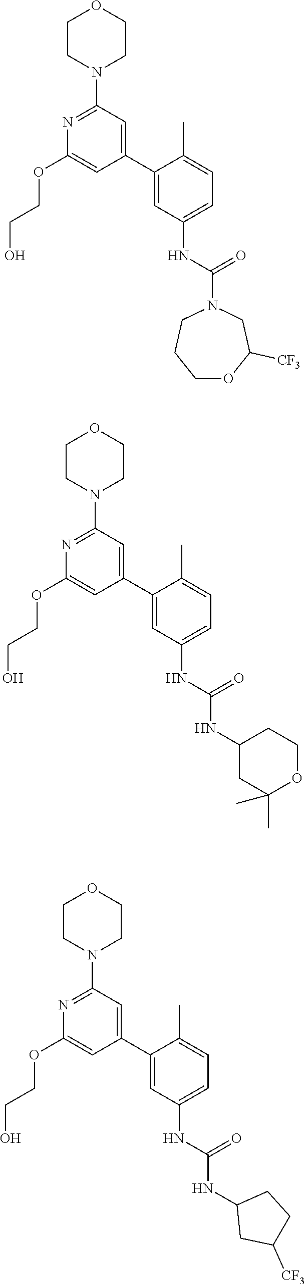 [SMILES: Cc1ccc(NC(=O)N2CCCOC(C(F)(F)F)C2)cc1-c1cc(OCCO)nc(N2CCOCC2)c1 ; Cc1ccc(NC(=O)NC2CCOC(C)(C)C2)cc1-c1cc(OCCO)nc(N2CCOCC2)c1 ; Cc1ccc(NC(=O)NC2CCC(C(F)(F)F)C2)cc1-c1cc(OCCO)nc(N2CCOCC2)c1]

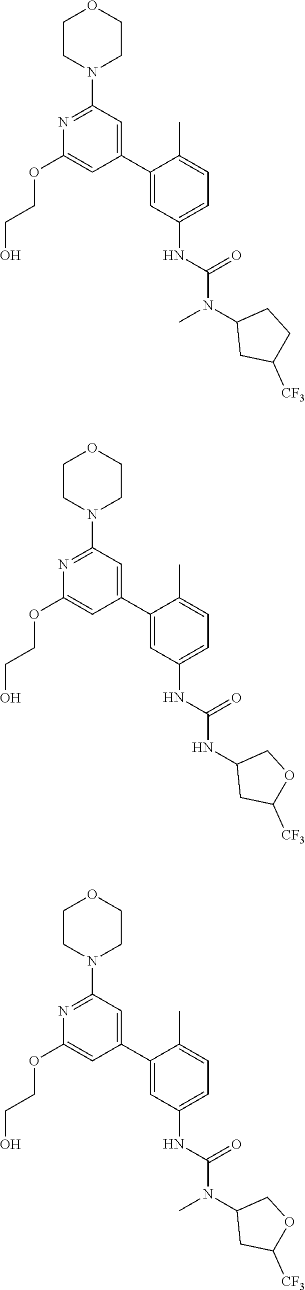 [SMILES: Cc1ccc(NC(=O)N(C)C2CCC(C(F)(F)F)C2)cc1-c1cc(OCCO)nc(N2CCOCC2)c1 ; Cc1ccc(NC(=O)NC2COC(C(F)(F)F)C2)cc1-c1cc(OCCO)nc(N2CCOCC2)c1 ; Cc1ccc(NC(=O)N(C)C2COC(C(F)(F)F)C2)cc1-c1cc(OCCO)nc(N2CCOCC2)c1]

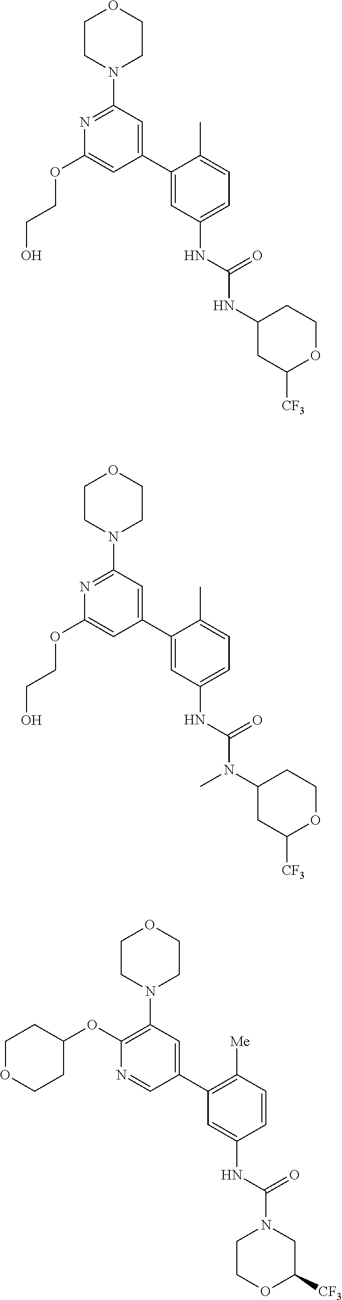 [SMILES: Cc1ccc(NC(=O)NC2CCOC(C(F)(F)F)C2)cc1-c1cc(OCCO)nc(N2CCOCC2)c1 ; Cc1ccc(NC(=O)N(C)C2CCOC(C(F)(F)F)C2)cc1-c1cc(OCCO)nc(N2CCOCC2)c1 ; Cc1ccc(NC(=O)N2CCO[C@H](C(F)(F)F)C2)cc1-c1cnc(OC2CCOCC2)c(N2CCOCC2)c1]

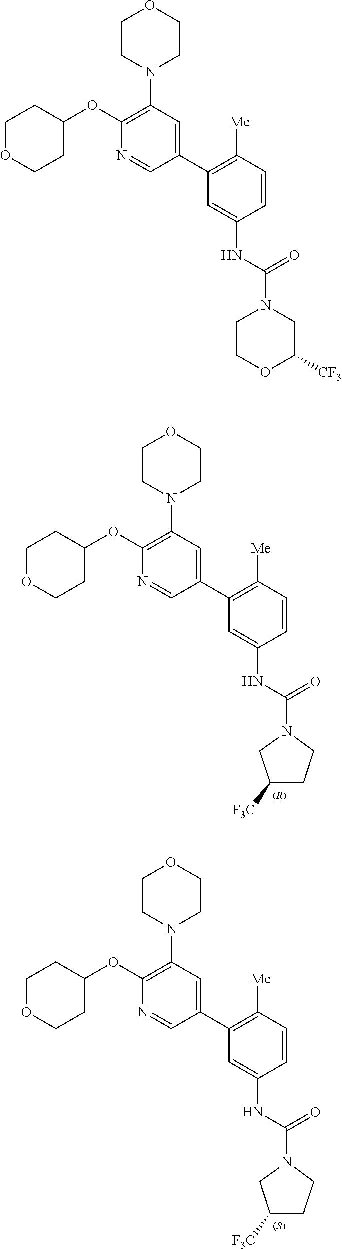

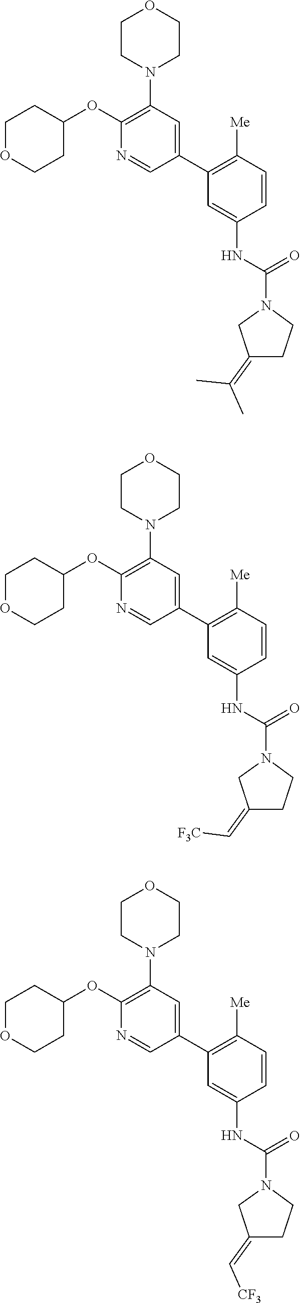 [SMILES: CC(C)=C1CCN(C(=O)Nc2ccc(C)c(-c3cnc(OC4CCOCC4)c(N4CCOCC4)c3)c2)C1 ; Cc1ccc(NC(=O)N2CC/C(=C/C(F)(F)F)C2)cc1-c1cnc(OC2CCOCC2)c(N2CCOCC2)c1 ; Cc1ccc(NC(=O)N2CC/C(=C\C(F)(F)F)C2)cc1-c1cnc(OC2CCOCC2)c(N2CCOCC2)c1]

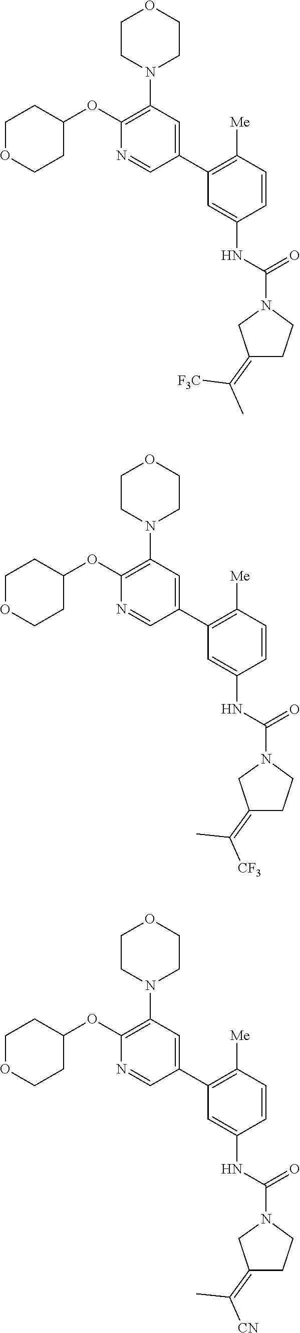 [SMILES: C/C(=C1\CCN(C(=O)Nc2ccc(C)c(-c3cnc(OC4CCOCC4)c(N4CCOCC4)c3)c2)C1)C(F)(F)F ; C/C(=C1/CCN(C(=O)Nc2ccc(C)c(-c3cnc(OC4CCOCC4)c(N4CCOCC4)c3)c2)C1)C(F)(F)F ; C/C(C#N)=C1/CCN(C(=O)Nc2ccc(C)c(-c3cnc(OC4CCOCC4)c(N4CCOCC4)c3)c2)C1]

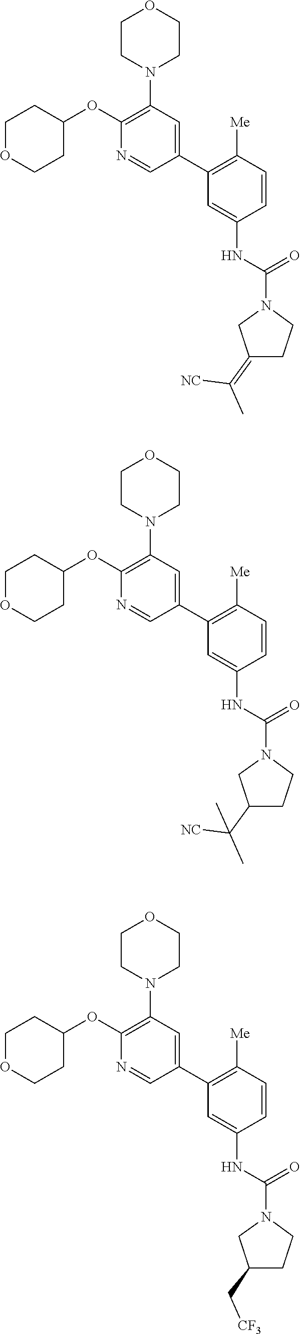 [SMILES: C/C(C#N)=C1\CCN(C(=O)Nc2ccc(C)c(-c3cnc(OC4CCOCC4)c(N4CCOCC4)c3)c2)C1 ; Cc1ccc(NC(=O)N2CCC(C(C)(C)C#N)C2)cc1-c1cnc(OC2CCOCC2)c(N2CCOCC2)c1 ; Cc1ccc(NC(=O)N2CC[C@@H](CC(F)(F)F)C2)cc1-c1cnc(OC2CCOCC2)c(N2CCOCC2)c1]

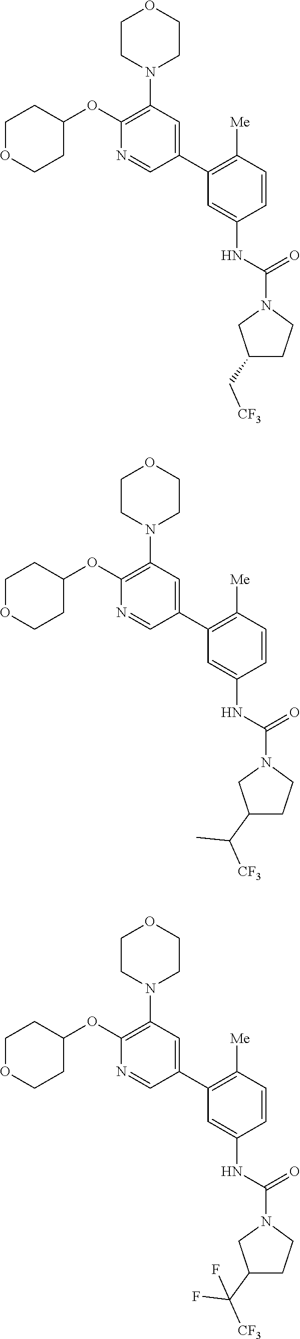 [SMILES: Cc1ccc(NC(=O)N2CC[C@H](CC(F)(F)F)C2)cc1-c1cnc(OC2CCOCC2)c(N2CCOCC2)c1 ; Cc1ccc(NC(=O)N2CCC(C(C)C(F)(F)F)C2)cc1-c1cnc(OC2CCOCC2)c(N2CCOCC2)c1 ; Cc1ccc(NC(=O)N2CCC(C(F)(F)C(F)(F)F)C2)cc1-c1cnc(OC2CCOCC2)c(N2CCOCC2)c1]

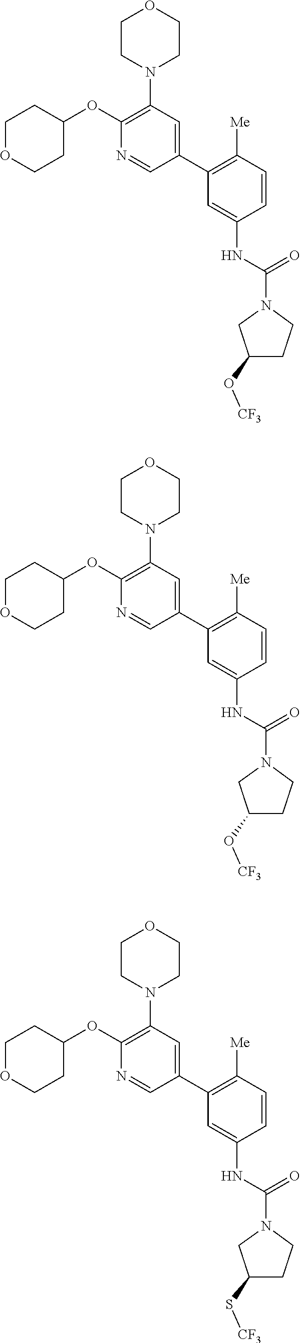 [SMILES: Cc1ccc(NC(=O)N2CC[C@@H](OC(F)(F)F)C2)cc1-c1cnc(OC2CCOCC2)c(N2CCOCC2)c1 ; Cc1ccc(NC(=O)N2CC[C@H](OC(F)(F)F)C2)cc1-c1cnc(OC2CCOCC2)c(N2CCOCC2)c1 ; Cc1ccc(NC(=O)N2CC[C@@H](SC(F)(F)F)C2)cc1-c1cnc(OC2CCOCC2)c(N2CCOCC2)c1]

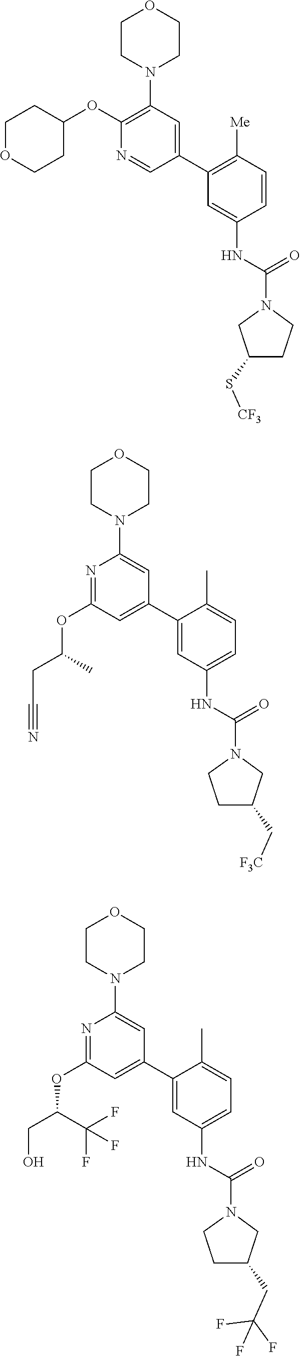 [SMILES: Cc1ccc(NC(=O)N2CC[C@H](SC(F)(F)F)C2)cc1-c1cnc(OC2CCOCC2)c(N2CCOCC2)c1 ; Cc1ccc(NC(=O)N2CC[C@@H](CC(F)(F)F)C2)cc1-c1cc(O[C@H](C)CC#N)nc(N2CCOCC2)c1 ; Cc1ccc(NC(=O)N2CC[C@@H](CC(F)(F)F)C2)cc1-c1cc(O[C@@H](CO)C(F)(F)F)nc(N2CCOCC2)c1]

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the heteroaromatic RAF kinase inhibitory compound described herein is administered as a pure chemical. In other embodiments, the heteroaromatic RAF kinase inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one heteroaromatic RAF kinase inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I)-(VI), or a pharmaceutically acceptable salt or solvate thereof.

One embodiment provides a method of preparing a pharmaceutical composition comprising mixing a compound of Formula (I)-(VI), or a pharmaceutically acceptable salt or solvate thereof, and a pharmaceutically acceptable carrier.

In certain embodiments, the heteroaromatic RAF kinase inhibitory compound as described by Formula (I)-(VI), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

In some embodiments, the heteroaromatic RAF kinase inhibitory compound as described by Formula (I)-(VI), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heteroaromatic RAF kinase inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt or solvate thereof. In some embodiments, described herein is a method of treating cancer in a patient in need thereof comprising administering to the patient a pharmaceutical composition comprising a compound of Formula (I), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (Ia), or a pharmaceutically acceptable salt or solvate thereof, for use in a method of treatment of the human or animal body.

One embodiment provides a compound of Formula (Ia), or a pharmaceutically acceptable salt or solvate thereof, for use in a method of treatment of cancer or neoplastic disease.

One embodiment provides a use of a compound of Formula (Ia), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (Ia), or a pharmaceutically acceptable salt or solvate thereof. In some embodiments, described herein is a method of treating cancer in a patient in need thereof comprising administering to the patient a pharmaceutical composition comprising a compound of Formula (Ia), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (II), or a pharmaceutically acceptable salt or solvate thereof, for use in a method of treatment of the human or animal body.

One embodiment provides a compound of Formula (II), or a pharmaceutically acceptable salt or solvate thereof, for use in a method of treatment of cancer or neoplastic disease.

One embodiment provides a use of a compound of Formula (II), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (II), or a pharmaceutically acceptable salt or solvate thereof. In some embodiments, described herein is a method of treating cancer in a patient in need thereof comprising administering to the patient a pharmaceutical composition comprising a compound of Formula (II), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (IIa), or a pharmaceutically acceptable salt or solvate thereof, for use in a method of treatment of the human or animal body.

One embodiment provides a compound of Formula (IIa), or a pharmaceutically acceptable salt or solvate thereof, for use in a method of treatment of cancer or neoplastic disease.

One embodiment provides a use of a compound of Formula (IIa), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (IIa), or a pharmaceutically acceptable salt or solvate thereof. In some embodiments, described herein is a method of treating cancer in a patient in need thereof comprising administering to the patient a pharmaceutical composition comprising a compound of Formula (IIa), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (III), or a pharmaceutically acceptable salt or solvate thereof, for use in a method of treatment of the human or animal body.

One embodiment provides a compound of Formula (III), or a pharmaceutically acceptable salt or solvate thereof, for use in a method of treatment of cancer or neoplastic disease.

One embodiment provides a use of a compound of Formula (III), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (III), or a pharmaceutically acceptable salt or solvate thereof. In some embodiments, described herein is a method of treating cancer in a patient in need thereof comprising administering to the patient a pharmaceutical composition comprising a compound of Formula (III), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (IV), or a pharmaceutically acceptable salt or solvate thereof, for use in a method of treatment of the human or animal body.

One embodiment provides a compound of Formula (IV), or a pharmaceutically acceptable salt or solvate thereof, for use in a method of treatment of cancer or neoplastic disease.

One embodiment provides a use of a compound of Formula (IV), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (IV), or a pharmaceutically acceptable salt or solvate thereof. In some embodiments, described herein is a method of treating cancer in a patient in need thereof comprising administering to the patient a pharmaceutical composition comprising a compound of Formula (IV), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (V), or a pharmaceutically acceptable salt or solvate thereof, for use in a method of treatment of the human or animal body.

One embodiment provides a compound of Formula (V), or a pharmaceutically acceptable salt or solvate thereof, for use in a method of treatment of cancer or neoplastic disease.

One embodiment provides a use of a compound of Formula (V), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (V), or a pharmaceutically acceptable salt or solvate thereof. In some embodiments, described herein is a method of treating cancer in a patient in need thereof comprising administering to the patient a pharmaceutical composition comprising a compound of Formula (V), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

One embodiment provides a compound of Formula (VI), or a pharmaceutically acceptable salt or solvate thereof, for use in a method of treatment of the human or animal body.

One embodiment provides a compound of Formula (VI), or a pharmaceutically acceptable salt or solvate thereof, for use in a method of treatment of cancer or neoplastic disease.

One embodiment provides a use of a compound of Formula (VI), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (VI), or a pharmaceutically acceptable salt or solvate thereof. In some embodiments, described herein is a method of treating cancer in a patient in need thereof comprising administering to the patient a pharmaceutical composition comprising a compound of Formula (VI), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### Examples

### I. Chemical Synthesis

In some embodiments, the heteroaromatic RAF kinase inhibitory compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
T temperature
TFA trifluoroacetic acid
THF tetrahydrofuran

### Intermediate 1: 2-fluoro-4-methyl-5-(5-morpholino-6-(2-((tetrahydro-2H-pyran-2-yl)oxy)ethoxy)pyridin-3-yl)aniline

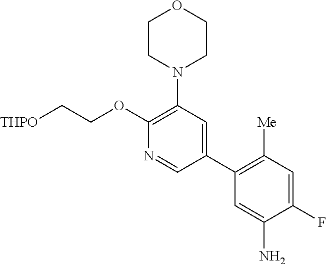

### Step 1: 4-(5-bromo-2-fluoropyridin-3-yl)morpholine

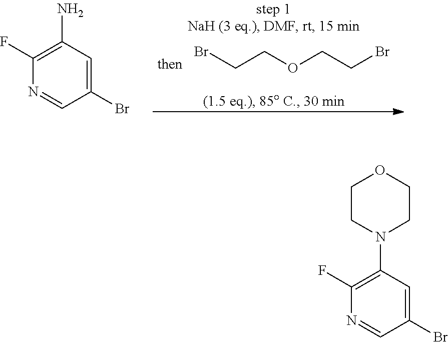 [SMILES: Nc1cc(Br)cnc1F ; BrCCOCCBr ; Fc1ncc(Br)cc1N1CCOCC1]

To a solution of 5-bromo-2-fluoropyridin-3-amine (6.00 g, 31.41 mmol) in DMF (60.00 mL) was added NaH (3.77 g, 94.24 mmol, 60%) at 0° C. The reaction mixture was stirred for 15 min. To the above mixture 1-bromo-2-(2-bromoethoxy)ethane (10.93 g, 47.12 mmol) was added. The reaction mixture was allowed to heat to 85° C. and stirred for 0.5 h. The resulting mixture was poured into water (250 mL). The resulting precipitate was collected by vacuum filtration. The filter cake was rinsed twice with water and heptanes. The solid was dried under high vacuum to give 4-(5-bromo-2-fluoropyridin-3-yl)morpholine (5.6 g, 68%) as a yellow solid. MS ESI calculated for C₉H₁₀FBrN₂O [M+H]⁺, 261.00, 262.99, found 261.05, 263.00. ¹H-NMR (400 MHz, d₆-DMSO) δ 7.86 (d, J=2.0 Hz, 1H), 7.64 (dd, J=9.2, 2.4 Hz, 1H), 3.82-3.64 (m, 4H), 3.10-3.05 (m, 4H).

### Step 2: 4-[5-bromo-2-[2-(oxan-2-yloxy)ethoxy]pyridin-3-yl]morpholine

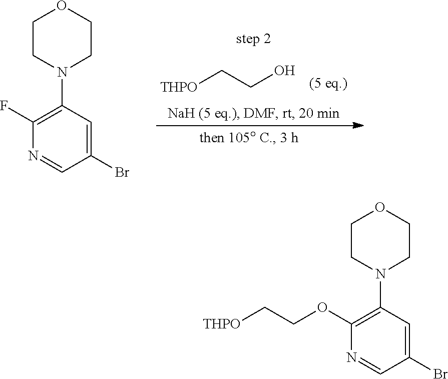

To a solution of 2-(oxan-2-yloxy)ethanol (4.62 g, 31.60 mmol) in 1,4-dioxane (60.00 mL) was added NaH (1.26 g, 31.60 mmol, 60%) at 0° C. The reaction mixture was stirred for 20 min at room temperature. To the above mixture 4-(5-bromo-2-fluoropyridin-3-yl)morpholine (1.65 g, 6.32 mmol) was added and the reaction mixture was allowed to heat to 105° C. and stirred for 3 h. The resulting mixture was cooled to room temperature and quenched with water (30 mL). The resulting mixture was extracted with EtOAc (2×50 mL). The combined organic layers was washed with water (2×10 mL), dried over anhydrous Na₂SO₄. After filtration, the filtrate was concentrated under reduced pressure. The residue was purified by silica gel column chromatography, eluted with (EtOAc:EtOH=3:1)/PE (10-50%). The fractions contained desired product were combined and concentrated to afford 4-[5-bromo-2-[2-(oxan-2-yloxy)ethoxy]pyridin-3-yl]morpholine (1.8 g, 73%) as a yellow oil. MS ESI calculated for C₁₆H₂₃BrN₂O₂[M+H]+, 387.08, 389.08; found 387.10, 389.10. ¹H-NMR (400 MHz, d₆-DMSO) δ 7.82 (d, J=2.0 Hz, 1H), 7.29 (d, J=2.4 Hz, 1H), 4.65 (d, J=3.6 Hz, 1H), 4.48-4.28 (m, 2H), 3.93-3.90 (m, 1H), 3.78-3.65 (m, 6H), 3.47-3.42 (m, 1H), 3.09-3.03 (m, 4H), 1.64-1.60 (m, 2H), 1.49-1.45 (m, 4H).

### Step 3: 2-fluoro-4-methyl-5-[5-(morpholin-4-yl)-6-[2-(oxan-2-yloxy)ethoxy]pyridin-3-yl]aniline

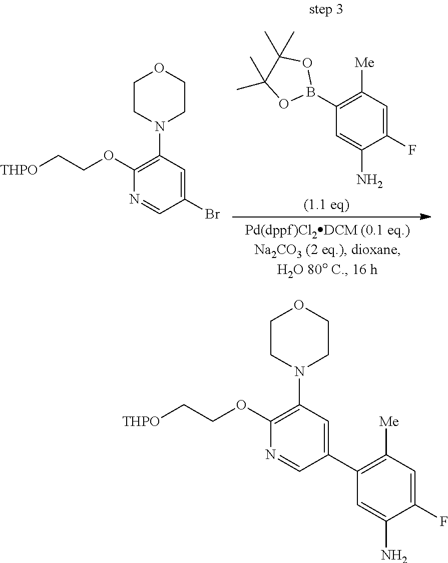

To a solution of 4-[5-bromo-2-[2-(oxan-2-yloxy)ethoxy]pyridin-3-yl]morpholine (550.00 mg, 1.42 mmol) and 2-fluoro-4-methyl-5-(4,4,5,5-tetramethyl-1,3,2-dioxaborolan-2-yl)aniline (534.93 mg, 2.13 mmol) in 1,4-dioxane (0.5 mL) and H₂O (0.1 mL) were added Na₂CO₃ (301.05 mg, 2.84 mmol) and 1,1-bis(diphenylphosphino)ferrocene-palladium(II) dichloride dichloromethane complex (463.91 mg, 0.57 mmol). The reaction mixture was degassed with nitrogen for three times and stirred for 16 h at 80° C. The resulting mixture was concentrated under reduced pressure. The residue was purified by silica gel column chromatography, eluted with MeOH in DCM (1-10%). The fractions contained desired product were combined and concentrated to afford 2-fluoro-4-methyl-5-[5-(morpholin-4-yl)-6-[2-(oxan-2-yloxy)ethoxy]pyridin-3-yl]aniline (580 mg, 95%) as a yellow solid. MS ESI calculated for C₂₃H₃₀FN₃O₄[M+H]⁺, 432.22; found 432.30. ¹H-NMR (400 MHz, d₆-DMSO) δ 7.63 (d, J=2.0 Hz, 1H), 7.05 (d, J=2.0 Hz, 1H), 6.93 (d, J=12.4 Hz, 1H), 6.65 (d, J=9.6 Hz, 1H), 4.96 (s, 2H), 4.48-4.44 (m, 2H), 3.92 (s, 1H), 3.82-3.68 (m, 6H), 3.50-3.46 (m, 1H), 3.10-3.06 (m, 4H), 2.08 (s, 3H), 1.73-1.58 (m, 2H), 1.57-1.45 (m, 5H).

### Intermediate 2: 2-fluoro-4-methyl-5-(5-morpholino-6-((tetrahydro-2H-pyran-4-yl)oxy)pyridin-3-yl)aniline

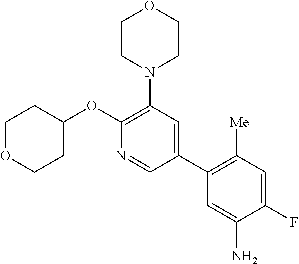 [SMILES: Cc1cc(F)c(N)cc1-c1cnc(OC2CCOCC2)c(N2CCOCC2)c1]

### Step 1: 4-[5-bromo-2-(oxan-4-yloxy)pyridin-3-yl]morpholine

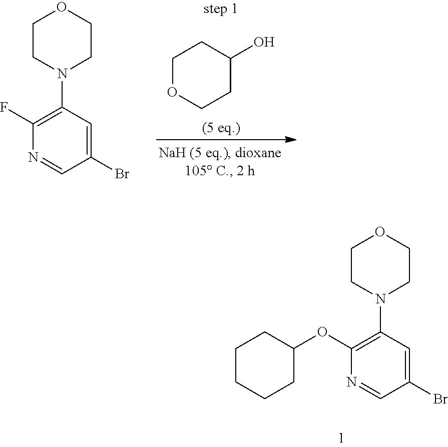 [SMILES: Fc1ncc(Br)cc1N1CCOCC1 ; OC1CCOCC1 ; Brc1cnc(OC2CCCCC2)c(N2CCOCC2)c1]

To a solution of oxan-4-ol (1.96 g, 19.15 mmol) in dioxane (40.0 mL) was added NaH (0.77 g, 19.15 mmol, 60%) at 0° C. The reaction mixture was stirred at 0° C. for 30 min. To the above mixture was added 4-(5-bromo-2-fluoropyridin-3-yl)morpholine (1.00 g, 3.83 mmol) and the reaction mixture was allowed to warm to 105° C. and stirred for 2 h. The resulting mixture was quenched by water (100 mL) and extracted with ethyl acetate (3×50 mL). The combined organic layers were washed with brine, dried over anhydrous Na₂SO₄. After filtration, the filtrate was concentrated under reduced pressure. The residue was purified by silica gel column chromatography, eluted with 20-50% EtOAc/PE. The fractions contained desired product were combined and concentrated to afford 4-[5-bromo-2-(oxan-4-yloxy)pyridin-3-yl]morpholine (0.95 g, 72%) as an off-white solid. MS ESI calculated for C₁₄H₁₉BrN₂O₃[M+H]⁺, 343.06, 345.06, found 343.05, 345.05. ¹H-NMR (300 MHz, d6-DMSO) δ 7.83 (d, J=2.1 Hz, 1H), 7.30 (d, J=2.1 Hz, 1H), 5.24 (t, J=8.1 Hz, 1H), 3.90-3.68 (m, 6H), 3.58-3.51 (m, 2H), 3.14-2.96 (m, 4H), 2.02-1.97 (m, 2H), 1.72-1.62 (m, 2H).

### Step 2: 2-fluoro-4-methyl-5-[5-(morpholin-4-yl)-6-(oxan-4-yloxy)pyridin-3-yl]aniline

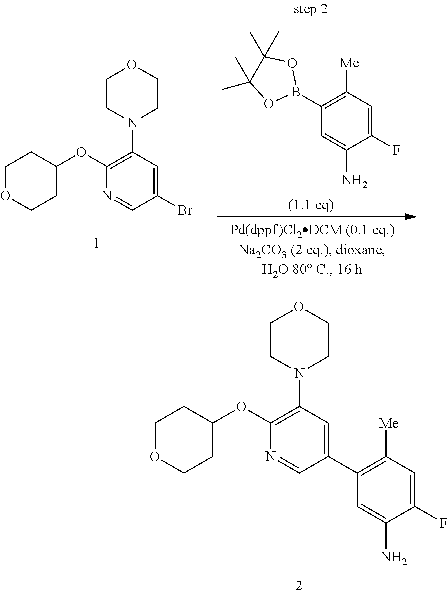 [SMILES: Brc1cnc(OC2CCOCC2)c(N2CCOCC2)c1 ; Cc1cc(F)c(N)cc1B1OC(C)(C)C(C)(C)O1 ; Cc1cc(F)c(N)cc1-c1cnc(OC2CCOCC2)c(N2CCOCC2)c1]

To a solution of 4-[5-bromo-2-(oxan-4-yloxy)pyridin-3-yl]morpholine (940.00 mg, 2.74 mmol) and 2-fluoro-4-methyl-5-(4,4,5,5-tetramethyl-1,3,2-dioxaborolan-2-yl)aniline (894.05 mg, 3.56 mmol) in dioxane (15.00 mL) and H₂O (3.00 mL) were added Na₂CO₃ (580.55 mg, 5.48 mmol) and 1,1-bis(diphenylphosphino)ferrocene-palladium(II)dichloride dichloromethane complex (223.66 mg, 0.27 mmol). The reaction mixture was degassed with nitrogen for three times and stirred at 80° C. for 3 h. The resulting mixture was concentrated under reduced pressure. The residue was purified by silica gel column chromatography, eluted with 0-10% MeOH in CH₂Cl₂. The fractions contained desired product were combined and concentrated to afford 2-fluoro-4-methyl-5-[5-(morpholin-4-yl)-6-(oxan-4-yloxy)pyridin-3-yl]aniline (0.86 g, 81%) as an off-white solid. MS ESI calculated for C₂₁H₂₆FN₃O₃[M+H]⁺, 388.20; found 388.20. ¹H-NMR (400 MHz, d6-DMSO) δ 7.62 (d, J=2.0 Hz, 1H), 7.04 (d, J=2.0 Hz, 1H), 6.91 (d, J=12.4 Hz, 1H), 6.63 (d, J=9.2 Hz, 1H), 5.31-5.30 (m, 1H), 4.96 (brs, 2H), 3.89-3.79 (m, 2H), 3.75-3.73 (m, 4H), 3.58-3.53 (m, 2H), 3.07-3.05 (m, 4H), 2.11-1.95 (m, 5H), 1.72-1.65 (m, 2H).

### Intermediate 3: 4-methyl-3-(5-morpholino-6-((tetrahydro-2H-pyran-4-yl)oxy)pyridin-3-yl)aniline

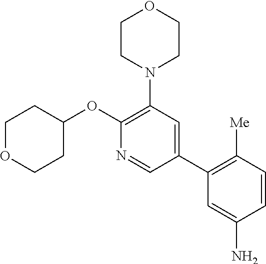 [SMILES: Cc1ccc(N)cc1-c1cnc(OC2CCOCC2)c(N2CCOCC2)c1]

The title compound was prepared using procedures similar to those described in Intermediate 2 using 4-methyl-3-(4,4,5,5-tetramethyl-1,3,2-dioxaborolan-2-yl)aniline instead of 2-fluoro-4-methyl-5-(4,4,5,5-tetramethyl-1,3,2-dioxaborolan-2-yl)aniline to afford the title compound as a solid.

### Intermediate 4: 2-fluoro-4-methyl-5-(2-morpholino-6-(2-((tetrahydro-2H-pyran-2-yl)oxy)ethoxy)pyridin-4-yl)aniline

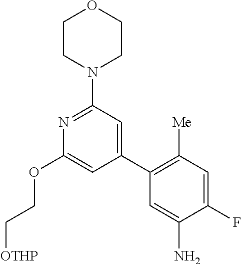

### Step 1: 4-(6-fluoro-4-iodopyridin-2-yl)morpholine

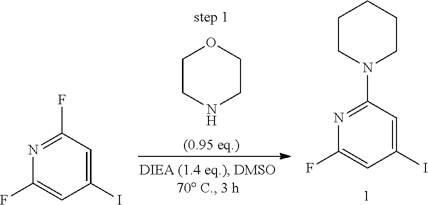 [SMILES: Fc1cc(I)cc(F)n1 ; C1COCCN1 ; Fc1cc(I)cc(N2CCCCC2)n1]

To a stirred solution of 2,6-difluoro-4-iodopyridine (16.00 g, 66.40 mmol) in DMSO (240.00 mL) were added morpholine (5.49 mL, 63.04 mmol) and DIEA (12.07 mL, 93.40 mmol) at room temperature under nitrogen atmosphere. The reaction mixture was stirred for 3 h at 70° C. The resulting mixture was diluted with water (150 mL) and extracted with EA (3×300 mL). The combined organic layers was washed with brine (4×100 mL), dried over anhydrous Na₂SO₄ and filtered. The filtrate was concentrated under reduced pressure. The residue was purified by silica gel column chromatography, eluted with 30% EA in PE. The fractions contained desired product were combined and concentrated to afford 4-(6-fluoro-4-iodopyridin-2-yl)morpholine (17.6 g, 86%) as an off-white solid. MS ESI calculated for C₉H₁₀FIN₂O [M+H]⁺, 308.98, found 309.10. H-NMR (300 MHz, CDCl₃) δ 6.77-6.76 (m, 1H), 6.60-6.59 (m, 1H), 3.78 (t, J=4.8 Hz, 4H), 3.49 (t, J=5.0 Hz, 4H).

### Step 2: 4-[4-iodo-6-[2-(oxan-2-yloxy)ethoxy]pyridin-2-yl]morpholine

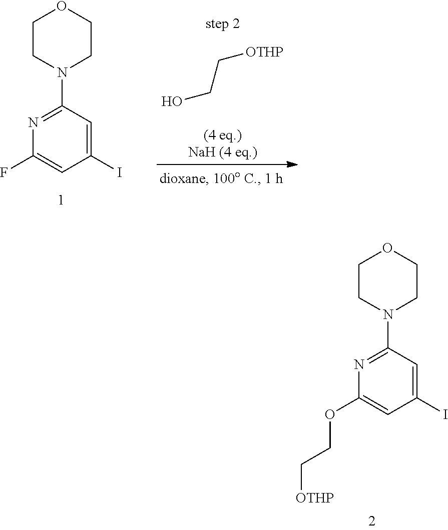

To a stirred solution of 4-(6-fluoro-4-iodopyridin-2-yl)morpholine (5.00 g, 16.23 mmol) and 2-(oxan-2-yloxy)ethanol (9.49 g, 64.92 mmol) in dioxane (100 mL) was added NaH (2.60 g, 64.92 mmol, 60%) in portions at 0° C. under nitrogen atmosphere. The reaction mixture was stirred for 1 h at 100° C. The resulting mixture was quenched with water (500 mL) at 0° C. and extracted with EA (3×300 mL). The combined organic layers was washed with brine (3×200 mL), dried over anhydrous Na₂SO₄ and filtered. The filtrate was concentrated under reduced pressure. The residue was purified by silica gel column chromatography, eluted with 30% EA in PE. The fractions contained desired product were combined and concentrated to afford 4-[4-iodo-6-[2-(oxan-2-yloxy)ethoxy]pyridin-2-yl]morpholine (5.85 g, 83%) as a yellow oil. MS ESI calculated for C₁₆H₂₃IN₂O₄[M+H]⁺, 434.07, found 435.10. H-NMR (400 MHz, CDCl₃) δ 6.56 (s, 1H), 6.50 (s, 1H), 4.67 (t, J=3.6 Hz, 1H), 4.50-4.36 (m, 2H), 4.03-3.98 (m, 1H), 3.91-3.85 (m, 1H), 3.80-3.70 (m, 5H), 3.53-3.49 (m, 1H), 3.45 (t, J=4.9 Hz, 4H), 1.87-1.51 (m, 6H).

### Step 3: 2-fluoro-4-methyl-5-[2-(morpholin-4-yl)-6-[2-(oxan-2-yloxy)ethoxy]pyridin-4-yl]aniline

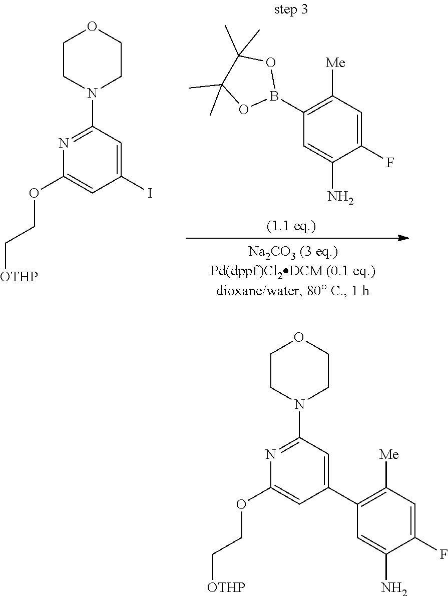

To a solution of 4-[4-iodo-6-[2-(oxan-2-yloxy)ethoxy]pyridin-2-yl]morpholine (5.75 g, 13.24 mmol) and 2-fluoro-4-methyl-5-(4,4,5,5-tetramethyl-1,3,2-dioxaborolan-2-yl)aniline (3.66 g, 14.56 mmol) in dioxane (170 mL) and water (40 mL) were added Na₂CO₃ (4.21 g, 39.72 mmol) and 1,1-bis(diphenylphosphino)ferrocene-palladium(II) dichloride dichloromethane complex (1.08 g, 1.32 mmol). The reaction mixture was degassed with nitrogen for three times and stirred for 1 h at 80° C. The resulting mixture was diluted with water (100 mL) and extracted with EA (3×150 mL). The combined organic layers was washed with brine (3×100 mL), dried over anhydrous Na₂SO₄ and filtered. The filtrate was concentrated under reduced pressure. The residue was purified by silica gel column chromatography, eluted with 0-40% EA in PE. The fractions contained desired product were combined and concentrated to afford 2-fluoro-4-methyl-5-[2-(morpholin-4-yl)-6-[2-(oxan-2-yloxy)ethoxy]pyridin-4-yl]aniline (4.4 g, 77%) as a yellow oil. MS ESI calculated for C₂₃H₃₀FN₃O₄[M+H]⁺, 432.22; found 432.25. H-NMR (300 MHz, CDCl₃) δ 6.87 (d, J=11.7 Hz, 1H), 6.65 (d, J=9.0 Hz, 1H), 6.11-6.07 (m, 2H), 4.70 (t, J=3.6 Hz, 1H), 4.54-4.41 (m, 2H), 4.11-4.02 (m, 1H), 3.94-3.77 (m, 6H), 3.54-3.47 (m, 5H), 2.14 (s, 3H), 1.89-1.43 (m, 6H).

### Intermediate 5: 4-methyl-3-(2-morpholino-6-(2-((tetrahydro-2H-pyran-2-yl)oxy)ethoxy)pyridin-4-yl)aniline

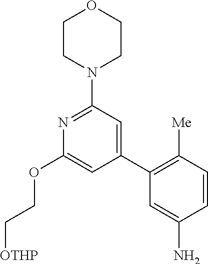

The title compound was prepared using procedures similar to those described in Intermediate 4 using 4-methyl-3-(4,4,5,5-tetramethyl-1,3,2-dioxaborolan-2-yl)aniline and 4-(4-iodo-6-(2-((tetrahydro-2H-pyran-2-yl)oxy)ethoxy)pyridin-2-yl)morpholine to afford the title compound as an oil.

### Intermediate 6: 2-fluoro-4-methyl-5-(2-((R)-3-methylmorpholino)-6-(2-((tetrahydro-2H-pyran-2-yl)oxy)ethoxy)pyridin-4-yl)aniline

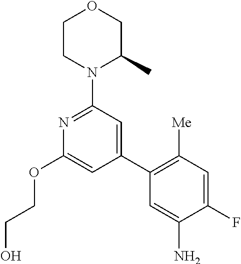 [SMILES: Cc1cc(F)c(N)cc1-c1cc(OCCO)nc(N2CCOC[C@H]2C)c1]

The title compound was prepared using procedures similar to those described in Intermediate 4 using (R)-3-methylmorpholine instead of morpholine to afford the title compound as a solid.

### Intermediate 7: 3-(2-[[(4S)-2,2-dimethyl-1,3-dioxolan-4-yl]methoxy]-6-(morpholin-4-yl)pyridin-4-yl)-4-methylaniline

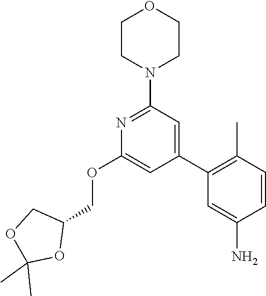 [SMILES: Cc1ccc(N)cc1-c1cc(OC[C@H]2COC(C)(C)O2)nc(N2CCOCC2)c1]

The title compound was prepared using procedures similar to those described in Intermediate 4 using [(4S)-2,2-dimethyl-1,3-dioxolan-4-yl]methanol instead of 2-(oxan-2-yloxy)ethanol to afford the title compound as a solid.

### Intermediate 8: 3-(2-[[(4R)-2,2-dimethyl-1,3-dioxolan-4-yl]methoxy]-6-(morpholin-4-yl)pyridin-4-yl)-4-methylaniline

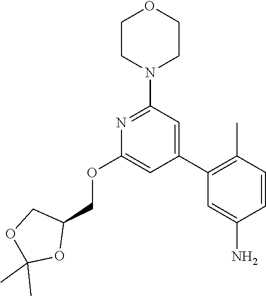 [SMILES: Cc1ccc(N)cc1-c1cc(OC[C@@H]2COC(C)(C)O2)nc(N2CCOCC2)c1]

The title compound was prepared using procedures similar to those described in Intermediate 4 using [(4R)-2,2-dimethyl-1,3-dioxolan-4-yl]methanol instead of 2-(oxan-2-yloxy)ethanol to afford the title compound as a solid.

### Intermediate 9 and 10: (2S)-1-[[4-iodo-6-(morpholin-4-yl)pyridin-2-yl]oxy]propan-2-ol and (2S)-2-[[4-iodo-6-(morpholin-4-yl)pyridin-2-yl]oxy]propan-1-ol

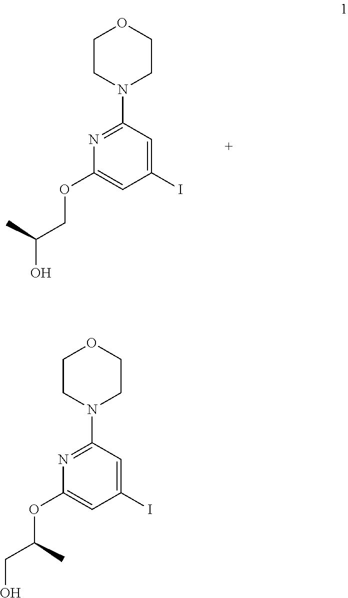 [SMILES: C[C@H](O)COc1cc(I)cc(N2CCOCC2)n1 ; C[C@@H](CO)Oc1cc(I)cc(N2CCOCC2)n1]

The title compound was prepared using procedures similar to those described in Intermediate 4 using (S)-1,2-propanediol instead of 2-(oxan-2-yloxy)ethanol to afford the title compound as a solid.

### Intermediate 11 and 12: (2R)-1-[[4-iodo-6-(morpholin-4-yl)pyridin-2-yl]oxy]propan-2-ol and (2R)-2-[[4-iodo-6-(morpholin-4-yl)pyridin-2-yl]oxy]propan-1-ol

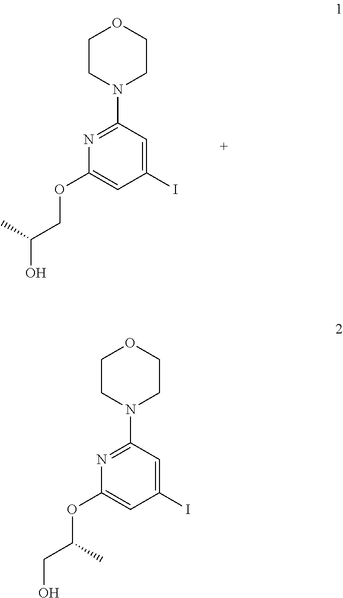 [SMILES: C[C@@H](O)COc1cc(I)cc(N2CCOCC2)n1 ; C[C@H](CO)Oc1cc(I)cc(N2CCOCC2)n1]

The title compound was prepared using procedures similar to those described in Intermediate 4 using (R)-1,2-propanediol instead of 2-(oxan-2-yloxy)ethanol to afford the title compound as a solid.

### Intermediate 13: 4-(4-iodo-6-[[2-methyl-1-(oxan-2-yloxy)propan-2-yl]oxy]pyridin-2-yl)morpholine

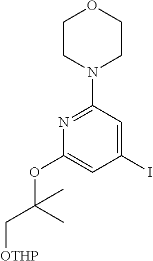

### Step 1: methyl 2-(oxan-2-yloxy)acetate

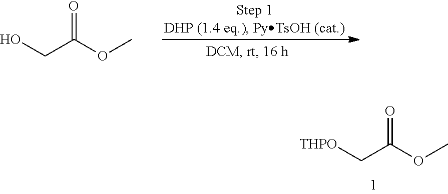

To a stirred solution of methyl 2-hydroxyacetate (6.30 g, 69.94 mmol) in DCM (100 mL) was added dihydropyran (8.93 mL, 97.88 mmol) and 4-methylbenzene-1-sulfonate; pyridin-1-ium (175.76 mg, 0.70 mmol) at 0° C. under nitrogen atmosphere. The resulting mixture was stirred for overnight at room temperature under nitrogen atmosphere. The reaction was quenched by the addition of water (100 mL) at room temperature. The resulting mixture was extracted with DCM (3×100 mL). The combined organic layers were washed with NaHCO₃ (sat., 2×200 mL) and brine (300 mL), dried over anhydrous Na₂SO₄. After filtration, the filtrate was concentrated under reduced pressure. The residue was purified by silica gel column chromatography, eluted with EtOAc in PE (0-50%) to afford methyl 2-(oxan-2-yloxy)acetate (8.6 g, 70%) as colorless oil. H-NMR (400 MHz, CDCl₃) δ 4.76-4.74 (m, 1H), 4.24 (s, 2H), 3.90-3.76 (m, 1H), 3.75 (s, 3H), 3.56-3.51 (m, 1H), 1.91-1.71 (m, 3H), 1.66-1.52 (m, 3H).

### Step 2: 2-methyl-1-(oxan-2-yloxy)propan-2-ol

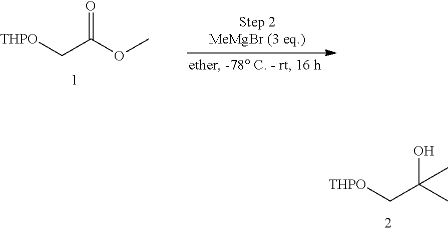

To a stirred solution of methyl 2-(oxan-2-yloxy)acetate (1.00 g, 5.74 mmol) in Et₂O (14 mL) was added CH₃MgBr (5.74 mL, 17.220 mmol, 1 M) dropwise at −70° C. under nitrogen atmosphere. The resulting mixture was stirred for 3 h at room temperature under nitrogen atmosphere. The reaction was quenched by the addition of NH₄Cl (sat., 20 mL). The resulting mixture was extracted with Et₂O (3×20 mL). The combined organic layers were washed with brine (50 mL), dried over anhydrous Na₂SO₄. After filtration, the filtrate was concentrated under reduced pressure. The residue was purified by silica gel column chromatography, eluted with EA in PE (0-100%) to afford 2-methyl-1-(oxan-2-yloxy)propan-2-ol (860 mg, 86%) as colorless oil. H-NMR (400 MHz, CDCl₃) δ 4.62-4.60 (m, 1H), 3.94-3.88 (m, 1H), 3.61-3.53 (m, 2H), 3.36-3.34 (m, 1H), 2.70 (brs, 1H), 1.93-1.74 (m, 3H), 1.68-1.53 (m, 3H), 1.22 (s, 6H).

### Step 3: 4-(4-iodo-6-[[2-methyl-1-(oxan-2-yloxy)propan-2-yl]oxy]pyridin-2-yl)morpholine

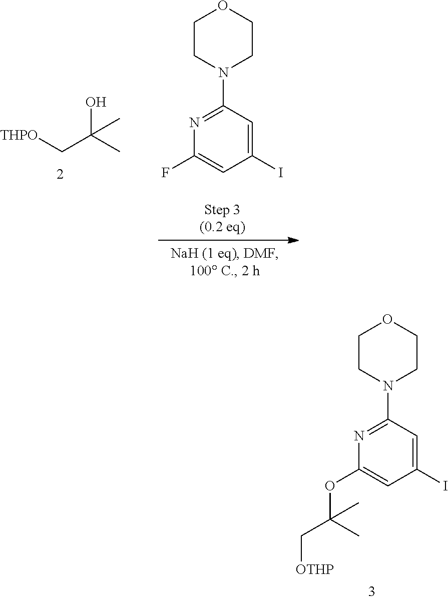

To a stirred mixture of 2-methyl-1-(oxan-2-yloxy)propan-2-ol (848.31 mg, 4.87 mmol) in DMF (1.50 mL) was added NaH (38.95 mg, 0.97 mmol, 60%) in portions at 0° C. under nitrogen atmosphere. To the above mixture was added 4-(6-fluoro-4-iodopyridin-2-yl)morpholine (300.00 mg, 0.97 mmol) at room temperature. The resulting mixture was stirred for additional 2 h at 100° C. The mixture was allowed to cool down to room temperature. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by silica gel column chromatography, eluted with 25% EA in PE to afford 4-(4-iodo-6-[[2-methyl-1-(oxan-2-yloxy)propan-2-yl]oxy]pyridin-2-yl)morpholine (190 mg, 42%) as light yellow oil. MS ESI calculated for C₁₈H₂₇IN₂O₄ [M+H]⁺, 463.10, found 463.10. H-NMR (400 MHz, CDCl₃) δ 6.52-6.51 (m, 2H), 4.64-4.63 (m, 1H), 3.96-3.94 (m, 1H), 3.85-3.79 (m, 5H), 3.72-3.70 (m, 1H), 3.53-3.42 (m, 5H), 1.83-1.47 (m, 12H).

### Intermediate 14: 1-[[4-iodo-6-(morpholin-4-yl)pyridin-2-yl]oxy]-2-methylpropan-2-ol

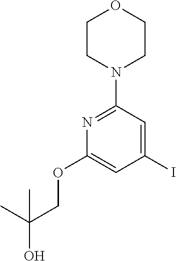 [SMILES: CC(C)(O)COc1cc(I)cc(N2CCOCC2)n1]

The title compound was prepared using procedures similar to those described in Intermediate 4 step 2 using 2-methyl-propane-1,2-diol instead of 2-(oxan-2-yloxy)ethanol to afford the title compound as a solid.

### Intermediate 15: 1-((4-iodo-6-morpholinopyridin-2-yl)oxy)-3-methoxypropan-2-ol

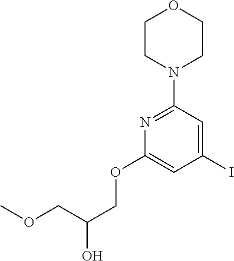 [SMILES: COCC(O)COc1cc(I)cc(N2CCOCC2)n1]

The title compound was prepared using procedures similar to those described in Intermediate 4 step 2 using 3-methoxypropane-1,2-diol instead of 2-(oxan-2-yloxy)ethanol to afford the title compound as a solid.

### Intermediate 16: (3R)-N-[4-methyl-3-(4,4,5,5-tetramethyl-1,3,2-dioxaborolan-2-yl)phenyl]-3-(trifluoromethoxy)pyrrolidine-1-carboxamide

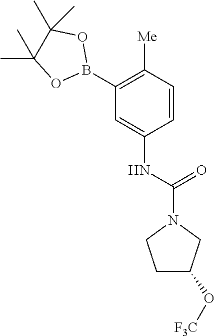 [SMILES: Cc1ccc(NC(=O)N2CC[C@@H](OC(F)(F)F)C2)cc1B1OC(C)(C)C(C)(C)O1]

### Step 1: (3R)-N-[4-methyl-3-(4,4,5,5-tetramethyl-1,3,2-dioxaborolan-2-yl)phenyl]-3-(trifluoromethoxy)pyrrolidine-1-carboxamide

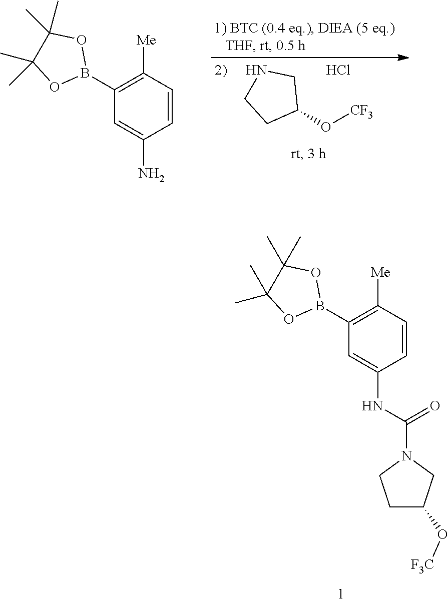 [SMILES: Cc1ccc(N)cc1B1OC(C)(C)C(C)(C)O1 ; FC(F)(F)O[C@@H]1CCNC1 ; Cl ; Cc1ccc(NC(=O)N2CC[C@@H](OC(F)(F)F)C2)cc1B1OC(C)(C)C(C)(C)O1]

To a stirred solution of (3R)-3-(trifluoromethoxy)pyrrolidine hydrochloride (150 mg, 0.64 mmol) and DIEA (415.80 mg, 3.22 mmol) in THF (5 mL) was added triphosgene (76.38 mg, 0.26 mmol) at room temperature. The resulting mixture was stirred for 0.5 h at room temperature. To this was added (3R)-3-(trifluoromethoxy)pyrrolidine hydrochloride (135.60 mg, 0.708 mmol) at room temperature. The solution was stirred for 1 h at room temperature. The resulting mixture was concentrated under vacuum. The residue was purified by silica gel column chromatography, eluted with EA/PE (0 to 60%) to afford (3R)-N-[4-methyl-3-(4,4,5,5-tetramethyl-1,3,2-dioxaborolan-2-yl)phenyl]-3-(trifluoromethoxy)pyrrolidine-1-carboxamide (250 mg, 94%) as an off-white solid. MS ESI calculated for C₁₉H₂₆BF₃N₂O₄ [M+H]⁺, 415.19, found 415.25. H-NMR (400 MHz, CDCl₃) δ 7.73-7.71 (m, 1H), 7.46-7.45 (m, 1H), 7.16-7.14 (m, 1H), 6.14 (s, 1H), 4.95-4.92 (m, 1H), 3.80-3.71 (m, 2H), 3.64-3.61 (m, 2H), 2.51 (s, 3H), 2.37-2.20 (m, 2H), 1.37 (s, 12H). F-NMR (376 MHz, CDCl₃) δ −58.70 (3F).

### Intermediate 17: (3S)-N-[4-methyl-3-(4,4,5,5-tetramethyl-1,3,2-dioxaborolan-2-yl)phenyl]-3-(trifluoromethoxy)pyrrolidine-1-carboxamide

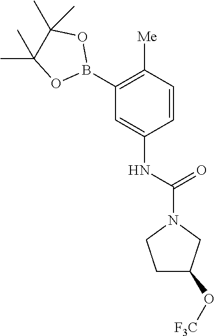 [SMILES: Cc1ccc(NC(=O)N2CC[C@H](OC(F)(F)F)C2)cc1B1OC(C)(C)C(C)(C)O1]

The title compound was prepared using procedures similar to those described in Intermediate 16 using (3S)-3-(trifluoromethoxy)pyrrolidine hydrochloride instead of (3R)-3-(trifluoromethoxy)pyrrolidine hydrochloride to afford the title compound as a solid.

### Intermediate 18: 3-methyl-1-[4-methyl-3-(4,4,5,5-tetramethyl-1,3,2-dioxaborolan-2-yl)phenyl]-3-[1-(trifluoromethyl)pyrazol-4-yl]urea

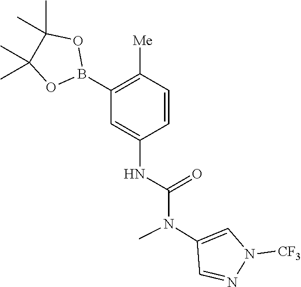 [SMILES: Cc1ccc(NC(=O)N(C)c2cnn(C(F)(F)F)c2)cc1B1OC(C)(C)C(C)(C)O1]

The title compound was prepared using procedures similar to those described in Intermediate 16 using N-methyl-1-(trifluoromethyl)pyrazol-4-amine instead of (3R)-3-(trifluoromethoxy)pyrrolidine hydrochloride to afford the title compound as a solid.

### Intermediate 19: N-(4-methyl-3-(4,4,5,5-tetramethyl-1,3,2-dioxaborolan-2-yl)phenyl)-3-(2,2,2-trifluoroethyl)pyrrolidine-1-carboxamide

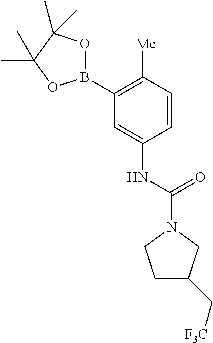 [SMILES: Cc1ccc(NC(=O)N2CCC(CC(F)(F)F)C2)cc1B1OC(C)(C)C(C)(C)O1]

The title compound was prepared using procedures similar to those described in Intermediate 16 using 3-(2,2,2-trifluoroethyl)pyrrolidine instead of (3R)-3-(trifluoromethoxy)pyrrolidine hydrochloride to afford the title compound as a solid.

### Intermediate 20: 3-(1,1-difluoroethyl)pyrrolidine hydrochloride

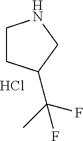

### Step 1: tert-butyl 3-(1,1-difluoroethyl)pyrrolidine-1-carboxylate

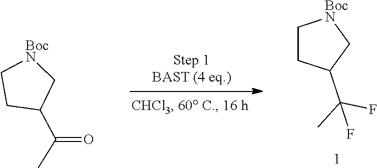 [SMILES: CC(=O)C1CCN(C(=O)OC(C)(C)C)C1 ; CC(C)(C)OC(=O)N1CCC(C(C)(F)F)C1]

To a stirred solution of tert-butyl 3-acetylpyrrolidine-1-carboxylate (600.00 mg, 2.81 mmol) in CHCl₃ (12.00 mL) was added BAST (2.49 g, 11.26 mmol) dropwise at 0° C. under nitrogen atmosphere. The reaction mixture was stirred overnight at 60° C. The resulting mixture was quenched with NaHCO₃ (sat.) at 0° C. and extracted with EA (3×40 mL). The combined organic layers was washed with brine (3×30 mL), dried over anhydrous Na₂SO₄, and filtered. The filtrate was concentrated under reduced pressure. The residue was purified by silica gel column chromatography, eluted with PE/EA (3:1). The fractions contained desired product were combined and concentrated to afford tert-butyl 3-(1,1-difluoroethyl)pyrrolidine-1-carboxylate (360 mg, 54%) as a yellow oil. H-NMR (400 MHz, CDCl₃) δ 3.57 (s, 2H), 3.33-3.28 (m, 2H), 2.64-2.60 (m, 1H), 2.04-1.92 (m, 2H), 1.61 (t, J=18.8 Hz, 3H), 1.46 (s, 9H).

### Step 2: 3-(1,1-difluoroethyl)pyrrolidine hydrochloride

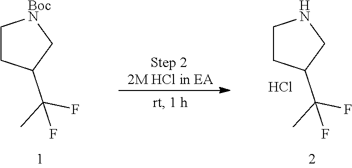

To a stirred solution of tert-butyl 3-(1,1-difluoroethyl)pyrrolidine-1-carboxylate (360 mg, 1.53 mmol) in EA (1.00 mL) was added HCl (10 mL, 2 M in EA) dropwise at 0° C. under nitrogen atmosphere. The reaction mixture was stirred for 1 h at room temperature. The resulting mixture was concentrated under reduced pressure to afford 3-(1,1-difluoroethyl)pyrrolidine hydrochloride (320 mg) as a yellow oil. It was used to next step without further purification. H-NMR (400 MHz, CDCl₃) δ 3.65-3.32 (m, 4H), 2.91-2.87 (m, 1H), 2.25-2.09 (m, 2H), 1.63 (t, J=18.0 Hz, 3H).

### Intermediate 21: 2-(1,1-difluoroethyl)morpholine hydrochloride

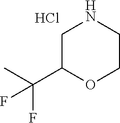

The title compound was prepared using procedures similar to those described in Intermediate 20 using tert-butyl 2-acetylmorpholine-4-carboxylate instead of tert-butyl 3-acetylpyrrolidine-1-carboxylate to afford the title compound as a yellow oil.

### Intermediate 22: 3-(trifluoromethyl)-2,5-dihydro-1H-pyrrole hydrochloride

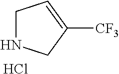

### Step 1: 3-(trifluoromethyl)-2,5-dihydro-1H-pyrrole hydrochloride

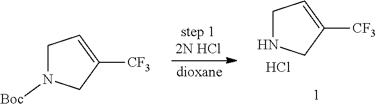 [SMILES: CC(C)(C)OC(=O)N1CC=C(C(F)(F)F)C1 ; Cl ; FC(F)(F)C1=CCNC1]

A solution of tert-butyl 3-(trifluoromethyl)-2,5-dihydropyrrole-1-carboxylate (100 mg, 0.42 mmol) and HCl (gas) in 1,4-dioxane (2 mL, 65.82 mmol, 2 N) was stirred for 16 h at room temperature under nitrogen atmosphere. The resulting mixture was concentrated under reduced pressure to afford 3-(trifluoromethyl)-2,5-dihydro-1H-pyrrole hydrochloride (80 mg, crude) as a yellow solid. It was used directly to next step without further purification. MS ESI calculated for C₅H₇ClF₃N [M+H−HCl]⁺, 138.05, found 138.20.

### Intermediate 23: 3-(1,1,2,2,2-pentafluoroethyl)pyrrolidine hydrochloride

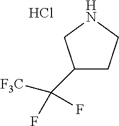 [SMILES: Cl ; FC(F)(F)C(F)(F)C1CCNC1]

### Step 1: tert-butyl 3-hydroxy-3-(1,1,2,2,2-pentafluoroethyl)pyrrolidine-1-carboxylate

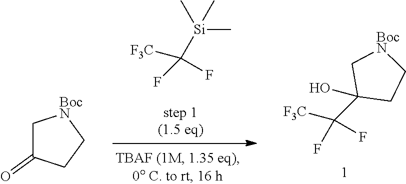 [SMILES: CC(C)(C)OC(=O)N1CCC(=O)C1 ; C[Si](C)(C)C(F)(F)C(F)(F)F ; CC(C)(C)OC(=O)N1CCC(O)(C(F)(F)C(F)(F)F)C1]

To a stirred solution of tert-butyl 3-oxopyrrolidine-1-carboxylate (2.00 g, 10.80 mmol) and trimethyl(1,1,2,2,2-pentafluoroethyl)silane (3.11 g, 16.20 mmol) in THE (20 mL) was added TBAF (14.58 mL, 14.58 mmol) dropwise at −40° C. under nitrogen atmosphere. The reaction mixture was stirred for 16 h at room temperature under nitrogen atmosphere. The resulting mixture was diluted with EA (100 mL). The resulting solution was washed with water (3×100 mL) and brine (100 mL), dried over anhydrous Na₂SO₄. After filtration, the filtrate was concentrated under reduced pressure to afford tert-butyl 3-hydroxy-3-(1,1,2,2,2-pentafluoroethyl)pyrrolidine-1-carboxylate (3.3 g, 80%) as a light brown oil. MS ESI calculated for C₁₁H₁₆F₅NO₃ [M−Boc+H]⁺, 206.11, found 206.05.

### Step 2: tert-butyl 3-(1,1,2,2,2-pentafluoroethyl)-2,5-dihydropyrrole-1-carboxylate

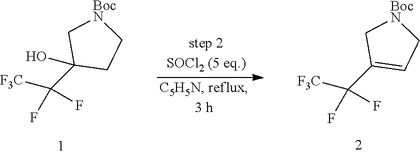 [SMILES: CC(C)(C)OC(=O)N1CCC(O)(C(F)(F)C(F)(F)F)C1 ; CC(C)(C)OC(=O)N1CC=C(C(F)(F)C(F)(F)F)C1]

To a stirred solution of tert-butyl 3-hydroxy-3-(1,1,2,2,2-pentafluoroethyl)pyrrolidine-1-carboxylate (2.10 g, 6.88 mmol) in pyridine (20 mL) was added SOCl₂ (4.09 g, 34.40 mmol) dropwise at room temperature under nitrogen atmosphere. The resulting mixture was stirred for 4 h at 80° C. under nitrogen atmosphere. The resulting mixture was concentrated under reduced pressure. The residue was purified by silica gel column chromatography, eluted with EA in PE (0-30%). The fractions contained desired product were combined and concentrated to afford tert-butyl 3-(1,1,2,2,2-pentafluoroethyl)-2,5-dihydropyrrole-1-carboxylate (1.25 g, 63%) as yellow oil. MS ESI calculated for C₁₁H₁₄F₅NO₂ [M-Boc+H]⁺, 188.04, found 187.85. H-NMR (400 MHz, CDCl₃) δ 6.43-6.38 (m, 1H), 4.35-4.30 (m, 4H), 1.51 (s, 9H). F-NMR (376 MHz, d₆-DMSO) δ −83.57 (3F), −113.39 (2F).

### Step 3: tert-butyl 3-(1,1,2,2,2-pentafluoroethyl)pyrrolidine-1-carboxylate

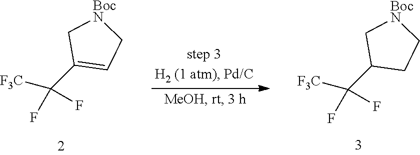 [SMILES: CC(C)(C)OC(=O)N1CC=C(C(F)(F)C(F)(F)F)C1 ; CC(C)(C)OC(=O)N1CCC(C(F)(F)C(F)(F)F)C1]

A mixture of tert-butyl 3-(1,1,2,2,2-pentafluoroethyl)-2,5-dihydropyrrole-1-carboxylate (1.25 g, 4.35 mmol) and Pd/C (Wet) (0.50 g, 4.70 mmol) in MeOH (15 mL) was stirred for 2 h at room temperature under hydrogen (2 atm) atmosphere. The resulting mixture was filtered, and the filter cake was washed with MeOH (30 mL). The filtrate was concentrated under reduced pressure to afford tert-butyl 3-(1,1,2,2,2-pentafluoroethyl)pyrrolidine-1-carboxylate (1.2 g, 95%) as a light yellow oil. MS ESI calculated for C₁₁H₁₆F₅NO₂ [M-Boc+H]⁺, 190.06, found 189.90. H-NMR (400 MHz, d₆-DMSO) δ 3.59-3.44 (m, 2H), 3.28-3.25 (m, 3H), 2.10-1.95 (m, 2H), 1.41 (s, 9H). F-NMR (376 MHz, d₆-DMSO) δ −83.29 (3F), −119.74-122.14 (2F).

### Step 4: 3-(1,1,2,2,2-pentafluoroethyl)pyrrolidine hydrochloride

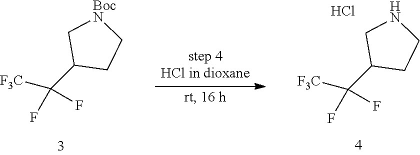 [SMILES: Cl ; CC(C)(C)OC(=O)N1CCC(C(F)(F)C(F)(F)F)C1 ; FC(F)(F)C(F)(F)C1CCNC1]

To a stirred solution of tert-butyl 3-(1,1,2,2,2-pentafluoroethyl)pyrrolidine-1-carboxylate (1.20 g, 4.15 mmol) in dioxane (10.00 mL) was added HCl (gas) in 1,4-dioxane (10.00 mL) dropwise at 0° C. The reaction mixture was stirred for 16 h at room temperature. The resulting mixture was concentrated under reduced pressure to afford 3-(1,1,2,2,2-pentafluoroethyl)pyrrolidine hydrochloride (930 mg, crude) as a yellow solid. It was used directly to next step without further purification. MS ESI calculated for C₆H₉ClF₅N [M−HCl+H]⁺, 190.06, found 190.10. H-NMR (400 MHz, d₆-DMSO) δ 9.72 (brs, 2H), 3.55-3.50 (m, 1H), 3.43-3.30 (m, 2H), 3.24-3.15 (m, 2H), 2.30-2.22 (m, 1H), 2.02-1.92 (m, 1H). F-NMR (376 MHz, d₆-DMSO) δ −83.30 (3F), −118.90, −121.75 (2F).

### Intermediate 24: 3-(trifluoromethyl)-1,2,5,6-tetrahydropyridine hydrochloride

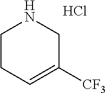

### Step 1: tert-butyl 3-(trifluoromethyl)-5,6-dihydro-2H-pyridine-1-carboxylate

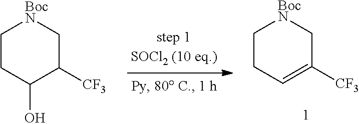 [SMILES: CC(C)(C)OC(=O)N1CCC(O)C(C(F)(F)F)C1 ; CC(C)(C)OC(=O)N1CCC=C(C(F)(F)F)C1]

To a stirred solution of tert-butyl 4-hydroxy-3-(trifluoromethyl)piperidine-1-carboxylate (0.50 g, 1.86 mmol) in pyridine (10 mL) was added SOCl₂ (1.35 mL, 18.61 mmol) dropwise at 0° C. under nitrogen atmosphere. The reaction mixture was stirred for 1 h at 80° C. The resulting mixture was quenched with water (50 mL) and extracted with EA (3×40 mL). The combined organic layers was washed with 1 M HCl (3×30 mL) and brine (30 mL), dried over anhydrous Na₂SO₄ and filtered. The filtrate was concentrated under reduced pressure. The residue was purified by Prep-TLC (PE/EA=3/1) to afford tert-butyl 3-(trifluoromethyl)-5,6-dihydro-2H-pyridine-1-carboxylate (0.30 g, 64%) as a yellow oil. H-NMR (400 MHz, CDCl₃) δ 6.47 (d, J=3.6 Hz, 1H), 4.02 (s, 2H), 3.50 (t, J=5.6 Hz, 2H), 2.27-2.24 (m, 2H), 1.48 (s, 9H). F-NMR (376 MHz, CDCl₃) δ −68.27.

### Step 2: 3-(trifluoromethyl)-1,2,5,6-tetrahydropyridine hydrochloride

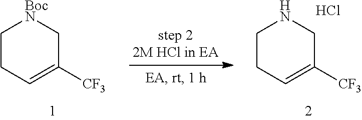 [SMILES: Cl ; CC(C)(C)OC(=O)N1CCC=C(C(F)(F)F)C1 ; FC(F)(F)C1=CCCNC1]

To a stirred solution of tert-butyl 3-(trifluoromethyl)-5,6-dihydro-2H-pyridine-1-carboxylate (0.17 g, 0.68 mmol) in EA (1 mL) was added 2 M HCl in EA (5 mL) dropwise at 0° C. under nitrogen atmosphere. The reaction mixture was stirred for 1 h at room temperature. The resulting mixture was concentrated under reduced pressure to afford 3-(trifluoromethyl)-1,2,5,6-tetrahydropyridine hydrochloride (0.13 g, 100%) which was directly used to next step without further purification. H-NMR (400 MHz, CDCl₃) δ 6.60 (s, 1H), 3.88 (s, 2H), 3.42-3.38 (m, 2H), 2.69-2.65 (m, 2H); F-NMR (376 MHz, CDCl₃) δ −68.58.

### Intermediate 25: 3-(2,2,2-trifluoroethyl)-2,5-dihydro-1H-pyrrole hydrochloride

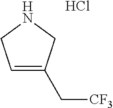

### Step 1: tert-butyl 3-(2,2,2-trifluoroethyl)-2,5-dihydropyrrole-1-carboxylate

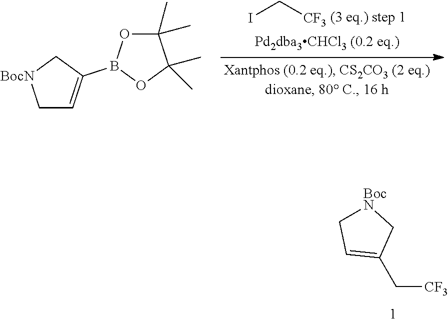 [SMILES: CC(C)(C)OC(=O)N1CC=C(B2OC(C)(C)C(C)(C)O2)C1 ; FC(F)(F)CI ; CC(C)(C)OC(=O)N1CC=C(CC(F)(F)F)C1]

To a solution of tert-butyl 3-(4,4,5,5-tetramethyl-1,3,2-dioxaborolan-2-yl)-2,5-dihydropyrrole-1-carboxylate (0.50 g, 1.69 mmol) and 1,1,1-trifluoro-2-iodoethane (1.07 g, 5.08 mmol) in 1,4-dioxane (10 mL) were added Cs₂CO₃ (2.21 g, 6.77 mmol) and XantPhos (0.196 g, 0.34 mmol) and Pd₂(dba)₃.CHCl₃ (0.35 g, 0.34 mmol) at room temperature. The reaction mixture was degassed with nitrogen for three times and stirred overnight at 80° C. The resulting mixture was concentrated under reduced pressure. The residue was purified by silica gel column chromatography, eluted with PE/EtOAc (1:2). The fractions contained desired product were combined and concentrated to afford tert-butyl 3-(2,2,2-trifluoroethyl)-2,5-dihydropyrrole-1-carboxylate (0.12 g, 25%) as a colorless oil. MS ESI calculated for C₁₁H₁₆F₃NO₂ [M+H-t-Bu]⁺, 196.11, found 196.10.

### Step 2: 3-(2,2,2-trifluoroethyl)-2,5-dihydro-1H-pyrrole hydrochloride

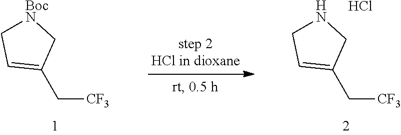 [SMILES: Cl ; CC(C)(C)OC(=O)N1CC=C(CC(F)(F)F)C1 ; FC(F)(F)CC1=CCNC1]

To a stirred solution of tert-butyl 3-(2,2,2-trifluoroethyl)-2,5-dihydropyrrole-1-carboxylate (0.12 g, 0.48 mmol) in DCM (2.00 mL) was added HCl (gas) in 1,4-dioxane (4 M, 2.00 mL, 0.05 mmol) dropwise at 0° C. under nitrogen atmosphere. The reaction mixture was stirred for 0.5 h at room temperature. The resulting mixture was concentrated under reduced pressure to afford 3-(2,2,2-trifluoroethyl)-2,5-dihydro-1H-pyrrole hydrochloride (85 mg, 100%) as a yellow solid. It was used in the next step directly without further purification. MS ESI calculated for C₆H₉ClF₃N [M+H]⁺, 152.06; found 152.10. ¹H NMR (400 MHz, CDCl₃) δ 10.43 (brs, 2H), 5.84 (s, 1H), 4.24-4.20 (m, 2H), 4.18-4.10 (m, 2H), 3.05 (q, J=10.4 Hz, 2H).

### Intermediate 26: 3-(1,1,2,2,2-pentafluoroethyl)-2,5-dihydro-1H-pyrrole hydrochloride

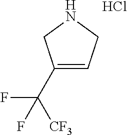 [SMILES: Cl ; FC(F)(F)C(F)(F)C1=CCNC1]

### Step 1: 3-(1,1,2,2,2-pentafluoroethyl)-2,5-dihydro-1H-pyrrole hydrochloride

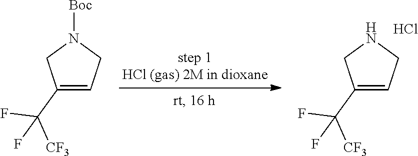 [SMILES: CC(C)(C)OC(=O)N1CC=C(C(F)(F)C(F)(F)F)C1 ; FC(F)(F)C(F)(F)C1=CCNC1 ; Cl]

To a stirred solution of tert-butyl 3-(1,1,2,2,2-pentafluoroethyl)-2,5-dihydropyrrole-1-carboxylate (0.1 g, 0.35 mmol) in dioxane (1 mL, 11.80 mmol) was added HCl (gas) in 1,4-dioxane (4 M) (1 mL, 32.91 mmol) dropwise at 0° C. The reaction mixture was stirred for 16 h at room temperature. The resulting mixture was concentrated under reduced pressure to afford 3-(1,1,2,2,2-pentafluoroethyl)-2,5-dihydro-1H-pyrrole hydrochloride (79 mg, 91%) as a yellow solid. It was used directly to next step without further purification. MS ESI calculated for C₆H₇ClF₅N [M−HCl+H]⁺, 188.11, found 187.90. H-NMR (400 MHz, CDCl₃) δ 10.19 (brs, 2H), 6.82 (s, 1H), 4.21-4.17 (m, 4H).

### Intermediate 27: 1,1-difluoro-6-azaspiro[3.4]octane

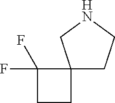

### Step 1: benzyl 1,1-difluoro-6-azaspiro[3.4]octane-6-carboxylate

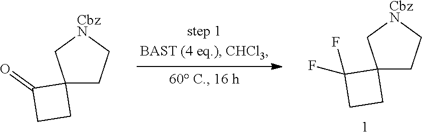 [SMILES: O=C(OCc1ccccc1)N1CCC2(CCC2=O)C1 ; O=C(OCc1ccccc1)N1CCC2(CCC2(F)F)C1]

To a stirred solution of benzyl 1-oxo-6-azaspiro[3.4]octane-6-carboxylate (2.00 g, 7.71 mmol) in CHCl₃ (20.00 mL) was added BAST (6.83 g, 30.852 mmol) dropwise at 0° C. under argon atmosphere. The reaction mixture was stirred for 20 h at 60° C. under argon atmosphere. The resulting mixture was neutralized to pH 7 with saturated NaHCO₃. The organic phase was concentrated under reduced pressure. The residue was purified by silica gel column chromatography, eluted with PE/EtOAc (3:1). The fractions contained desired product were combined and concentrated to afford benzyl 1,1-difluoro-6-azaspiro[3.4]octane-6-carboxylate (1 g, 46.09%) as a yellow oil. MS ESI calculated for C₁₅H₁₇F₂NO₂ [M+H]⁺, 282.1, found 282.3.

### Step 2: 1,1-difluoro-6-azaspiro[3.4]octane

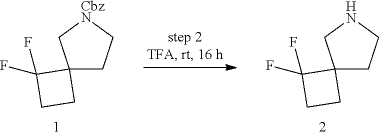 [SMILES: O=C(OCc1ccccc1)N1CCC2(CCC2(F)F)C1 ; FC1(F)CCC12CCNC2]

A solution of benzyl 1,1-difluoro-6-azaspiro[3.4]octane-6-carboxylate (75.00 mg, 0.267 mmol) in CF₃COOH (5.00 mL) was stirred for 2 h at 70° C. under argon atmosphere. The solution was concentrated under reduced pressure. The residue was diluted with EA (10 mL). The resulting mixture was washed with sat. aqueous NaHCO₃ (10 mL×2). The organic layer was dried over anhydrous Na₂SO₄ and filtered. The filtrate was concentrated under reduced pressure to afford 1,1-difluoro-6-azaspiro[3.4]octane (35 mg, 90%) as an off-white semi-solid. The crude product was used directly to next step without further purification. ¹H NMR (400 MHz, DMSO-d6) δ 3.12-2.99 (m, 2H), 3.02-2.90 (m, 2H), 2.10-1.83 (m, 3H), 1.82-1.67 (m, 4H).

### Intermediate 28: (Z)-3-(2,2,2-trifluoroethylidene)pyrrolidine hydrochloride

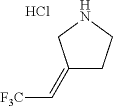

### Step 1: tert-butyl (3Z)-3-(bromomethylidene)pyrrolidine-1-carboxylate and tert-butyl (3E)-3-(bromomethylidene)pyrrolidine-1-carboxylate

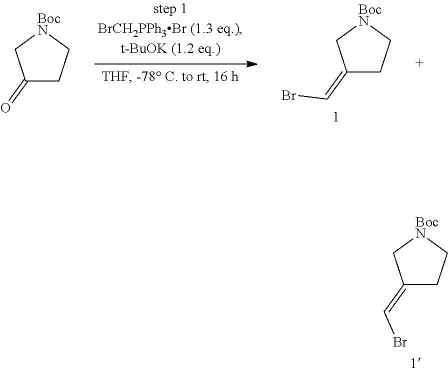

To a solution of (bromomethyl)triphenylphosphanium bromide (30.61 g, 70.18 mmol) in THF (220 mL) was added t-BuOK (1 M in THF) (64.78 mL, 64.78 mmol) dropwise at −78° C. The reaction mixture was stirred for 1.5 h at −78° C. To the above solution was added tert-butyl 3-oxopyrrolidine-1-carboxylate (10 g, 53.99 mmol) in THF (40 mL). The reaction mixture was allowed to gradually warm to room temperature and stirred overnight. The resulting mixture was quenched by the addition of water (500 mL) at room temperature and extracted with DCM (2×500 mL). The combined organic layers was washed with brine (2×300 mL), dried over anhydrous Na₂SO₄ and filtered. The filtrate was concentrated under reduced pressure. The residue was purified by silica gel column chromatography, eluted with EA/PE (0 to 20%). The fractions contained desired product were combined and concentrated to afford tert-butyl (3Z)-3-(bromomethylidene)pyrrolidine-1-carboxylate (1.5 g, 11%) as a yellow oil and tert-butyl (3E)-3-(bromomethylidene)pyrrolidine-1-carboxylate (1.8 g, 13%) as a yellow oil. Tert-butyl (3Z)-3-(bromomethylidene)pyrrolidine-1-carboxylate, MS ESI calculated for C₁₀H₁₆BrNO₂ [M-t-Bu]⁺, 205.97, 207.97, found 205.85, 207.85. ¹H-NMR (400 MHz, d₆-DMSO) δ 6.43-6.40 (m, 1H), 3.93-3.91 (m, 2H), 3.45-3.42 (m, 2H), 2.58-2.55 (m, 2H), 1.41 (s, 9H). Tert-butyl (3E)-3-(bromomethylidene)pyrrolidine-1-carboxylate, MS ESI calculated for C₁₀H₁₆BrNO₂ [M-t-Bu]⁺, 205.97, 207.97, found 205.85, 207.85. ¹H-NMR (400 MHz, d₆-DMSO) δ 6.37-6.34 (m, 1H), 3.87-3.84 (m, 2H), 3.47-3.44 (m, 2H), 2.60-2.57 (m, 2H), 1.42 (s, 9H).

### Step 2: tert-butyl (3Z)-3-(2,2,2-trifluoroethylidene)pyrrolidine-1-carboxylate

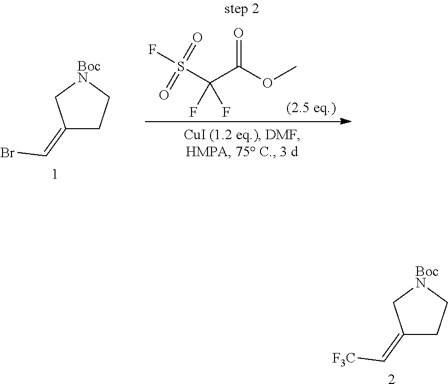

To a mixture of tert-butyl (3Z)-3-(bromomethylidene)pyrrolidine-1-carboxylate (0.39 g, 1.49 mmol) and CuI (0.68 g, 3.571 mmol) in HMPA (2.50 mL, 14.29 mmol) and DMF (2.50 mL, 32.30 mmol) was added a solution of methyl 2,2-difluoro-2-sulfoacetate (2.86 g, 14.88 mmol) and DMF (2.50 mL, 0.034 mmol) dropwise over 1 h at 75° C. The reaction mixture was degassed with nitrogen and stirred for 3 days at 75° C. The resulting mixture was concentrated under reduced pressure. The residue was purified by silica gel column chromatography, eluted with PE/EtOAc (4:1). The fractions contained desired product were combined and concentrated to afford tert-butyl (3Z)-3-(2,2,2-trifluoroethylidene)pyrrolidine-1-carboxylate (66 mg, 18%) as a colorless oil. MS ESI calculated for C₁₁H₁₆F₃NO₂ [M-tBu]⁺, 195.11, found 195.90.

### Step 3: (Z)-3-(2,2,2-trifluoroethylidene)pyrrolidine hydrochloride

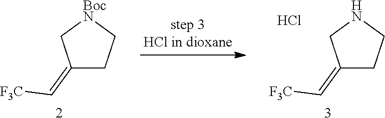

To a mixture of tert-butyl (3Z)-3-(2,2,2-trifluoroethylidene)pyrrolidine-1-carboxylate (66.00 mg, 0.263 mmol) in 1,4-dioxane (2.00 mL, 23.608 mmol) was HCl (gas) in 1,4-dioxane (1.00 mL, 32.912 mmol). The reaction mixture was stirred overnight at room temperature under nitrogen atmosphere. The resulting mixture was concentrated under vacuum to afford (3Z)-3-(2,2,2-trifluoroethylidene) pyrrolidine hydrochloride (48 mg, 97.41%) as a light yellow solid. The crude product was used in the next step directly without further purification. MS ESI calculated for C₆H₉ClF₃N [M+H—HCl]⁺, 152.06; found 151.90.

### Intermediate 29: (3E)-3-(2,2,2-trifluoroethylidene)pyrrolidine hydrochloride

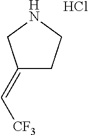

The title compound was prepared using procedures similar to those described in Intermediate 28 using (3E)-3-(bromomethylidene)pyrrolidine-1-carboxylate instead of (3Z)-3-(bromomethylidene)pyrrolidine-1-carboxylate to afford the title compound as a yellow solid.

### Intermediate 30: (Z)-3-(1,1,1-trifluoropropan-2-ylidene)pyrrolidine hydrochloride

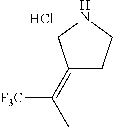

### Step 1: 2,2,2-trifluoro-1-[3-2,2,2-trifluoroacetyl)pyrrolidin-1-yl]ethanone

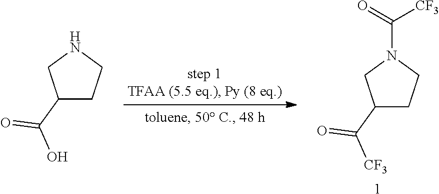

To a stirred mixture of pyrrolidine-3-carboxylic acid (4.00 g, 34.74 mmol) in Toluene (60.00 mL) were added TFAA (26.58 mL, 126.55 mmol) dropwise and Pyridine (22.37 mL, 282.835 mmol) dropwise at 0° C. under nitrogen atmosphere. The reaction mixture was stirred for 48 h at 50° C. The resulting mixture was quenched with water (50 mL) at 0° C. and stirred for additional 2 h at 45° C. The resulting mixture was extracted with EtOAc (3×100 mL). The combined organic layers was washed with brine (100 mL), dried over anhydrous Na₂SO₄ and filtered. The filtrate was concentrated under vacuum to afford 2,2,2-trifluoro-1-[3-(2,2,2-trifluoroacetyl)pyrrolidin-1-yl]ethanone (10 g, crude) as brown oil. The crude product was used directly to next step without further purification. MS ESI calculated for C₈H₇F₆NO₂ [M+H+H₂O]⁺, 282.04, found 282.00.

### Step 2: tert-butyl 3-(2,2,2-trifluoroacetyl)pyrrolidine-1-carboxylate

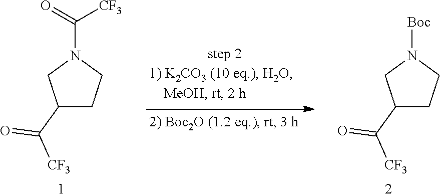

To a stirred mixture of 2,2,2-trifluoro-1-[3-(2,2,2-trifluoroacetyl)pyrrolidin-1-yl]ethanone (2.30 g, 8.74 mmol), MeOH (15.00 mL) and H₂O (0.47 mL, 26.222 mmol) was added K₂CO₃ (2.42 g, 17.481 mmol) at room temperature under nitrogen atmosphere. The reaction mixture was stirred for 2 h at room temperature. To the above mixture was added (Boc)₂O (2.29 g, 10.493 mmol) dropwise at room temperature. The resulting mixture was stirred for additional 16 h at room temperature. The resulting mixture was concentrated and extracted with EtOAc (3×100 mL). The combined organic layers was washed with brine (100 mL), dried over anhydrous Na₂SO₄ and filtered. The filtrate was concentrated under reduced pressure. The residue was purified by silica gel column chromatography, eluted with EA/PE (0 to 70%). The fractions contained desired product were combined and concentrated to afford tert-butyl 3-(2,2,2-trifluoroacetyl)pyrrolidine-1-carboxylate (1.87 g, 80%) as a brown oil. MS ESI calculated for C₁₁H₁₆F₃NO₃ [M-t-Bu+H]⁺, 212.05, found 212.05.

### Step 3: tert-butyl 3-(1,1,1-trifluoro-2-hydroxypropan-2-yl)pyrrolidine-1-carboxylate

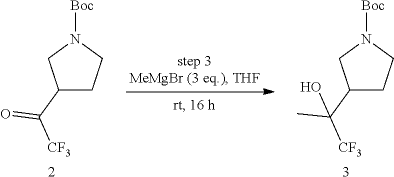

To a stirred solution of tert-butyl 3-(2,2,2-trifluoroacetyl)pyrrolidine-1-carboxylate (5.8 g, 21.70 mmol) in THF (60.00 mL) was added MeMgBr (21.70 mL, 65.109 mmol) dropwise at −70° C. under nitrogen atmosphere. The reaction mixture was stirred for 16 h at room temperature under nitrogen atmosphere. The reaction was quenched by the addition of NH₄Cl (aq.) (sat., 200 mL). The resulting mixture was extracted with EtOAc (3×200 mL). The combined organic layers was washed with brine (1×500 mL), dried over anhydrous Na₂SO₄ and filtered. The filtrate was concentrated under reduced pressure. The residue was purified by silica gel column chromatography, eluted with EtOAc/PE (0-100%). The fractions contained desired product were combined and concentrated to afford tert-butyl 3-(1,1,1-trifluoro-2-hydroxypropan-2-yl)pyrrolidine-1-carboxylate (0.87 g, 14%) as a light yellow solid. MS ESI calculated for C₁₂H₂₀F₃NO₃ [M-t-Bu+H]⁺, 228.14, found 228.05. H-NMR (400 MHz, d₆-DMSO) δ 6.02-5.99 (m, 1H), 3.43-3.40 (m, 2H), 3.18-3.01 (m, 2H), 2.45-2.41 (m, 1H), 1.92-1.80 (m, 2H), 1.40 (s, 9H), 1.27 (s, 3H).

### Step 4: tert-butyl (3Z)-3-(1,1,1-trifluoropropan-2-ylidene)pyrrolidine-1-carboxylate and tert-butyl (3E)-3-(1,1,1-trifluoropropan-2-ylidene)pyrrolidine-1-carboxylate

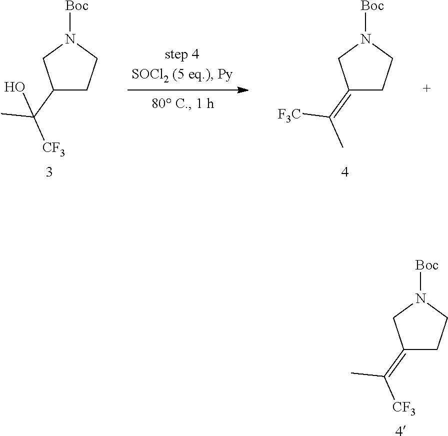

To a stirred solution of tert-butyl 3-(1,1,1-trifluoro-2-hydroxypropan-2-yl)pyrrolidine-1-carboxylate (1.13 g, 3.99 mmol) in Pyridine (11.00 mL) was added SOCl₂ (2372.76 mg, 19.944 mmol) dropwise at room temperature under nitrogen atmosphere. The reaction mixture was stirred for 1 h at 80° C. under nitrogen atmosphere. The resulting mixture was allowed to cool down to room temperature and concentrated under reduced pressure. The residue was purified by silica gel column chromatography, eluted with EA in PE (0-30%). The fractions contained desired product were combined and concentrated to afford tert-butyl (3Z)-3-(1,1,1-trifluoropropan-2-ylidene)pyrrolidine-1-carboxylate (0.20 g, 19%) as a yellow oil and tert-butyl (3E)-3-(1,1,1-trifluoropropan-2-ylidene)pyrrolidine-1-carboxylate (0.70 g, 66%) as a yellow oil. Tert-butyl (3Z)-3-(1,1,1-trifluoropropan-2-ylidene)pyrrolidine-1-carboxylate: MS ESI calculated for C₁₂H₁₈F₃NO₂ [M-t-Bu+H]⁺, 210.13, found 209.95. H-NMR (400 MHz, d₆-DMSO) δ 4.23-4.19 (m, 2H), 3.56-3.54 (m, 2H), 2.68-2.66 (m, 2H), 1.82 (s, 3H), 1.56 (s, 9H).

Tert-butyl (3E)-3-(1,1,1-trifluoropropan-2-ylidene)pyrrolidine-1-carboxylate: MS ESI calculated for C₁₂H₁₈F₃NO₂ [M-t-Bu+H]⁺, 210.13, found 209.95. H-NMR (400 MHz, d₆-DMSO) δ 4.08-4.06 (m, 2H), 3.59-3.57 (m, 2H), 2.84-2.82 (m, 2H), 1.80 (s, 3H), 1.53 (s, 9H).

### Step 5: (3Z)-3-(1,1,1-trifluoropropan-2-ylidene)pyrrolidine hydrochloride

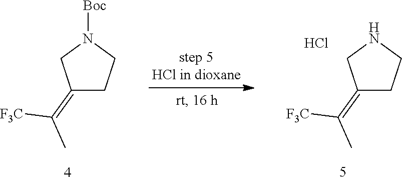

To a stirred solution of tert-butyl (3Z)-3-(1,1,1-trifluoropropan-2-ylidene)pyrrolidine-1-carboxylate (80.00 mg, 0.302 mmol) in dioxane (1.50 mL) was added HCl (gas) in 1,4-dioxane (1.50 mL) dropwise at 0° C. The reaction solution was stirred for 16 h at room temperature. The resulting mixture was concentrated under reduced pressure to afford (3Z)-3-(1,1,1-trifluoropropan-2-ylidene)pyrrolidine hydrochloride (58 mg) as a yellow solid. The crude product was used directly to next step without further purification. MS ESI calculated for C₇H₁₁ClF₃N [M−HCl+H]⁺, 166.08, found 166.10. H-NMR (400 MHz, d₆-DMSO) δ 9.57 (brs, 2H), 4.02-3.99 (m, 2H), 3.58-3.40 (m, 2H), 2.74-2.72 (m, 2H), 1.85 (s, 3H).

### Intermediate 31: (E)-3-(1,1,1-trifluoropropan-2-ylidene)pyrrolidine hydrochloride

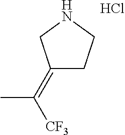

The title compound was prepared using procedures similar to those described in Intermediate 30 step 5 using tert-butyl (3E)-3-(1,1,1-trifluoropropan-2-ylidene)pyrrolidine-1-carboxylate instead of tert-butyl (3Z)-3-(1,1,1-trifluoropropan-2-ylidene)pyrrolidine-1-carboxylate to afford the title compound as a light yellow solid.

### Intermediate 32: 3-(2,2,2-trifluoroethyl)-2,5-dihydro-1H-pyrrole hydrochloride

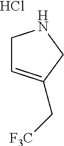

### Step 1: Tert-butyl 3-(2,2,2-trifluoroethyl)-2,5-dihydropyrrole-1-carboxylate

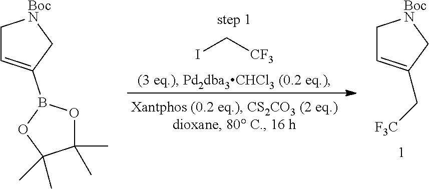 [SMILES: CC(C)(C)OC(=O)N1CC=C(B2OC(C)(C)C(C)(C)O2)C1 ; FC(F)(F)CI ; CC(C)(C)OC(=O)N1CC=C(CC(F)(F)F)C1]

To a solution of tert-butyl 3-(4,4,5,5-tetramethyl-1,3,2-dioxaborolan-2-yl)-2,5-dihydropyrrole-1-carboxylate (0.50 g, 1.69 mmol) and 1,1,1-trifluoro-2-iodoethane (1.07 g, 5.08 mmol) in 1,4-dioxane (10 mL) were added Cs₂CO₃ (2.21 g, 6.78 mmol), XantPhos (0.196 g, 0.339 mmol) and Pd₂(dba)₃.CHCl₃ (0.35 g, 0.339 mmol) at room temperature. The reaction mixture was degassed with nitrogen for three times and stirred overnight at 80° C. The resulting mixture was concentrated under reduced pressure. The residue was purified by silica el column chromatography, eluted with PE/EtOAc (1:2). The fractions contained desired product were combined and concentrated to afford tert-butyl 3-(2,2,2-trifluoroethyl)-2,5-dihydropyrrole-1-carboxylate (0.12 g, 25%) as a white oil. MS ESI calculated for C₁₁H₁₆F₃NO₂ [M+H-tBu], 196.05, found 196.10.

### Step 2: 3-(2,2,2-trifluoroethyl)-2,5-dihydro-1H-pyrrole hydrochloride

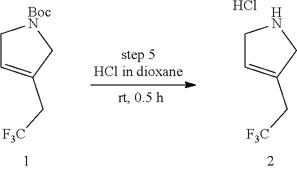 [SMILES: Cl ; CC(C)(C)OC(=O)N1CC=C(CC(F)(F)F)C1 ; FC(F)(F)CC1=CCNC1]

To a stirred solution of tert-butyl 3-(2,2,2-trifluoroethyl)-2,5-dihydropyrrole-1-carboxylate (0.12 g, 0.48 mmol) in DCM (2.00 mL) was added HCl (gas) in 1,4-dioxane (4 M) (2.00 mL, 0.055 mmol) at 0° C. under nitrogen atmosphere. The reaction mixture was stirred for 0.5 h at room temperature. The resulting mixture was concentrated under reduced pressure to afford 3-(2,2,2-trifluoroethyl)-2,5-dihydro-1H-pyrrole hydrochloride (90 mg, 100%) as a yellow solid. The crude product was used directly in next step without further purification. MS ESI calculated for C₆H₉ClF₃N [M+H—HCl]⁺, 152.06; found 152.10.

### Intermediate 33: (Z)-2-(Pyrrolidin-3-ylidene)propanenitrile hydrochloride

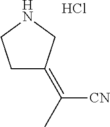

### Step 1: (3Z)-3-(1-cyanoethylidene)pyrrolidine-1-carboxylate/(3E)-3-(1-cyanoethylidene)pyrrolidine-1-carboxylate

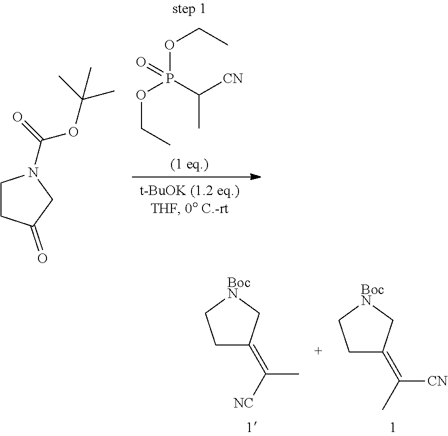

To a solution of diethyl 1-cyanoethylphosphonate (1.15 g, 5.99 mmol) in THF (30 mL) was added t-BuOK (0.81 g, 7.19 mmol) at 0° C. The reaction mixture was stirred for 10 min. After which time, a solution of tert-butyl 3-oxopyrrolidine-1-carboxylate (1.11 g, 5.99 mmol) in THF (6 mL) was added dropwise. The reaction mixture was allowed to warm to room temperature and stirred for 16 h. The resulting mixture was concentrated under reduced pressure. The residue was purified by silica gel column chromatography, eluted with 20-35% EA in PE. The fractions contained desired product were combined and concentrated to afford tert-butyl (3E)-3-(1-cyanoethylidene)pyrrolidine-1-carboxylate (0.50 g, 38%) as a colorless oil and tert-butyl (3Z)-3-(1-cyanoethylidene)pyrrolidine-1-carboxylate (0.48 g, 36%) as a colorless oil. Tert-butyl (3E)-3-(1-cyanoethylidene)pyrrolidine-1-carboxylate: MS ESI calculated for C₁₂H₁₈N₂O₂[M+H]⁺, 223.14; found 223.05. ¹H-NMR (400 MHz, d6-DMSO) δ 4.03 (m, 2H), 3.46 (t, J=7.2 Hz, 2H), 2.79 (s, 2H), 1.84-1.78 (m, 3H), 1.41 (s, 9H). Tert-butyl (3Z)-3-(1-cyanoethylidene)pyrrolidine-1-carboxylate: MS ESI calculated for C₁₂H₁₈N₂O₂[M+H]⁺, 223.14; found 223.05. ¹H-NMR (400 MHz, d6-DMSO) δ 4.02 (s, 2H), 3.40 (t, J=7.2 Hz, 2H), 2.64 (s, 2H), 1.78 (s, 3H), 1.34 (s, 9H).

### Step 2: (Z)-2-(pyrrolidin-3-ylidene)propanenitrile hydrochloride

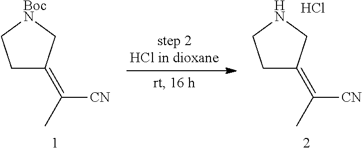

To a stirred solution of tert-butyl (3Z)-3-(1-cyanoethylidene)pyrrolidine-1-carboxylate (0.23 g, 1.03 mmol) in dioxane (5.75 mL, 65.26 mmol) was added HCl (4M in 1,4-dioxane) (5.75 mL) dropwise at 25° C. The reaction mixture was stirred for 16 h. The resulting mixture was concentrated under vacuum to afford (Z)-2-(pyrrolidin-3-ylidene)propanenitrile hydrochloride (165 mg, 100%) as a yellow solid. The crude product was used in the next step directly without further purification. MS ESI calculated for C₇H₁₁ClN₂ [M+H—HCl]⁺, 123.08; found 123.20.

### Intermediate 34: (E)-2-(pyrrolidin-3-ylidene)propanenitrile hydrochloride

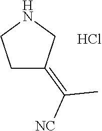

The title compound was prepared using procedures similar to those described in Intermediate 33 step 2 using tert-butyl (3E)-3-(1-cyanoethylidene)pyrrolidine-1-carboxylate instead of tert-butyl (3Z)-3-(1-cyanoethylidene)pyrrolidine-1-carboxylate to afford the title compound as a light yellow solid.

### Intermediate 35: 2-methyl-2-(pyrrolidin-3-yl)propanenitrile hydrochloride

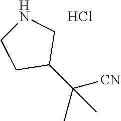

### Step 1: Tert-butyl 3-(1-cyano-1-methylethyl)pyrrolidine-1-carboxylate

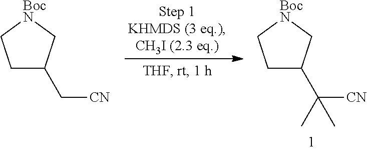 [SMILES: CC(C)(C)OC(=O)N1CCC(CC#N)C1 ; CC(C)(C)OC(=O)N1CCC(C(C)(C)C#N)C1]

To a stirred solution of tert-butyl 3-(cyanomethyl)pyrrolidine-1-carboxylate (0.5 g, 2.38 mmol) in THF (4 mL) was added KHMDS (1.43 mL, 1.43 mmol) dropwise at −5° C. under nitrogen atmosphere. The reaction mixture was stirred for 10 minutes, after which time a solution of CH₃I (0.776 g, 5.47 mmol) in THF (0.70 mL) was added slowly over a period of 10 minutes. The reaction mixture was stirred for another 1 h. The resulting mixture was quenched with NH₄Cl (sat.) at 0° C. and extracted with EA (3×30 mL). The combined organic layers was washed with brine (2×30 mL), dried over anhydrous Na₂SO₄ and filtered. The filtrate was concentrated under reduced pressure. The residue was purified by silica gel column chromatography, eluted with 30% EA in PE. The fractions contained desired product were combined and concentrated to afford tert-butyl 3-(1-cyano-1-methylethyl)pyrrolidine-1-carboxylate (0.23 g, 41%) as a colorless oil. H-NMR (400 MHz, CDCl₃) δ 3.60 (s, 2H), 3.31-3.15 (m, 2H), 2.20-1.85 (m, 3H), 1.47 (s, 9H), 1.40 (s, 3H), 1.37 (s, 3H).

### Step 2: 2-Methyl-2-(pyrrolidin-3-yl)propanenitrile hydrochloride

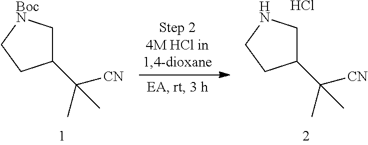

To a stirred solution of tert-butyl 3-(1-cyano-1-methylethyl)pyrrolidine-1-carboxylate (0.23 g, 0.98 mmol) in EA (1.00 mL, 10.22 mmol) was added 4 M HCl in 1,4-dioxane dropwise at 0° C. under nitrogen atmosphere. The reaction mixture was stirred for 3 h at ambient temperature. The resulting mixture was concentrated under reduced pressure to afford 2-methyl-2-(pyrrolidin-3-yl)propanenitrile hydrochloride (0.16 g, 94%) as an off-white solid. H-NMR (400 MHz, CDCl₃) δ 3.66-3.62 (m, 1H), 3.54-3.50 (m, 1H), 3.44-3.40 (m, 1H), 3.12-3.08 (m, 1H), 2.26-2.24 (m, 1H), 2.01-1.93 (m, 2H), 1.43-1.42 (m, 6H).

### Intermediate 36: 3-(1,1,1-trifluoropropan-2-yl)pyrrolidine hydrochloride

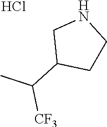

### Step 1: Tert-butyl 3-(1,1,1-trifluoropropan-2-yl)pyrrolidine-1-carboxylate

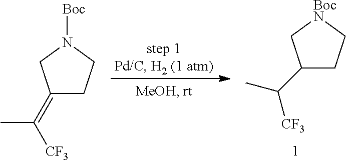 [SMILES: C/C(=C1/CCN(C(=O)OC(C)(C)C)C1)C(F)(F)F ; CC(C1CCN(C(=O)OC(C)(C)C)C1)C(F)(F)F]

To a solution of tert-butyl (3E)-3-(1,1,1-trifluoropropan-2-ylidene)pyrrolidine-1-carboxylate (0.35 g, 1.32 mmol) in MeOH (5.00 mL) was added Pd/C (10%) (0.15 g, 1.41 mmol). The reaction mixture was degassed with hydrogen and stirred for 1 h at room temperature under (1 atm) hydrogen atmosphere. The resulting mixture was filtered, the filter cake was washed with MeOH (4×5 mL). The filtrate was concentrated under reduced pressure. This resulted in tert-butyl 3-(1,1,1-trifluoropropan-2-yl)pyrrolidine-1-carboxylate (320 mg) as colorless oil. The crude product was used directly to next step without further purification. MS ESI calculated for C₁₂H₂₀F₃NO₂ [M-Boc+H]⁺, 168.09, found 167.95. ¹H-NMR (400 MHz, CDCl₃) δ 3.68-3.51 (m, 2H), 3.32-3.18 (m, 1H), 3.04-2.94 (m, 1H), 2.35-2.03 (m, 3H), 1.74-1.52 (m, 1H), 1.51 (s, 9H), 1.22-1.08 (m, 3H).

### Step 2: 3-(1,1,1-Trifluoropropan-2-yl)pyrrolidine hydrochloride

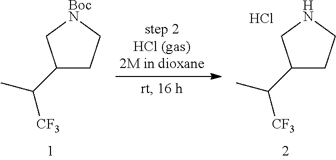 [SMILES: CC(C1CCN(C(=O)OC(C)(C)C)C1)C(F)(F)F ; CC(C1CCNC1)C(F)(F)F ; Cl]

To a stirred solution of tert-butyl 3-(1,1,1-trifluoropropan-2-yl)pyrrolidine-1-carboxylate (0.32 g, 1.20 mmol) in dioxane (3.00 mL) was added HCl (gas, 2 M) in 1,4-dioxane (3.00 mL) dropwise at 0° C. The reaction mixture was stirred for 16 h at room temperature. The resulting mixture was concentrated under reduced pressure to afford 3-(1,1,1-trifluoropropan-2-yl)pyrrolidine hydrochloride (280 mg) as colorless oil. The crude product was used directly to next step without further purification. MS ESI calculated for C₇H₁₃ClF₃N [M−HCl+H]⁺, 168.09, found 168.15. H-NMR (400 MHz, d₆-DMSO) δ 9.44-9.37 (m, 2H), 3.52-3.40 (m, 2H), 3.14-3.03 (m, 1H), 2.90-2.81 (m, 1H), 2.68-2.57 (m, 1H), 2.39-2.25 (m, 1H), 2.09-2.04 (m, 1H), 1.75-1.61 (m, 1H), 1.12-1.08 (m, 3H).

### Intermediate 37: 3-(2,2-difluorocyclopropyl)pyrrolidine hydrochloride

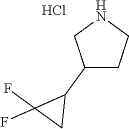

### Step 1: Tert-butyl 3-(2,2-difluorocyclopropyl)pyrrolidine-1-carboxylate

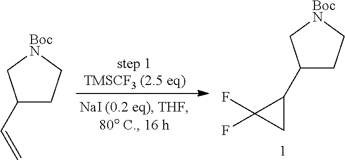 [SMILES: C=CC1CCN(C(=O)OC(C)(C)C)C1 ; CC(C)(C)OC(=O)N1CCC(C2CC2(F)F)C1]

To a mixture of tert-butyl 3-ethenylpyrrolidine-1-carboxylate (0.20 g, 1.01 mmol), and NaI (30.39 mg, 0.20 mmol) in THF (1.00 mL) was added trifluoromethyltrimethylsilane (0.36 g, 2.53 mmol). The reaction mixture was stirred overnight at 80° C. under nitrogen atmosphere. The resulting mixture was concentrated under vacuum. The residue was purified by silica gel column chromatography, eluted with EA/PE (0 to 55%). The fractions contained desired product were combined and concentrated to afford tert-butyl 3-(2,2-difluorocyclopropyl)pyrrolidine-1-carboxylate (74 mg, 50% purity, 30% yield) as a yellow oil. It was used directly for next step without further purification. MS ESI calculated for C₁₂H₁₉F₂NO₂ [M-tBu+CH₃CN+H]⁺, 233.14, found 233.05.

### Step 2: 3-(2,2-difluorocyclopropyl)pyrrolidine hydrochloride

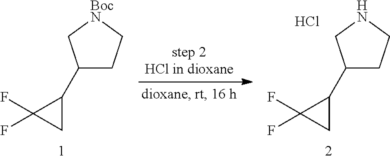 [SMILES: CC(C)(C)OC(=O)N1CCC(C2CC2(F)F)C1 ; FC1(F)CC1C1CCNC1 ; Cl]

To a stirred solution of tert-butyl 3-(2,2-difluorocyclopropyl)pyrrolidine-1-carboxylate (74.00 mg, 0.299 mmol) in dioxane (1.00 mL) was added 4 M HCl in 1,4-dioxane (1.00 mL) dropwise at 0° C. The reaction solution was stirred for 16 h at room temperature. The resulting mixture was concentrated under reduced pressure to afford 3-(2,2-difluorocyclopropyl)pyrrolidine hydrochloride (55 mg, 100%) as yellow oil. MS ESI calculated for C₇H₁₂ClF₂N [M−HCl+H]⁺, 148.09, found 148.10.

### Intermediate 38: 3-[(trifluoromethyl)sulfanyl]pyrrolidine hydrochloride

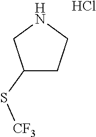

### Step 1: Tert-butyl 3-[(trifluoromethyl)sulfanyl]pyrrolidine-1-carboxylate

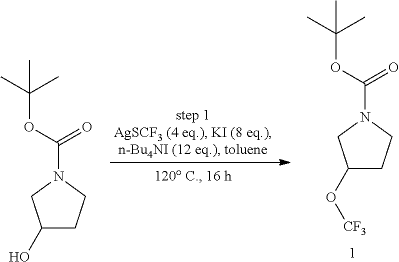 [SMILES: CC(C)(C)OC(=O)N1CCC(O)C1 ; CC(C)(C)OC(=O)N1CCC(OC(F)(F)F)C1]

To a mixture of tert-butyl 3-hydroxypyrrolidine-1-carboxylate (0.60 g, 3.20 mmol), tetrabutylammonium iodide (14.2 g, 38.45 mmol, 12.00 equiv), KI (4.2 g, 25.64 mmol) in toluene (30 mL) was added [(trifluoromethyl)sulfanyl]silver (0.89 g, 4.27 mmol). The reaction mixture was stirred for 16 h at 120° C. The resulting mixture was concentrated under reduced pressure. The residue was purified by silica gel column chromatography, eluted with EA/PE (0 to 15%). The fractions contained desired product were combined and concentrated to afford tert-butyl 3-[(trifluoromethyl)sulfanyl]pyrrolidine-1-carboxylate (1 g, crude) as brown oil. MS ESI calculated for C₁₀H₁₆F₃NO₂S [M+H-t-Bu]⁺, 216.09, found 215.95.

### Step 2: 3-[(trifluoromethyl)sulfanyl]pyrrolidine hydrochloride

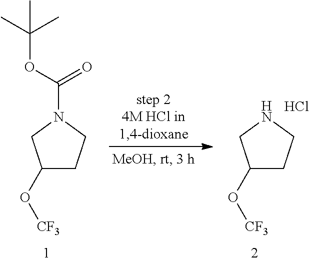 [SMILES: CC(C)(C)OC(=O)N1CCC(OC(F)(F)F)C1 ; FC(F)(F)OC1CCNC1 ; Cl]

To a mixture of tert-butyl 3-[(trifluoromethyl)sulfanyl]pyrrolidine-1-carboxylate (1.00 g, 3.69 mmol) in MeOH (4.00 mL) was added 4 M HCl in 1,4-dioxane (4.00 mL). The reaction mixture was stirred for additional 16 h at room temperature. The resulting mixture was concentrated under reduced pressure to afford 3-[(trifluoromethyl)sulfanyl]pyrrolidine hydrochloride (1 g, crude) as a brown oil. The crude product was used directly to next step without further purification. MS ESI calculated for C₅H₉ClF₃NS [M+H—HCl]⁺, 172.03, found 172.05.

### Intermediate 39: (3R)-3-(2,2,2-trifluoroethoxy)pyrrolidine

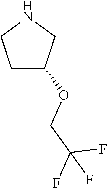 [SMILES: FC(F)(F)CO[C@@H]1CCNC1]

### Step 1: Tert-butyl 3-[(trifluoromethyl)sulfanyl]pyrrolidine-1-carboxylate

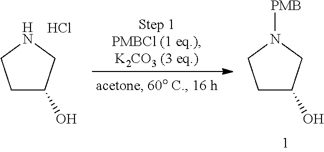

A mixture of (3R)-pyrrolidin-3-ol hydrochloride (5.00 g, 40.46 mmol), 4-methoxybenzyl chloride (6336.38 mg, 40.46 mmol) and K₂CO₃ (16775.23 mg, 121.38 mmol) in acetone (50 mL) was stirred for 16 h at 60 degrees C. under nitrogen atmosphere. The solid was filtered out. The filtrate was concentrated under vacuum. The residue was purified by silica gel column chromatography, eluted with MeOH with 10% NH₃.H₂O/DCM (0 to 8%) to afford (3R)-1-[(4-methoxyphenyl)methyl]pyrrolidin-3-ol (6.4 g, 76%) as brown oil. MS ESI calculated for C₁₂H₁₇NO₂ [M+H-t-Bu]⁺, 208.13, found 208.00.

### Step 2: (3R)-1-[(4-methoxyphenyl)methyl]-3-(2,2,2-trifluoroethoxy)pyrrolidine

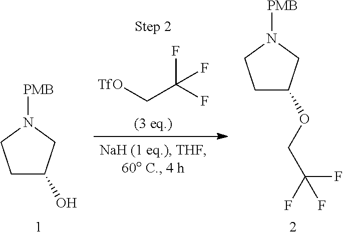

To a stirred solution of (3R)-1-[(4-methoxyphenyl)methyl]pyrrolidin-3-ol (4.80 g, 23.16 mmol) and THF (50 mL) was added NaH (926 mg, 23.16 mmol, 60%) in portions at 0 degrees C. under nitrogen atmosphere. The resulting mixture was stirred for 0.5 h at room temperature under nitrogen atmosphere. To the above mixture was added 2,2,2-trifluoroethyl trifluoromethanesulfonate (88.06 g, 34.74 mmol) dropwise over 5 min at 0 degrees C. The resulting mixture was stirred for additional 4 h at 60 degrees C. The resulting mixture was concentrated under vacuum. The residue was purified by silica gel column chromatography, eluted with MeOH/DCM (0 to 8%) to afford (3R)-1-[(4-methoxyphenyl)methyl]-3-(2,2,2-trifluoroethoxy)pyrrolidine (4 g, 60%) as brown oil. MS ESI calculated for C₁₄H₁₈F₃NO₂ [M+H]⁺, 290.13, found 290.00.

### Step 3: (3R)-3-(2,2,2-trifluoroethoxy)pyrrolidine

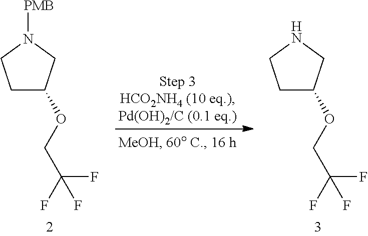

A mixture of (3R)-1-[(4-methoxyphenyl)methyl]-3-(2,2,2-trifluoroethoxy)pyrrolidine (1.00 g, 3.46 mmol), ammonium formate (1089.81 mg, 17.28 mmol), Pd(OH)₂/C (24 mg, 0.03 mmol, 20%) and MeOH (1.00 mL) was stirred for 16 h at 60 degrees C. The solid was filtered out and washed with MeOH (3×10 mL). The resulting mixture was concentrated under vacuum to afford (3R)-3-(2,2,2-trifluoroethoxy)pyrrolidine (580 mg, crude) as brown oil. MS ESI calculated for C₆H₁₀F₃NO [M+H]⁺, 170.07, found 169.90.

### Intermediate 40: (3S)-3-(2,2,2-trifluoroethoxy)pyrrolidine

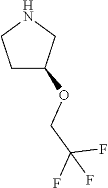 [SMILES: FC(F)(F)CO[C@H]1CCNC1]

The Intermediate 27 was prepared using procedures similar to that described in Intermediate 39 using appropriate starting materials.

### Intermediate 41: 3-trifluoromethanesulfonylpyrrolidine hydrochloride

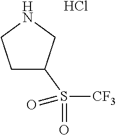 [SMILES: Cl ; O=S(=O)(C1CCNC1)C(F)(F)F]

### Step 1: tert-butyl 3-trifluoromethanesulfonylpyrrolidine-1-carboxylate

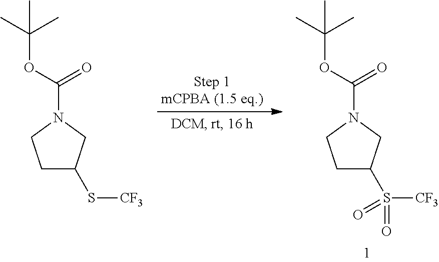 [SMILES: CC(C)(C)OC(=O)N1CCC(SC(F)(F)F)C1 ; CC(C)(C)OC(=O)N1CCC(S(=O)(=O)C(F)(F)F)C1]

A solution of tert-butyl 3-[(trifluoromethyl)sulfanyl]pyrrolidine-1-carboxylate (200.00 mg, 0.74 mmol) and MCPBA (299.33 mg, 1.47 mmol, 85%) in DCM (2.00 mL) was stirred for 16 h at 25 degrees C. The resulting mixture was concentrated under vacuum. To the reside was added MCPBA (299.33 mg, 1.47 mmol, 2.00 equiv, 85%) in DCM (2.00 mL), the resulting mixture was stirred for 16 h at 25 degrees C. The resulting mixture was diluted with CH₂Cl₂ (20 mL), washed with sat. NaHCO₃, dried over anhydrous Na₂SO₄. After filtration, the filtrate was concentrated under reduced pressure. The residue was purified by silica gel column chromatography, eluted with PE/EtOAc (4:1) to afford tert-butyl 3-trifluoromethanesulfonyl pyrrolidine-1-carboxylate (120 mg, 54%) as an off-white solid. MS ESI calculated for: C₁₀H₁₆F₃NO₄S [M+H]⁺, 304.08; found 304.10. ¹H-NMR (400 MHz, d₆-DMSO) δ 4.69 (s, 1H), 3.76-3.67 (m, 2H), 3.48 (t, J=10.4 Hz, 1H), 2.41 (s, 3H), 1.41 (s, 9H).

### Step 2: (3R)-1-[(4-methoxyphenyl)methyl]-3-(2,2,2-trifluoroethoxy)pyrrolidine

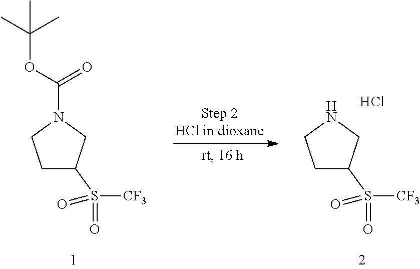 [SMILES: CC(C)(C)OC(=O)N1CCC(S(=O)(=O)C(F)(F)F)C1 ; O=S(=O)(C1CCNC1)C(F)(F)F ; Cl]

A mixture of tert-butyl 3-trifluoromethanesulfonylpyrrolidine-1-carboxylate (120.00 mg, 0.40 mmol), HCl (4 N in 1,4-dioxane, 4.00 mL) and MeOH (4.00 mL) was stirred for 16 h at room temperature. The resulting mixture was concentrated under reduced pressure. The crude product was used in the next step directly without further purification. MS ESI calculated for: C₅H₉ClF₃NO₂S [M+H—Cl]⁺, 204.02; found 204.10.

### Intermediate 42: (3S)-3-isopropoxypyrrolidine

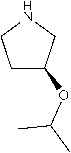

### Step 1: benzyl (3S)-3-isopropoxypyrrolidine-1-carboxylate

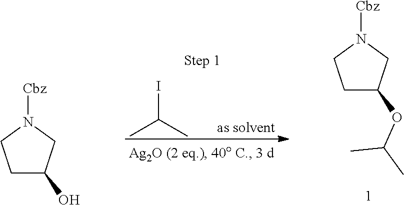

To a stirred mixture of benzyl (3S)-3-hydroxypyrrolidine-1-carboxylate (3.00 g, 13.56 mmol), Ag₂O (6284.19 mg, 27.12 mmol) and 2-iodopropane (30 mL) was stirred for 48 h at 40 degrees C. under nitrogen atmosphere. The resulting mixture was concentrated under vacuum. The residue was purified by silica gel column chromatography, eluted with EA/PE (0 to 30%) to afford benzyl (3S)-3-isopropoxypyrrolidine-1-carboxylate (2 g, 56%) as a light yellow oil. MS ESI calculated for: C₁₅H₂₁NO₃ [M+H]⁺, 264.15; found 264.00. ¹H-NMR (400 MHz, d₆-DMSO) δ 7.40-7.32 (m, 5H), 5.16-5.15 (m, 2H), 4.15-4.14 (m, 1H), 3.66-3.64 (m, 1H), 3.57-3.46 (m, 4H), 1.98-1.95 (m, 2H), 1.18-1.15 (m, 6H).

### Step 2: (3S)-3-isopropoxypyrrolidine

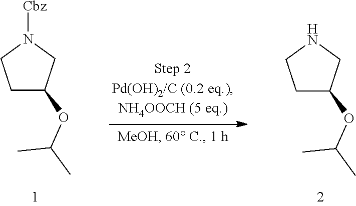

A mixture of benzyl (3S)-3-isopropoxypyrrolidine-1-carboxylate (500.00 mg, 1.90 mmol), ammonium formate (598.62 mg, 9.49 mmol), Pd(OH)₂/C (266.64 mg, 0.38 mmol, 20%) and MeOH (5.00 mL) was stirred for 1 h at 60 degrees C. The solid was filtered out. The resulting filtrate was concentrated under vacuum to afford (3S)-3-isopropoxypyrrolidine (320 mg, crude) as light yellow oil. MS ESI calculated for: C₇H₁₅NO [M+H]⁺, 130.12; found 129.95.

### Intermediate 43: (3S)-3-(1,1-difluoroethoxy)pyrrolidine

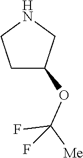

### Step 1: (3S)-1-[(4-methoxyphenyl)methyl]pyrrolidin-3-ol

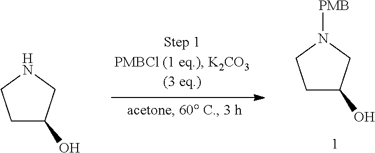

A mixture of (3S)-pyrrolidin-3-ol hydrochloride (20 g, 161.84 mmol), 4-methoxybenzyl chloride (25345.53 mg, 161.84 mmol) and K₂CO₃ (67100.91 mg, 485.51 mmol) in acetone (200 mL) was stirred for 3 h at 60 degrees C. under nitrogen atmosphere. The resulting mixture was filtered. The filtrate was concentrated under reduced pressure to afford (3S)-1-[(4-methoxyphenyl)methyl]pyrrolidin-3-ol (35 g, crude) as brown oil. MS ESI calculated for: C₁₂H₁₇NO₂ [M+H]⁺, 208.13; found 208.00.

### Step 2: (3S)-1-[(4-methoxyphenyl)methyl]pyrrolidin-3-yl acetate

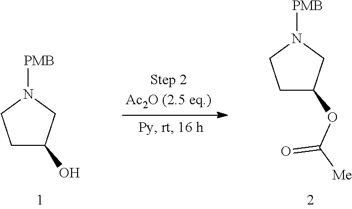

To a solution of (3S)-1-[(4-methoxyphenyl)methyl]pyrrolidin-3-ol (35 g, crude) in Pyridine (350 mL) was added Ac₂O (34477.24 mg, 337.72 mmol) dropwise at 0 degrees C. under nitrogen atmosphere. The resulting mixture was stirred for 16 h at room temperature under nitrogen atmosphere. The reaction solution was concentrated under reduced pressure. The residue was diluted with EA (500 mL) and washed with sat. Na₂CO₃ (3×250 mL). The organic layer was dried over anhydrous Na₂SO₄. After filtration, the filtrate was concentrated under reduced pressure. The residue was purified by silica gel column chromatography, eluted with DCM/MeOH (10:1) to afford (3S)-1-[(4-methoxyphenyl)methyl]pyrrolidin-3-yl acetate (17 g, 40%) as brown oil. MS ESI calculated for: C₁₄H₁₉NO₃ [M+H]⁺, 250.14; found 250.00. ¹H-NMR (400 MHz, CDCl₃) δ 7.27-7.23 (m, 2H), 6.89-6.86 (m, 2H), 5.20-5.16 (m, 1H), 3.82 (s, 3H), 3.82-3.52 (m, 2H), 2.81-2.83 (m, 2H), 2.66-2.63 (m, 1H), 2.43-2.41 (m, 1H), 2.30-2.25 (m, 1H), 2.05 (s, 3H), 1.88-1.84 (m, 1H).

### Step 3: (3S)-1-[(4-methoxyphenyl)methyl]pyrrolidin-3-yl ethanethioate

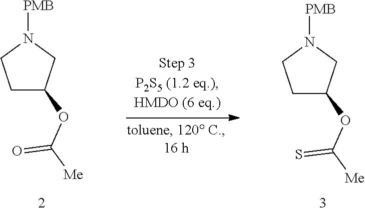

A mixture of (3S)-1-[(4-methoxyphenyl)methyl]pyrrolidin-3-yl acetate (1 g, 4.01 mmol), hexamethyldisiloxane (3907.88 mg, 24.07 mmol), P₂S₅ (1069.86 mg, 4.81 mmol) and Toluene (20 mL) was stirred for 16 h at 120 degrees C. under nitrogen atmosphere. The residue was purified by silica gel column chromatography, eluted with MeOH/DCM (0 to 7%) to afford (3S)-1-[(4-methoxyphenyl)methyl]pyrrolidin-3-yl ethanethioate (120 mg, 11%) as yellow oil. MS ESI calculated for: C₁₄H₁₉NO₂S [M+H]⁺, 266.11; found 266.05. ¹H-NMR (400 MHz, CDCl₃) δ 7.35-7.33 (m, 2H), 6.92-6.90 (m, 2H), 5.73-5.71 (m, 1H), 3.83 (s, 3H), 3.76-3.73 (m, 3H), 3.05-2.95 (m, 3H), 2.57 (s, 3H), 2.48-2.42 (m, 1H), 2.15-2.05 (m, 1H).

### Step 4: (3S)-3-(1,1-difluoroethoxy)-1-[(4-methoxyphenyl)methyl]pyrrolidine

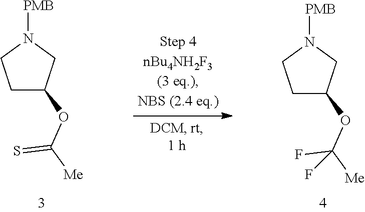

To a stirred solution of (3S)-1-[(4-methoxyphenyl)methyl]pyrrolidin-3-yl ethanethioate (120.00 mg, 0.45 mmol) and tetrabutylammonium ion dihydrofluoride fluoride (408.99 mg, 1.36 mmol) in DCM (4.00 mL) was added NBS (32.19 mg, 0.18 mmol) in portions at room temperature under nitrogen atmosphere. The resulting mixture was stirred for 1 h at room temperature under nitrogen atmosphere. The reaction was quenched by the addition of sat. NaHCO₃ (50 mL) at 0 degrees C. The resulting mixture was extracted with DCM (2×50 mL). The combined organic layers were dried over anhydrous Na₂SO₄. After filtration, the filtrate was concentrated under reduced pressure. The residue was purified by silica gel column chromatography, eluted with MeOH/DCM (0 to 5%) to afford (3S)-3-(1,1-difluoroethoxy)-1-[(4-methoxyphenyl)methyl]pyrrolidine (50 mg, 41%) as a brown oil. MS ESI calculated for: C₁₄H₁₉F₂NO₂ [M+H]⁺, 272.14; found 272.20. ¹H-NMR (400 MHz, CDCl₃) δ 7.28-7.25 (m, 2H), 6.90-6.87 (m, 2H), 4.85-4.83 (m, 1H), 3.83 (s, 3H), 2.86-2.22 (m, 5H), 1.95-1.75 (m, 1H), 1.73 (t, J=13.2 Hz, 1H). F-NMR (376 MHz, CDCl₃) δ −66.52 (2F).

### Step 5: (3S)-3-(1,1-difluoroethoxy)pyrrolidine

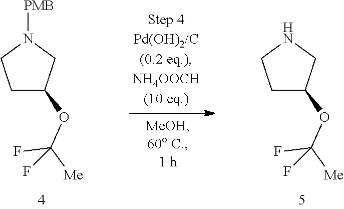

A mixture of (3S)-3-(1,1-difluoroethoxy)-1-[(4-methoxyphenyl)methyl]pyrrolidine (50.00 mg, 0.18 mmol), ammonium formate (116.21 mg, 1.84 mmol), Pd(OH)₂/C (25.88 mg, 0.04 mmol, 20%) and MeOH (2.00 mL) was stirred for 1 h at 60 degrees C. The solid was filtered out. The filtrate was concentrated under reduced pressure to afford (3S)-3-(1,1-difluoroethoxy)pyrrolidine (40 mg, crude) as yellow oil. MS ESI calculated for: C₆H₁₁F₂NO [M+H]⁺, 152.08; found 152.05.

### Intermediate 44: (3S)-3-(1,1-difluoroethoxy)pyrrolidine

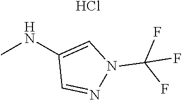

### Step 1: tert-butyl N-[1-(trifluoromethyl)pyrazol-4-yl]carbamate

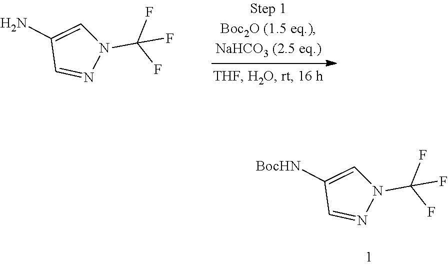 [SMILES: Nc1cnn(C(F)(F)F)c1 ; CC(C)(C)OC(=O)Nc1cnn(C(F)(F)F)c1]

To a stirred solution of 1-(trifluoromethyl)pyrazol-4-amine (0.30 g, 1.99 mmol) and di-tert-butyl dicarbonate (0.69 g, 3.18 mmol) in THF (9 mL) was added NaHCO₃ (417.0 mg, 4.96 mmol) in water (3 mL) at room temperature. The reaction mixture was stirred for 16 h at room temperature. The resulting mixture was concentrated under reduced pressure. The mixture was diluted with water (25 mL) and extracted with EtOAc (3×40 mL). The combined organic layers were dried over anhydrous Na₂SO₄. After filtration, the filtrate was concentrated under reduced pressure. The residue was purified by silica gel column chromatography, eluted with PE/EtOAc (9:1) to afford tert-butyl N-[1-(trifluoromethyl)pyrazol-4-yl]carbamate (0.46 g, 92%) as a yellow solid. MS ESI calculated for C₉H₁₂F₃N₃O₂[M+H]⁺, 252.09, found 252.10. H-NMR (400 MHz, Chloroform-d) δ 8.11 (s, 1H), 7.63 (s, 1H), 6.42 (s, 1H), 1.54 (s, 9H). F-NMR (376 MHz, CDCl₃) δ −60.69 (3F).

### Step 2: tert-butyl N-methyl-N-[1-(trifluoromethyl)pyrazol-4-yl]carbamate

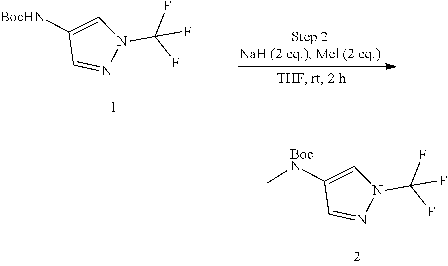 [SMILES: CC(C)(C)OC(=O)Nc1cnn(C(F)(F)F)c1 ; CN(C(=O)OC(C)(C)C)c1cnn(C(F)(F)F)c1]

To a stirred solution of NaH (99.35 mg, 4.14 mmol, 60%) in THF (5.00 mL) was added tert-butyl N-[1-(trifluoromethyl)pyrazol-4-yl]carbamate (0.52 g, 2.07 mmol) in THF (2.0 mL) dropwise at 0° C. The resulting mixture was stirred for 1 h at 0° C. To the above mixture was added Mel (0.59 g, 4.14 mmol) at 0° C. The resulting mixture was stirred for additional 1 h at room temperature. The resulting mixture was quenched with water (20 mL) and extracted with EA (3×60 mL). The combined organic layers were dried over anhydrous Na₂SO₄. After filtration, the filtrate was concentrated under reduced pressure. The residue was purified by silica gel column chromatography, eluted with PE/EA (5:1) to afford tert-butyl N-methyl-N-[1-(trifluoromethyl)pyrazol-4-yl]carbamate (0.50 g, 91%) as a yellow solid. MS ESI calculated for C₁₀H₁₄F₃N₃O₂[M+H]⁺, 266.10, found 266.20. H-NMR (400 MHz, Chloroform-d) δ 8.10 (s, 1H), 7.83 (s, 1H), 3.27 (s, 3H), 1.54 (s, 9H).

### Step 3: (3S)-1-[(4-methoxyphenyl)methyl]pyrrolidin-3-yl ethanethioate

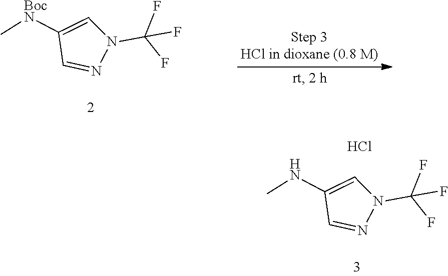 [SMILES: CN(C(=O)OC(C)(C)C)c1cnn(C(F)(F)F)c1 ; Cl ; CNc1cnn(C(F)(F)F)c1]

To a solution of tert-butyl N-methyl-N-[1-(trifluoromethyl)pyrazol-4-yl]carbamate (0.60 g, 2.26 mmol) in dioxane (1.0 mL) was added HCl (4 M in dioxane, 5.00 mL) at room temperature. The reaction mixture was stirred for 2 h at room temperature. The resulting mixture was concentrated under reduced pressure to afford N-methyl-1-(trifluoromethyl)pyrazol-4-amine hydrochloride (0.40 g, crude) as a yellow solid. MS ESI calculated for C₅H₇ClF₃N₃[M+H—HCl]⁺, 166.05, found 166.00. H-NMR (400 MHz, d₆-DMSO) δ 8.77 (s, 2H), 8.38 (s, 1H), 7.98 (s, 1H), 2.81 (s, 3H).

### Intermediate 45: 4-(2,2,2-trifluoroethyl)pyrrolidin-3-yl benzoate

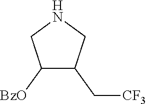

### Step 1: Tert-butyl 3-hydroxy-4-methylidenepyrrolidine-1-carboxylate

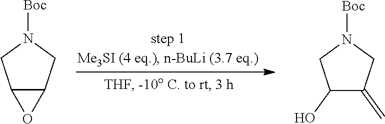

To a stirred mixture of (CH₃)₃Si (24.46 g, 119.86 mmol) in THE (225.00 mL) was added n-BuLi (44.35 mL, 110.88 mmol) dropwise at −10° C. under nitrogen atmosphere. The reaction mixture was stirred for 30 min at −10° C. under nitrogen atmosphere. To the above mixture was added a solution of tert-butyl 6-oxa-3-azabicyclo[3.1.0]hexane-3-carboxylate (5.55 g, 29.96 mmol) in THE (45.00 mL) dropwise at −10° C. The reaction mixture was slowly allowed to warm to room temperature over 1 h and then was stirred for 3 h at room temperature under nitrogen atmosphere. The resulting mixture was quenched by the addition of water (300 mL). The resulting mixture was extracted with EA (3×200 mL). The combined organic layers was washed with brine (300 mL), dried over anhydrous Na₂SO₄ and filtered. The filtrate was concentrated under reduced pressure. The residue was purified by silica gel column chromatography, eluted with EA in PE (0-100%). The fractions contained desired product were combined and concentrated to afford tert-butyl 3-hydroxy-4-methylidenepyrrolidine-1-carboxylate (2.5 g, 42%) as light brown oil. MS ESI calculated for C₁₀H₁₇NO₃ [M-t-Bu+H]⁺, 144.06, found 144.10.

### Step 2: Tert-butyl 3-hydroxy-4-(2,2,2-trifluoroethyl)pyrrolidine-1-carboxylate

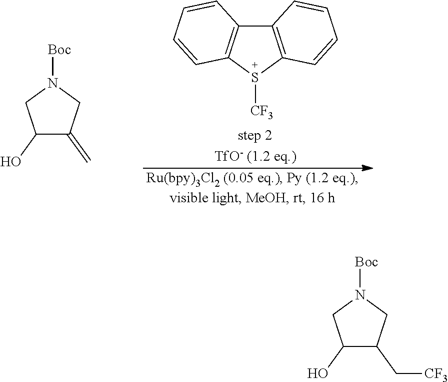

To a solution of tert-butyl 3-hydroxy-4-methylidenepyrrolidine-1-carboxylate (1.00 g, 5.02 mmol), tris(2,2-bipyridine)ruthenium dichloride (0.16 g, 0.25 mmol) and 8-(trifluoromethyl)-8-thiatricyclo[7.4.0.0\hat{ }[2,7]]trideca-1(13),2,4,6,9,11-hexaen-8-ium; trifluoromethanesulfonic acid (2.43 g, 6.02 mmol) in MeOH (15.00 mL) was added pyridine (0.48 g, 6.02 mmol). The reaction mixture was irradiated with a fluorescent lamp and stirred for 16 h at room temperature under argon atmosphere. The resulting mixture was quenched by the addition of aqueous NaHCO₃ (sat., 100 mL). The resulting mixture was extracted with EA (3×50 mL). The combined organic layers were washed with water (50 mL) and brine (1×50 mL), dried over anhydrous Na₂SO₄ and filtered. The filtrate was concentrated under reduced pressure. The residue was purified by silica gel column chromatography, eluted with PE/EA (0-100%). The fractions contained desired product were combined and concentrated to afford tert-butyl 3-hydroxy-4-(2,2,2-trifluoroethyl)pyrrolidine-1-carboxylate (480 mg, 36%) as a yellow oil. MS ESI calculated for C₁₁H₁₈F₃NO₃ [M-t-Bu+H]⁺, 214.06, found 214.05.

### Step 3: Tert-butyl 3-(benzoyloxy)-4-(2,2,2-trifluoroethyl)pyrrolidine-1-carboxylate

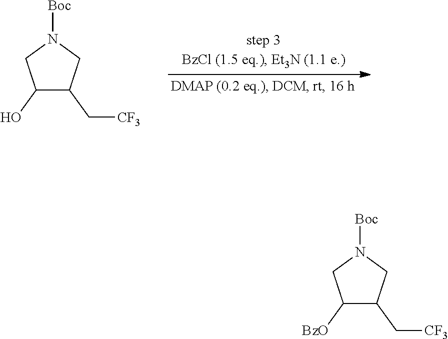

To a stirred solution of tert-butyl 3-hydroxy-4-(2,2,2-trifluoroethyl)pyrrolidine-1-carboxylate (65.00 mg, 0.24 mmol) in DCM (3.50 mL) were added TEA (122.14 mg, 1.207 mmol), DMAP (32.44 mg, 0.266 mmol) and benzoyl chloride (67.87 mg, 0.483 mmol) at 0° C. under nitrogen atmosphere. The reaction mixture was stirred for 16 h at room temperature under nitrogen atmosphere. The resulting mixture was quenched by the addition of water (20 mL). The resulting mixture was extracted with DCM (3×20 mL). The combined organic layers was washed with brine (20 mL), dried over anhydrous Na₂SO₄ and filtered. The filtrate was concentrated under reduced pressure. The residue was purified by silica gel column chromatography, eluted with EA in PE (0-50%). The fractions contained desired product were combined and concentrated to afford tert-butyl 3-(benzoyloxy)-4-(2,2,2-trifluoroethyl)pyrrolidine-1-carboxylate (60 mg, 67%) as a light yellow solid. MS ESI calculated for C₁₈H₂₂F₃NO₄ [M-t-Bu+H]⁺, 318.09, found 318.05.

### Step 4: 4-(2,2,2-trifluoroethyl)pyrrolidin-3-yl benzoate; trifluoroacetic acid

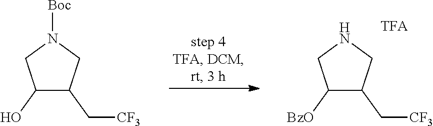

To a stirred solution of tert-butyl 3-(benzoyloxy)-4-(2,2,2-trifluoroethyl)pyrrolidine-1-carboxylate (60.00 mg, 0.161 mmol) in DCM (2.50 mL) was added TFA (0.50 mL) dropwise at room temperature. The reaction solution was stirred for 2 h at room temperature. The resulting mixture was concentrated under reduced pressure to afford 4-(2,2,2-trifluoroethyl)pyrrolidin-3-yl benzoate; trifluoroacetic acid (65 mg) as a brown oil. The product was used directly to next step without further purification. MS ESI calculated for C₁₅H₁₅F₆NO₄ [M-TFA+H]⁺, 274.10, found 274.05.

### Intermediate 46: (3R)-3-(trifluoromethoxy)pyrrolidine hydrochloride

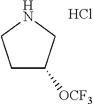

### Step 1: benzyl (3R)-3-(trifluoromethoxy)pyrrolidine-1-carboxylate

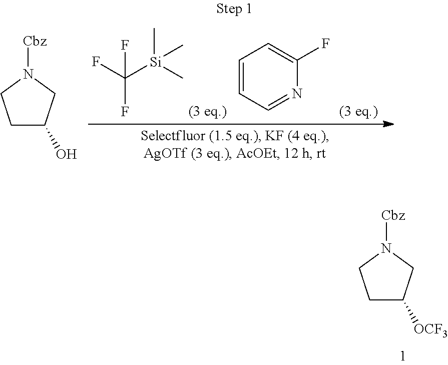

To a stirred mixture of AgOTf (34.84 g, 135.59 mmol) and KF (10.50 g, 180.79 mmol), Selectfluor (24.02 g, 67.79 mmol), benzyl (3R)-3-hydroxypyrrolidine-1-carboxylate (10.00 g, 45.20 mmol) in EtOAc (270 mL) were added 2-fluoropyridine (13.16 g, 135.54 mmol) and TMSCF₃ (19.28 g, 135.59 mmol) dropwise at 0 degrees C. under nitrogen atmosphere. The resulting mixture was stirred for 12 h at room temperature under nitrogen atmosphere. The resulting mixture was filtered, and the filter cake was washed with EtOAc (100 mL). The filtrate was concentrated under reduced pressure. The residue was purified by silica gel column chromatography, eluted with PE/EtOAc (5:1) to afford benzyl (3R)-3-(trifluoromethoxy) pyrrolidine-1-carboxylate (2.6 g, 15%) as colorless oil. MS ESI calculated for C₁₃H₁₄F₃NO₃ [M+H]⁺, 290.09, found 290.10. ¹H-NMR (400 MHz, d₆-DMSO): δ 7.38-7.30 (m, 5H), 5.13-5.03 (m, 3H), 3.66-3.22 (m, 4H), 2.20-2.13 (m, 2H). ¹⁹F-NMR (376 MHz, d₆-DMSO): −56.83.

### Step 2: (3R)-3-(trifluoromethoxy)pyrrolidine hydrochloride

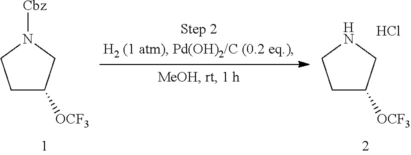 [SMILES: O=C(OCc1ccccc1)N1CC[C@@H](OC(F)(F)F)C1 ; FC(F)(F)O[C@@H]1CCNC1 ; Cl]

To a stirred solution of benzyl (3R)-3-(trifluoromethoxy)pyrrolidine-1-carboxylate (1.00 g, 3.28 mmol) in MeOH (10 mL) was added Pd(OH)₂/C (46.12 mg, 0.33 mmol, 20%) at room temperature under nitrogen atmosphere. The resulting mixture was stirred for 1 h at room temperature under hydrogen atmosphere. The resulting mixture was filtered, and the filter cake was washed with MeOH (5×10 mL). The filtrate was treated with HCl (2 mL, 4 M in dioxane) and concentrated under reduced pressure to afforded (3R)-3-(trifluoromethoxy)pyrrolidine hydrochloride (600 mg, crude) as a light yellow solid. MS ESI calculated for C₅H₉ClF₃NO [M+H—HCl]⁺, 156.12, found 156.10. H-NMR (400 MHz, DMSO-d₆) δ 9.97-9.77 (m, 2H), 5.25-5.22 (m, 1H), 3.50-3.18 (m, 4H), 2.26-2.15 (m, 2H).

### Intermediate 47: (3S)-3-(trifluoromethoxy)pyrrolidine hydrochloride

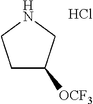

The title compound was prepared using procedures similar to those described in Intermediate 35 using benzyl (3S)-3-hydroxypyrrolidine-1-carboxylate instead of benzyl (3R)-3-hydroxypyrrolidine-1-carboxylate to afford the title compound as a solid.

### Intermediate 48: 3-cyclopropylidenepyrrolidine hydrochloride

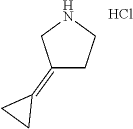

### Step 1: tert-butyl 3-cyclopropylidenepyrrolidine-1-carboxylate

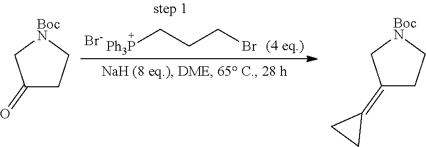

To a mixture of (3-bromopropyl)triphenylphosphanium bromide (10.02 g, 21.60 mmol) and in DME (23.00 mL) was added NaH (1.73 g, 43.25 mmol, 60%) at 0° C. The reaction mixture was stirred for 6 h at 65° C. To the above mixture was added tert-butyl 3-oxopyrrolidine-1-carboxylate (1.00 g, 5.40 mmol) at 65° C. The resulting mixture was stirred for additional 22 h at 65° C. The reaction was quenched by the addition of Water/Ice (30 mL) at 0° C. The resulting mixture was extracted with EtOAc (2×50 mL). The combined organic layers was washed with brine (10 mL), dried over anhydrous Na₂SO₄ and filtered. The filtrate was concentrated under reduced pressure. The residue was purified by silica gel column chromatography, eluted with PE/EtOAc (5:1). The fractions contained desired product were combined and concentrated to afford tert-butyl 3-cyclopropylidenepyrrolidine-1-carboxylate (0.49 g, 39%) as a light yellow oil. MS ESI calculated for C₁₂H₁₉NO₂ [M+H-56]⁺, 154.14, found 153.90.

### Step 2: 3-cyclopropylidenepyrrolidine hydrochloride

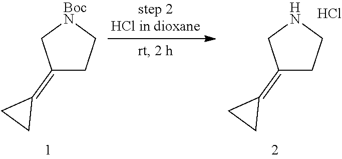 [SMILES: Cl ; CC(C)(C)OC(=O)N1CCC(=C2CC2)C1 ; C1CC(=C2CC2)CN1]

To a stirred mixture of tert-butyl 3-cyclopropylidenepyrrolidine-1-carboxylate (0.49 g, 2.34 mmol) in 1,4-dioxane (2.00 mL) was added HCl (gas) in 1,4-dioxane (2.00 mL) dropwise at 0° C. under nitrogen atmosphere. The reaction mixture was stirred for 2 h at room temperature under nitrogen atmosphere. The resulting mixture was concentrated under vacuum. This resulted in 3-cyclopropylidenepyrrolidine hydrochloride (0.46 g, 67%) as a brown oil. MS ESI calculated for C₇H₁₂ClN [M+H—HCl]⁺, 110.09; found 110.20.

### Intermediate 49: benzyl 4-(trifluoromethoxy)pyrazolidine-1-carboxylate 2,2,2-trifluoroacetate

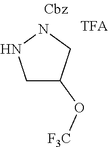

### Step 1: N-(tert-butoxycarbonyl)benzyloxycarbohydrazide

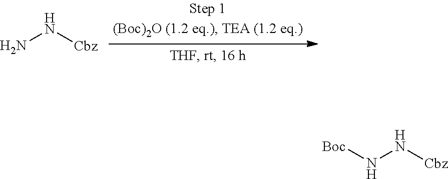 [SMILES: NNC(=O)OCc1ccccc1 ; CC(C)(C)OC(=O)NNC(=O)OCc1ccccc1]

To a stirred solution of benzyloxycarbohydrazide (5.00 g, 30.09 mmol) and TEA (3.65 g, 36.11 mmol) in THF (15.00 mL) was added a solution of (Boc)₂O (7.88 g, 36.11 mmol) in THF (10.00 mL) dropwise at room temperature under nitrogen atmosphere. The reaction mixture was stirred overnight at room temperature under nitrogen atmosphere. The resulting mixture was concentrated under vacuum. The residue was purified by trituration with hexane (100 mL) to afford N-(tert-butoxycarbonyl)benzyloxycarbohydrazide (7.1 g, 89%) as an off-white solid. MS ESI calculated for C₁₃H₁₈N₂O₄[M-Boc+H]⁺, 167.07, found 167.10.

### Step 2: Tert-butyl[(1,3-dibromopropan-2-yl)oxy]dimethylsilane

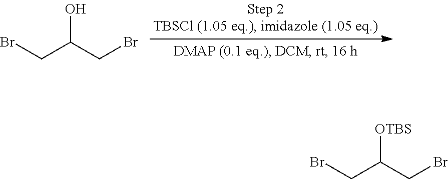 [SMILES: OC(CBr)CBr ; CC(C)(C)[Si](C)(C)OC(CBr)CBr]

To a stirred solution of 1,3-dibromo-2-propanol (10.00 g, 45.90 mmol) in DCM (50.00 mL) were added 1H-imidazole (3.28 g, 48.19 mmol), TBS-Cl (7.26 g, 48.19 mmol) and DMAP (0.56 g, 4.59 mmol) at 0° C. under nitrogen atmosphere. The reaction mixture was stirred for 16 h at room temperature under nitrogen atmosphere. The reaction was quenched by the addition of water (100 mL) at room temperature. The resulting mixture was extracted with DCM (3×50 mL). The combined organic layers was washed with water (100 mL) and brine (100 mL), dried over anhydrous Na₂SO₄ and filtered. The filtrate was concentrated under reduced pressure. The residue was purified by silica gel column chromatography, eluted with EA/PE (0-20%). The fractions contained desired product were combined and concentrated to afford tert-butyl[(1,3-dibromopropan-2-yl)oxy]dimethylsilane (14.3 g, 94%) as a light yellow oil. H-NMR (400 MHz, CDCl₃) δ 4.08-4.01 (m, 1H), 3.55-3.48 (m, 4H), 0.94 (s, 9H), 0.17 (s, 6H).

### Step 3: 1-Benzyl 2-tert-butyl 4-hydroxypyrazolidine-1,2-dicarboxylate

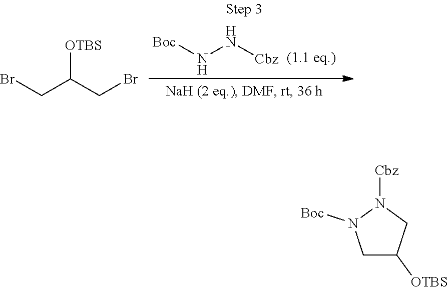 [SMILES: CC(C)(C)[Si](C)(C)OC(CBr)CBr ; CC(C)(C)OC(=O)NNC(=O)OCc1ccccc1 ; CC(C)(C)OC(=O)N1CC(O[Si](C)(C)C(C)(C)C)CN1C(=O)OCc1ccccc1]

To a stirred mixture of NaH (3.61 g, 90.32 mmol) in DMF (30.00 mL) was added a solution of N-(tert-butoxycarbonyl)benzyloxycarbohydrazide (10.58 g, 39.74 mmol) in DMF (48.00 mL) dropwise at 0° C. under nitrogen atmosphere. The reaction mixture was stirred for 45 min at room temperature under nitrogen atmosphere. To the above mixture was added tert-butyl[(1,3-dibromopropan-2-yl)oxy]dimethylsilane (12.00 g, 36.13 mmol) dropwise at room temperature. The reaction mixture was stirred for additional 36 h at room temperature. The reaction was quenched with aqueous NH₄Cl (sat., 500 mL) at room temperature. The resulting mixture was extracted with EtOAc (3×200 mL). The combined organic layers was washed with brine (300 mL), dried over anhydrous Na₂SO₄ and filtered. The filtrate was concentrated under reduced pressure. The residue was purified by silica gel column chromatography, eluted with EA in PE (0-50%). The fractions contained desired product were combined and concentrated to afford 1-benzyl 2-tert-butyl 4-hydroxypyrazolidine-1,2-dicarboxylate (2.3 g, 20%) as a light brown oil. MS ESI calculated for C₂₂H₃₆N₂O₅Si [M-Boc+H]⁺, 337.20, found 337.10.

### Step 4: 1-Benzyl 2-tert-butyl 4-hydroxypyrazolidine-1,2-dicarboxylate

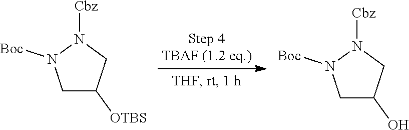 [SMILES: CC(C)(C)OC(=O)N1CC(O[Si](C)(C)C(C)(C)C)CN1C(=O)OCc1ccccc1 ; CC(C)(C)OC(=O)N1CC(O)CN1C(=O)OCc1ccccc1]

To a stirred solution of 1-benzyl 2-tert-butyl 4-[(tert-butyldimethylsilyl)oxy]pyrazolidine-1,2-dicarboxylate (2.2 g, 5.04 mmol) in THF (30.00 mL) was added TBAF (6.05 mL, 6.05 mmol) dropwise at 0° C. under nitrogen atmosphere. The reaction solution was stirred for 1 h at room temperature under nitrogen atmosphere. The resulting mixture was concentrated under vacuum. The residue was purified by silica gel column chromatography, eluted with EA in PE (0-100%). The fractions contained desired product were combined and concentrated to afford 1-benzyl 2-tert-butyl 4-hydroxypyrazolidine-1,2-dicarboxylate (1.55 g, 95%) as a yellow oil. MS ESI calculated for C₁₆H₂₂N₂O₅ [M+H]⁺, 323.15, found 323.10.

### Step 5: 1-Benzyl 2-tert-butyl 4-(trifluoromethoxy)pyrazolidine-1,2-dicarboxylate

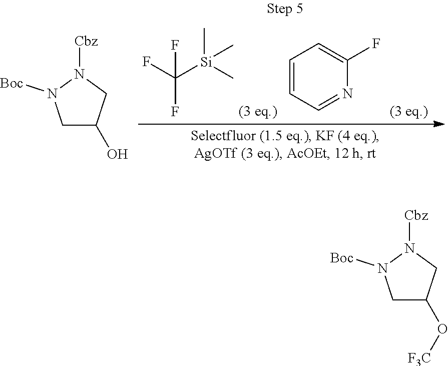

To a stirred solution of argentio trifluoromethanesulfonate (3.71 g, 14.44 mmol) and KF (1.12 g, 19.28 mmol), 4-(chloromethyl)-1-fluoro-1,4-diazabicyclo[2.2.2]octane-1,4-diium; bis(tetrafluoroboranuide) (2.56 g, 7.23 mmol), 1-benzyl 2-tert-butyl 4-hydroxypyrazolidine-1,2-dicarboxylate (1.55 g, 4.81 mmol) in EA (25.00 mL) were added 2-fluoropyridine (1.40 g, 14.42 mmol) and TMSCF₃ (2.05 g, 14.42 mmol) dropwise at room temperature under nitrogen atmosphere. The reaction mixture was stirred for 12 h at room temperature under nitrogen atmosphere. The resulting mixture was filtered. The filter cake was washed with EtOAc (3×25 mL). The filtrate was concentrated under reduced pressure. The residue was purified by silica gel column chromatography, eluted with EA in PE (0-40%). The fractions contained desired product were combined and concentrated to afford 1-benzyl 2-tert-butyl 4-(trifluoromethoxy)pyrazolidine-1,2-dicarboxylate (0.35 g, 19%) as a colorless oil. MS ESI calculated for C₁₇H₂₁F₃N₂O₅[M-Boc+H]⁺, 291.09, found 291.05.

### Step 6: benzyl 4-(trifluoromethoxy)pyrazolidine-1-carboxylate 2,2,2-trifluoroacetate

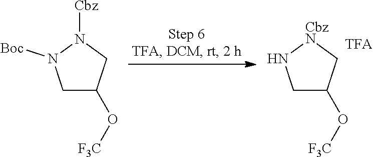 [SMILES: CC(C)(C)OC(=O)N1CC(OC(F)(F)F)CN1C(=O)OCc1ccccc1 ; O=C(OCc1ccccc1)N1CC(OC(F)(F)F)CN1]

To a stirred solution of 1-benzyl 2-tert-butyl 4-(trifluoromethoxy)pyrazolidine-1,2-dicarboxylate (70.00 mg, 0.18 mmol) in DCM (2.50 mL) was added TFA (0.50 mL) dropwise at room temperature. The reaction solution was stirred for 2 h at room temperature. The resulting solution was concentrated under reduced pressure to afford benzyl 4-(trifluoromethoxy)pyrazolidine-1-carboxylate 2,2,2-trifluoroacetate (65 mg, 93%) as light brown oil. MS ESI calculated for C₁₄H₁₄F₆N₂O₅[M-TFA+H]⁺, 291.09, found 290.95.

### Intermediate 50: 2-fluoro-4-methyl-5-(4,4,5,5-tetramethyl-1,3,2-dioxaborolan-2-yl)aniline

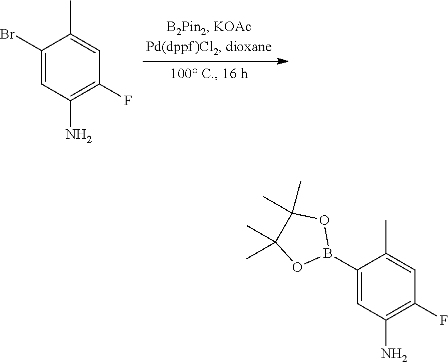

To a solution of 5-bromo-2-fluoro-4-methylaniline (2.0 g, 9.8 mmol), 4,4,4′,4′,5,5,5′,5′-octamethyl-2,2′-bi(1,3,2-dioxaborolane) (2.7 g, 10.8 mmol) and KOAc (2.9 g, 29.4 mmol) in dioxane (20 mL) was added Pd(dppf)Cl₂ (359 mg, 0.49 mmol) under N₂, and the mixture was stirred at 100° C. for 16 h. The reaction was cooled down to rt and the mixture was filtered. The filtrate was concentrated to give 2-fluoro-4-methyl-5-(4,4,5,5-tetramethyl-1,3,2-dioxaborolan-2-yl)aniline (crude), which was used in the next step without any further purification. MS Calcd.: 251, MS Found: 252 ([M+H]⁺).

### Intermediate 51: 4-(6-(3-(benzyloxy)cyclobutoxy)-4-iodopyridin-2-yl)morpholine

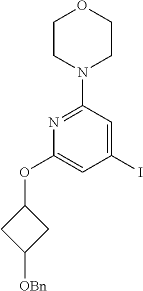 [SMILES: Ic1cc(OC2CC(OCc3ccccc3)C2)nc(N2CCOCC2)c1]

The title compound was prepared using procedures similar to those described in Intermediate 54 step 2 using 3-(benzyloxy)cyclobutan-1-ol instead of 2-(oxan-2-yloxy)ethanol to afford the title compound as a solid.

### Intermediate 52: 3-((4-iodo-6-morpholinopyridin-2-yl)oxy)cyclopentan-1-ol

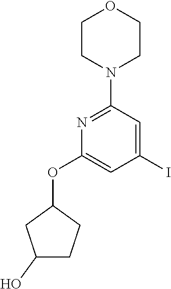 [SMILES: OC1CCC(Oc2cc(I)cc(N3CCOCC3)n2)C1]

The title compound was prepared using procedures similar to those described in Intermediate 54 step 2 using cyclopentane-1,3-diol instead of 2-(oxan-2-yloxy)ethanol to afford the title compound as a solid.

### Intermediates 53 and 54: (1S,4S)-4-[[4-iodo-6-(morpholin-4-yl)pyridin-2-yl]oxy]-1-methylcyclohexan-1-ol (cis) and (1R,4R)-4-[[4-iodo-6-(morpholin-4-yl)pyridin-2-yl]oxy]-1-methylcyclohexan-1-ol (trans)

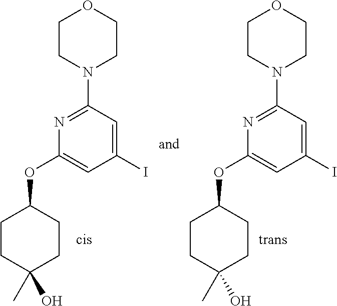 [SMILES: C[C@]1(O)CC[C@@H](Oc2cc(I)cc(N3CCOCC3)n2)CC1 ; C[C@]1(O)CC[C@H](Oc2cc(I)cc(N3CCOCC3)n2)CC1]

### Step 1: 4-(benzyloxy)-1-methylcyclohexan-1-ol

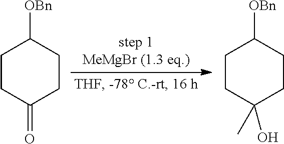 [SMILES: O=C1CCC(OCc2ccccc2)CC1 ; CC1(O)CCC(OCc2ccccc2)CC1]

To a stirred solution of 4-(benzyloxy)cyclohexan-1-one (2.50 g, 12.24 mmol) in THF (25.00 mL) was added 1 M CH₃MgBr in THF (3.18 mL, 3.18 mmol) dropwise at −70° C. under nitrogen atmosphere. The reaction mixture was stirred for 16 h at room temperature under nitrogen atmosphere. The resulting mixture was quenched with aqueous NH₄Cl (sat., 100 ml) at 0° C. The resulting mixture was extracted with EtOAc (3×50 mL). The combined organic layers was washed with brine (50 mL), dried over anhydrous Na₂SO₄. After filtration, the filtrate was concentrated under reduced pressure. The residue was purified by silica gel column chromatography, eluted with EA in PE (50%). The fractions contained desired product were combined and concentrated to afford 4-(benzyloxy)-1-methylcyclohexan-1-ol (1.05 g, 39%) as light yellow oil. H-NMR (400 MHz, CDCl₃) δ 7.40-7.33 (m, 5H), 4.59-4.52 (m, 2H), 3.61-3.55 (m, 1H), 1.90-1.69 (m, 6H), 1.50-1.40 (m, 2H), 1.29-1.25 (m, 3H).

### Step 2: 1-methylcyclohexane-1,4-diol

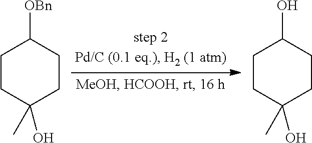

A mixture of 4-(benzyloxy)-1-methylcyclohexan-1-ol (1.05 g, 4.77 mmol), Pd/C (0.50 g, 0.47 mmol, 10%), HCOOH (0.75 mL) and MeOH (5.00 mL) was stirred for 16 h at room temperature under hydrogen (2 atm) atmosphere. The resulting mixture was filtered. The filter cake was washed with MeOH (4×10 mL). The combined filtrate was concentrated under reduced pressure to afford 1-methylcyclohexane-1,4-diol (0.60 g, 97%) as light yellow oil. H-NMR (400 MHz, CDCl₃) δ 3.93-3.62 (m, 1H), 2.50-1.43 (m, 8H), 1.30-1.25 (m, 3H).

### Step 3: (1S,4S)-4-[[4-iodo-6-(morpholin-4-yl)pyridin-2-yl]oxy]-1-methylcyclohexan-1-ol (cis): (1R,4R)-4-[[4-iodo-6-(morpholin-4-yl)pyridin-2-yl]oxy]-1-methylcyclohexan-1-ol (trans)

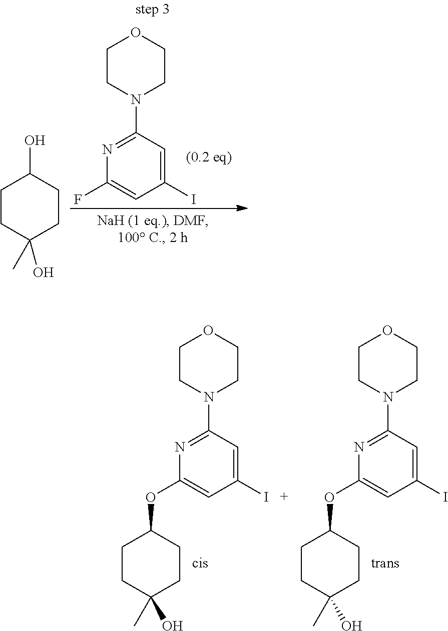

To a stirred mixture of NaH (0.16 g, 3.90 mmol) in DMF (4.00 mL) was added a solution of 1-methylcyclohexane-1,4-diol (0.51 g, 3.90 mmol) in DMF (4.00 mL) dropwise at 0° C. under nitrogen atmosphere. The reaction mixture was stirred for 1 h at room temperature under nitrogen atmosphere. To the above mixture was added a solution of 4-(6-fluoro-4-iodopyridin-2-yl)morpholine (0.40 g, 1.30 mmol) in DMF (2.00 mL) at room temperature. The reaction mixture was stirred for additional 2 h at 100° C. The resulting mixture was allowed to cool down to room temperature and quenched with water (100 mL). The resulting mixture was extracted with EtOAc (3×50 mL). The combined organic layers was washed with Brine (100 mL), dried over anhydrous Na₂SO₄. After filtration, the filtrate was concentrated under reduced pressure. The residue was purified by silica gel column chromatography, eluted with EA in PE (0-70%). The fractions contained desired product were combined and concentrated to afford (1S,4S)-4-[[4-iodo-6-(morpholin-4-yl)pyridin-2-yl]oxy]-1-methylcyclohexan-1-ol (0.13 g, 46%) (cis) as an off-white solid. MS ESI calculated for C₁₆H₂₃IN₂O₃[M+H]⁺, 419.08, found 419.05. H-NMR (400 MHz, d₆-DMSO) δ 6.68-6.65 (m, 1H), 6.44-6.39 (m, 1H), 4.84-4.75 (m, 1H), 4.14 (s, 1H), 3.72-3.66 (m, 4H), 3.42-3.37 (m, 4H), 1.77-1.67 (m, 4H), 1.62-1.58 (m, 2H), 1.45-1.33 (m, 2H), 1.12 (s, 3H). And also to afford (1R,4R)-4-[[4-iodo-6-(morpholin-4-yl)pyridin-2-yl]oxy]-1-methylcyclohexan-1-ol (0.10 g, 37%) (trans) as an off-white solid. MS ESI calculated for C₁₆H₂₃IN₂O₃[M+H]⁺, 419.08, found 419.10. H-NMR (400 MHz, d₆-DMSO) δ 6.66-6.65 (m, 1H), 6.44-6.43 (m, 1H), 4.98-4.92 (m, 1H), 4.10 (s, 1H), 3.72-3.66 (m, 4H), 3.42-3.39 (m, 4H), 1.94-1.84 (m, 2H), 1.62-1.52 (m, 4H), 1.45-1.39 (m, 2H), 1.15 (s, 3H).

### Intermediate 55: (4-[[4-iodo-6-(morpholin-4-yl)pyridin-2-yl]oxy]-1-methylcyclohexan-1-ol

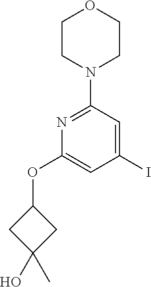 [SMILES: CC1(O)CC(Oc2cc(I)cc(N3CCOCC3)n2)C1]

### Step 1: [3-(benzyloxy)-1-methylcyclobutoxy](tert-butyl)diphenylsilane

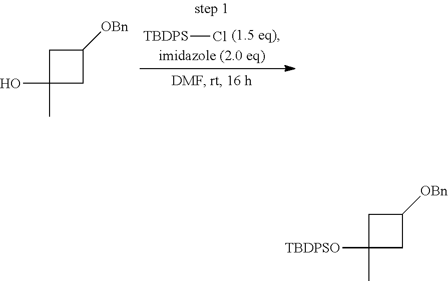

To a stirred solution of 3-(benzyloxy)-1-methylcyclobutan-1-ol (1.00 g, 5.20 mmol) and imidazole (0.71 g, 10.40 mmol) in DMF (10.00 mL) was added tert-butyl(chloro)diphenylsilane (2.02 mL, 7.79 mmol) at 0° C. The reaction mixture was stirred for 18 h at 20° C. The resulting mixture was diluted with water (100 mL) and extracted with EA (4×100 mL). The combined organic layers was washed with brine (100 mL), dried over anhydrous Na₂SO₄ and filtered. The filtrate was concentrated under reduced pressure. The residue was purified by silica gel column chromatography, eluted with EA/PE (0-12%). The fractions contained desired product were combined and concentrated to afford [3-(benzyloxy)-1-methylcyclobutoxy](tert-butyl)diphenylsilane (1.25 g, 56%) as a colorless crude oil. H-NMR (400 MHz, DMSO-d₆) δ 7.76-7.27 (m, 15H), 4.33-4.31 (m, 2H), 3.60-3.53 (m, 1H), 2.27-2.16 (m, 4H), 1.19-1.18 (m, 3H), 1.06-1.05 (m, 9H).

### Step 2: 3-[(tert-butyldiphenylsilyl)oxy]-3-methylcyclobutan-1-ol

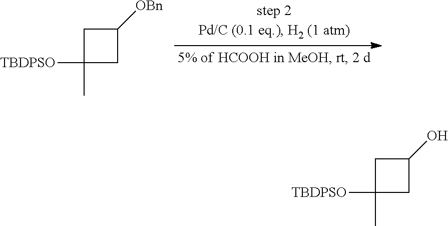

To a mixture of [3-(benzyloxy)-1-methylcyclobutoxy](tert-butyl)diphenylsilane (1.25 g, 2.90 mmol) and Pd/C (0.31 g, 0.29 mmol, 10%) in MeOH (20.00 mL) was added HCO₂H (1.00 mL). The reaction mixture was degassed with H₂ for three times and stirred for 2 days at room temperature. The resulting mixture was filtered and the filter cake was washed with MeOH (3×20 mL). The filtrate was concentrated under reduced pressure. The residue was purified by silica gel column chromatography, eluted with PE/EtOAc (4:1). The fractions contained desired product were combined and concentrated to afford 3-[(tert-butyldiphenylsilyl)oxy]-3-methylcyclobutan-1-ol (0.60 g, 61%) as a colorless oil. H-NMR (400 MHz, CDCl₃) δ 7.76-7.71 (m, 4H), 7.47-7.38 (m, 6H), 3.81-3.74 (m, 1H), 2.31-2.25 (m, 2H), 2.13-2.07 (m, 2H), 1.21 (m, 3H), 1.06-1.04 (m, 9H).

### Step 3: 4-(6-[3-[(tert-butyldiphenylsilyl)oxy]-3-methylcyclobutoxy]-4-iodopyridin-2-yl)morpholine and 3-[[4-iodo-6-(morpholin-4-yl)pyridin-2-yl]oxy]-1-methylcyclobutan-1-ol

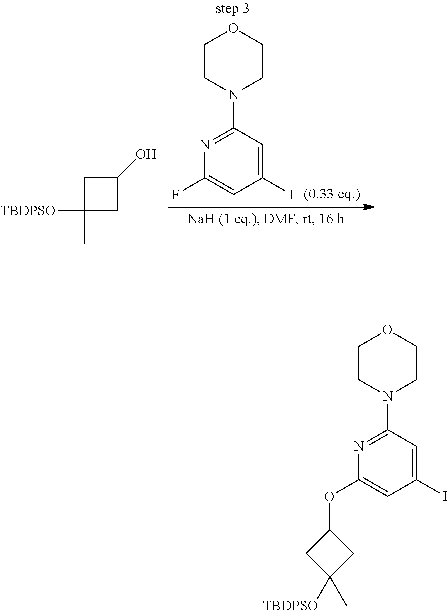

To a stirred solution of 3-[(tert-butyldiphenylsilyl)oxy]-3-methylcyclobutan-1-ol (0.35 g, 1.03 mmol) in DMF (3.00 mL) was added NaH (41.11 mg, 1.03 mmol, 60%) at 0° C. under nitrogen atmosphere. The reaction mixture was stirred for 1 h at room temperature. To the above mixture was added 4-(6-fluoro-4-iodopyridin-2-yl)morpholine (0.10 g, 0.34 mmol) at room temperature. The reaction mixture was stirred for additional 16 h at room temperature. The resulting mixture was quenched by the addition of saturated aqueous NaHCO₃ (100 mL) at room temperature. The resulting mixture was extracted with EA (3×100 mL). The combined organic layers was washed with brine (3×50 mL), dried over anhydrous Na₂SO₄ and filtered. The filtrate was concentrated under reduced pressure. The residue was purified by silica gel column chromatography, eluted with PE/EA (2:1). The fractions contained desired product were combined and concentrated to afford 4-(6-[3-[(tert-butyldiphenylsilyl)oxy]-3-methylcyclobutoxy]-4-iodopyridin-2-yl)morpholine (0.13 g, 18%) as an off-white solid. MS ESI calculated for C₃₀H₃₇IN₂O₃Si [M+H]⁺, 629.16, found 629.00.

### Step 4: 3-[[4-iodo-6-(morpholin-4-yl)pyridin-2-yl]oxy]-1-methylcyclobutan-1-ol and 3-[[4-iodo-6-(morpholin-4-yl)pyridin-2-yl]oxy]-1-methylcyclobutan-1-ol

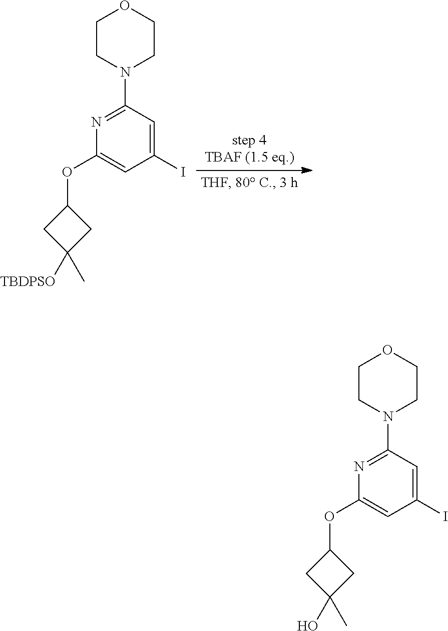

To a stirred solution of 4-(6-[3-[(tert-butyldiphenylsilyl)oxy]-3-methylcyclobutoxy]-4-iodopyridin-2-yl)morpholine (0.15 g, 0.24 mmol) in THF (2.00 mL) was added TBAF (0.36 mL, 0.36 mmol, 1 M) at ° C. under nitrogen atmosphere. The reaction mixture was stirred for 3 h at 80° C. under nitrogen atmosphere. The resulting mixture was quenched by the addition of water (100 mL). The resulting mixture was extracted with EA (3×50 mL). The combined organic layers was washed with brine (3×50 mL), dried over anhydrous Na₂SO₄ and filtered. The filtrate was concentrated under reduced pressure. The residue was purified by silica gel column chromatography, eluted with PE/(EA/EtOH=3:1) (2:1). The fractions contained desired product were combined and concentrated to afford 3-[[4-iodo-6-(morpholin-4-yl)pyridin-2-yl]oxy]-1-methylcyclobutan-1-ol (30 mg, 32%) as an off-white solid. MS ESI calculated for C₁₄H₁₉IN₂O₃[M+H]⁺, 391.04, found 390.90. ¹H-NMR (400 MHz, DMSO-d₆) δ 6.67 (s, 1H), 6.42 (s, 1H), 5.08 (s, 1H), 4.68-4.61 (m, 1H), 3.68-3.65 (m, 4H), 3.41-3.39 (m, 4H), 2.48-2.43 (m, 2H), 2.09-2.04 (m, 2H), 1.25 (s, 3H).

### Intermediate 56: 1-([[4-iodo-6-(morpholin-4-yl)pyridin-2-yl]oxy]methyl)cyclopropan-1-ol

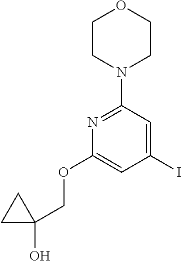 [SMILES: OC1(COc2cc(I)cc(N3CCOCC3)n2)CC1]

### Step 1: 1-(hydroxymethyl)cyclopropan-1-ol

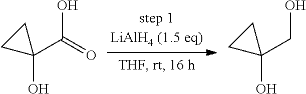 [SMILES: O=C(O)C1(O)CC1 ; OCC1(O)CC1]

To a stirred solution of 1-hydroxycyclopropane-1-carboxylic acid (0.6 g, 5.88 mmol) in THF (10.00 mL) was added LiAlH₄ (0.33 g, 8.82 mmol) in portions at 0° C. under nitrogen atmosphere. The reaction mixture was stirred for 16 h at room temperature. The resulting mixture was quenched with water (0.66 mL), NaOH (aq. 10%, 1.32 mL) and water (1.98 mL) in sequence at 0° C. The resulting mixture was filtered and the filter cake was washed with THE (3×10 mL). The filtrate was concentrated under reduced pressure. The residue was purified by silica gel column chromatography, eluted with EA/EtOH (3/1) in PE (60%). The fractions contained desired product were combined and concentrated to afford 1-(hydroxymethyl)cyclopropan-1-ol (0.62 g, 60%) as a colorless oil. H-NMR (400 MHz, d₆-DMSO) δ 5.14 (s, 1H), 4.53 (t, J=5.6 Hz, 1H), 3.40 (d, J=5.6 Hz, 2H), 0.51-0.42 (m, 4H).

### Step 2: 1-[[(6-fluoro-4-iodopyridin-2-yl)oxy]methyl]cyclopropan-1-ol

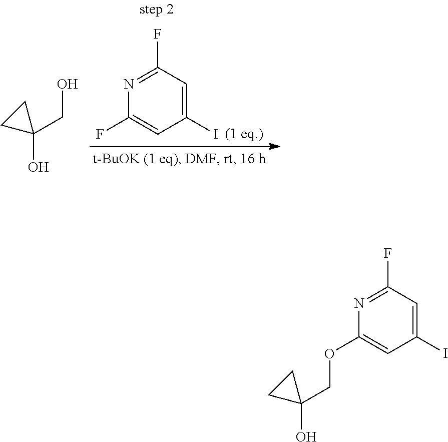

To a stirred solution of 1-(hydroxymethyl)cyclopropan-1-ol (0.62 g, 7.05 mmol) in DMF (8.00 mL) was added t-BuOK (1 M in THF, 7.03 mL, 7.03 mmol) dropwise at 0° C. under nitrogen atmosphere. The reaction mixture was stirred for 1 h at room temperature. To the above mixture was added a solution of 2,6-difluoro-4-iodopyridine (1.70 g, 7.03 mmol) in DMF (5.00 mL) at room temperature. The reaction mixture was stirred for additional 16 h at room temperature. The resulting mixture was quenched with water (150 mL). The resulting mixture was extracted with EA (3×50 mL). The combined organic layers was washed with brine (100 mL), dried over anhydrous Na₂SO₄ and filtered. The filtrate was concentrated under reduced pressure. The residue was purified by silica gel column chromatography, eluted with 32% EA in PE. The fractions contained desired product were combined and concentrated to afford 1-[[(6-fluoro-4-iodopyridin-2-yl)oxy]methyl]cyclopropan-1-ol (0.32 g, 13%) as a yellow oil. MS ESI calculated for C₉H₉₁NO₂ [M+H]⁺, 309.97, found 309.90.

### Step 3: 1-([[4-iodo-6-(morpholin-4-yl)pyridin-2-yl]oxy]methyl)cyclopropan-1-ol

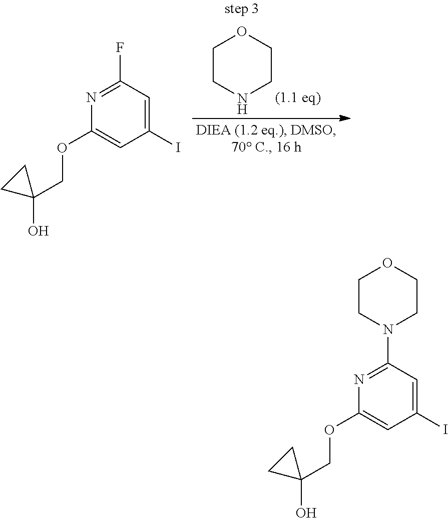 [SMILES: OC1(COc2cc(I)cc(F)n2)CC1 ; C1COCCN1 ; OC1(COc2cc(I)cc(N3CCOCC3)n2)CC1]

To a stirred solution of 1-[[(6-fluoro-4-iodopyridin-2-yl)oxy]methyl]cyclopropan-1-ol (0.32 g, 1.02 mmol) in DMSO (6.00 mL) were added morpholine (97.67 mg, 1.12 mmol) and DIEA (0.16 g, 1.22 mmol) at room temperature under nitrogen atmosphere. The reaction mixture was stirred for 16 h at 70° C. The resulting mixture was diluted with water (60 mL) and extracted with EA (3×30 mL). The combined organic layers was washed with brine (50 mL), dried over anhydrous Na₂SO₄ and filtered. The filtrate was concentrated under reduced pressure. The residue was purified by silica gel column chromatography, eluted with EA (50%) in PE. The fractions contained desired product were combined and concentrated to afford 1-([[4-iodo-6-(morpholin-4-yl)pyridin-2-yl]oxy]methyl)cyclopropan-1-ol (0.28 g, 73%) as a light yellow solid. MS ESI calculated for C₁₃H₁₇IN₂O₃[M+H]⁺, 377.03, found 377.00. H-NMR (400 MHz, d₆-DMSO) δ 6.67 (s, 1H), 6.48 (s, 1H), 5.49 (s, 1H), 4.18 (s, 2H), 3.67-3.65 (m, 4H), 3.42-3.39 (m, 4H), 0.66-0.57 (m, 4H).

### Intermediate 57: 4-[6-[(3,3-difluorocyclopentyl)oxy]-4-iodopyridin-2-yl]morpholine

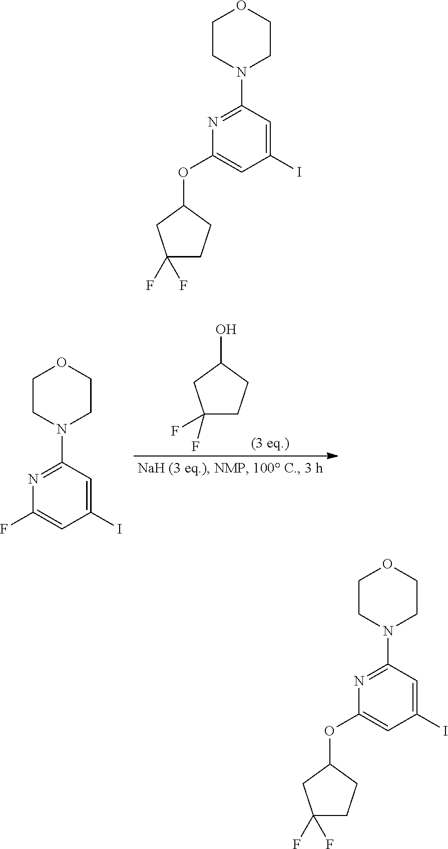 [SMILES: FC1(F)CCC(Oc2cc(I)cc(N3CCOCC3)n2)C1 ; Fc1cc(I)cc(N2CCOCC2)n1 ; OC1CCC(F)(F)C1 ; FC1(F)CCC(Oc2cc(I)cc(N3CCOCC3)n2)C1]

To a stirred solution of 3,3-difluorocyclopentan-1-ol (0.24 g, 1.95 mmol) in NMP (2.00 mL) was added NaH (46.73 mg, 1.95 mmol) at 0° C. under nitrogen atmosphere. The reaction mixture was stirred for 1 h at 25° C. To the above mixture was added 4-(6-fluoro-4-iodopyridin-2-yl)morpholine (0.20 g, 0.65 mmol) at 25° C. The reaction mixture was stirred for additional 3 h at 100° C. The reaction was quenched by the addition of saturated aqueous NaHCO₃ (100 mL) at 0° C. The resulting mixture was extracted with EA (3×100 mL). The combined organic layers was washed with saturated brine (3×50 mL), dried over anhydrous Na₂SO₄ and filtered. The filtrate was concentrated under reduced pressure. The residue was purified by silica gel column chromatography, eluted with PE/EA (2:1). The fractions contained desired product were combined and concentrated to afford 4-[6-[(3,3-difluorocyclopentyl)oxy]-4-iodopyridin-2-yl]morpholine (0.14 g, 52%) as an off-white solid. MS ESI calculated for C₁₄H₁₇F₂IN₂O₂[M+H]⁺, 411.03, found 411.05. ¹H-NMR (400 MHz, DMSO-d₆) δ 6.71 (m, 1H), 6.45 (m, 1H), 5.36-5.31 (m, 1H), 3.69-3.65 (m, 4H), 3.46-3.41 (m, 4H), 2.70-2.56 (m, 1H), 2.31-2.12 (m, 3H), 1.96-1.85 (m, 2H).

### Intermediate 58: Imino(2-[[4-iodo-6-(morpholin-4-yl)pyridin-2-yl]oxy]ethyl)methyl-λ⁶-sulfanone

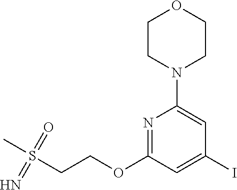 [SMILES: CS(=N)(=O)CCOc1cc(I)cc(N2CCOCC2)n1]

### Step 1: 4-[4-iodo-6-[2-(methylsulfanyl)ethoxy]pyridin-2-yl]morpholine

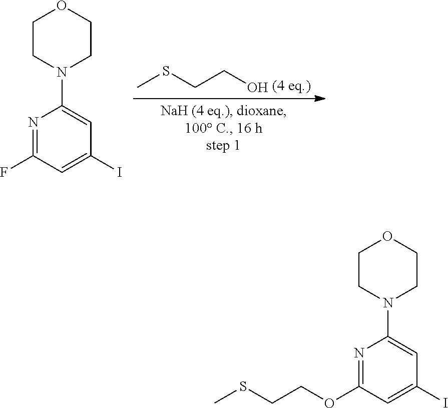

To a stirred solution of 2-(methylthio)ethanol (1 g, 11.04 mmol) in dioxane (20 mL) was added NaH (0.44 g, 11.04 mmol, 60%) at 0° C. under nitrogen atmosphere. The reaction mixture was stirred for 0.5 h at room temperature. To the above mixture was added 4-(6-fluoro-4-iodopyridin-2-yl)morpholine (0.85 g, 2.76 mmol) at room temperature. The reaction mixture was stirred for additional 16 h at 100° C. The resulting mixture was concentrated under reduced pressure. The residue was purified by silica gel column chromatography, eluted with EA/PE (0 to 100%). The fractions contained desired product were combined and concentrated to afford 4-[4-iodo-6-[2-(methylsulfanyl)ethoxy]pyridin-2-yl]morpholine (1 g, 95%) as a grey solid. MS ESI calculated for C₁₂H₁₇IN₂O₂S [M+H]⁺, 381.01, found 380.95.

### Step 2: 4-[4-iodo-6-(2-methanesulfinylethoxy)pyridin-2-yl]morpholine

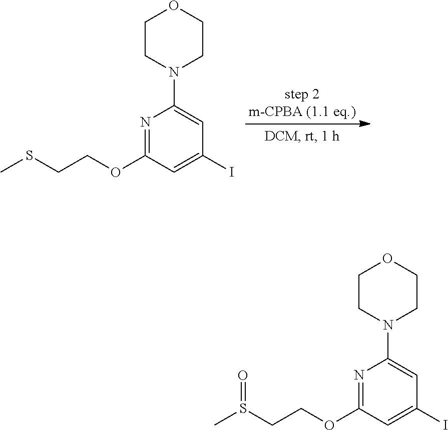 [SMILES: CSCCOc1cc(I)cc(N2CCOCC2)n1 ; CS(=O)CCOc1cc(I)cc(N2CCOCC2)n1]

To a stirred solution of 4-[4-iodo-6-[2-(methylsulfanyl)ethoxy]pyridin-2-yl]morpholine (1 g, 2.63 mmol) in DCM (20 mL) was added m-CPBA (0.59 g, 2.89 mmol, 85%) at 0° C. under nitrogen atmosphere. The reaction mixture was stirred for 1 h at room temperature. The resulting mixture was concentrated under reduced pressure. The residue was purified by silica gel column chromatography, eluted with EA:EtOH=3:1/PE (0 to 100%). The fractions contained desired product were combined and concentrated to afford 4-[4-iodo-6-(2-methanesulfinylethoxy)pyridin-2-yl]morpholine (0.90 g, 86%) as a grey solid. MS ESI calculated for C₁₂H₁₇IN₂O₃S [M+H]⁺, 397.00, found 396.90.

### Step 3: imino(2-[[4-iodo-6-(morpholin-4-yl)pyridin-2-yl]oxy]ethyl)methyl-λ⁶-sulfanone

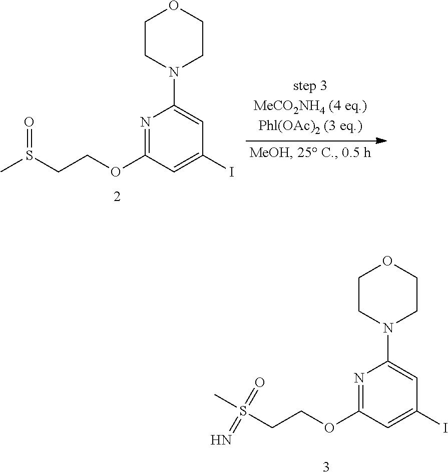 [SMILES: CS(=O)CCOc1cc(I)cc(N2CCOCC2)n1 ; CS(=N)(=O)CCOc1cc(I)cc(N2CCOCC2)n1]

To a solution of 4-[4-iodo-6-(2-methanesulfinylethoxy)pyridin-2-yl]morpholine (0.5 g, 1.26 mmol) and ammonium acetate (0.39 g, 5.05 mmol) in MeOH (2.5 mL) was added PhI(OAc)₂ (1.2 g, 3.79 mmol). The reaction mixture was stirred for 0.5 h at room temperature under nitrogen atmosphere. The resulting mixture was purified by reverse flash chromatography with the following conditions: column, C₁₈ silica gel; mobile phase, ACN in water, 20% to 50% gradient in 20 min; detector, UV 254/220 nm. The fractions contained desired product were combined and concentrated to afford imino(2-[[4-iodo-6-(morpholin-4-yl)pyridin-2-yl]oxy]ethyl)methyl-λ⁶-sulfanone (80 mg, 15%) as a brown solid. MS ESI calculated for C₁₂H₁₈IN₃O₃S [M+H]⁺, 412.01; found 411.95.

### Intermediate 59: 3-((4-iodo-6-morpholinopyridin-2-yl)oxy)-1-methylcyclopentan-1-ol

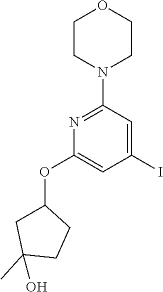 [SMILES: CC1(O)CCC(Oc2cc(I)cc(N3CCOCC3)n2)C1]

The title compound was prepared using procedures similar to those described in Intermediate 4 step 2 using 1-methylcyclopentane-1,3-diol instead of 2-(oxan-2-yloxy)ethanol to afford the title compound as a solid.

### Intermediate 60: 3-(((4-iodo-6-morpholinopyridin-2-yl)oxy)methyl)oxetan-3-ol

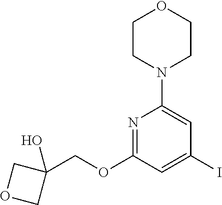 [SMILES: OC1(COc2cc(I)cc(N3CCOCC3)n2)COC1]

The title compound was prepared using procedures similar to those described in Intermediate 54 step 2 using 3-(hydroxymethyl)oxetan-3-ol instead of 2-(oxan-2-yloxy)ethanol to afford the title compound as a solid.

### Intermediate 61: 3-(2-((4-iodo-6-morpholinopyridin-2-yl)oxy)ethyl)oxetan-3-ol

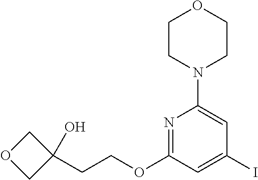 [SMILES: OC1(CCOc2cc(I)cc(N3CCOCC3)n2)COC1]

The title compound was prepared using procedures similar to those described in Intermediate 54 step 2 using 3-(2-hydroxyethyl)oxetan-3-ol instead of 2-(oxan-2-yloxy)ethanol to afford the title compound as a solid.

### Intermediates 12 and 13: 4-[6-chloro-2-[(3,3-difluorocyclopentyl)oxy]pyrimidin-4-yl]morpholine

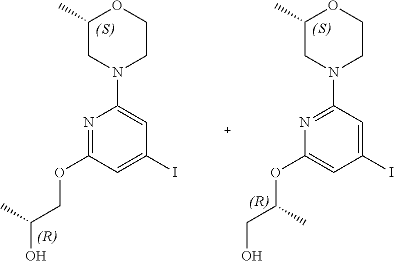

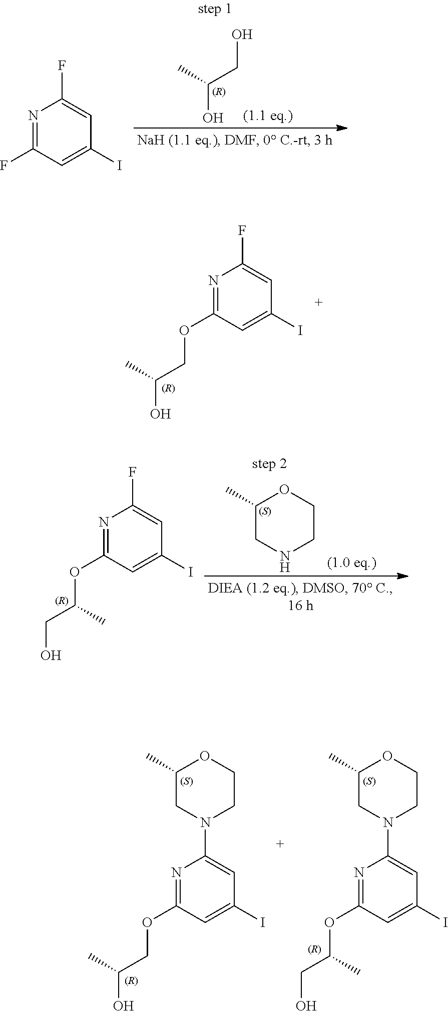

### Step 1: (2R)-1-[(6-fluoro-4-iodopyridin-2-yl)oxy]propan-2-ol and (2R)-2-[(6-fluoro-4-iodopyridin-2-yl)oxy]propan-1-ol

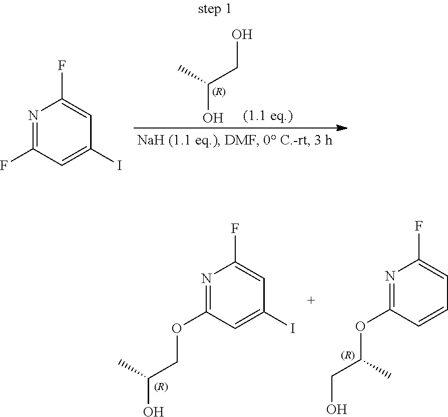

To a solution of R-1,2-propanediol (5.00 g, 65.707 mmol, 1.10 equiv) in DMF (120 mL) was added NaH (2.63 g, 65.756 mmol, 1.10 equiv, 60%) at 0 degrees C. The mixture was stirred for 45 min. 2,6-difluoro-4-iodopyridine (14.40 g, 59.756 mmol, 1.00 equiv) was added and the mixture was allowed to warm to RT and stirred for 3 h. The reaction was quenched by the addition of sat. NH₄Cl (aq.) (100 mL) at 0 degrees C. The resulting mixture was extracted with DCM (4×80 mL). The combined organic layers were washed with brine (400 mL), dried over anhydrous Na₂SO₄. After filtration, the filtrate was concentrated under reduced pressure. The residue was purified by silica gel column chromatography, eluted with 30% EtOAc in PE to afford a mixture of (2R)-1-[(6-fluoro-4-iodopyridin-2-yl)oxy]propan-2-ol (4.8 g, 27%) and (2R)-2-[(6-fluoro-4-iodopyridin-2-yl)oxy]propan-1-ol (7.0 g, 40%, ratio ˜2:1) as a light yellow oil. MS ESI calculated for C₈H₉FINO₂ [M+H]⁺, 297.97; found 298.00.

### Step 2: (2R)-1-[(6-fluoro-4-iodopyridin-2-yl)oxy]propan-2-ol and (2R)-2-[(6-fluoro-4-iodopyridin-2-yl)oxy]propan-1-ol

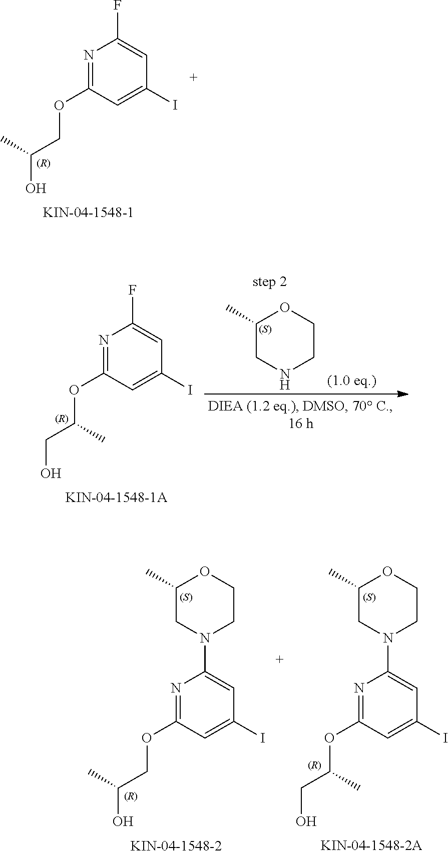

To a mixture of (2R)-1-[(6-fluoro-4-iodopyridin-2-yl)oxy]propan-2-ol and (2R)-2-[(6-fluoro-4-iodopyridin-2-yl)oxy]propan-1-ol (1.00 g, 3.366 mmol, 1.00 equiv) in DMSO (10 mL) were added (2S)-2-methylmorpholine (340.49 mg, 3.366 mmol, 1.00 equiv) and DIEA (522.08 mg, 4.039 mmol, 1.20 equiv). The resulting mixture was stirred for 2 h at 70 degrees C. The resulting mixture was quenched with H₂O (100 mL), then extracted with and EtOAc (4×50 mL). The combined organic layers were washed with brine (5×50 mL), dried over anhydrous Na₂SO₄. After filtration, the filtrate was concentrated under reduced pressure. The residue was purified by silica gel column chromatography, eluted with PE/EtOAc (4:1) to afford a mixture of (2R)-2-([4-iodo-6-[(2S)-2-methylmorpholin-4-yl]pyridin-2-yl]oxy)propan-1-ol and (2R)-1-([4-iodo-6-[(2S)-2-methylmorpholin-4-yl]pyridin-2-yl]oxy)propan-2-ol (1.2 g, 93%) as an oyster white oil. MS ESI calculated for C₁₃H₁₉IN₂O₃[M+H]⁺, 379.04; found 378.95.

### Intermediate 64: (R)-1-((4-iodo-6-((R)-2-methylmorpholino)pyridin-2-yl)oxy)propan-2-ol

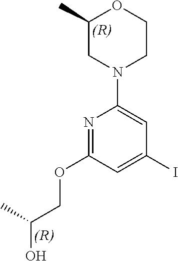

The title compound was prepared using procedures similar to those described in Intermediate 63 and 2 using (2R)-2-methylmorpholine instead of (2S)-2-methylmorpholine to afford the title compound as a solid.

### Intermediate 65: (2R)-1-((6-(2-oxa-5-azabicyclo[4.1.0]heptan-5-yl)-4-iodopyridin-2-yl)oxy)propan-2-ol

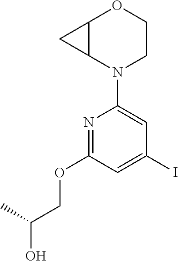 [SMILES: C[C@@H](O)COc1cc(I)cc(N2CCOC3CC32)n1]

The title compound was prepared using procedures similar to those described in Intermediate 63 and 2 using 2-oxa-5-azabicyclo[4.1.0]heptane hydrochloride instead of (2S)-2-methylmorpholine to afford the title compound as a solid.

### Intermediate 66: (S)-N-(3-(2-chloro-6-((R)-2-hydroxypropoxy)pyridin-4-yl)-4-methylphenyl)-3-(2,2,2-trifluoroethyl)pyrrolidine-1-carboxamide

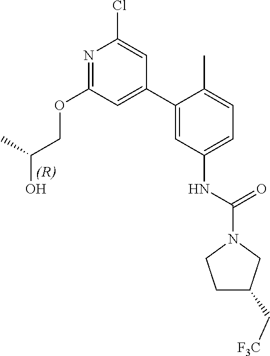

### Step 1: (R)-1-((6-chloro-4-iodopyridin-2-yl)oxy)propan-2-ol and (R)-2-((6-chloro-4-iodopyridin-2-yl)oxy)propan-1-ol

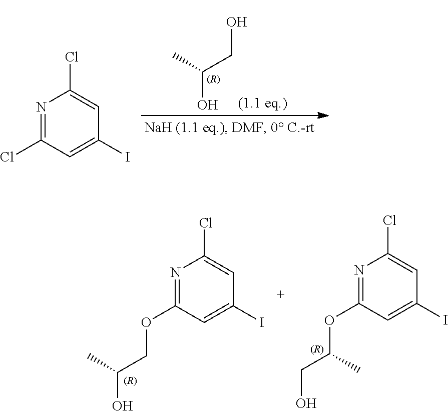

To a solution of R-1,2-propanediol (1.53 g, 20.082 mmol, 1.10 equiv) in DMF (50 mL) was added NaH (0.80 g, 20.082 mmol, 1.10 equiv, 60%) at 0° C. The mixture was stirred for 1 h at 25° C. 2,6-dichloro-4-iodopyridine (5.00 g, 18.256 mmol, 1.00 equiv) was added and the mixture was stirred for 2 h at 25° C. The resulting mixture was diluted water and extracted with EtOAc. The combined organic layers were washed with brine, and dried over anhydrous Na₂SO₄. After filtration, the filtrate was concentrated under reduced pressure. The residue was purified by silica gel column chromatography, eluted with PE/EtOAc (1:1) to afford a mixture of (R)-1-((6-chloro-4-iodopyridin-2-yl)oxy)propan-2-ol and (R)-2-((6-chloro-4-iodopyridin-2-yl)oxy)propan-1-ol (1.2 g, 21%) as an off-white oil. MS ESI calculated for C₈H₉ClINO₂ [M+H]⁺, 313.94, found 313.95. H-NMR (300 MHz, Chloroform-d) δ 7.33-7.27 (m, 1H), 7.12 (dd, J=12.6, 1.1 Hz, 1H), 6.82 (d, J=5.4 Hz, 1H), 4.25-4.09 (m, 2H), 1.49 (d, J=6.4 Hz, 1H), 1.36-1.26 (m, 3H).

### Step 2: (3S)-N-(3-[2-chloro-6-[(2R)-2-hydroxypropoxy]pyridin-4-yl]-4-methylphenyl)-3-(2,2,2-trifluoroethyl)pyrrolidine-1-carboxamide

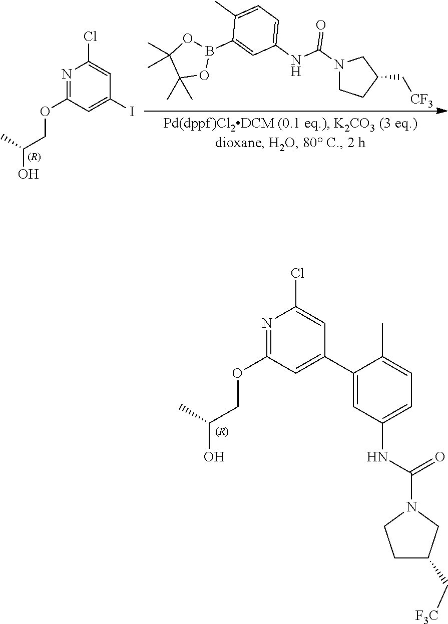

A mixture of (2R)-1-[(6-chloro-4-iodopyridin-2-yl)oxy]propan-2-ol and (2R)-2-[(6-fluoro-4-iodopyridin-2-yl)oxy]propan-1-ol (1.20 g, 3.828 mmol, 1.00 equiv), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (1.58 g, 3.828 mmol, 1 equiv), Pd(dppf)Cl₂—CH₂Cl₂ (0.31 g, 0.383 mmol, 0.1 equiv) and Na₂CO₃ (1.22 g, 11.483 mmol, 3 equiv) in dioxane (12.00 mL) and H₂O (1.20 mL) was stirred for 2 h at 80° C. under nitrogen atmosphere. The reaction mixture was concentrated under reduced pressure. The compound was separated by Prep-Chiral HPLC with the following conditions: (Column: CHIRALPAK IG, 5*25 cm, 10 um; Mobile Phase A: CO₂, Mobile Phase B: MeOH (0.1% 2M NH3-MeOH); Flow rate: 180 mL/min; Gradient: 45% B; 220 nm; RT1: 5.32; RT2: 6.85; Injection Volume: 2.5 ml; Number Of Runs: 16). The collected fractions were combined and concentrated under reduced pressure to afford (3S)-N-(3-[2-chloro-6-[(2R)-2-hydroxypropoxy]pyridin-4-yl]-4-methylphenyl)-3-(2,2,2-trifluoroethyl)pyrrolidine-1-carboxamide (650 mg, 36%) as a light yellow solid. MS ESI calculated for C₂₂H₂₅ClF₃N₃O₃ [M+H]⁺, 472.15, found 472.15. H-NMR (300 MHz, Chloroform-d) δ 7.37-7.28 (m, 2H), 7.20 (d, J=8.3 Hz, 1H), 6.92 (d, J=1.1 Hz, 1H), 6.67 (d, J=1.1 Hz, 1H), 6.27 (s, 1H), 4.38 (t, J=7.5 Hz, 1H), 4.30-4.15 (m, 2H), 3.82 (t, J=8.6 Hz, 1H), 3.65 (t, J=9.0 Hz, 1H), 3.45 (q, J=9.2 Hz, 1H), 3.13 (t, J=9.4 Hz, 1H), 2.56 (d, J=9.5 Hz, 2H), 2.34-2.23 (m, 1H), 2.24 (s, 3H), 1.74 (q, J=10.0, 9.6 Hz, 1H), 1.30 (t, J=6.0 Hz, 3H).

### Intermediate 67: (2R,3R)-3-((4-iodo-6-morpholinopyridin-2-yl)oxy)butan-2-ol

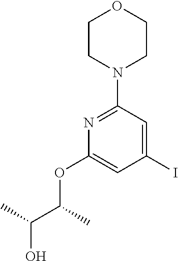 [SMILES: C[C@@H](O)[C@@H](C)Oc1cc(I)cc(N2CCOCC2)n1]

### Step 1: 4-(6-fluoro-4-iodopyridin-2-yl)morpholine

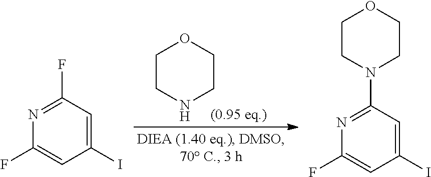 [SMILES: Fc1cc(I)cc(F)n1 ; C1COCCN1 ; Fc1cc(I)cc(N2CCOCC2)n1]

To a stirred solution of 2,6-difluoro-4-iodopyridine (16.00 g, 66.40 mmol) in DMSO (240 mL) were added morpholine (5.49 mL, 63.04 mmol) and DIEA (12.07 mL, 93.40 mmol) at room temperature under nitrogen atmosphere. The reaction mixture was stirred at 70° C. for 3 h. The resulting mixture was cooled to rt, diluted with water (150 mL) and extracted with EA (300 mL×3). The combined organic layers was washed with brine (100 mL×4), dried over anhydrous Na₂SO₄, filtered and concentrated under reduced pressure. The residue was purified by silica gel column chromatography eluting with 30% EA in PE to afford 4-(6-fluoro-4-iodopyridin-2-yl)morpholine (17.60 g, 86%) as an off-white solid. MS ESI calculated for C₉H₁₀FIN₂O [M+H]⁺, 308.98, found 309.10.

### Step 2: (2R,3R)-3-((4-iodo-6-morpholinopyridin-2-yl)oxy)butan-2-ol

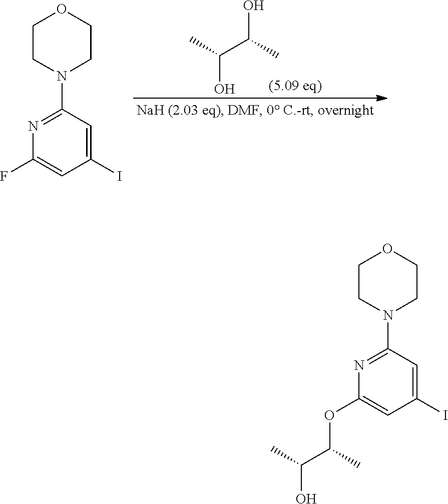

To a solution of (2R,3R)-butane-2,3-diol (1.40 g, 16.31 mmol) in DMF (20 mL) was added NaH (260 mg, 60%, 6.50 mmol) at 0° C. The reaction mixture was stirred at 0° C. for 30 min. Then 4-(6-fluoro-4-iodopyridin-2-yl)morpholine (1.00 g, 3.20 mmol) was added to the reaction mixture. The mixture was stirred at room temperature overnight. The reaction mixture was poured into ice water (30 mL) and extracted with DCM (30 mL×2). The combined organic layers were dried over Na₂SO₄, filtered and concentrated in vacuum. The residue was purified by silica gel column chromatography eluting with 20% EA in PE to give (2R,3R)-3-((4-iodo-6-morpholinopyridin-2-yl)oxy)butan-2-ol (1.20 g, 80%) as yellow oil. MS ESI calculated for C₁₉H₁₉IN₂O₃[M+H]⁺, 379.04, found 379.10.

### Intermediate 68: (S)-3-(difluoromethoxy)-N-(4-methyl-3-(4,4,5,5-tetramethyl-1,3,2-dioxaborolan-2-yl)phenyl)pyrrolidine-1-carboxamide

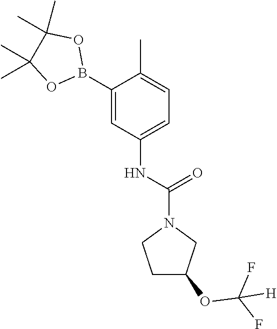 [SMILES: Cc1ccc(NC(=O)N2CC[C@H](OC(F)F)C2)cc1B1OC(C)(C)C(C)(C)O1]

### Step 1: (S)-3-(difluoromethoxy)pyrrolidine

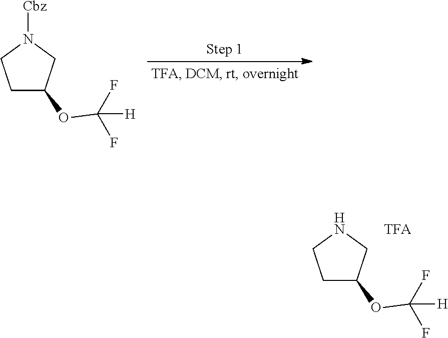 [SMILES: O=C(OCc1ccccc1)N1CC[C@H](OC(F)F)C1 ; FC(F)O[C@H]1CCNC1]

To a solution of (S)-benzyl 3-(difluoromethoxy)pyrrolidine-1-carboxylate (7.00 g, 25.80 mmol) in DCM (100 mL) was added TFA (20 mL). The reaction mixture was stirred at room temperature overnight. The solution was concentrated in vacuum to afford the TFA salt of (S)-3-(difluoromethoxy)pyrrolidine (7.01 g, crude) as yellow oil.

### Step 2: (S)-3-(difluoromethoxy)-N-(4-methyl-3-(4,4,5,5-tetramethyl-1,3,2-dioxaborolan-2-yl)phenyl)pyrrolidine-1-carboxamide

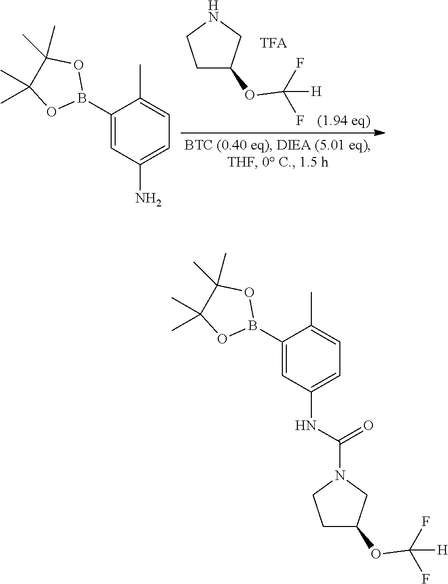 [SMILES: Cc1ccc(N)cc1B1OC(C)(C)C(C)(C)O1 ; FC(F)O[C@H]1CCNC1 ; Cc1ccc(NC(=O)N2CC[C@H](OC(F)F)C2)cc1B1OC(C)(C)C(C)(C)O1]

To a solution of 4-methyl-3-(4,4,5,5-tetramethyl-1,3,2-dioxaborolan-2-yl)aniline (1.01 g, 4.29 mmol) and DIEA (2.80 g, 21.50 mmol) in THF (50 mL) was added BTC (510 mg, 1.72 mmol) at 0° C. under nitrogen atmosphere. The reaction mixture was stirred at 0° C. for 30 min. Then the TFA salt of (S)-3-(difluoromethoxy)pyrrolidine (2.10 g, 8.33 mmol) was added to the mixture. The mixture was stirred at 0° C. for another 1 h. The mixture was concentrated and purified by silica gel column chromatography eluting with 50% EA in PE to afford (S)-3-(difluoromethoxy)-N-(4-methyl-3-(4,4,5,5-tetramethyl-1,3,2-dioxaborolan-2-yl)phenyl)pyrrolidine-1-carboxamide (300 mg, 17%) as a white solid. MS ESI calculated for C₁₆H₂₃IN₂O₄[M+H]⁺, 397.20, found 397.30.

### Intermediate 69: N-(3-bromo-4-methylphenyl)-3-(tert-butyl)-1H-pyrrole-1-carboxamide

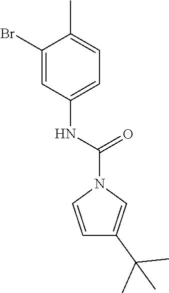 [SMILES: Cc1ccc(NC(=O)n2ccc(C(C)(C)C)c2)cc1Br]

### N-(3-bromo-4-methylphenyl)-3-(tert-butyl)-1H-pyrrole-1-carboxamide

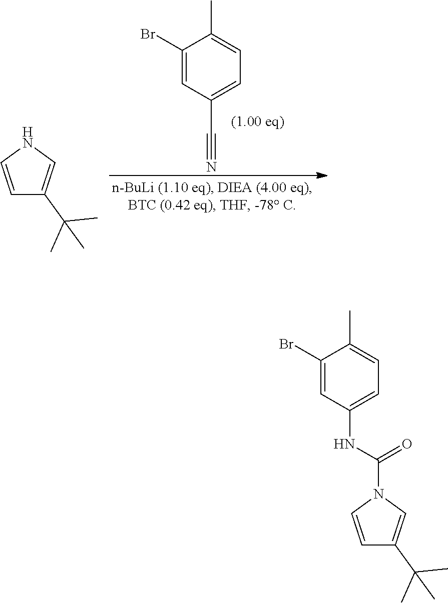

To a solution of 3-(tert-butyl)-1H-pyrrole (300 mg, 2.44 mmol) in THF (20 mL) was added n-BuLi (1.07 mL, 2.68 mmol) at 0° C. Then the reaction mixture was stirred at rt for 30 min. To another flask was added BTC (288 mg, 1.04 mmol), DIEA (1.26 g, 9.76 mmol) and THF (20 mL). Then the mixture was stirred for 5 min at −78° C. After stirring, the mixture was added to the previous solution and stirred for 30 min at −78° C. Then the mixture was stirred at rt for another 30 min. The resulting mixture was quenched with water (100 mL) and extracted with EtOAc (50 mL×3). The combined organic layers were washed with brine (100 mL), dried over anhydrous Na₂SO₄, filtered and concentrated under reduced pressure. The residue was purified by silica gel column chromatography eluting with EA in PE (0-50%) to afford N-(3-bromo-4-methylphenyl)-3-(tert-butyl)-1H-pyrrole-1-carboxamide (117 mg, 14%) as a yellow solid. MS ESI calculated for C₁₆H₁₉BrN₂O [M+H]⁺, 335.07, found 335.10.

### Intermediate 70: (R)-4-(6-((2,2-dimethyl-1,3-dioxolan-4-yl)methoxy)-4-iodopyridin-2-yl)morpholine

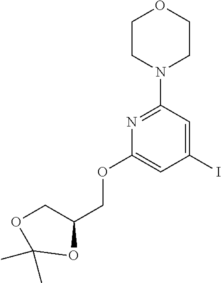 [SMILES: CC1(C)OC[C@@H](COc2cc(I)cc(N3CCOCC3)n2)O1]

The title compound was prepared using procedures similar to those described in Intermediate 67 step 2 using (R)-(2,2-dimethyl-1,3-dioxolan-4-yl)methanol to afford the title compound as a solid.

### Intermediate 71: (S)-4-(6-((2,2-dimethyl-1,3-dioxolan-4-yl)methoxy)-4-iodopyridin-2-yl)morpholine

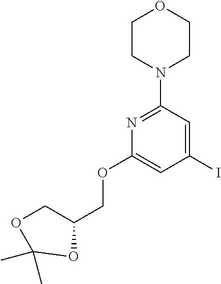 [SMILES: CC1(C)OC[C@H](COc2cc(I)cc(N3CCOCC3)n2)O1]

The title compound was prepared using procedures similar to those described in Intermediate 67 step 2 using (S)-(2,2-dimethyl-1,3-dioxolan-4-yl)methanol to afford the title compound as a solid.

### Intermediate 72: N-(4-methyl-3-(4,4,5,5-tetramethyl-1,3,2-dioxaborolan-2-yl)phenyl)-3-(2,2,2-trifluoroethyl)pyrrolidine-1-carboxamide

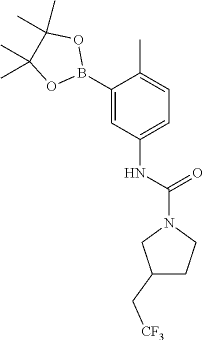 [SMILES: Cc1ccc(NC(=O)N2CCC(CC(F)(F)F)C2)cc1B1OC(C)(C)C(C)(C)O1]

The title compound was prepared using procedures similar to those described in Intermediate 68 step 2 using 3-(2,2,2-trifluoroethyl)pyrrolidine to afford the title compound as a solid.

### Intermediate 73: 4-(4-iodo-6-(((2S)-1-((tetrahydro-2H-pyran-2-yl)oxy)propan-2-yl)oxy)pyridin-2-yl)morpholine

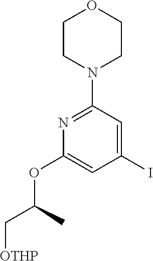

The title compound was prepared using procedures similar to those described in Intermediate 67 step 2 using (2S)-1-((tetrahydro-2H-pyran-2-yl)oxy)propan-2-ol to afford the title compound as a solid.

### Intermediate 74: (R)-tert-butyl 3-((4-iodo-6-morpholinopyridin-2-yl)oxy)pyrrolidine-1-carboxylate

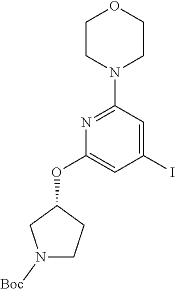 [SMILES: CC(C)(C)OC(=O)N1CC[C@@H](Oc2cc(I)cc(N3CCOCC3)n2)C1]

The title compound was prepared using procedures similar to those described in Intermediate 67 step 2 using (R)-tert-butyl 3-hydroxypyrrolidine-1-carboxylate to afford the title compound as a solid.

### Intermediate 75: tert-butyl 3-((4-iodo-6-morpholinopyridin-2-yl)oxy)azetidine-1-carboxylate

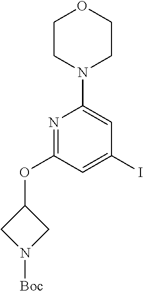 [SMILES: CC(C)(C)OC(=O)N1CC(Oc2cc(I)cc(N3CCOCC3)n2)C1]

The title compound was prepared using procedures similar to those described in Intermediate 67 using tert-butyl 3-hydroxyazetidine-1-carboxylate to afford the title compound as a solid.

### Example 1 and Example 2: Synthesis of (R)-N-(2-fluoro-5-(2-(2-hydroxyethoxy)-6-morpholinopyridin-4-yl)-4-methylphenyl)-3-(trifluoromethyl)pyrrolidine-1-carboxamide; (S)-N-(2-fluoro-5-(2-(2-hydroxyethoxy)-6-morpholinopyridin-4-yl)-4-methylphenyl)-3-(trifluoromethyl)pyrrolidine-1-carboxamide

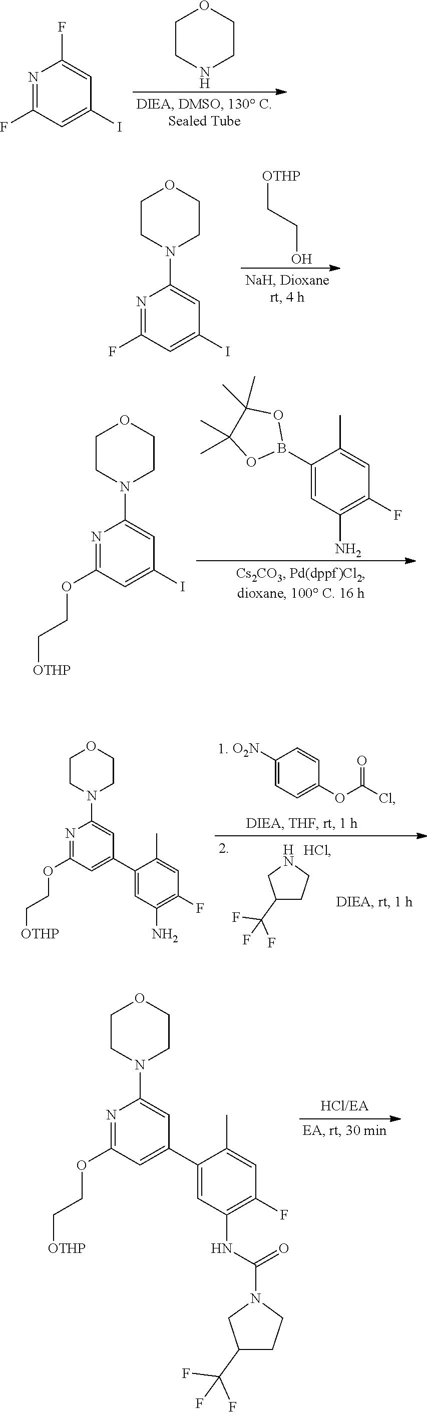

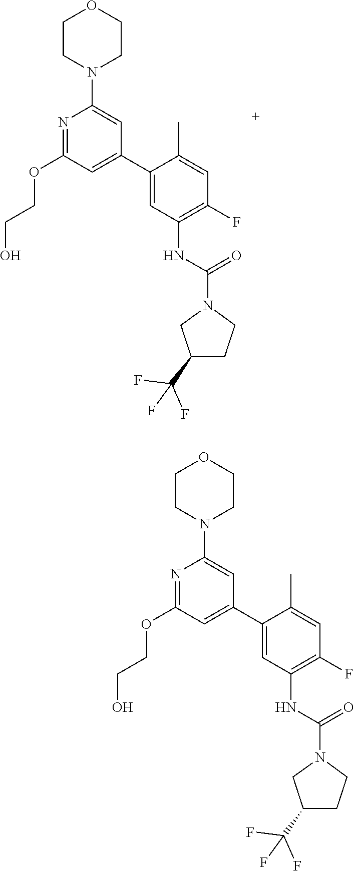 [SMILES: Cc1cc(F)c(NC(=O)N2CC[C@@H](C(F)(F)F)C2)cc1-c1cc(OCCO)nc(N2CCOCC2)c1 ; Cc1cc(F)c(NC(=O)N2CC[C@H](C(F)(F)F)C2)cc1-c1cc(OCCO)nc(N2CCOCC2)c1]

### Step 1:

To a solution of 2,6-difluoro-4-iodopyridine (10.0 g, 41.5 mmol) and morpholine (3.6 g, 41.5 mmol) in DMSO (100 mL) was added DIEA (10.7 g, 83.0 mmol) at rt. The mixture was stirred at 130° C. for 16 h in a sealed tube. The reaction was cooled down to rt, diluted with water (200 mL) and extracted with DCM (200 mL*3). The combined organic layers were washed with H₂O (200 mL*2) and brine (200 mL), dried over Na₂SO₄, filtered and concentrated. The residue was purified by flash chromatography on silica gel (PE:EA=20:1 to PE:EA=10:1) to afford 4-(6-fluoro-4-iodopyridin-2-yl)morpholine (10.4 g, 81.7%). MS Calcd.: 308, MS Found: 309 ([M+H]⁺).

### Step 2:

To a solution of 2-((tetrahydro-2H-pyran-2-yl)oxy)ethanol (24.8 g, 169.5 mmol) in dioxane (150 mL) was added NaH (6.8 g, 169.5 mmol, 60% in mineral oil) at 0° C., and the mixture was stirred at rt for 15 min, 4-(6-fluoro-4-iodopyridin-2-yl)morpholine (10.4 g, 33.9 mmol) was added and the mixture was stirred at 100° C. for 2 h. The reaction was cooled down to rt, diluted with water (200 mL) and extracted with DCM (200 mL*3). The combined organic layers were washed with H₂O (200 mL*2) and brine (200 mL), dried over Na₂SO₄, filtered and concentrated. The residue was purified by flash chromatography on silica gel (PE:EA=20:1 to PE:EA=5:1) to afford 4-(4-iodo-6-(2-((tetrahydro-2H-pyran-2-yl)oxy)ethoxy)pyridin-2-yl)morpholine (10.7 g, 72.8%). MS Calcd.: 434, MS Found: 435 ([M+H]⁺).

### Step 3:

To a solution of 4-(4-iodo-6-(2-((tetrahydro-2H-pyran-2-yl)oxy)ethoxy)pyridin-2-yl)morpholine (3.9 g, 8.9 mmol), 2-fluoro-4-methyl-5-(4,4,5,5-tetramethyl-1,3,2-dioxaborolan-2-yl)aniline (crude, 9.8 mmol) and Cs₂CO₃ (8.7 g, 26.7 mmol) in dioxane (100 mL) and water (10 mL) was added Pd(dppf)Cl₂ (652 mg, 0.89 mmol) at rt under N₂. The mixture was stirred at 100° C. for 16 h. The reaction was cooled down to rt, filtered and concentrated. The residue was purified by flash chromatography on silica gel (PE:EA=5:1 to PE:EA=2:1) to afford 2-fluoro-4-methyl-5-(2-morpholino-6-(2-((tetrahydro-2H-pyran-2-yl)oxy)ethoxy)pyridin-4-yl)aniline (2.7 g, 69.3%). MS Calcd.: 431 Found: 432 ([M+H]⁺).

### Step 4:

To a solution of 2-fluoro-4-methyl-5-(2-morpholino-6-(2-((tetrahydro-2H-pyran-2-yl)oxy)ethoxy)pyridin-4-yl)aniline (492 mg, 1.14 mmol) in THE (20 mL) was added DIEA (221 mg, 1.71 mmol) and 4-nitrophenyl carbonochloridate (230 mg, 1.14 mmol) at 0° C., and the mixture was stirred at rt for 1 h. The reaction was cooled down to 0° C., DIEA (442 mg, 3.42 mmol) and 3-(trifluoromethyl)pyrrolidine hydrochloride (200 mg, 1.14 mmol) were added. The reaction mixture was stirred at rt for 1 h and then concentrated. The residue was purified by flash chromatography on silica gel (PE:EA=5:1 to PE:EA=2:1) to give N-(2-fluoro-4-methyl-5-(2-morpholino-6-(2-((tetrahydro-2H-pyran-2-yl)oxy)ethoxy)pyridin-4-yl)phenyl)-3-(trifluoromethyl)pyrrolidine-1-carboxamide (480 mg, 70.6%). MS Calcd.: 596 MS Found: 597 ([M+H]⁺).

### Step 5:

To a solution of N-(2-fluoro-4-methyl-5-(2-morpholino-6-(2-((tetrahydro-2H-pyran-2-yl)oxy)ethoxy)pyridin-4-yl)phenyl)-3-(trifluoromethyl)pyrrolidine-1-carboxamide (280 mg, 0.47 mmol) in EtOAc (15 mL) was added HCl/EtOAc (10 mL, 2 M) at rt, and the reaction was stirred for 30 min. The mixture was concentrated and the residue was purified by flash chromatography on silica gel (PE:EA=2:1 to PE:EA=1:1) to yield the racemate mixture (120 mg, 49.8%). The mixture was then separated by chiral HPLC (Daicel Chiralpak IH: 20*250 mm L, 5 μm; CO₂:MeOH=75:25, 50 g/min, 230 nm) to give the two enantiomers: 50.8 mg (21.1%) of the first isomer eluted at 7.18 min (ee>98%) and 51.6 mg (21.5%) of the second isomer eluted at 9.03 min (ee>98%). ¹H NMR (400 MHz, DMSO-d₆) of first eluted isomer: δ 1.98-2.03 (m, 1H), 2.17-2.20 (m, 4H), 3.43-3.53 (m, 8H), 3.64-3.72 (m, 7H), 4.24 (t, J=5.6 Hz, 2H), 4.78 (t, J=5.6 Hz, 1H), 5.98 (s, 1H), 6.19 (s, 1H), 7.13 (d, J=11.6 Hz, 1H), 7.32 (d, J=8.4 Hz, 1H), 8.04 (s, 1H). MS Calcd.: 512 Found: 513 ([M+H]⁺). ¹H NMR (400 MHz, DMSO-d₆) of the of the second eluted isomer: δ 1.98-2.03 (m, 1H), 2.17-2.20 (m, 4H), 3.43-3.53 (m, 8H), 3.68-3.72 (m, 7H), 4.24 (t, J=5.6 Hz, 2H), 5.98 (s, 1H), 6.02 (brs, 1H), 6.19 (s, 1H), 7.13 (d, J=11.6 Hz, 1H), 7.32 (d, J=8.0 Hz, 1H), 8.04 (s, 1H). MS Calcd.: 512 Found: 513 ([M+H]⁺).

### Example 3: (RS)-N-(2-fluoro-5-(2-(2-hydroxyethoxy)-6-morpholinopyridin-4-yl)-4-methylphenyl)-3-(trifluoromethyl)pyrrolidine-1-carboxamide

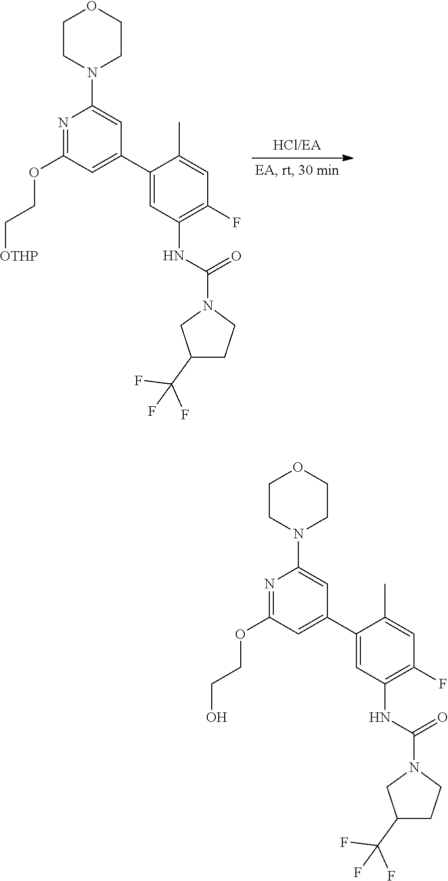

To a solution of N-(2-fluoro-4-methyl-5-(2-morpholino-6-(2-((tetrahydro-2H-pyran-2-yl)oxy)ethoxy)pyridin-4-yl)phenyl)-3-(trifluoromethyl)pyrrolidine-1-carboxamide (280 mg, 0.47 mmol) in EtOAc (15 mL) was added HCl/EtOAc (10 mL, 2 M) at rt, and the mixture was stirred for 30 min. The mixture was concentrated and the residue was purified by Prep-HPLC (Gilson-5 Xbridge, C8 5 μm 19*150 mm 30-70% B, A: H₂O (0.1% NH₄HCO₃), B: ACN, UV: 214 nm, Flowrate 15 mL/min, GT=8 min) to give N-(2-fluoro-5-(2-(2-hydroxyethoxy)-6-morpholinopyridin-4-yl)-4-methylphenyl)-3-(trifluoromethyl)pyrrolidine-1-carboxamide (106.6 mg, 45.9%). ¹H NMR (400 MHz, DMSO-d₆): δ 1.98-2.04 (m, 1H), 2.17-2.20 (m, 4H), 3.43-3.54 (m, 8H), 3.64-3.72 (m, 7H), 4.24 (t, J=5.6 Hz, 2H), 4.78 (t, J=5.6 Hz, 1H), 5.98 (s, 1H), 6.19 (s, 1H), 7.14 (d, J=11.6 Hz, 1H), 7.32 (d, J=8.4 Hz, 1H), 8.04 (s, 1H). MS Caled.: 512 Found: 513 ([M+H]⁺).

### Example 4: (RS)-N-(2-fluoro-5-(2-(2-hydroxyethoxy)-6-morpholinopyridin-4-yl)-4-methylphenyl)-3-(trifluoromethyl)piperidine-1-carboxamide

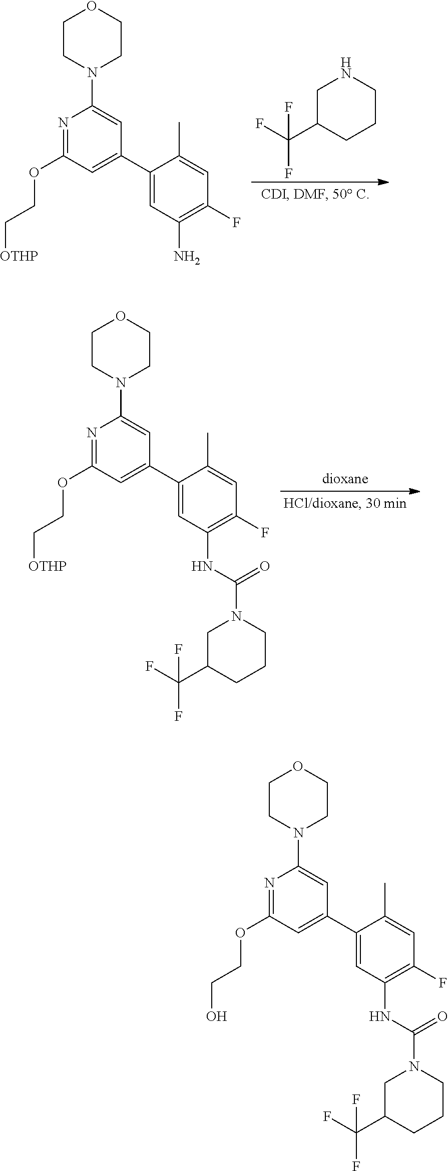

### Step 1:

A solution of 2-fluoro-4-methyl-5-{2-morpholin-4-yl-6-[2-(tetrahydro-pyran-2-yloxy)-ethoxy]-pyridin-4-yl}-phenylamine (150 mg, 0.34 mmol), CDI (74 mg, 0.45 mmol) and DIEA (147 mg, 1.14 mmol) in DMF (10 mL) was stirred at 50° C. for 2 h. 3-Trifluoromethyl-piperidine (59 mg, 0.38 mmol) was added and the reaction was stirred at 50° C. overnight. The mixture was concentrated and the residue was purified by flash chromatography on silica gel (PE:EA=5:1 to PE:EA=2:1) to afford 3-trifluoromethyl-piperidine-1-carboxylic acid (2-fluoro-4-methyl-5-{2-morpholin-4-yl-6-[2-(tetrahydro-pyran-2-yloxy)-ethoxy]-pyridin-4-yl}-phenyl)-amide (101 mg, 47.6%). MS Calcd.: 610 Found: 611 ([M+H]⁺).

### Step 2:

To a solution of N-(2-fluoro-4-methyl-5-(2-morpholino-6-(2-((tetrahydro-2H-pyran-2-yl)oxy)ethoxy)pyridin-4-yl)phenyl)-3-(trifluoromethyl)piperidine-1-carboxamide (101 mg, 0.16 mmol) in dioxane (10 mL) was added HCl/dioxane (3 mL, 2 M) at rt, and the mixture was stirred for 30 min. The mixture was concentrated and the residue was purified by Prep-HPLC (Waters-2 Sunfire, C8 5 μm 19*150 mm 35-70% B, A: H₂O (0.1% HCOOH), B: ACN, UV: 214 nm, Flowrate 15 mL/min, GT=10 min) to give N-(2-fluoro-5-(2-(2-hydroxyethoxy)-6-morpholinopyridin-4-yl)-4-methylphenyl)-3-(trifluoromethyl)piperidine-1-carboxamide (38.3 mg, 44.5%). ¹H NMR (400 MHz, DMSO-d₆): δ 1.44-1.52 (m, 2H), 1.70-1.73 (m, 1H), 1.94-1.96 (m, 1H), 2.20 (s, 3H), 2.79-2.87 (m, 2H), 3.43-3.45 (m, 5H), 3.67-3.70 (m, 7H), 3.97-4.01 (m, 1H), 4.20-4.25 (m, 2H), 4.76-4.79 (m, 1H), 5.97 (s, 1H), 6.18 (s, 1H), 7.12 (d, J=11.6 Hz, 1H), 7.22 (d, J=8.4 Hz, 1H), 8.41 (s, 1H). MS Calcd.: 526; MS Found: 527 ([M+H]⁺)

### Example 5: (RS)-3-(tert-butyl)-N-(2-fluoro-5-(2-(2-hydroxyethoxy)-6-morpholinopyridin-4-yl)-4-methylphenyl)pyrrolidine-1-carboxamide

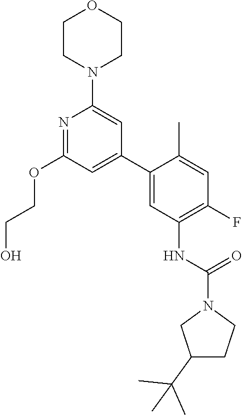 [SMILES: Cc1cc(F)c(NC(=O)N2CCC(C(C)(C)C)C2)cc1-c1cc(OCCO)nc(N2CCOCC2)c1]

The title compound was prepared using general procedure of N-(2-fluoro-5-(2-(2-hydroxyethoxy)-6-morpholinopyridin-4-yl)-4-methylphenyl)-3-(trifluoromethyl)pyrrolidine-1-carboxamide. ¹H NMR (400 MHz, DMSO-d₆): δ 0.90 (s, 9H), 1.54-1.65 (m, 1H), 1.80-1.86 (m, 1H), 1.99-2.07 (m, 1H), 2.20 (s, 3H), 3.05 (t, J=10.2 Hz, 1H), 3.20-3.27 (m, 1H), 3.43-3.47 (m, 5H), 3.54 (t, J=9.2 Hz, 1H), 3.68-3.72 (m, 6H), 4.24 (t, J=5.6 Hz, 2H), 4.78 (t, J=5.6 Hz, 1H), 5.98 (s, 1H), 6.19 (s, 1H), 7.12 (d, J=12.0 Hz, 1H), 7.36 (d, J=8.4 Hz, 1H), 7.79 (s, 1H). MS Calcd.: 500; MS Found: 501 ([M+H]2).

### Example 6: 1-(3,3-dimethylbutyl)-3-(2-fluoro-5-(2-(2-hydroxyethoxy)-6-morpholinopyridin-4-yl)-4-methylphenyl)urea

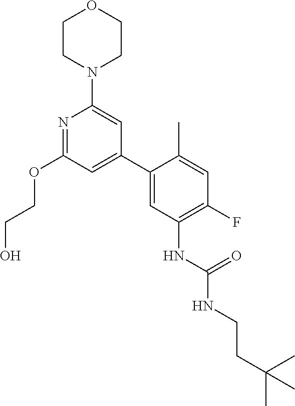 [SMILES: Cc1cc(F)c(NC(=O)NCCC(C)(C)C)cc1-c1cc(OCCO)nc(N2CCOCC2)c1]

The title compound was prepared using general procedure of N-(2-fluoro-5-(2-(2-hydroxyethoxy)-6-morpholinopyridin-4-yl)-4-methylphenyl)-3-(trifluoromethyl)pyrrolidine-1-carboxamide. ¹H NMR (400 MHz, DMSO-d₆): δ 0.89 (s, 9H), 1.32-1.36 (m, 2H), 2.15 (s, 3H), 3.05-3.10 (m, 2H), 3.44 (t, J=4.8 Hz, 4H), 3.68-3.72 (m, 6H), 4.24 (t, J=5.2 Hz, 2H), 4.78 (t, J=5.6 Hz, 1H), 5.95 (s, 1H), 6.17 (s, 1H), 6.46 (t, J=5.4 Hz, 1H), 7.10 (d, J=12.4 Hz, 1H), 7.97 (d, J=8.8 Hz, 1H), 8.20 (s, 1H). MS Calcd.: 474; MS Found: 475 ([M+H]⁺).

### Example 7: (RS)-N-(2-fluoro-5-(2-(2-hydroxyethoxy)-6-morpholinopyridin-4-yl)-4-methylphenyl)-3-(trifluoromethyl)piperazine-1-carboxamide

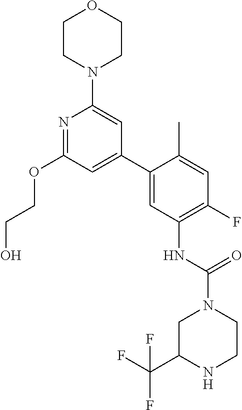 [SMILES: Cc1cc(F)c(NC(=O)N2CCNC(C(F)(F)F)C2)cc1-c1cc(OCCO)nc(N2CCOCC2)c1]

The title compound was using general procedure of N-(2-fluoro-5-(2-(2-hydroxyethoxy)-6-morpholinopyridin-4-yl)-4-methylphenyl)-3-(trifluoromethyl)piperidine-1-carboxamide. ¹H NMR (400 MHz, DMSO-d₆): δ 2.20 (s, 3H), 2.64-2.67 (m, 1H), 2.90-2.99 (m, 4H), 3.43-3.46 (m, 4H), 3.68-3.70 (m, 6H), 3.77-3.81 (m, 1H), 4.03 (dd, J=2.8, 12.4 Hz, 1H), 4.24 (t, J=5.2 Hz, 2H), 4.78 (t, J=5.6 Hz, 1H), 5.98 (s, 1H), 6.19 (s, 1H), 7.13 (d, J=11.6 Hz, 1H), 7.23 (d, J=8.0 Hz, 1H), 8.41 (s, 1H). MS Calcd.: 527; MS Found: 528 ([M+H]⁺).

### Example 8: (RS)-3-(tert-butyl)-N-(2-fluoro-5-(2-(2-hydroxyethoxy)-6-morpholinopyridin-4-yl)-4-methylphenyl)piperidine-1-carboxamide

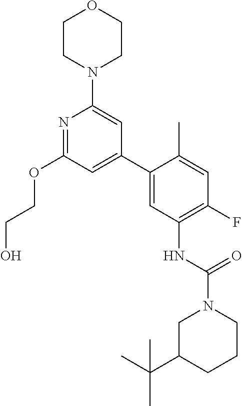 [SMILES: Cc1cc(F)c(NC(=O)N2CCCC(C(C)(C)C)C2)cc1-c1cc(OCCO)nc(N2CCOCC2)c1]

The title compound was prepared using general procedure of N-(2-fluoro-5-(2-(2-hydroxyethoxy)-6-morpholinopyridin-4-yl)-4-methylphenyl)-3-(trifluoromethyl)piperidine-1-carboxamide. ¹H NMR (400 MHz, DMSO-d₆): δ 0.87 (s, 9H), 1.11-1.18 (m, 2H), 1.40-1.43 (m, 1H), 1.66-1.69 (m, 1H), 1.79-1.82 (m, 1H), 2.19 (s, 3H), 2.42-2.50 (m, 1H), 2.62-2.66 (m, 1H), 3.43-3.46 (m, 4H), 3.68-3.69 (m, 6H), 4.03 (d, J=13.2 Hz, 1H), 4.16 (d, J=12.8 Hz, 1H), 4.24 (d, J=5.6 Hz, 2H), 4.77 (brs, 1H), 5.97 (s, 1H), 6.19 (s, 1H), 7.11 (d, J=11.6 Hz, 1H), 7.24 (d, J=8.4 Hz, 1H), 8.16 (s, 1H). MS Calcd.: 514; MS Found: 515 ([M+H]⁺).

### Example 9: 2-(tert-butyl)-N-(2-fluoro-5-(2-(2-hydroxyethoxy)-6-morpholinopyridin-4-yl)-4-methylphenyl)morpholine-4-carboxamide

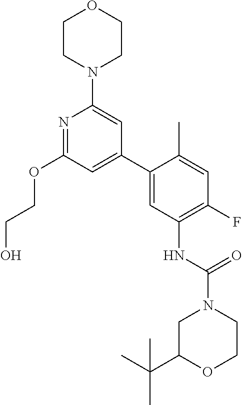 [SMILES: Cc1cc(F)c(NC(=O)N2CCOC(C(C)(C)C)C2)cc1-c1cc(OCCO)nc(N2CCOCC2)c1]

The title compound was prepared using general procedure of N-(2-fluoro-5-(2-(2-hydroxyethoxy)-6-morpholinopyridin-4-yl)-4-methylphenyl)-3-(trifluoromethyl)piperidine-1-carboxamide. ¹H NMR (400 MHz, DMSO-d₆): δ 0.91 (s, 9H), 2.20 (s, 3H), 2.57-2.68 (m, 1H), 2.81-2.88 (m, 1H), 2.96-2.99 (m, 1H), 3.39-3.46 (m, 5H), 3.68-3.72 (m, 6H), 3.83-3.92 (m, 2H), 3.96 (d, J=12.8 Hz, 1H), 4.24 (t, J=5.6 Hz, 2H), 4.78 (t, J=5.6 Hz, 1H), 5.98 (s, 1H), 6.19 (s, 1H), 7.13 (d, J=12.0 Hz, 1H), 7.24 (d, J=8.4 Hz, 1H), 8.33 (s, 1H). MS Calcd.: 516; MS Found: 517 ([M+H]⁺).

### Example 10: (RS)-N-(2-fluoro-5-(2-(2-hydroxyethoxy)-6-morpholinopyridin-4-yl)-4-methylphenyl)-2-(trifluoromethyl)morpholine-4-carboxamide

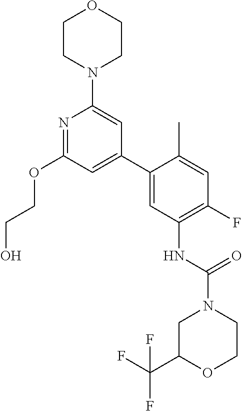 [SMILES: Cc1cc(F)c(NC(=O)N2CCOC(C(F)(F)F)C2)cc1-c1cc(OCCO)nc(N2CCOCC2)c1]

The title compound was prepared using general procedure of N-(2-fluoro-5-(2-(2-hydroxyethoxy)-6-morpholinopyridin-4-yl)-4-methylphenyl)-3-(trifluoromethyl)piperidine-1-carboxamide. ¹H NMR (400 MHz, DMSO-d₆): δ 2.21 (s, 3H), 2.93-3.07 (m, 2H), 3.44 (t, J=4.8 Hz, 4H), 3.58-3.64 (m, 1H), 3.68-3.72 (m, 6H), 3.93 (d, J=13.6 Hz, 1H), 4.00 (d, J=10.4 Hz, 1H), 4.15-4.24 (m, 4H), 4.78 (t, J=5.6 Hz, 1H), 5.98 (s, 1H), 6.19 (s, 1H), 7.15 (d, J=12.0 Hz, 1H), 7.25 (d, J=8.4 Hz, 1H), 8.54 (s, 1H). MS Calcd.: 528; MS Found: 529 ([M+H]⁺).

### Example 11: (RS)-3-(tert-butyl)-N-(2-fluoro-5-(2-(2-hydroxyethoxy)-6-morpholinopyridin-4-yl)-4-methylphenyl)piperazine-1-carboxamide

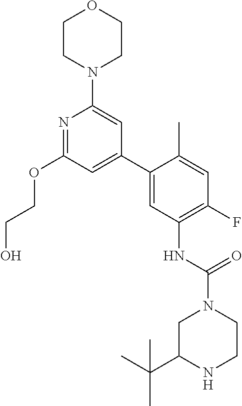 [SMILES: Cc1cc(F)c(NC(=O)N2CCNC(C(C)(C)C)C2)cc1-c1cc(OCCO)nc(N2CCOCC2)c1]

The title compound was prepared using general procedure of N-(2-fluoro-5-(2-(2-hydroxyethoxy)-6-morpholinopyridin-4-yl)-4-methylphenyl)-3-(trifluoromethyl)piperidine-1-carboxamide. ¹H NMR (400 MHz, DMSO-d₆): δ 0.90 (s, 9H), 2.14-2.20 (m, 4H), 2.44 (t, J=11.8 Hz, 1H), 2.54-2.60 (m, 1H), 2.67-2.73 (m, 1H), 2.93 (d, J=11.6 Hz, 1H), 3.44 (t, J=4.8 Hz, 4H), 3.68-3.72 (m, 6H), 3.90 (d, J=12.4 Hz, 1H), 4.02 (d, J=12.0 Hz, 1H), 4.24 (t, J=5.6 Hz, 2H), 4.78 (t, J=5.6 Hz, 1H), 5.98 (s, 1H), 6.19 (s, 1H), 7.12 (d, J=12.0 Hz, 1H), 7.23 (d, J=8.4 Hz, 1H), 8.19 (s, 1H). MS Calcd.: 515; MS Found: 516 ([M+H]⁺).

### Example 12 and 13: (3R)-N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide and (3S)-N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide

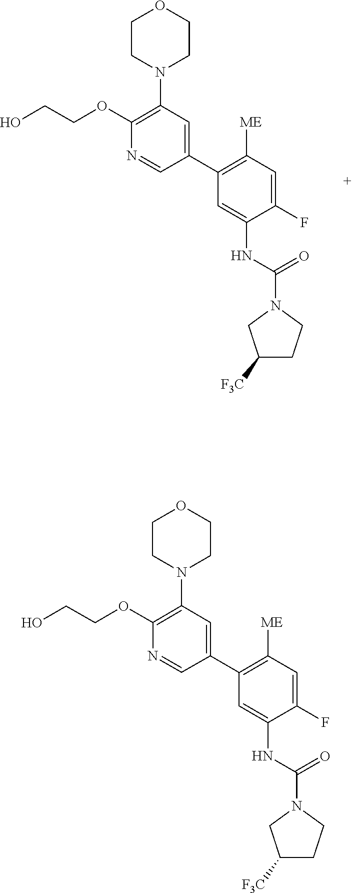

### Step 1: N-[2-fluoro-4-methyl-5-[5-(morpholin-4-yl)-6-[2-(oxan-2-yloxy)ethoxy]pyridin-3-yl]phenyl]-3-(trifluoromethyl)pyrrolidine-1-carboxamide

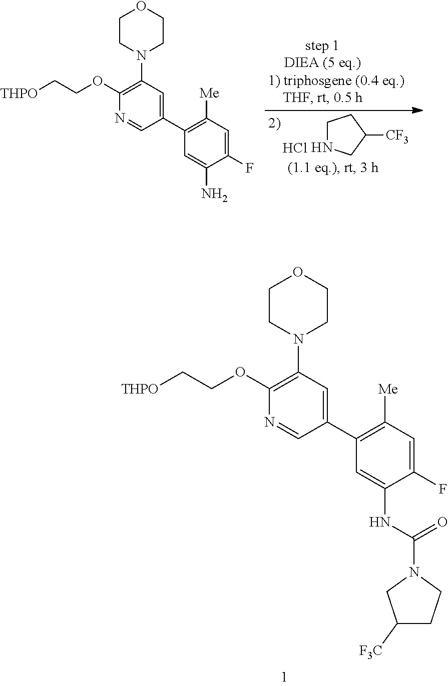

To a solution of 2-fluoro-4-methyl-5-[5-(morpholin-4-yl)-6-[2-(oxan-2-yloxy)ethoxy]pyridin-3-yl]aniline (560.00 mg, 1.30 mmol) in THF (25.00 mL) were added triphosgene (154.04 mg, 0.52 mmol) and DIEA (838.64 mg, 6.49 mmol) under nitrogen atmosphere. The reaction mixture was stirred at room temperature for 0.5 h. To the above mixture 3-(trifluoromethyl)pyrrolidine hydrochloride (227.86 mg, 1.30 mmol) was added and the reaction mixture was stirred for 4 h at room temperature. The resulting mixture was concentrated under vacuum. The residue was diluted with water (20 mL) and extracted with EA (3×10 mL). The combined organic layers was washed with brine (20 mL), dried over anhydrous Na₂SO₄ and filtered. The filtrate was concentrated under reduced pressure. The residue was purified by silica gel column chromatography, eluted with MeOH in DCM (1-10%). The fractions contained desired product were combined and concentrated to afford N-[2-fluoro-4-methyl-5-[5-(morpholin-4-yl)-6-[2-(oxan-2-yloxy)ethoxy]pyridin-3-yl]phenyl]-3-(trifluoromethyl)pyrrolidine-1-carboxamide (550 mg, 71%) as a yellow solid. MS ESI calculated for C₂₉H₃₆F₄N₄O₅[M+H]⁺, 597.26, found 597.25. ¹H-NMR (300 MHz, d6-DMSO) δ 8.07 (s, 1H), 7.68 (d, J=2.0 Hz, 1H), 7.33 (d, J=8.4 Hz, 1H), 7.17 (d, J=11.2 Hz, 1H), 7.10 (d, J=2.0 Hz, 1H), 4.70 (d, J=3.6 Hz, 1H), 4.50-4.46 (m, 2H), 4.02-3.90 (m, 1H), 3.83-3.63 (m, 7H), 3.48 (m, 5H), 3.13-3.08 (m, 4H), 2.25-2.20 (m, 4H), 2.07-2.02 (m, 1H), 1.82-1.56 (m, 2H), 1.52-1.46 (m, 4H).

### Step 2: N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide

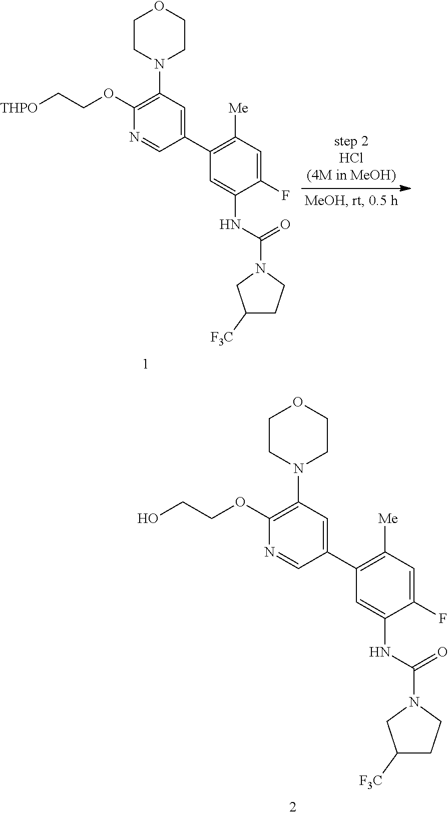

To a solution of N-[2-fluoro-4-methyl-5-[5-(morpholin-4-yl)-6-[2-(oxan-2-yloxy)ethoxy]pyridin-3-yl]phenyl]-3-(trifluoromethyl)pyrrolidine-1-carboxamide (300.00 mg, 0.50 mmol) in MeOH (4.50 mL) was added HCl (4 M in 1,4-dioxane) (1.50 mL). The reaction solution was stirred for 30 min at 25° C. The resulting mixture was concentrated under reduced pressure. The residue was purified by reverse flash chromatography with the following conditions: column, C₁₈ silica gel; mobile phase, CH₃CN in water, 0% to 100% gradient in 25 min; detector, UV 254 nm. The fractions contained desired product were combined and concentrated to afford to give N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide (210 mg, 90%) as an off-white solid. MS ESI calculated for C₂₄H₂₈F₄N₄O₄[M+H]⁺, 513.20; found 513.35.

### Step 3: (3R)-N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide and (3S)-N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide

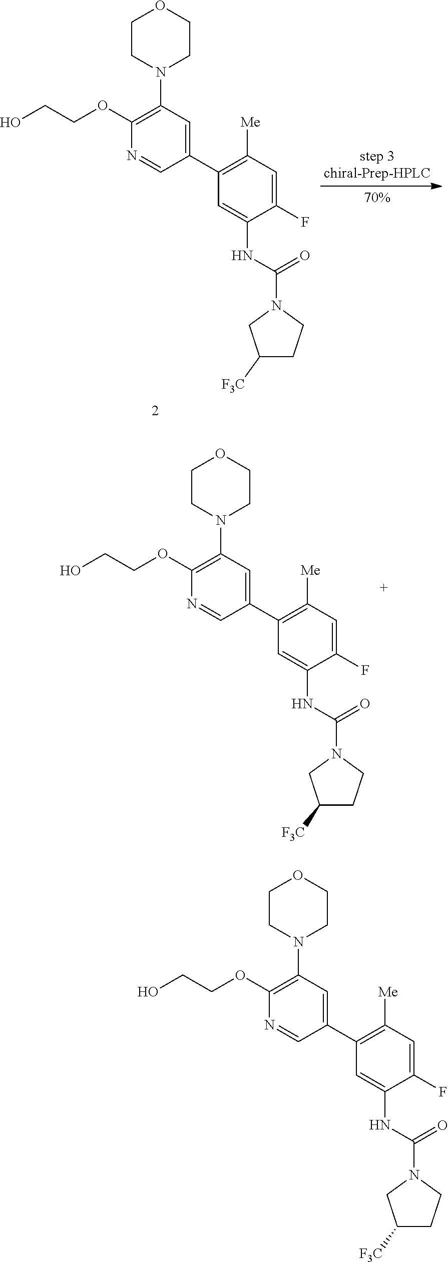

N-[2-fluoro-5-[6-(2-hydroxyethoxy)-5-(morpholin-4-yl)pyridin-3-yl]-4-methylphenyl]-3-(trifluoromethyl)pyrrolidine-1-carboxamide (210 mg) was separated by Prep-Chiral HPLC with the following conditions: Column: CHIRAL ART Cellulose-SB, 2*25 cm, 5 um; Mobile Phase A: Hex:DCM=5:1 (10 mM NH3-MEOH), Mobile Phase B: EtOx; Flow rate: 20 mL/min; Gradient: 10 B to 10 B in 11 mi; 254/220 nm. The fractions contained desired product were concentrated to give the two enantiomers: (71.8 mg) of the first isomer eluted at 8.296 me (ee>98%) and (75.9 mg) of the second isomer eluted at 9.553 min (ee>98%). ¹H-NMR (400 MHz, d₆-DMSO) of the first eluted isomer: δ 8.04 (s, 1H), 7.65 (d, J=1.6 Hz, 1H), 7.32 (d, J=8.0 Hz, 1H), 7.16 (d, J=11.6 Hz, 1H), 7.06 (d, J=1.6 Hz, 1H), 4.79 (t, J=4.2 Hz, 1H), 4.36 (t, J=4.2 Hz, 2H), 3.77-3.74 (m, 7H), 3.69-3.57 (i, 3H), 3.34-3.28 (m, 1H), 3.09-3.07 (m, 4H), 2.33-2.15 (m, 4H), 2.05-1.98 (in, 1H). MS ESI calculated for C₂₄H₂₈F₄N₄O₄[M+H]⁺, 513.20, found 513.15. ¹H-NMR (400 MHz, d₆-DMSO) of the first eluted isomer: δ 8.04 (s, 1H), 7.65 (d, J=1.6 Hz, 1H), 7.32 (d, J=8.0 Hz, 1H), 7.16 (d, J=11.6 Hz, 1H), 7.06 (d, J=1.6 Hz, 1H), 4.79 (t, J=4.2 Hz, 1H), 4.36 (t, J=4.2 Hz, 2H), 3.77-3.74 (m, 7H), 3.69-3.57 (m, 3H), 3.34-3.28 (m, 1H), 3.09-3.07 (m, 4H), 2.33-2.15 (m, 4H), 2.05-1.98 (m, 1H). MS ESI calculated for C₂₄H₂₈F₄N₄O₄[M+H]⁺, 513.20, found 513.15.

The following compounds in Table 3 were prepared using procedures similar to those described in Example 12 and 13 using appropriate starting materials. Racemic products were separated using chiral columns specified in Table 3.

TABLE 3

| Example | | Exact Mass | Chiral |
|---|---|---|---|
| Number | IUPAC Name | [M + H]⁺ | column |
| 14 and 15 | (3R)-N-[3-[6-(2-hydroxyethoxy)-5-(morpholin-4- | Calc'd | CHIRALPAK AD-H |
| | yl)pyridin-3-yl]-4-methylphenyl]-3- | 495.21, | 2.0 cm × 25 cm |
| | (trifluoromethyl)pyrrolidine-1-carboxamide and | found | Example 14: |
| | (3S)-N-[3-[6-(2-hydroxyethoxy)-5-(morpholin-4- | 495.20 | First eluting peak |
| | yl)pyridin-3-yl]-4-methylphenyl]-3- | | Example 15: |
| | (trifluoromethyl)pyrrolidine-1-carboxamide | | Second eluting peak |
| 16 and 17 | (3R)-N-[6′-(2-hydroxyethoxy)-2-methyl-5′-(morpholin- | Calc'd | CHIRALPAK IG |
| | 4-yl)-[3,3′-bipyridin]-5-yl]-3- | 496.21, | 2 × 25 cm, 5 um |
| | (trifluoromethyl)pyrrolidine-1-carboxamide and | found | Example 16: |
| | (3S)-N-[6′-(2-hydroxyethoxy)-2-methyl-5′-(morpholin- | 496.10 | First eluting peak |
| | 4-yl)-[3,3′-bipyridin]-5-yl]-3- | | Example 17: |
| | (trifluoromethyl)pyrrolidine-1-carboxamide | | Second eluting peak |
| 18 | N-[2-fluoro-5-[2-(2-hydroxyethoxy)-6-(morpholin-4- | Calc'd | NA |
| | yl)pyridin-4-yl]-4-methylphenyl]-5- | 471.23, | |
| | azaspiro[2.4]heptane-5-carboxamide | found | |
| | | 471.25 | |
| 19 | N-[2-fluoro-5-[2-(2-hydroxyethoxy)-6-(morpholin-4- | Calc'd | NA |
| | yl)pyridin-4-yl]-4-methylphenyl]-3,3- | 473.25, | |
| | dimethylpyrrolidine-1-carboxamide | found | |
| | | 473.30 | |
| 20 | 4,4-difluoro-N-[2-fluoro-5-[2-(2-hydroxyethoxy)-6- | Calc'd | NA |
| | (morpholin-4-yl)pyridin-4-yl]-4- | 495.21, | |
| | methylphenyl]piperidine-1-carboxamide | found | |
| | | 495.21 | |
| 21 | 3-(difluoromethyl)-N-[2-fluoro-5-[2-(2- | Calc'd | NA |
| | hydroxyethoxy)-6-(morpholin-4-yl)pyridin-4-yl]-4- | 481.20, | |
| | methylphenyl]azetidine-1-carboxamide | found | |
| | | 481.20 | |
| 22 | 3,3,4,4-tetrafluoro-N-[2-fluoro-5-[2-(2- | Calc'd | NA |
| | hydroxyethoxy)-6-(morpholin-4-yl)pyridin-4-yl]-4- | 517.18, | |
| | methylphenyl]pyrrolidine-1-carboxamide | found | |
| | | 517.15 | |
| 23 | 3,3,4,4-tetrafluoro-N-[2-fluoro-5-[2-(2- | Calc'd | NA |
| | hydroxyethoxy)-6-(morpholin-4-yl)pyridin-4-yl]-4- | 521.23, | |
| | methylphenyl]pyrrolidine-1-carboxamide | found | |
| | | 521.20 | |
| 24 | 3,3-difluoro-N-(2-fluoro-5-(2-(2-hydroxyethoxy)-6- | Calc'd | NA |
| | morpholinopyridin-4-yl)-4-methylphenyl)piperidine-1- | 495.21, | |
| | carboxamide | found | |
| | | 495.20 | |
| 25 | N-(2-fluoro-5-(2-(2-hydroxyethoxy)-6- | Calc'd | NA |
| | morpholinopyridin-4-yl)-4-methylphenyl)-3-hydroxy- | 543.22, | |
| | 3-(trifluoromethyl)piperidine-1-carboxamide | found | |
| | | 543.30 | |
| 26 | N-[2-fluoro-5-[2-(2-hydroxyethoxy)-6-(morpholin-4- | Calc'd | NA |
| | yl)pyridin-4-yl]-4-methylphenyl]-6- | 485.25, | |
| | azaspiro[3.4]octane-6-carboxamide | found | |
| | | 485.25 | |
| 27 | N-[2-fluoro-5-[2-(2-hydroxyethoxy)-6-(morpholin-4- | Calc'd | NA |
| | yl)pyridin-4-yl]-4-methylphenyl]-2-oxa-6- | 501.24, | |
| | azaspiro[3.5]nonane-6-carboxamide | found | |
| | | 501.30 | |
| 28 | 6,6-difluoro-N-[2-fluoro-5-[2-(2-hydroxyethoxy)-6- | Calc'd | NA |
| | (morpholin-4-yl)pyridin-4-yl]-4-methylphenyl]-3- | 493.20, | |
| | azabicyclo[3.1.0]hexane-3-carboxamide | found | |
| | | 493.20 | |
| 29 and 30 | (3R)-N-[2-fluoro-5-[2-(2-hydroxyethoxy)-6-[(3R)-3- | Calc'd | CHIRAL ART |
| | methylmorpholin-4-yl]pyridin-4-yl]-4-methylphenyl]- | 527.22, | Cellulose-SB, |
| | 3-(trifluoromethyl) pyrrolidine-1-carboxamide and | found | 2 × 25 cm, 5 um |
| | (3S)-N-[2-fluoro-5-[2-(2-hydroxyethoxy)-6-[(3R)-3- | 527.20 | Example 29: |
| | methylmorpholin-4-yl]pyridin-4-yl]-4-methylphenyl]- | | First eluting peak |
| | 3-(trifluoromethyl)pyrrolidine-1-carboxamide | | Example 30: |
| | | | Second eluting peak |
| 31 and 32 | (3R)-N-[2-fluoro-4-methyl-5-[5-(morpholin-4-yl)-6- | Calc'd | CHIRAL ART |
| | (oxan-4-yloxy)pyridin-3-yl]phenyl]-3- | 553.24, | Cellulose-SB, |
| | (trifluoromethyl)pyrrolidine-1-carboxamide and | found | 2 × 25 cm, 5 um |
| | (3S)-N-[2-fluoro-4-methyl-5-[5-(morpholin-4-yl)-6- | 553.20 | Example 31: |
| | (oxan-4-yloxy)pyridin-3-yl]phenyl]-3- | | First eluting peak |
| | (trifluoromethyl)pyrrolidine-1-carboxamide | | Example 32: |
| | | | Second eluting peak |
| 33 and 34 | (3R)-3-(1,1-difluoroethyl)-N-[2-fluoro-5-[2-(2- | Calc'd | CHIRAL ART |
| | hydroxyethoxy)-6-(morpholin-4-yl)pyridin-4-yl]-4- | 509.23, | Cellulose-SB, |
| | methylphenyl]pyrrolidine-1-carboxamide and | found | 2 × 25 cm, 5 um |
| | (3S)-3-(1,1-difluoroethyl)-N-[2-fluoro-5-[2-(2- | 509.45 | Example 33: |
| | hydroxyethoxy)-6-(morpholin-4-yl)pyridin-4-yl]-4- | | First eluting peak |
| | methylphenyl]pyrrolidine-1-carboxamide | | Example 34: |
| | | | Second eluting peak |
| 35 | 1-[2-fluoro-5-[2-(2-hydroxyethoxy)-6-(morpholin-4- | Calc'd | NA |
| | yl)pyridin-4-yl]-4-methylphenyl]-3-(2,2,3,3,3- | 523.17; | |
| | pentafluoropropyl)urea | found | |
| | | 523.15 | |
| 36 and 37 | (3R)-N-[2-fluoro-5-[2-(2-hydroxyethoxy)-6- | Calc'd | Chiralpak AD-H |
| | (morpholin-4-yl)pyridin-4-yl]-4-methylphenyl]-3- | 527.22,; | 2 × 25 cm, 5 um |
| | (2,2,2-trifluoroethyl)pyrrolidine-1-carboxamide and | found | |
| | (3R)-N-[2-fluoro-5-[2-(2-hydroxyethoxy)-6- | 527.25 | |
| | (morpholin-4-yl)pyridin-4-yl]-4-methylphenyl]-3- | | |
| | (2,2,2-trifluoroethyl)pyrrolidine-1-carboxamide | | |
| 38 | N-[2-fluoro-5-[2-(2-hydroxyethoxy)-6-(morpholin-4- | Calc'd | NA |
| | yl)pyridin-4-yl]-4-methylphenyl]-6- | 499.26; | |
| | azaspiro[3.5]nonane-6-carboxamide | found | |
| | | 499.30 | |
| 39 | (trans)-N-[2-fluoro-5-[2-(2-hydroxyethoxy)-6- | Calc'd | NA |
| | (morpholin-4-yl)pyridin-4-yl]-4-methylphenyl]-3- | 545.21, | |
| | (fluoromethyl)-4-(trifluoromethyl) pyrrolidine-1- | found | |
| | carboxamide | 545.20 | |
| 40 | (3S,5R)-3-amino-N-[2-fluoro-5-[2-(2-hydroxyethoxy)- | Calc'd | NA |
| | 6-(morpholin-4-yl)pyridin-4-yl]-4-methylphenyl]-5- | 542.23; | |
| | (trifluoromethyl)piperidine-1-carboxamide | found | |
| | | 542.25 | |
| 41 | N-[2-fluoro-5-[2-(2-hydroxyethoxy)-6-(morpholin-4- | Calc'd | NA |
| | yl)pyridin-4-yl]-4-methylphenyl]-2-oxa-6- | 487.23, | |
| | azaspiro[3.4]octane-6-carboxamide | found | |
| | | 487.30 | |
| 42 | N-[2-fluoro-5-[2-(2-hydroxyethoxy)-6-(morpholin-4- | Calc'd | NA |
| | yl)pyridin-4-yl]-4-methylphenyl]-3-methoxy-3- | 557.23; | |
| | (trifluoromethyl) piperidine-1-carboxamide | found | |
| | | 557.25 | |
| 43 | 1-[2-fluoro-5-[2-(2-hydroxyethoxy)-6-(morpholin-4- | Calc'd | NA |
| | yl)pyridin-4-yl]-4-methylphenyl]-3-[2- | 499.19, | |
| | (trifluoromethyl) cyclopropyl]urea | found | |
| | | 499.25 | |
| 44 | (3S,5R)-N-[2-fluoro-5-[2-(2-hydroxyethoxy)-6- | Calc'd | NA |
| | (morpholin-4-yl)pyridin-4-yl]-4-methylphenyl]-3- | 543.22, | |
| | hydroxy-5-(trifluoromethyl) piperidine-1-carboxamide | found | |
| | | 543.30 | |
| 45 | N-[2-fluoro-5-[2-(2-hydroxyethoxy)-6-(morpholin-4- | Calc'd | NA |
| | yl)pyridin-4-yl]-4-methylphenyl]-3-(fluoromethyl)-3- | 545.21, | |
| | (trifluoromethyl)pyrrolidine-1-carboxamide | found | |
| | | 545.30 | |
| 46 | 3,3-difluoro-N-[2-fluoro-5-[2-(2-hydroxyethoxy)-6- | Calc'd | NA |
| | (morpholin-4-yl)pyridin-4-yl]-4- | 509.22, | |
| | methylphenyl]azepane-1-carboxamide | found | |
| | | 509.23 | |
| 47 and 48 | (3R)-3-cyclopropyl-N-[2-fluoro-5-[2-(2- | Calc'd | Chiralpak AD-H |
| | hydroxyethoxy)-6-(morpholin-4-yl)pyridin-4-yl]-4- | 485.25, | 2 × 25 cm, 5 um |
| | methylphenyl]pyrrolidine-1-carboxamide and | found | Example 47: |
| | (3S)-3-cyclopropyl-N-[2-fluoro-5-[2-(2- | 485.25 | First eluting peak |
| | hydroxyethoxy)-6-(morpholin-4-yl)pyridin-4-yl]-4- | | Example 48: |
| | methylphenyl]pyrrolidine-1-carboxamide | | Second eluting peak |
| 49 | 1-[2-fluoro-5-[2-(2-hydroxyethoxy)-6-(morpholin-4- | Calc'd | NA |
| | yl)pyridin-4-yl]-4-methylphenyl]-3-[[1- | 527.22; | |
| | (trifluoromethyl) cyclobutyl]methyl]urea | found | |
| | | 527.20 | |
| 50 and 51 | (1R,5R)-N-(2-fluoro-5-(2-(2-hydroxyethoxy)-6- | Calc'd | CHIRALPAK IF, |
| | morpholinopyridin-4-yl)-4-methylphenyl)-1- | 525.20; | 2 × 25 cm, 5 um |
| | (trifluoromethyl)-3-azabicyclo[3.1.0]hexane-3- | found | Example 50: |
| | carboxamide and | 525.30 | First eluting peak |
| | (1S,5S)-N-(2-fluoro-5-(2-(2-hydroxyethoxy)-6- | | Example 51: |
| | morpholinopyridin-4-yl)-4-methylphenyl)-1- | | Second eluting peak |
| | (trifluoromethyl)-3-azabicyclo[3.1.0]hexane-3- | | |
| | carboxamide | | |
| 52 | N-[2-fluoro-5-[2-(2-hydroxyethoxy)-6-(morpholin-4- | Calc'd | CHIRALPAK IG |
| | yl)pyridin-4-yl]-4-methylphenyl]-3-methyl-4- | 527.22; | 2 × 25 cm, 5 um |
| | (trifluoromethyl) pyrrolidine-1-carboxamide | found | First eluting peak |
| | | 527.30 | |
| 53 | N-[2-fluoro-5-[2-(2-hydroxyethoxy)-6-(morpholin-4- | Calc'd | CHIRALPAK IG |
| | yl)pyridin-4-yl]-4-methylphenyl]-3-methyl-4- | 527.22; | 2 × 25 cm, 5 um |
| | (trifluoromethyl) pyrrolidine-1-carboxamide | found | Second eluting peak |
| | | 527.30 | |
| 54 | 3-amino-N-[2-fluoro-5-[2-(2-hydroxyethoxy)-6- | Calc'd | NA |
| | (morpholin-4-yl)pyridin-4-yl]-4-methylphenyl]-3- | 542.23; | |
| | (trifluoromethyl)piperidine-1-carboxamide | found | |
| | | 542.20 | |
| 55 | (3R,5S)-3-amino-N-[2-fluoro-5-[2-(2-hydroxyethoxy)- | Calc'd | NA |
| | 6-(morpholin-4-yl)pyridin-4-yl]-4-methylphenyl]-5- | 542.23; | |
| | (trifluoromethyl)piperidine-1-carboxamide | found | |
| | | 542.35 | |
| 56 | N-[2-fluoro-5-[2-(2-hydroxyethoxy)-6-(morpholin-4- | Calc'd | NA |
| | yl)pyridin-4-yl]-4-methylphenyl]-3-(fluoromethyl)-3- | 507.21, | |
| | (trifluoromethyl) pyrrolidine-1-carboxamide | found | |
| | | 507.10 | |
| 57 | 1-[2-fluoro-5-[2-(2-hydroxyethoxy)-6-(morpholin-4- | Calc'd | NA |
| | yl)pyridin-4-yl]-4-methylphenyl]-3-(4,4,4- | 501.20; | |
| | trifluorobutan-2-yl)urea | found | |
| | | 501.25 | |
| 58 and 59 | (2R)-2-(1,1-difluoroethyl)-N-[2-fluoro-5-[2-(2- | Calc'd | CHIRALPAK IA |
| | hydroxyethoxy)-6-(morpholin-4-yl)pyridin-4-yl]-4- | 525.22, | 2 × 25 cm, 5 um |
| | methylphenyl]morpholine-4-carboxamide and | found | Example 58: |
| | (2R)-2-(1,1-difluoroethyl)-N-[2-fluoro-5-[2-(2- | 525.30 | First eluting peak |
| | hydroxyethoxy)-6-(morpholin-4-yl)pyridin-4-yl]-4- | | Example 59: |
| | methylphenyl]morpholine-4-carboxamide | | Second eluting peak |
| 60 | (cis)-N-[2-fluoro-5-[2-(2-hydroxyethoxy)-6- | Calc'd | CHIRALPAK IG |
| | (morpholin-4-yl)pyridin-4-yl]-4-methylphenyl]-2- | 527.22; | 2 × 25 cm, 5 um |
| | methyl-4-(trifluoromethyl)pyrrolidine-1-carboxamide | found | First eluting peak |
| | | 527.25 | |
| 61 | (cis)-N-[2-fluoro-5-[2-(2-hydroxyethoxy)-6- | Calc'd | CHIRALPAK IG |
| | (morpholin-4-yl)pyridin-4-yl]-4-methylphenyl]-2- | 527.22; | 2 × 25 cm, 5 um |
| | methyl-4-(trifluoromethyl)pyrrolidine-1-carboxamide | found | Second eluting peak |
| | | 527.25 | |
| 62 and 63 | (3R)-1,1-difluoro-N-[2-fluoro-5-[2-(2-hydroxyethoxy)- | Calc'd | CHIRALPAK IG |
| | 6-(morpholin-4-yl)pyridin-4-yl]-4-methylphenyl]-5- | 507.21; | 2 × 25 cm, 5 um |
| | azaspiro[2.4]heptane-5-carboxamide and | found | Example 58: |
| | (3S)-1,1-difluoro-N-[2-fluoro-5-[2-(2-hydroxyethoxy)- | 507.25 | First eluting peak |
| | 6-(morpholin-4-yl)pyridin-4-yl]-4-methylphenyl]-5- | | Example 59: |
| | azaspiro[2.4]heptane-5-carboxamide | | Second eluting peak |
| 64 | N-[2-fluoro-5-[2-(2-hydroxyethoxy)-6-(morpholin-4- | Calc'd | CHIRALPAK IG |
| | yl)pyridin-4-yl]-4-methylphenyl]-2-methyl-3- | 527.22, | 2 × 25 cm, 5 um |
| | (trifluoromethyl)pyrrolidine-1-carboxamide | found | First eluting peak |
| | | 527.25 | |
| 65 | N-[2-fluoro-5-[2-(2-hydroxyethoxy)-6-(morpholin-4- | Calc'd | CHIRALPAK IG |
| | yl)pyridin-4-yl]-4-methylphenyl]-2-methyl-3- | 527.22, | 2 × 25 cm, 5 um |
| | (trifluoromethyl)pyrrolidine-1-carboxamide | found | Second eluting peak |
| | | 527.25 | |
| 66 | N-[2-fluoro-5-[2-(2-hydroxyethoxy)-6-(morpholin-4- | Calc'd | CHIRALPAK IG |
| | yl)pyridin-4-yl]-4-methylphenyl]-2-methyl-3- | 527.22, | 2 × 25 cm, 5 um |
| | (trifluoromethyl)pyrrolidine-1-carboxamide | found | Third eluting peak |
| | | 527.25 | |
| 67 | N-[2-fluoro-5-[2-(2-hydroxyethoxy)-6-(morpholin-4- | Calc'd | CHIRALPAK IG |
| | yl)pyridin-4-yl]-4-methylphenyl]-2-methyl-3- | 527.22, | 2 × 25 cm, 5 um |
| | (trifluoromethyl)pyrrolidine-1-carboxamide | found | Fourth eluting peak |
| | | 527.25 | |
| 68 | N-[2-fluoro-5-[2-(2-hydroxyethoxy)-6-(morpholin-4- | Calc'd | CHIRAL ART |
| | yl)pyridin-4-yl]-4-methylphenyl]-6-(trifluoromethyl)- | 525.20, | Cellulose-SB, |
| | 2-azabicyclo[3.1.0]hexane-2-carboxamide | found | 2 × 25 cm, 5 um |
| | | 525.20 | First eluting peak |
| 69 | N-[2-fluoro-5-[2-(2-hydroxyethoxy)-6-(morpholin-4- | Calc'd | CHIRAL ART |
| | yl)pyridin-4-yl]-4-methylphenyl]-6-(trifluoromethyl)- | 525.20, | Cellulose-SB, |
| | 2-azabicyclo[3.1.0]hexane-2-carboxamide | found | 2 × 25 cm, 5 um |
| | | 525.20 | Second eluting peak |
| 70 | N-[2-fluoro-5-[2-(2-hydroxyethoxy)-6-(morpholin-4- | Calc'd | CHIRALPAK IA |
| | yl)pyridin-4-yl]-4-methylphenyl]-6-(trifluoromethyl)- | 525.20, | 2 × 25 cm, 5 um |
| | 2-azabicyclo[3.1.0]hexane-2-carboxamide | found | Third eluting peak |
| | | 525.20 | |
| 71 | N-[2-fluoro-5-[2-(2-hydroxyethoxy)-6-(morpholin-4- | Calc'd | CHIRALPAK IA |
| | yl)pyridin-4-yl]-4-methylphenyl]-6-(trifluoromethyl)- | 525.20, | 2 × 25 cm, 5 um |
| | 2-azabicyclo[3.1.0]hexane-2-carboxamide | found | Fourth eluting peak |
| | | 525.20 | |
| 72 | N-[2-fluoro-5-[2-(2-hydroxyethoxy)-6-(morpholin-4- | Calc'd | NA |
| | yl)pyridin-4-yl]-4-methylphenyl]-3-(trifluoromethyl)- | 511.19; | |
| | 2,5-dihydropyrrole-1-carboxamide | found | |
| | | 511.30 | |
| 73 | N-(2-fluoro-5-(2-(2-hydroxyethoxy)-6- | Calc'd | NA |
| | morpholinopyridin-4-yl)-4-methylphenyl)-2- | 499.26; | |
| | azaspiro[4.4]nonane-2-carboxamide | found | |
| | | 499.30 | |
| 74 and 75 | (3R)-N-[2-fluoro-5-[2-(2-hydroxyethoxy)-6- | Calc'd | Chiralpak AD-H |
| | (morpholin-4-yl)pyridin-4-yl]-4-methylphenyl]-3- | 527.22, | 2 × 25 cm, 5 um |
| | (2,2,2-trifluoroethyl)pyrrolidine-1-carboxamide and | found | Example 74: |
| | (3S)-N-[2-fluoro-5-[2-(2-hydroxyethoxy)-6- | 527.25 | First eluting peak |
| | (morpholin-4-yl)pyridin-4-yl]-4-methylphenyl]-3- | | Example 75: |
| | (2,2,2-trifluoroethyl)pyrrolidine-1-carboxamide | | Second eluting peak |
| 76 | N-[2-fluoro-5-[2-(2-hydroxyethoxy)-6-(morpholin-4- | Calc'd | NA |
| | yl)pyridin-4-yl]-4-methylphenyl]-3-(2,2,2-trifluoro-1- | 543.22, | |
| | hydroxyethyl)pyrrolidine-1-carboxamide | found | |
| | | 543.20 | |
| 77 and 78 | (3R)-N-[2-fluoro-5-[2-(2-hydroxyethoxy)-6- | Calc'd | CHIRAL ART |
| | (morpholin-4-yl)pyridin-4-yl]-4-methylphenyl]-3- | 529.20; | Cellulose-SB, |
| | (trifluoromethoxy)pyrrolidine-1-carboxamide and | found | 2 × 25 cm, 5 um |
| | (3S)-N-[2-fluoro-5-[2-(2-hydroxyethoxy)-6- | 529.20 | Example 77: |
| | (morpholin-4-yl)pyridin-4-yl]-4-methylphenyl]-3- | | First eluting peak |
| | (trifluoromethoxy)pyrrolidine-1-carboxamide | | Example 78: |
| | | | Second eluting peak |
| 79 | N-[2-fluoro-5-[2-(2-hydroxyethoxy)-6-(morpholin-4- | Calc'd | NA |
| | yl)pyridin-4-yl]-4-methylphenyl]-3-(1,1,2,2,2- | 561.19, | |
| | pentafluoroethyl)-2,5-dihydropyrrole-1-carboxamide | found | |
| | | 561.20 | |
| 80 | N-[2-fluoro-5-[2-(2-hydroxyethoxy)-6-(morpholin-4- | Calc'd | NA |
| | yl)pyridin-4-yl]-4-methylphenyl]-3-(trifluoromethyl)- | 525.20, | |
| | 5,6-dihydro-2H-pyridine-1-carboxamide | found | |
| | | 525.20 | |
| 81 | N-[2-fluoro-5-[2-(2-hydroxyethoxy)-6-(morpholin-4- | Calc'd | NA |
| | yl)pyridin-4-yl]-4-methylphenyl]-3-(2,2,2- | 525.20, | |
| | trifluoroethyl)-2,5-dihydropyrrole-1-carboxamide | found | |
| | | 525.20 | |
| 82 | N-[2-fluoro-5-[2-(2-hydroxyethoxy)-6-(morpholin-4- | Calc'd | CHIRALPAK IG |
| | yl)pyridin-4-yl]-4-methylphenyl]-1-(trifluoromethyl)- | 539.22, | 20 × 250 mm, 5 um |
| | 3-azabicyclo[3.2.0]heptane-3-carboxamide | found | First eluting peak |
| | | 539.25 | |
| 83 | N-[2-fluoro-5-[2-(2-hydroxyethoxy)-6-(morpholin-4- | Calc'd | CHIRALPAK IG |
| | yl)pyridin-4-yl]-4-methylphenyl]-1-(trifluoromethyl)- | 539.22, | 20 × 250 mm, 5 um |
| | 3-azabicyclo[3.2.0]heptane-3-carboxamide | found | First eluting peak |
| | | 539.25 | |
| 84 and 85 | (3R)-N-[2-fluoro-5-[2-(2-hydroxyethoxy)-6- | Calc'd | (R,R)Whelk-O 1 |
| | (morpholin-4-yl)pyridin-4-yl]-4-methylphenyl]-3- | 563.20, | 21.1 × 250 mm, 5 um |
| | (1,1,2,2,2-pentafluoroethyl)pyrrolidine-1-carboxamide | found | Example 84: |
| | and (3S)-N-[2-fluoro-5-[2-(2-hydroxycthoxy)-6- | 563.25 | First eluting peak |
| | (morpholin-4-yl)pyridin-4-yl]-4-methylphenyl]-3- | | Example 85: |
| | (1,1,2,2,2-pentafluoroethyl)pyrrolidine-1-carboxamide | | Second eluting peak |
| 86 | (2S)-N-[2-fluoro-4-methyl-5-[5-(morpholin-4-yl)-6- | Calc'd | NA |
| | (morpholin-4-yloxy)pyridin-3-yl]phenyl]-2- | 569.23, | |
| | (trifluoromethyl)morpholine-4-carboxamide | found | |
| | | 569.35 | |
| 87 | (2R)-N-[2-fluoro-4-methyl-5-[5-(morpholin-4-yl)-6- | Calc'd | NA |
| | (oxan-4-yloxy)pyridin-3-yl]phenyl]-2- | 569.23, | |
| | (trifluoromethyl)morpholine-4-carboxamide | found | |
| | | 569.35 | |
| 88 | (2S)-N-[4-methyl-3-[5-(morpholin-4-yl)-6-(morpholin- | Calc'd | NA |
| | 4-yloxy)pyridin-3-yl]phenyl]-2- | 551.24, | |
| | (trifluoromethyl)morpholine-4-carboxamide | found | |
| | | 551.30 | |
| 89 | (2R)-N-[4-methyl-3-[5-(morpholin-4-yl)-6-(morpholin- | Calc'd | NA |
| | 4-yloxy)pyridin-3-yl]phenyl]-2- | 551.24, | |
| | (trifluoromethyl)morpholine-4-carboxamide | found | |
| | | 551.30 | |
| 90 | 1,1-difluoro-N-[2-fluoro-5-[2-(2-hydroxyethoxy)-6- | Calc'd | CHIRALPAK IG, |
| | (morpholin-4-yl)pyridin-4-yl]-4-methylphenyl]-6- | 521.23, | 20 × 250 mm, 5 um |
| | azaspiro[3,4]octane-6-carboxamide | found | First eluting peak |
| | | 521.25. | |
| 91 | 1,1-difluoro-N-[2-fluoro-5-[2-(2-hydroxyethoxy)-6- | Calc'd | CHIRALPAK IG, |
| | (morpholin-4-yl)pyridin-4-yl]-4-methylphenyl]-6- | 521.23, | 20 × 250 mm, 5 um |
| | azaspiro[3.4]octane-6-carboxamide | found | Second eluting peak |
| | | 521.25. | |
| 92 | (Z)-N-(2-fluoro-5-(2-(2-hydroxyethoxy)-6- | Calc'd | NA |
| | morpholinopyridin-4-yl)-4-methylphenyl)-3-(2,2,2- | 525.20, | |
| | trifluoroethylidene)pyrrolidine-1-carboxamide | found | |
| | | 525.15. | |
| 93 | (E)-N-(2-fluoro-5-(2-(2-hydroxyethoxy)-6- | Calc'd | NA |
| | morpholinopyridin-4-yl)-4-methylphenyl)-3-(2,2,2- | 525.20, | |
| | trifluoroethylidene)pyrrolidine-1-carboxamide | found | |
| | | 525.15. | |
| 94 | (3Z)-N-[2-fluoro-5-[2-(2-hydroxyethoxy)-6- | Calc'd | NA |
| | (morpholin-4-yl)pyridin-4-yl]-4-methylphenyl]-3- | 539.22, | |
| | (1,1,1-trifluoropropan-2-ylidene)pyrrolidine-1- | found | |
| | carboxamide | 539.25. | |
| 95 | (3E)-N-[2-fluoro-5-[2-(2-hydroxyethoxy)-6- | Calc'd | NA |
| | (morpholin-4-yl)pyridin-4-yl]-4-methylphenyl]-3- | 539.22, | |
| | (1,1,1-trifluoropropan-2-ylidene)pyrrolidine-1- | found | |
| | carboxamide | 539.20. | |
| 96 | N-[2-fluoro-5-[2-(2-hydroxyethoxy)-6-(morpholin-4- | Calc'd | NA |
| | yl)pyridin-4-yl]-4-methylphenyl]-3-(2,2,2- | 525.20, | |
| | trifluoroethyl)-2,5-dihydropyrrole-1-carboxamide | found | |
| | | 525.20 | |
| 97 | (3E)-3-(1-cyanoethylidene)-N-[2-fluoro-5-[2-(2- | Calc'd | NA |
| | hydroxyethoxy)-6-(morpholin-4-yl)pyridin-4-yl]-4- | 496.23; | |
| | methylphenyl]pyrrolidine-1-carboxamide | found | |
| | | 496.35 | |
| 98 | (3E)-3-(1-cyanoethylidene)-N-[2-fluoro-5-[2-(2- | Calc'd | NA |
| | hydroxyethoxy)-6-(morpholin-4-yl)pyridin-4-yl]-4- | 496.23; | |
| | methylphenyl]pyrrolidine-1-carboxamide | found | |
| | | 496.35 | |
| 99 | 3-(1-cyano-1-mcthylcthyl)-N-[2-fluoro-5-[2-(2- | Calc'd | NA |
| | hydroxyethoxy)-6-(morpholin-4-yl)pyridin-4-yl]-4- | 512.26, | |
| | methylphenyl]pyrrolidine-1-carboxamide | found | |
| | | 512.30 | |
| 100 | N-[2-fluoro-5-[2-(2-hydroxyethoxy)-6-(morpholin-4- | Calc'd | Chiralpak AD-H, |
| | yl)pyridin-4-yl]-4-methylphenyl]-3-(1,1,1- | 541.24, | 2 × 25 cm (5 um) |
| | trifluoropropan-2-yl)pyrrolidine-1-carboxamide | found | First eluting peak |
| | | 541.25. | |
| 101 | N-[2-fluoro-5-[2-(2-hydroxyethoxy)-6-(morpholin-4- | Calc'd | Chiralpak AD-H, |
| | yl)pyridin-4-yl]-4-methylphenyl]-3-(1,1,1- | 541.24, | 2 × 25 cm (5 um) |
| | trifluoropropan-2-yl)pyrrolidine-1-carboxamide | found | Second eluting peak |
| | | 541.25. | |
| 102 | N-[2-fluoro-5-[2-(2-hydroxyethoxy)-6-(morpholin-4- | Calc'd | Chiralpak AD-H, |
| | yl)pyridin-4-yl]-4-methylphenyl]-3-(1,1,1- | 541.24, | 2 × 25 cm (5 um) |
| | trifluoropropan-2-yl)pyrrolidine-1-carboxamide | found | Third eluting peak |
| | | 541.25. | |
| 103 | N-[2-fluoro-5-[2-(2-hydroxyethoxy)-6-(morpholin-4- | Calc'd | Chiralpak AD-H, |
| | yl)pyridin-4-yl]-4-methylphenyl]-3-(1,1,1- | 541.24, | 2 × 25 cm (5 um) |
| | trifluoropropan-2-yl)pyrrolidine-1-carboxamide | found | Fourth eluting peak |
| | | 541.25. | |
| 104 | 4,4-difluoro-N-[2-fluoro-5-[2-(2-hydroxyethoxy)-6- | Calc'd | NA |
| | (morpholin-4-yl)pyridin-4-yl]-4-methylphenyl]-3- | 563.20; | |
| | (trifluoromethyl)piperidine-1-carboxamide | found | |
| | | 563.25. | |
| 105 and | (4R)-1,1,2,2-tetrafluoro-N-[2-fluoro-5-[2-(2- | Calc'd | Lux 5u Cellulose-4, |
| 106 | hydroxyethoxy)-6-(morpholin-4-yl)pyridin-4-yl]-4- | 563.20; | AXIA Packed, |
| | methylphenyl]-6-azaspiro[3.4]octane-6-carboxamide | found | 2.12 × 25 cm, 5 um |
| | and (4S)-1,1,2,2-tetrafluoro-N-[2-fluoro-5-[2-(2- | 563.25. | Example 105: |
| | hydroxyethoxy)-6-(morpholin-4-yl)pyridin-4-yl]-4- | | First eluting peak |
| | methylphenyl]-6-azaspiro[3.4]octane-6-carboxamide | | Example 106: |
| | | | First eluting peak |
| 107 | N-[2-fluoro-5-[2-(2-hydroxyethoxy)-6-(morpholin-4- | Calc'd | NA |
| | yl)pyridin-4-yl]-4-methylphenyl]-1-(trifluoromethyl)- | 541.20, | |
| | 2-oxa-5-azabicyclo[2.2.1]heptane-5-carboxamide | found | |
| | | 541.25. | |
| 108 | N-[2-fluoro-5-[2-(2-hydroxyethoxy)-6-(morpholin-4- | Calc'd | CHIRALPAK IG |
| | yl)pyridin-4-yl]-4-methylphenyl]-7-(trifluoromethyl)- | 539.22, | 2 × 25 cm, 5 um |
| | 2-azabicyclo[4.1.0]heptane-2-carboxamide | found | First eluting peak |
| | | 539.25. | |
| 109 | N-[2-fluoro-5-[2-(2-hydroxyethoxy)-6-(morpholin-4- | Calc'd | CHIRALPAK IG |
| | yl)pyridin-4-yl]-4-methylphenyl]-7-(trifluoromethyl)- | 539.22, | 2 × 25 cm, 5 um |
| | 2-azabicyclo[4.1.0]heptane-2-carboxamide | found | Second eluting peak |
| | | 539.25. | |
| 110 | N-[2-fluoro-5-[2-(2-hydroxyethoxy)-6-(morpholin-4- | Calc'd | CHIRALPAK IG |
| | yl)pyridin-4-yl]-4-methylphenyl]-7-(trifluoromethyl)- | 539.22, | 2 × 25 cm, 5 um |
| | 2-azabicyclo[4.1.0]heptane-2-carboxamide | found | Third eluting peak |
| | | 539.25. | |
| 111 | N-[2-fluoro-5-[2-(2-hydroxyethoxy)-6-(morpholin-4- | Calc'd | CHIRALPAK IG |
| | yl)pyridin-4-yl]-4-methylphenyl]-7-(trifluoromethyl)- | 539.22, | 2 × 25 cm, 5 um |
| | 2-azabicyclo[4.1.0]heptane-2-carboxamide | found | Fourth eluting peak |
| | | 539.25. | |
| 112 | (2R,3R)-N-(2-fluoro-5-(2-(2-hydroxyethoxy)-6- | Calc'd | CHIRALPAK IG, |
| | morpholinopyridin-4-yl)-4-methylphenyl)-3-methyl-2- | 543.22; | 2 × 25 cm, 5 um |
| | (trifluoromethyl)morpholine-4-carboxamide | found | First eluting peak |
| | | 543.25. | |
| 113 | (2S,3S)-N-(2-fluoro-5-(2-(2-hydroxyethoxy)-6- | Calc'd | CHIRALPAK IG, |
| | morpholinopyridin-4-yl)-4-methylphenyl)-3-methyl-2- | 543.22; | 2 × 25 cm, 5 um |
| | (trifluoromethyl)morpholine-4-carboxamide | found | Second eluting peak |
| | | 543.25. | |
| 114 | 3-(2,2-difluorocyclopropyl)-N-[3-[2-(2- | Calc'd | NA |
| | hydroxyethoxy)-6-(morpholin-4-yl)pyridin-4-yl]-4- | 503.24, | |
| | methylphenyl]pyrrolidine-1-carboxamide | found | |
| | | 503.25. | |
| 115 and | (3R)-N-[3-[2-(2-hydroxyethoxy)-6-(morpholin-4- | Calc'd | CHIRALPAK-AD-H-UL001, |
| 116 | yl)pyridin-4-yl]-4-methylphenyl]-3-(2,2,2- | 509.23, | 20 × 250 mm, 5 um |
| | trifluoroethyl)pyrrolidine-1-carboxamide | found | Example 115: |
| | and (3S)-N-[3-[2-(2-hydroxyethoxy)-6-(morpholin-4- | 509.25. | First eluting peak |
| | yl)pyridin-4-yl]-4-methylphenyl]-3-(2,2,2- | | Example 116: |
| | trifluoroethyl)pyrrolidine-1-carboxamide | | Second eluting peak |
| 117 and | (3R)-N-[3-[2-(2-hydroxyethoxy)-6-(morpholin-4- | Calc'd | CHIRALPAK IG, |
| 118 | yl)pyridin-4-yl]-4-methylphenyl]-3- | 527.19; | 2 × 25 cm, 5 um |
| | [(trifluoromethyl)sulfanyl]pyrrolidine-1-carboxamide | found | Example 117: |
| | and (3S)-N-[3-[2-(2-hydroxyethoxy)-6-(morpholin-4- | 527.15. | First eluting peak |
| | yl)pyridin-4-yl]-4-methylphenyl]-3- | | Example 118: |
| | [(trifluoromethyl)sulfanyl]pyrrolidine-1-carboxamide | | Second eluting peak |
| 119 and | (3R)-N-[4-methyl-3-[5-(morpholin-4-yl)-6-(oxan-4- | Calc'd | Chiralpak AD-H, |
| 120 | yloxy)pyridin-3-yl]phenyl]-3- | 535.25; | 2 × 25 cm (5 um) |
| | (trifluoromethyl)pyrrolidine-1-carboxamide | found | Example 119: |
| | and (3S)-N-[4-methyl-3-[5-(morpholin-4-yl)-6-(oxan-4- | 535.25. | First eluting peak |
| | yloxy)pyridin-3-yl]phenyl]-3- | | Example 120: |
| | (trifluoromethyl)pyrrolidine-1-carboxamide | | Second eluting peak |
| 121 | 1,1-difluoro-N-[3-[2-(2-hydroxyethoxy)-6-(morpholin- | Calc'd | Phenomenex Lux 5u |
| | 4-yl)pyridin-d-yl]-4-methylphenyl]-6- | 503.24, | Cellulose-4, |
| | azaspiro[3.4]octane-6-carboxamide | found | AXIA Packed, |
| | | 503.35. | 2.12 × 25 cm, 5 um |
| | | | First eluting peak |
| 122 | 1,1-difluoro-N-[3-[2-(2-hydroxyethoxy)-6-(morpholin- | Calc'd | Phenomenex Lux 5u |
| | 4-yl)pyridin-4-yl]-4-methylphenyl]-6- | 503.24, | Cellulose-4, |
| | azaspiro[3.4]octane-6-carboxamide | found | AXIA Packed, |
| | | 503.35. | 2.12 × 25 cm, 5 um |
| | | | Second eluting peak |
| 123 | N-[4-methyl-3-[5-(morpholin-4-yl)-6-(oxan-4- | Calc'd | NA |
| | yloxy)pyridin-3-yl]phenyl]-3-(2,2,2- | 549.26, | |
| | trifluoroethyl)pyrrolidine-1-carboxamide | found | |
| | | 549.40. | |
| 124 and | (3R)-N-[3-[2-(2-hydroxyethoxy)-6-(morpholin-4- | Calc'd | CHIRAL ART |
| 125 | yl)pyridin-4-yl]-4-methylphenyl]-3- | 511.21, | Cellulose-SB, |
| | (trifluoromethoxy)pyrrolidine-1-carboxamide | found | 2 × 25 cm, 5 um |
| | and (3S)-N-[3-[2-(2-hydroxyethoxy)-6-(morpholin-4- | 511.25. | Example 124: |
| | yl)pyridin-4-yl]-4-methylphenyl]-3- | | First eluting peak |
| | (trifluoromethoxy)pyrrolidine-1-carboxamide | | Example 125: |
| | | | Second eluting peak |
| 125 | 1-[3-[2-(2-hydroxyethoxy)-6-(morpholin-4-yl)pyridin- | Calc'd | NA |
| | 4-yl]-4-methylphenyl]-3-[1-(trifluoromethyl)pyrazol- | 507.19, | |
| | 4-yl]urea | found | |
| | | 507.15. | |
| 126 | (3R)-N-[2-fluoro-4-methyl-5-[5-(morpholin-4-yl)-6- | Calc'd | NA |
| | (oxan-4-yloxy)pyridin-3-yl]phenyl]-3- | 569.23, | |
| | (trifluoromethoxy)pyrrolidine-1-carboxamide | found | |
| | | 569.40. | |
| 127 | (3S)-N-[2-fluoro-4-methyl-5-[5-(morpholin-4-yl)-6- | Calc'd | NA |
| | (oxan-4-yloxy)pyridin-3-yl]phenyl]-3- | 569.23, | |
| | (trifluoromethoxy)pyrrolidine-1-carboxamide | found | |
| | | 569.40. | |
| 128 | (3R)-N-[4-methyl-3-[5-(morpholin-4-yl)-6-(oxan-4- | Calc'd | NA |
| | yloxy)pyridin-3-yl]phenyl]-3- | 551.24; | |
| | (trifluoromethoxy)pyrrolidine-1-carboxamide | found | |
| | | 551.40. | |
| 129 | (3S)-N-[4-methyl-3-[5-(morpholin-4-yl)-6-(oxan-4- | Calc'd | NA |
| | yloxy)pyridin-3-yl]phenyl]-3- | 551.24; | |
| | (trifluoromethoxy)pyrrolidine-1-carboxamide | found | |
| | | 551.20. | |
| 130 | 1-[4-methyl-3-[5-(morpholin-4-yl)-6-(oxan-4- | Calc'd | NA |
| | yloxy)pyridin-3-yl]phenyl]-3-[1- | 547.22, | |
| | (trifluoromethyl)pyrazol-4-yl]urea | found | |
| | | 547.35. | |
| 131 | (3R)-N-[4-methyl-3-[5-(morpholin-4-yl)-6-(oxan-4- | Calc'd; | NA |
| | yloxy)pyridin-3-yl]phenyl]-3- | 567.64 | |
| | [(trifluoromethyl)sulfanyl]pyrrolidine-1-carboxamide | found | |
| | | 567.35 | |
| 132 | (3S)-N-[4-methyl-3-[5-(morpholin-4-yl)-6-(oxan-4- | Calc'd; | NA |
| | yloxy)pyridin-3-yl]phenyl]-3- | 567.64 | |
| | [(trifluoromethyl)sulfanyl]pyrrolidine-1-carboxamide | found | |
| | | 567.35 | |
| 133 | (3R)-N-[3-[2-(2-hydroxyethoxy)-6-(morpholin-4- | Calc'd | NA |
| | yl)pyridin-4-yl]-4-methylphenyl]-3-(2,2,2- | 525.22, | |
| | trifluoroethoxy)pyrrolidine-1-carboxamide | found | |
| | | 525.25. | |
| 134 | N-[3-[2-(2-hydroxyethoxy)-6-(morpholin-4-yl)pyridin- | Calc'd | NA |
| | 4-yl]-4-methylphenyl]-3- | 559.18; | |
| | trifluoromethanesulfonylpyrrolidine-1-carboxamide | found | |
| | | 559.20. | |
| 135 | 1-[3-[2-(2-hydroxyethoxy)-6-(morpholin-4-yl)pyridin- | Calc'd | NA |
| | 4-yl]-4-methylphenyl]-3-[1-(2,2,2- | 521.20; | |
| | trifluoroethyl)pyrazol-4-yl]urea | found | |
| | | 521.15. | |
| 136 | (3E)-N-[3-[2-(2-hydroxyethoxy)-6-(morpholin-4- | Calc'd | NA |
| | yl)pyridin-4-yl]-4-methylphenyl]-3-(2,2,2- | 507.21, | |
| | trifluoroethylidene)pyrrolidine-1-carboxamide | found | |
| | | 507.15. | |
| 137 | (3S)-N-[3-[2-(2-hydroxyethoxy)-6-(morpholin-4- | Calc'd | NA |
| | yl)pyridin-4-yl]-4-methylphenyl]-3-(2,2,2- | 525.22, | |
| | trifluoroethoxy)pyrrolidine-1-carboxamide | found | |
| | | 525.10. | |
| 138 | (3S)-N-[3-[2-(2-hydroxyethoxy)-6-(morpholin-4- | Calc'd | NA |
| | yl)pyridin-4-yl]-4-methylphenyl]-3- | 485.27, | |
| | isopropoxypyrrolidine-1-carboxamide | found | |
| | | 485.15. | |
| 139 | 1-[3-[2-(2-hydroxyethoxy)-6-(morpholin-4-yl)pyridin- | Calc'd | NA |
| | 4-yl]-4-methylphenyl]-3-(1-isopropylpyrazol-4-yl)urea | 481.25, | |
| | | found | |
| | | 481.25. | |
| 140 | (3S)-3-(1,1-difluorocthoxy)-N-[3-[2-(2- | Calc'd | NA |
| | hydroxyethoxy)-6-(morpholin-4-yl)pyridin-4-yl]-4- | 507.23, | |
| | methylphenyl]pyrrolidine-1-carboxamide | found | |
| | | 507.20. | |
| 141 | 1-[3-[2-(2-hydroxyethoxy)-6-(morpholin-4-yl)pyridin- | Calc'd | NA |
| | 4-yl]-4-methylphenyl]-3-methyl-3-[1- | 521.20, | |
| | (trifluoromethyl)pyrazol-4-yl]urea | found | |
| | | 521.15. | |
| 142 | (3S)-N-(3-[2-[(2R)-2,3-dihydroxypropoxy]-6- | Calc'd | NA |
| | (morpholin-4-yl)pyridin-4-yl]-4-methylphenyl)-3- | 541.22; | |
| | (trifluoromethoxy)pyrrolidine-1-carboxamide | found | |
| | | 541.30. | |
| 143 | (3S)-N-(3-[2-[(2S)-2,3-dihydroxypropoxy]-6- | Calc'd | NA |
| | (morpholin-4-yl)pyridin-4-yl]-4-methylphenyl)-3- | 541.22; | |
| | (trifluoromethoxy)pyrrolidine-1-carboxamide | found | |
| | | 541.30. | |
| 144 | (4R)-N-[3-[2-(2-hydroxyethoxy)-6-(morpholin-4- | Calc'd | NA |
| | yl)pyridin-4-yl]-4-methylphenyl]-4- | 513.19; | |
| | (trifluoromethoxy)-1,2-oxazolidine-2-carboxamide | found | |
| | | 513.20 | |
| 145 | 3-cyclopropylidene-N-[3-[2-(2-hydroxyethoxy)-6- | Calc'd | NA |
| | (morpholin-4-yl)pyridin-4-yl]-4- | 465.24; | |
| | methylphenyl]pyrrolidine-1-carboxamide | found | |
| | | 465.20 | |
| 146 | N-[3-[2-(2-hydroxyethoxy)-6-(morpholin-4-yl)pyridin- | Calc'd | NA |
| | 4-yl]-4-methylphenyl]-4-(trifluoromethoxy) | 512.20; | |
| | pyrazolidine-1-carboxamide | found | |
| | | 512.10 | |
| 147 | N-[3-[2-(2-hydroxyethoxy)-6-(morpholin-4-yl)pyridin- | Calc'd | NA |
| | 4-yl]-4-methylphenyl]-3-(2,2,2-trifluoroacetyl)pyrrole- | 519.18, | |
| | 1-carboxamide | found | |
| | | 519.30 | |
| 148 | 3-hydroxy-N-[3-[2-(2-hydroxyethoxy)-6-(morpholin- | Calc'd | NA |
| | 4-yl)pyridin-4-yl]-4-methylphenyl]-4-(2,2,2- | 525.22, | |
| | trifluoroethyl)pyrrolidine-1-carboxamide | found | |
| | | 525.30 | |

### Example 149: (3R)-N-[3-[2-(2-hydroxy-2-methylpropoxy)-6-(morpholin-4-yl)pyridin-4-yl]-4-methylphenyl]-3-(trifluoromethoxy)pyrrolidine-1-carboxamide

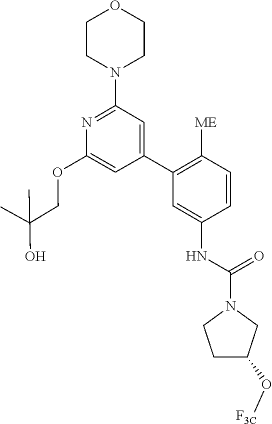

### (3R)-N-[3-[2-(2-hydroxy-2-methylpropoxy)-6-(morpholin-4-yl)pyridin-4-yl]-4-methylphenyl]-3-(trifluoromethoxy)pyrrolidine-1-carboxamide

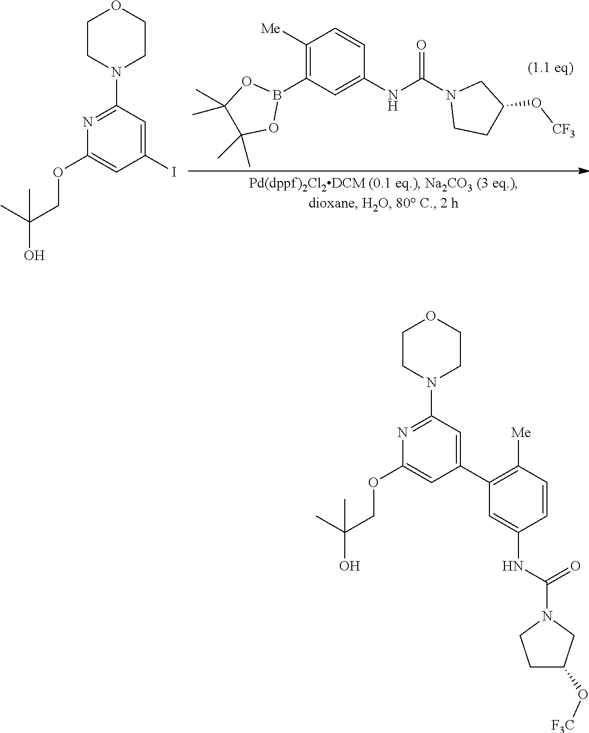

A mixture of 1-[[4-iodo-6-(morpholin-4-yl)pyridin-2-yl]oxy]-2-methylpropan-2-ol (100.00 mg, 0.26 mmol), (3R)-N-[4-methyl-3-(4,4,5,5-tetramethyl-1,3,2-dioxaborolan-2-yl)phenyl]-3-(trifluoromethoxy)pyrrolidine-1-carboxamide (120.48 mg, 0.29 mmol), Pd(dppf)Cl₂.CH₂Cl₂ (21.59 mg, 0.03 mmol), Na₂CO₃ (84.07 mg, 0.79 mmol), 1,4-dioxane (4 mL) and water (1 mL) was stirred for 2 h at 80° C. under nitrogen atmosphere. The resulting mixture was cooled to room temperature and concentrated under reduced pressure. The residue was purified by silica gel column chromatography, eluted with EA:EtOH (3:1)/PE (0 to 100%). The crude product was purified by Prep-HPLC with following conditions: Column: XBridge BEH C18 OBD Prep Column, 5 um, 19 mm×250 mm; Mobile Phase A: water (10 mmol/L NH₄HCO₃), Mobile Phase B: acetonitrile; Flow rate: 20 mL/min; Gradient: 35 B to 65 B in 5 min; 254 nm; RT1: 4.5 min to afford (3R)-N-[3-[2-(2-hydroxy-2-methylpropoxy)-6-(morpholin-4-yl)pyridin-4-yl]-4-methylphenyl]-3-(trifluoromethoxy)pyrrolidine-1-carboxamide (66.7 mg, 470) as an off-white solid. MS ESI calculated for C₂₆H₃₃F₃N₄O₆[M+H]⁺, 539.24, found 539.20. H-NMR (400 z, d₆-DMSO) δ 8.25 (s, 1H), 7.46-7.43 (m, 1H), 7.38-7.37 (m, 1H), 7.15 (d, J=8.4 Hz, 1H), 6.21 (s, 1H), 5.99 (s, 1H), 5.15-5.14 (m, 1H), 4.57 (s, 1H), 4.01 (s, 2H), 3.71-3.55 (m, 7H), 3.45-3.40 (m, 5H), 2.24-2.17 (m, 5H), 1.18 (s, 6H). F-NM/R (376 MHz, d₆-DMSO) δ −56.71 (3F).

The following compounds in Table 4 were prepared using procedures similar to those described in Example 149 using appropriate starting materials. Racemic products were separated using chiral columns specified in Table 4.

TABLE 4

| Example | | Exact Mass | Chiral |
|---|---|---|---|
| Number | IUPAC Name | [M + H]⁺ | column |
| 150 | (3S)-N-(3-[2-[(2N)-2-hydroxypropoxy]-6-(morpholin-4- | Calc'd | NA |
| | yl)pyridin-4-yl]-4-methylphenyl)-3- | 525.22; | |
| | (trifluoromethoxy)pyrrolidine-1-carboxamide | found | |
| | | 525.15 | |
| 151 | (3S)-N-[3-(2-[[(2S)-1-hydroxypropan-2-yl[oxy[-6-(morpholin- | Calc'd | NA |
| | 4-yl)pyridin-4-yl)-4-methylphenyl]-3- | 525.22; | |
| | (trifluoromethoxy)pyrrolidine-1-carboxamide | found | |
| | | 525.25 | |
| 152 | (3S)-N-(3-[2-[(2R)-2-hydroxypropoxy[-6-(morpholin-4- | Calc'd | NA |
| | yl)pyridin-4-yl]-4-methylphenyl)-3- | 525.22; | |
| | (trifluoromethoxy)pyrrolidine-1-carboxamide | found | |
| | | 525.15 | |
| 153 | (3S)-N-[3-(2-[[(2R)-1-hydroxypropan-2-yl]oxy]-6-(morpholin- | Calc'd | NA |
| | 4-yl)pyridin-4-yl)-4-methylphenyl]-3- | 525.22; | |
| | (trifluoromethoxy)pyrrolidine-1-carboxamide | found | |
| | | 525.30 | |
| 154 | (3S)-N-[3-[2-(2-hydroxy-2-methylpropoxy)-6-(morpholin-4- | Calc'd | NA |
| | yl)pyridin-4-yl]-4-methylphenyl]-3- | 539.24; | |
| | (trifluoromethoxy)pyrrolidine-1-carboxamide | found | |
| | | 539.30 | |
| 155 | (3S)-N-(3-[2-[(2R)-2-hydroxypropoxy]-6-(morpholin-4- | Calc'd | NA |
| | yl)pyridin-4-yl]-4-methylphenyl)-3- | 525.22; | |
| | (trifluoromethoxy)pyrrolidine-1-carboxamide | found | |
| | | 525.30. | |
| 156 | 1-(3-[2-[(2R)-2-hydroxypropoxy]-6-(morpholin-4-yl)pyridin- | Calc'd | NA |
| | 4-yl]-4-methylphenyl)-3-methyl-3-[1-(trifluoromethyl)pyrazol- | 535.22, | |
| | 4-yl]urea | found | |
| | | 535.15. | |
| 157 and | (R)-N-(3-(2-((R)-2-hydroxypropoxy)-6-morpholinopyridin-4- | Calc'd | Chiralpak ID-2 |
| 158 | yl)-4-methylphenyl)-3-(2,2,2-trifluoroethyl)pyrrolidine-1- | 523.24, | 2 × 25 cm, 5 um |
| | carboxamide and | found | Example 157 |
| | (S)-N-(3-(2-((R)-2-hydroxypropoxy)-6-morpholinopyridin-4- | 523.30 | First eluting peak |
| | yl)-4-methylphenyl)-3-(2,2,2-trifluoroethyl)pyrrolidine-1- | | Example 158 |
| | carboxamide | | Second eluting peak |
| 159 and | (R)-N-(3-(2-((S)-2-hydroxypropoxy)-6-morpholinopyridin-4- | Calc'd | Chiralpak ID-2 |
| 160 | yl)-4-methylphenyl)-3-(2,2,2-trifluoroethyl)pyrrolidine-1- | 523.24, | 2 × 25 cm, 5 um |
| | carboxamide and | found | Example 158 |
| | (S)-N-(3-(2-((S)-2-hydroxypropoxy)-6-morpholinopyridin-4- | 523.30 | First eluting peak |
| | yl)-4-methylphenyl)-3-(2,2,2-trifluoroethyl)pyrrolidine-1- | | Example 159 |
| | carboxamide | | Second eluting peak |
| 161 and | (3R)-N-[3-[2-(2-hydroxy-3-methoxypropoxy)-6-(morpholin-4- | Calc'd | Chiralpak ID-2 |
| 162 | yl)pyridin-4-yl]-4-methylphenyl]-3-(2,2,2- | 553.26, | 2 × 25 cm, 5 um |
| | trifluoroethyl)pyrrolidine-1-carboxamide and | found | Example 161 |
| | (3S)-N-[3-[2-(2-hydroxy-3-methoxypropoxy)-6-(morpholin-4- | 553.20 | First eluting peak |
| | yl)pyridin-4-yl]-4-methylphenyl]-3-(2,2,2- | | Example 162 |
| | trifluoroethyl)pyrrolidine-1-carboxamide | | Second eluting peak |
| 163, 164, | (3R)-N-(3-[2-[(2R)-2-hydroxy-3-methoxypropoxy]-6- | Calc'd | Lux 5u |
| 165 and | (morpholin-4-yl)pyridin-4-yl]-4-methylphenyl)-3-(2,2,2- | 553.26, | Cellulose-4, |
| 166 | trifluoroethyl)pyrrolidine-1-carboxamide and | found | AXIA Packed, |
| | (3R)-N-(3-[2-[(2S)-2-hydroxy-3-methoxypropoxy]-6- | 553.20 | 2.12 × 25 cm, 5 um |
| | (morpholin-4-yl)pyridin-4-yl]-4-methylphenyl)-3-(2,2,2- | | Example 163 |
| | trifluoroethyl)pyrrolidine-1-carboxamide and | | First eluting peak |
| | (3S)-N-(3-[2-[(2R)-2-hydroxy-3-methoxypropoxy]-6- | | Example 164 |
| | (morpholin-4-yl)pyridin-4-yl]-4-methylphenyl)-3-(2,2,2- | | Second eluting peak |
| | trifluoroethyl)pyrrolidine-1-carboxamide and | | Example 165 |
| | (3S)-N-(3-[2-[(2S)-2-hydroxy-3-methoxypropoxy]-6- | | Third eluting peak |
| | (morpholin-4-yl)pyridin-4-yl]-4-methylphenyl)-3-(2,2,2- | | Example 166 |
| | trifluoroethyl)pyrrolidine-1-carboxamide | | Fourth eluting peak |
| 167 | 2,2-difluoro-N-(3-(2-(2-hydroxyethoxy)-6-morpholinopyridin- | Calc'd | NA |
| | 4-yl)-4-methylphenyl)-6-azaspiro[3.4]octane-6-carboxamide | 503.23; | |
| | | found | |
| | | 503.2 | |
| 168 | (S)-N-(2-fluoro-5-(2-(2-hydroxyethoxy)-6-morpholinopyridin- | Calc'd | NA |
| | 4-yl)-4-methylphenyl)-2-(trifluoromethyl)thiomorpholine-4- | 545.18; | |
| | carboxamide | found | |
| | | 545.1 | |
| 169 | (R)-N-(2-fluoro-5-(2-(2-hydroxyethoxy)-6-morpholinopyridin- | Calc'd | NA |
| | 4-yl)-4-methylphenyl)-2-(trifluoromethyl)thiomorpholine-4- | 545.18; | |
| | carboxamide | found | |
| | | 545.1 | |

### Example 170: (3S)-N-(3-[2-[(1-hydroxy-2-methyl propan-2-yl)oxy]-6-(morpholin-4-yl)pyridin-4-yl]-4-methyl phenyl)-3-(trifluoromethoxy)pyrrolidine-1-carboxamide

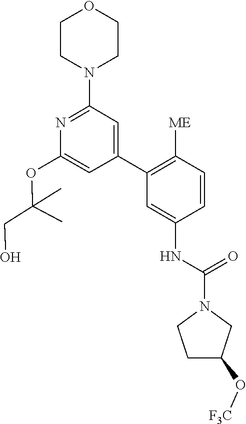

### Step 1: (3S)-N-[4-methyl-3-(2-[[2-methyl-1-(oxan-2-yloxy)propan-2-yl]oxy]-6-(morpholin-4-yl)pyridin-4-yl)phenyl]-3-(trifluoromethoxy)pyrrolidine-1-carboxamide

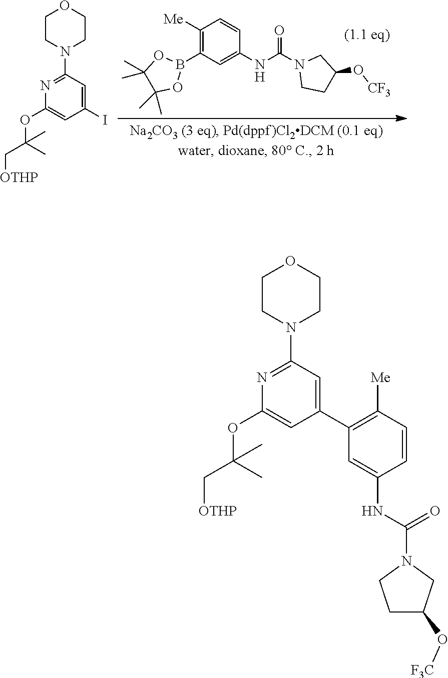

A mixture of 4-(4-iodo-6-[[2-methyl-1-(oxan-2-yloxy)propan-2-yl]oxy]pyridin-2-yl)morpholine (100.00 mg, 0.22 mmol), (3S)-N-[4-methyl-3-(4,4,5,5-tetramethyl-1,3,2-dioxaborolan-2-yl)phenyl]-3-(trifluoromethoxy)pyrrolidine-1-carboxamide (98.56 mg, 0.24 mmol), Pd(dppf)Cl₂.CH₂Cl₂ (17.66 mg, 0.02 mmol), Na₂CO₃ (68.77 mg, 0.65 mmol), 1,4-dioxane (4.00 mL) and water (1.00 mL) was stirred for 2 h at 80° C. under nitrogen atmosphere. The resulting mixture was cooled to room temperature and concentrated under reduced pressure. The residue was purified by silica gel column chromatography, eluted with EA/PE (0 to 100%) to afford (3S)-N-[4-methyl-3-(2-[[2-methyl-1-(oxan-2-yloxy)propan-2-yl]oxy]-6-(morpholin-4-yl)pyridin-4-yl)phenyl]-3-(trifluoromethoxy)pyrrolidine-1-carboxamide (100 mg, 74.25%) as a light yellow solid. MS ESI calculated for C₃₁H₄₁F₃N₄O₆[M+H]⁺, 623.30, found 623.20.

### Step 2: (3S)-N-(3-[2-[(1-hydroxy-2-methylpropan-2-yl)oxy]-6-(morpholin-4-yl)pyridin-4-yl]-4-methylphenyl)-3-(trifluoromethoxy)pyrrolidine-1-carboxamide

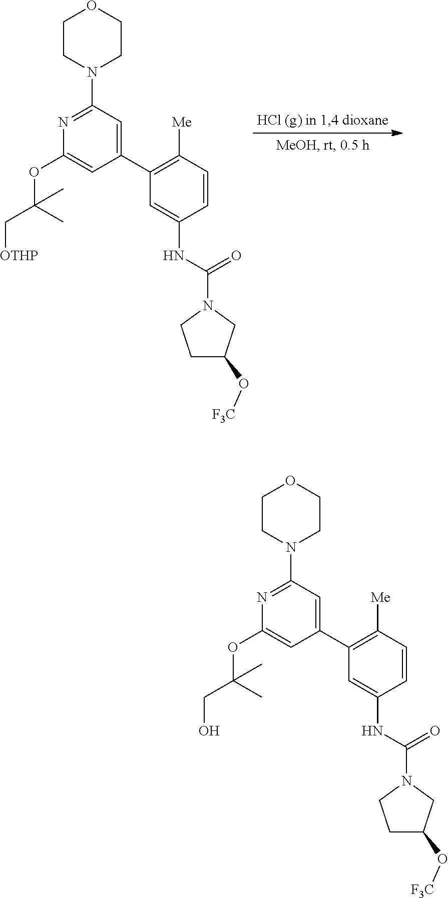

To a stirred solution of (3S)-N-[4-methyl-3-(2-[[2-methyl-1-(oxan-2-yloxy)propan-2-yl]oxy]-6-(morpholin-4-yl)pyridin-4-yl)phenyl]-3-(trifluoromethoxy)pyrrolidine-1-carboxamide (100.00 mg, 0.16 mmol) in MeOH (3 mL) was added HCl (gas) in 1,4-dioxane (1.00 mL, 4 M) dropwise at room temperature. The resulting solution was stirred for 0.5 h at room temperature. The reaction solution was basified to pH ˜8 with saturated NaHCO₃ (aq.). The resulting mixture was extracted with EtOAc (3×50 mL). The combined organic layers was washed with brine (100 mL), dried over anhydrous Na₂SO₄. After filtration, the filtrate was concentrated under reduced pressure. The crude product was purified by Prep-HPLC with following conditions: Column: XBridge C18 OBD Prep Column, 100 Å, 10 μm, 19 mm×250 mm; Mobile Phase A: water (10 mmoL/L NH₄HCO₃), Mobile Phase B: ACN; Flow rate: 20 mL/min; Gradient: 50 B to 80 B in 5.8 m; 254/210 nm; RT1: 5.75 mi to afford (3S)-N-(3-[2-[(1-hydroxy-2-methylpropan-2-yl)oxy]-6-(morpholin-4-yl)pyridin-4-yl]-4-methylphenyl)-3-(trifluoromethoxy)pyrrolidine-1-carboxamide (35.3 mg, 410) as an off-white solid. MS ESI calculated for C₂₆H₃₃F₃N₄O₅[M+H]f, 539.24, found 539.20. H-NM/R (400 MHz, d₆-DMSO) δ 8.25 (s, 1H), 7.46-7.43 (m, 1H), 7.39-7.38 (m, 1H), 7.15 (d, J=8.4 Hz, 1H), 6.22 (s, 1H), 5.93 (s, 1H), 5.15-5.14 (m, 1H), 4.86 (t, J=6.0 Hz, 1H), 3.74-3.56 (m, 9H), 3.48-3.40 (m, 5H), 2.25-2.19 (m, 5H), 1.51 (s, 6H). F-NM/R (376 MHz, d₆-DMSO) δ −56.71 (3F).

The following compounds in Table 5 were prepared using procedures similar to those described in Example 149 and related examples using appropriate starting materials. Racemic products were separated using chiral columns specified in Table 5.

TABLE 5

| Example | | Exact Mass | Chiral |
|---|---|---|---|
| Number | IUPAC Name | [M + H]⁺ | column |
| 171 | N-(3-(2-(((2R,3R)-3-hydroxybutan-2-yl)oxy)-6- | Calc'd | NA |
| | morpholinopyridin-4-yl)-4-methylphenyl)-3-(trifluoromethyl)- | 521.23; | |
| | 2,5-dihydro-1H-pyrrole-1-carboxamide | found | |
| | | 521.20 | |
| 172 | (S)-N-(3-(2-(((2R,3R)-3-hydroxybutan-2-yl)oxy)-6- | Calc'd | NA |
| | morpholinopyridin-4-yl)-4-methylphenyl)-3- | 539.24 | |
| | (trifluoromethoxy)pyrrolidine-1-carboxamide | found | |
| | | 539.30 | |
| 173 | 3-(tert-butyl)-N-(3-(2-(2-hydroxyethoxy)-6-morpholinopyridin- | Calc'd | NA |
| | 4-yl)-4-methylphenyl)-1H-pyrrole-1-carboxamide | 479.26; | |
| | | found | |
| | | 479.20 | |
| 174 | (S)-3-(difluoromethoxy)-N-(3-(2-(((2R,3R)-3-hydroxybutan-2- | Calc'd | NA |
| | yl)oxy)-6-morpholinopyridin-4-yl)-4-methylphenyl)pyrrolidine- | 521.25; | |
| | 1-carboxamide | found | |
| | | 521.20 | |
| 175 | (S)-N-(3-(2-((R)-2-hydroxypropoxy)-6-morpholinopyridin-4- | Calc'd | Chiralpak IG-2 |
| | yl)-4-methylphenyl)-3-(2,2,2-trifluoroethyl)pyrrolidine-1- | 523.25; | 2 × 25 cm, 5 um |
| | carboxamide | found | Example 5 |
| | | 523.30 | Second eluting peak |
| 176 | (1R,5S,6r)-N-(3-(2-(2-hydroxyethoxy)-6-morpholinopyridin-4- | Calc'd | NA |
| | yl)-4-methylphenyl)-6-(trifluoromethyl)-3- | 507.21; | |
| | azabicyclo[3.1.0]hexane-3-carboxamide | found | |
| | | 507.20 | |
| 177 and | (S)-N-(3-(2-(2-hydroxy-2-methylpropoxy)-6- | Calc'd | Chiralpak IG-2 |
| 178 | morpholinopyridin-4-yl)-4-methylphenyl)-3-(2,2,2- | 537.26; | 2 × 25 cm, 5 um |
| | trifluoroethyl)pyrrolidine-1-carboxamide and | found | Example 7 |
| | (R)-N-(3-(2-(2-hydroxy-2-methylpropoxy)-6- | 537.20 | second eluting peak |
| | morpholinopyridin-4-yl)-4-methylphenyl)-3-(2,2,2- | | Example 8 |
| | trifluoroethyl)pyrrolidine-1-carboxamide | | first eluting peak |
| 179 | N-(3-(2-((S)-2,3-dihydroxypropoxy)-6-morpholinopyridin-4- | Calc'd | NA |
| | yl)-4-methylphenyl)-3-(2,2,2-trifluoroethyl)pyrrolidine-1- | 539.24; | |
| | carboxamide | found | |
| | | 539.30 | |
| 180 | N-(3-(2-((R)-2,3-dihydroxypropoxy)-6-morpholinopyridin-4- | Calc'd | NA |
| | yl)-4-methylphenyl)-3-(2,2,2-trifluoroethyl)pyrrolidine-1- | 539.24; | |
| | carboxamide | found | |
| | | 539.30 | |
| 181, 182, | (S)-N-(2-fluoro-5-(2-((R)-2-hydroxypropoxy)-6- | Calc'd | Chiralpak IG-4 |
| 183 and | morpholinopyridin-4-yl)-4-methylphenyl)-3-(2,2,2- | 541.24; | 2 × 25 cm, 5 um |
| 184 | trifluoroethyl)pyrrolidine-1-carboxamide and | found | Example 11 |
| | (S)-N-(2-fluoro-5-(2-(((R)-1-hydroxypropan-2-yl)oxy)-6- | 541.30 | Forth eluting peak |
| | morpholinopyridin-4-yl)-4-methylphenyl)-3-(2,2,2- | | Example 12 |
| | trifluoroethyl)pyrrolidine-1-carboxamide and | | Second eluting peak |
| | (R)-N-(2-fluoro-5-(2-((R)-2-hydroxypropoxy)-6- | | Example 13 |
| | morpholinopyridin-4-yl)-4-methylphenyl)-3-(2,2,2- | | Third eluting peak |
| | trifluoroethyl)pyrrolidine-1-carboxamide and | | Example 14 |
| | (R)-N-(2-fluoro-5-(2-(((R)-1-hydroxypropan-2-yl)oxy)-6- | | First eluting peak |
| | morpholinopyridin-4-yl)-4-methylphenyl)-3-(2,2,2- | | |
| | trifluoroethyl)pyrrolidine-1-carboxamide | | |
| 185 and | (S)-N-(3-(2-((S)-2-hydroxypropoxy)-6-morpholinopyridin-4- | Calc'd | Chiralpak IG-2 |
| 186 | yl)-4-methylphenyl)-3-(2,2,2-trifluoroethyl)pyrrolidine-1- | 523.25; | 2 × 25 cm, 5 um |
| | carboxamide and | found | Example 15 |
| | (R)-N-(3-(2-((S)-2-hydroxypropoxy)-6-morpholinopyridin-4- | 523.30 | Second eluting peak |
| | yl)-4-methylphenyl)-3-(2,2,2-trifluoroethyl)pyrrolidine-1- | | Example 16 |
| | carboxamide | | First eluting peak |
| 187 | N-(4-methyl-3-(2-morpholino-6-((tetrahydro-2H-pyran-4- | Calc'd | NA |
| | yl)oxy)pyridin-4-yl)phenyl)-3-(2,2,2-trifluoroethyl)pyrrolidine- | 549.26; | |
| | 1-carboxamide | found | |
| | | 549.30 | |
| 188 | N-(4-methyl-3-(2-morpholino-6-(((S)-tetrahydrofuran-3- | Calc'd | NA |
| | yl)oxy)pyridin-4-yl)phenyl)-3-(2,2,2-trifluoroethyl)pyrrolidine- | 535.25; | |
| | 1-carboxamide | found | |
| | | 535.30 | |
| 189 | N-(3-(2-(3-hydroxypropoxy)-6-morpholinopyridin-4-yl)-4- | Calc'd | NA |
| | methylphenyl)-3-(2,2,2-trifluoroethyl)pyrrolidine-1- | 523.25; | |
| | carboxamide | found | |
| | | 523.30 | |
| 190 | N-(4-methyl-3-(2-((1-methylpiperidin-4-yl)oxy)-6- | Calc'd | NA |
| | morpholinopyridin-4-yl)phenyl)-3-(2,2,2- | 562.29; | |
| | trifluoroethyl)pyrrolidine-1-carboxamide | found | |
| | | 562.40 | |
| 191 and | (S)-N-(3-(2-(((R)-1-hydroxypropan-2-yl)oxy)-6- | Calc'd | Chiralpak AD-H-2 |
| 192 | morpholinopyridin-4-yl)-4-methylphenyl)-3-(2,2,2- | 523.25; | 2 × 25 cm, 5 um |
| | trifluoroethyl)pyrrolidine-1-carboxamide and | found | Example 21 |
| | (R)-N-(3-(2-(((R)-1-hydroxypropan-2-yl)oxy)-6- | 523.30 | Second eluting peak |
| | morpholinopyridin-4-yl)-4-methylphenyl)-3-(2,2,2- | | Example 22 |
| | trifluoroethyl)pyrrolidine-1-carboxamide | | First eluting peak |
| 193 and | (S)-N-(4-methyl-3-(5-morpholino-6-((tetrahydro-2H-pyran-4- | Calc'd | Chiralpak IC-2 |
| 194 | yl)oxy)pyridin-3-yl)phenyl)-3-(2,2,2-trifluoroethyl)pyrrolidine- | 549.26; | 2 × 25 cm, 5 um |
| | 1-carboxamide and | found | Example 24 |
| | (R)-N-(4-methyl-3-(5-morpholino-6-((tetrahydro-2H-pyran-4- | 549.30 | First eluting peak |
| | yl)oxy)pyridin-3-yl)phenyl)-3-(2,2,2-trifluoroethyl)pyrrolidine- | | Example 25 |
| | 1-carboxamide | | Second eluting peak |
| 195 | N-(4-methyl-3-(2-(((S)-1-methylpyrrolidin-3-yl)oxy)-6- | Calc'd | NA |
| | morpholinopyridin-4-yl)phenyl)-3-(2,2,2- | 548.28; | |
| | trifluoroethyl)pyrrolidine-1-carboxamide | found | |
| | | 548.30 | |
| 196 | N-(3-(2-(3-hydroxy-2,2-dimethylpropoxy)-6- | Calc'd | NA |
| | morpholinopyridin-4-yl)-4-methylphenyl)-3-(2,2,2- | 551.28; | |
| | trifluoroethyl)pyrrolidine-1-carboxamide | found | |
| | | 551.30 | |
| 197 and | (S)-N-(3-(2-(((S)-1-hydroxypropan-2-yl)oxy)-6- | Calc'd | Chiralpak IC-2 |
| 198 | morpholinopyridin-4-yl)-4-methylphenyl)-3-(2,2,2- | 523.25; | 2 × 25 cm, 5 um |
| | trifluoroethyl)pyrrolidine-1-carboxamide and | found | Example 28 |
| | (R)-N-(3-(2-(((S)-1-hydroxypropan-2-yl)oxy)-6- | 523.30 | First eluting peak |
| | morpholinopyridin-4-yl)-4-methylphenyl)-3-(2,2,2- | | Example 29 |
| | trifluoroethyl)pyrrolidine-1-carboxamide | | Second eluting peak |
| 199 | N-(3-(2-(3-hydroxy-2-methylpropoxy)-6-morpholinopyridin-4- | Calc'd | NA |
| | yl)-4-methylphenyl)-3-(2,2,2-trifluoroethyl)pyrrolidine-1- | 537.26; | |
| | carboxamide | found | |
| | | 537.30 | |
| 200 | N-(4-methyl-3-(2-morpholino-6-(((R)-tetrahydroluran-3- | Calc'd | NA |
| | yl)oxy)pyridin-4-yl)phenyl)-3-(2,2,2-trifluoroethyl)pyrrolidine- | 535.25; | |
| | 1-carboxamide | found | |
| | | 535.30 | |
| 201 | N-(4-methyl-3-(2-morpholino-6-((S)-pyrrolidin-3- | Calc'd | NA |
| | yloxy)pyridin-4-yl)phenyl)-3-(2,2,2-trifluoroethyl)pyrrolidine- | 534.26; | |
| | 1-carboxamide | found | |
| | | 534.30 | |
| 202 | N-(4-methyl-3-(2-(((R)-1-methylpyrrolidin-3-yl)oxy)-6- | Calc'd | NA |
| | morpholinopyridin-4-yl)phenyl)-3-(2,2,2- | 548.28; | |
| | trifluoroethyl)pyrrolidine-1-carboxamide | found | |
| | | 548.30 | |
| 203 | N-(4-methyl-3-(2-morpholino-6-((R)-pyrrolidin-3- | Calc'd | NA |
| | yloxy)pyridin-4-yl)phenyl)-3-(2,2,2-trifluoroethyl)pyrrolidine- | 534.26; | |
| | 1-carboxamide | found | |
| | | 534.30 | |
| 204 | (S)-N-(3-(2-(azetidin-3-yloxy)-6-morpholinopyridin-4-yl)-4- | Calc'd | NA |
| | methylphenyl)-3-(2,2,2-trifluoroethyl)pyrrolidine-1- | 520.25; | |
| | carboxamide | found | |
| | | 519.90 | |
| 205 | (R)-N-(3-(2-(azetidin-3-yloxy)-6-morpholinopyridin-4-yl)-4- | Calc'd | NA |
| | methylphenyl)-3-(trifluoromethoxy)pyrrolidine-1-carboxamide | 522.22; | |
| | | found | |
| | | 521.90 | |
| 206 | (3S)-N-[3-[2-(3-hydroxycyclobutoxy)-6-(morpholin-4- | Calc'd | NA |
| | yl)pyridin-4-yl]-4-methylphenyl]-3-(2,2,2- | 535.25, | |
| | trifluoroethyl)pyrrolidine-1-carboxamide | found | |
| | | 535.25. | |
| 207, 208, | (3S)-N-[3-(2-[[(1S,3S)-3-hydroxycyclopentyl]oxy]-6- | Calc'd | CHIRALPAK ADH, |
| 209 and | (morpholin-4-yl)pyridin-4-yl)-4-methylphenyl]-3-(2,2,2- | 549.26, | 2 × 25 cm, 5 um |
| 210 | trifluoroethyl)pyrrolidine-1-carboxamide and | found | |
| | (3S)-N-[3-(2-[[(1R,3R)-3-hydroxycyclopentyl]oxy]-6- | 549.30. | |
| | (morpholin-4-yl)pyridin-4-yl)-4-methylphenyl]-3-(2,2,2- | | |
| | trifluoroethyl)pyrrolidine-1-carboxamide and | | |
| | (3S)-N-[3-(2-[[(1S,3R)-3-hydroxycyclopentyl]oxy]-6- | | |
| | (morpholin-4-yl)pyridin-4-yl)-4-methylphenyl]-3-(2,2,2- | | |
| | trifluoroethyl)pyrrolidine-1-carboxamide and | | |
| | (3S)-N-[3-(2-[[(1R,3S)-3-hydroxycyclopentyl]oxy]-6- | | |
| | (morpholin-4-yl)pyridin-4-yl)-4-methylphenyl]-3-(2,2,2- | | |
| | trifluoroethyl)pyrrolidine-1-carboxamide | | |
| 211 | (3S)-N-(3-[2-[(4-hydroxy-4-methylcyclohexyl)oxy]-6- | Calc'd | NA |
| | (morpholin-4-yl)pyridin-4-yl]-4-methylphenyl)-3-(2,2,2- | 577.29, | |
| | trifluoroethyl)pyrrolidine-1-carboxamide (cis) | found | |
| | | 577.35. | |
| 212 | (3S)-N-(3-[2-[(4-hydroxy-4-methylcyclohexyl)oxy]-6- | Calc'd | NA |
| | (morpholin-4-yl)pyridin-4-yl]-4-methylphenyl)-3-(2,2,2- | 577.29, | |
| | trifluoroethyl)pyrrolidine-1-carboxamide (trans) | found | |
| | | 577.35. | |
| 213 | (3R)-N-(3-[2-[(2S)-2-hydroxypropoxy]-6-(morpholin-4- | Calc'd | NA |
| | yl)pyridin-4-yl]-4-methylphenyl)-3- | 525.22; | |
| | (trifluoromethoxy)pyrrolidine-1-carboxamide | found | |
| | | 525.30 | |
| 214 | (3R)-N-[3-(2-[[(2R)-1-hydroxypropan-2-yl]oxy]-6-(morpholin- | Calc'd | NA |
| | 4-yl)pyridin-4-yl)-4-methylphenyl]-3- | 525.22; | |
| | (trifluoromethoxy)pyrrolidine-1-carboxamide | found | |
| | | 525.10 | |
| 215 | (3R)-N-[3-(2-[[(2S)-1-hydroxypropan-2-yl]oxy]-6-(morpholin- | Calc'd | NA |
| | 4-yl)pyridin-4-yl)-4-methylphenyl]-3- | 525.22; | |
| | (trifluoromethoxy)pyrrolidine-1-carboxamide | found | |
| | | 525.20 | |
| 216 | N-[3-[2-(2-hydroxy-2-methylpropoxy)-6-(morpholin-4- | Calc'd | NA |
| | yl)pyridin-4-yl]-4-methylphenyl]-3-(2,2,2-trifluoroethyl)-2,5- | 535.25; | |
| | dihydropyrrole-1-carboxamide | found | |
| | | 535.25 | |
| 217 | (3S)-N-[3-[2-(3-hydroxy-3-methylcyclobutoxy)-6-(morpholin- | Calc'd | NA |
| | 4-yl)pyridin-4-yl]-4-methylphenyl]-3-(2,2,2- | 549.26; | |
| | trifluoroethyl)pyrrolidine-1-carboxamide | found | |
| | | 549.15 | |
| 218 | (3S)-N-(3-[2-[(1-hydroxycyclopropyl)methoxy]-6-(morpholin- | Calc'd | NA |
| | 4-yl)pyridin-4-yl]-4-methylphenyl)-3-(2,2,2- | 535.25; | |
| | trifluoroethyl)pyrrolidine-1-carboxamide | found | |
| | | 535.30 | |
| 219 | (3S)-N-[3-(2-[[(1R)-3,3-difluorocyclopentyl]oxy]-6-(morpholin- | Calc'd | CHIRALPAK ADH, |
| | 4-yl)pyridin-4-yl)-4-methylphenyl]-3-(2,2,2- | 569.25; | 2 × 25 cm, 5 um |
| | trifluoroethyl)pyrrolidine-1-carboxamide | found | |
| | | 569.25 | |
| 220 | (3S)-N-[3-(2-[[(1S)-3,3-difluorocyclopentyl]oxy]-6-(morpholin- | Calc'd | CHIRALPAK ADH, |
| | 4-yl)pyridin-4-yl)-4-methylphenyl]-3-(2,2,2- | 569.25, | 2 × 25 cm, 5 um |
| | trifluoroethyl)pyrrolidine-1-carboxamide | found | |
| | | 569.20 | |
| 221 | (3S)-N-[3-(2-[2-[imino(mcthyl)oxo-λ⁶-sulfanyl[ethoxy[-6- | Calc'd | NA |
| | (morpholin-4-yl)pyridin-4-yl)-4-methylphenyl]-3-(2,2,2- | 570.23; | |
| | trifluoroethyl)pyrrolidine-1-carboxamide | found | |
| | | 570.25 | |
| 222 | (R)-N-(3-(2-(2-hydroxypropoxy)-6-morpholinopyridin-4-yl)-4- | Calc'd | NA |
| | methylphenyl)-3-(2,2,2-trifluoroethyl)-2,5-dihydro-1H-pyrrole- | 521.23, | |
| | 1-carboxamide | found | |
| | | 521.15 | |
| 223 | (3S)-N-(3-[2-[(3-hydroxy-3-methylcyclopentyl)oxy]-6- | Calc'd | NA |
| | (morpholin-4-yl)pyridin-4-yl]-4-methylphenyl)-3-(2,2,2- | 563.28, | |
| | trifluoroethyl)pyrrolidine-1-carboxamide | found | |
| | | 563.30 | |
| 224 | (3S)-N-(3-[2-[(3-hydroxyoxctan-3-yl)methoxy[-6-(morpholin-4- | Calc'd | NA |
| | yl)pyridin-4-yl]-4-methylphenyl)-3-(2,2,2- | 551.24, | |
| | trifluoroethyl)pyrrolidine-1-carboxamide | found | |
| | | 551.25 | |
| 225 | (3S)-N-(3-[2-[2-(3-hydroxyoxetan-3-yl)ethoxy]-6-(morpholin- | Calc'd | NA |
| | 4-yl)pyridin-4-yl]-4-methylphenyl)-3-(2,2,2- | 565.26, | |
| | trifluoroethyl)pyrrolidine-1-carboxamide | found | |
| | | 565.20 | |
| 226 | (3S)-N-[4-methyl-3-[5-(morpholin-4-yl)pyridin-3-yl]phcnyl]-3- | Calc'd | NA |
| | (2,2,2-trifluoroethyl)pyrrolidine-1-carboxamide | 449.21, | |
| | | found | |
| | | 449.25 | |

### Example 227: (3S)-N-(3-[2-[(2R)-2-hydroxypropoxyl-6-[(2S)-2-methylmorpholin-4-yl]pyridin-4-yl]-4-methylphenyl)-3-(2,2,2-trifluoroethyl)pyrrolidine-1-carboxamide

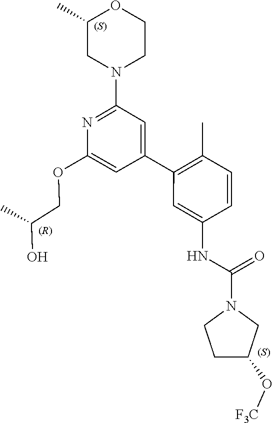

### Step 1: (3S)-N-(3-[2-[(2R)-2-hydroxypropoxy]-6-[(2S)-2-methylmompholin-4-yl]pyridin-4-yl]-4-methylphenyl)-3-(2,2,2-trifluoroethyl)pyrrolidine-1-carboxamide

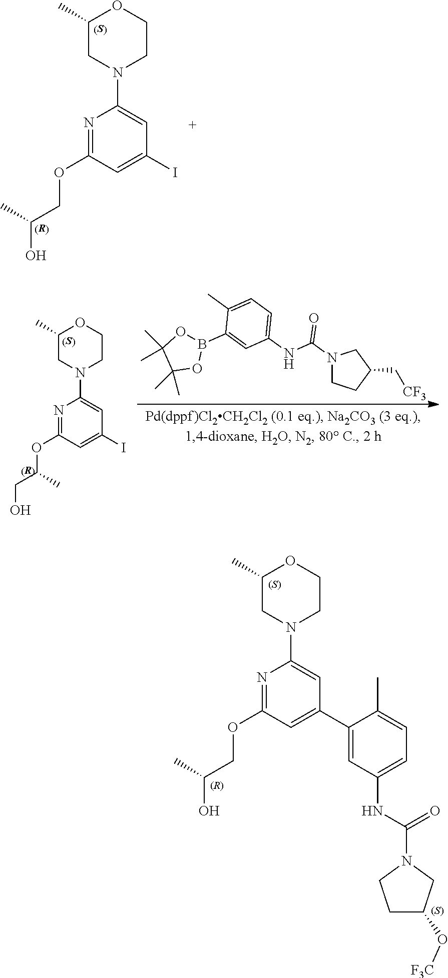

A mixture of (2R)-1-([4-iodo-6-[(2S)-2-methylmorpholin-4-yl]pyridin-2-yl]oxy)propan-2-ol (240 mg, 0.635 mmol, 1.00 equiv) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (261.61 mg, 0.635 mmol, 1.00 equiv), 1,4-dioxane (4.00 mL), H₂O (1.00 mL), Na₂CO₃ (201.77 mg, 1.904 mmol, 3.00 equiv) and Pd(dppf)Cl₂.DCM (51.82 mg, 0.063 mmol, 0.10 equiv) stirred for 2 h at 80 degrees C. under N₂ atmosphere. The resulting mixture was concentrated under reduced pressure. The residue was purified by silica gel column chromatography, eluted with 60% EtOAc in PE. The crude was purified under following conditions: Column: GreenSep Basic, 30*150 mm Sum; Mobile Phase A: CO₂, Mobile Phase B: IPA(0.5% 2M NH3-MeOH); Flow rate: 50 mL/min; Gradient: 35% B; 254 nm; RT1:5.08; RT2:5.45; Injection Volume: 0.6 ml; Number Of Runs: 20; to afford (3S)-N-(3-[2-[(2R)-2-hydroxypropoxy]-6-[(2S)-2-methylmorpholin-4-yl]pyridin-4-yl]-4-methylphenyl)-3-(2,2,2-trifluoroethyl)pyrrolidine-1-carboxamide (39.9 mg, 12%) as a white solid. MS ESI calculated for C₂₇H₃₅F₃N₄O₄[M+H]⁺, 537.26, found 537.30.

The following compounds in Table 6 were prepared using procedures similar to those described in Example 227 using appropriate starting materials. Racemic products were separated using chiral columns specified in the table.

TABLE 6

| Example | | Exact Mass | Chiral |
|---|---|---|---|
| Number | IUPAC Name | [M + H]⁺ | column |
| 228 | (S)-N-(3-(2-((R)-2-hydroxypropoxy)-6-((R)-2- | Calc'd | NA |
| | methylmorpholino)pyridin-4-yl)-4-methylphenyl)-3- | 537.26, | |
| | (2,2,2-trifluoroethyl)pyrrolidine-1-carboxamide | found | |
| | | 537.30 | |
| 229 | (3S)-N-(3-[2-[(2R)-2-hydroxypropoxy]-6-[2-oxa-6- | Calc'd | NA |
| | azaspiro[3.3]heptan-6-yl]pyridin-4-yl]-4- | 535.25, | |
| | methylphenyl)-3-(2,2,2-trifluoroethyl)pyrrolidine-1- | found | |
| | carboxamide | 535.25 | |
| 230 | (3S)-N-(3-(2-(2-oxa-5-azabicyclo[4.1.0]heptan-5-yl)-6- | Calc'd | NA |
| | ((R)-2-hydroxypropoxy)pyridin-4-yl)-4- | 535.25, | |
| | methylphenyl)-3-(2,2,2-trifluoroethyl)pyrrolidine-1- | found | |
| | carboxamide | 535.30 | |
| 231 | (S)-N-(3-(2-(1-(hydroxymethyl)cyclopropoxy)-6- | Calc'd | NA |
| | morpholinopyridin-4-yl)-4-methylphenyl)-3-(2,2,2- | 535.24, | |
| | trifluoroethyl)pyrrolidine-1-carboxamide | found | |
| | | 535.20 | |
| 232 | (S)-N-(3-(2-((1-hydroxy-2-methylpropan-2-yl)oxy)-6- | Calc'd | NA |
| | morpholinopyridin-4-yl)-4-methylphenyl)-3-(2,2,2- | 537.26, | |
| | trifluoroethyl)pyrrolidine-1-carboxamide | found | |
| | | 537.20 | |
| 233 | (S)-N-(3-(2-(((2R,3R)-3-hydroxybutan-2-yl)oxy)-6- | Calc'd | NA |
| | morpholinopyridin-4-yl)-4-methylphenyl)-3-(2,2,2- | 537.26, | |
| | trifluoroethyl)pyrrolidine-1-carboxamide | found | |
| | | 537.20 | |

### II. Biological Evaluation

### Example 1: Kinase Assay Protocol

Protein kinase assay: Assay platform was used to measure kinase/inhibitor interactions as described previously (Anastassiadis et al., 2011). In brief, for each reaction, kinase and substrate were mixed in a buffer containing 20 mM HEPES (pH 7.5), 10 mM MgCl2, 1 mM EGTA, 0.02% Brij35, 0.02 mg/mL BSA, 0.1 mM Na3VO4, 2 mM DTT, and 100 DMSO. All compounds were solubilized in DMSO. Compounds were then added to each reaction mixture via acoustic dispense using an ECHO 550 nanoliter dispenser. For human RAF1 testing, human MEK1 (K97R) was used as a substrate at a concentration of 3 micromolar, with a final ATP concentration of 10 micromolar. For human BRAF testing, human MEK1 (K97R) was used as a substrate at 1 micromolar concentration, with a final ATP concentration of 25 micromolar. Compounds were tested in 10-dose IC₅₀ mode with a 3-fold serial dilution starting at 10 micromolar. After a 20-min incubation, ATP (Sigma-Aldrich, St. Louis, Mo. 63178) and [g33P] ATP (specific activity 10 microCi/microliter) purchased at PerkinElmer (Boston, Mass., 02118 Cat #BLU 003H250UC) were added at a final total concentration of 10 mM. Reactions were carried out at room temperature for 2 hr and spotted onto P81 ion exchange cellulose chromatography paper (Reaction Biology). Filter paper was washed in 0.75% phosphoric acid to remove unincorporated ATP. The percent remaining kinase activity relative to a vehicle-containing (DMSO) kinase reaction was calculated for each kinase/inhibitor pair. IC₅₀ values were calculated using Prism 5 (GraphPad).

Representative data for exemplary compounds are presented in Table 5.

TABLE 5

| Synthetic Chemistry | RAF-1 | B-RAF |
|---|---|---|
| Example | IC₅₀ | IC₅₀ |
| 1 | A | B |
| 2 | A | B |
| 3 | A | B |
| 4 | A | B |
| 5 | A | B |
| 6 | A | B |
| 7 | A | C |
| 8 | A | B |
| 9 | A | B |
| 10 | A | B |
| 11 | B | C |
| 12 | A | — |
| 13 | A | — |
| 14 | A | — |
| 15 | A | — |
| 16 | A | — |
| 17 | A | — |
| 18 | A | — |
| 19 | A | — |
| 20 | B | — |
| 21 | B | — |
| 22 | A | — |
| 23 | A | — |
| 24 | B | — |
| 25 | B | — |
| 26 | A | — |
| 27 | B | — |
| 28 | B | — |
| 29 | A | — |
| 30 | A | — |
| 31 | A | — |
| 32 | A | — |
| 33 | A | — |
| 34 | A | — |
| 35 | A | — |
| 36 | A | — |
| 37 | A | — |
| 38 | A | — |
| 39 | A | — |
| 40 | A | — |
| 41 | B | — |
| 42 | A | — |
| 43 | A | — |
| 44 | A | — |
| 45 | A | — |
| 46 | B | — |
| 47 | A | — |
| 48 | A | — |
| 49 | A | — |
| 50 | A | — |
| 51 | A | — |
| 52 | A | — |
| 53 | A | — |
| 54 | A | — |
| 55 | A | — |
| 56 | B | — |
| 57 | A | — |
| 58 | A | — |
| 59 | A | — |
| 60 | A | — |
| 61 | A | — |
| 62 | A | — |
| 63 | A | — |
| 64 | C | — |
| 65 | B | — |
| 66 | A | — |
| 67 | A | — |
| 68 | A | — |
| 69 | A | — |
| 70 | B | — |
| 71 | C | — |
| 72 | A | — |
| 73 | A | — |
| 74 | A | — |
| 75 | A | — |
| 76 | A | — |
| 77 | A | — |
| 78 | A | — |
| 79 | A | — |
| 80 | A | — |
| 81 | A | — |
| 82 | A | — |
| 83 | A | — |
| 84 | A | — |
| 85 | A | — |
| 86 | A | — |
| 87 | A | — |
| 88 | A | — |
| 89 | A | — |
| 90 | A | — |
| 91 | A | — |
| 92 | A | — |
| 93 | A | — |
| 94 | A | — |
| 95 | A | — |
| 96 | A | — |
| 97 | A | — |
| 98 | A | — |
| 99 | A | — |
| 100 | A | — |
| 101 | A | — |
| 102 | A | — |
| 103 | A | — |
| 104 | A | — |
| 105 | A | — |
| 106 | A | — |
| 107 | A | — |
| 108 | D | — |
| 109 | A | — |
| 110 | A | — |
| 111 | A | — |
| 112 | A | — |
| 113 | A | — |
| 114 | A | — |
| 115 | A | — |
| 116 | A | — |
| 117 | A | — |
| 118 | A | — |
| 119 | A | — |
| 120 | A | — |
| 121 | A | — |
| 122 | A | — |
| 123 | A | — |
| 124 | A | — |
| 125 | A | — |
| 126 | A | — |
| 127 | A | — |
| 128 | A | — |
| 129 | A | — |
| 130 | A | — |
| 131 | A | — |
| 132 | A | — |
| 133 | A | — |
| 134 | A | — |
| 135 | A | — |
| 136 | A | — |
| 137 | A | — |
| 138 | A | — |
| 139 | A | — |
| 140 | A | — |
| 141 | A | — |
| 142 | A | — |
| 143 | A | — |
| 144 | A | — |
| 145 | A | — |
| 146 | A | — |
| 147 | D | — |
| 148 | A | — |
| 149 | A | — |
| 150 | A | — |
| 151 | A | — |
| 152 | A | — |
| 153 | A | — |
| 154 | A | — |
| 155 | A | — |
| 156 | A | — |
| 157 | A | — |
| 158 | A | — |
| 167 | A | — |
| 168 | A | — |
| 169 | A | — |
| 170 | A | — |
| 171 | A | — |
| 172 | A | — |
| 173 | A | — |
| 174 | A | — |
| 175 | A | — |
| 176 | A | — |
| 177 and 178 | A | — |
| 179 | A | — |
| 180 | A | — |
| 181, 182, | A, A, | — |
| 183 and 184 | A and A | |
| 185 and 186 | A and A | — |
| 187 | A | — |
| 188 | A | — |
| 189 | A | — |
| 190 | A | — |
| 191 and 192 | A and A | — |
| 193 and 194 | A and A | — |
| 195 | A | — |
| 196 | A | — |
| 197 and 198 | A and A | — |
| 199 | A | — |
| 200 | A | — |
| 201 | A | — |
| 202 | A | — |
| 203 | A | — |
| 204 | A | — |
| 205 | A | — |
| 206 | A | — |
| 207, 208, | A, A, | — |
| 209 and 210 | A and A | |
| 211 | A | — |
| 212 | A | — |
| 213 | A | — |
| 214 | A | — |
| 215 | A | — |
| 216 | A | — |
| 217 | A | — |
| 218 | A | — |
| 219 | A | — |
| 220 | A | — |
| 221 | A | — |
| 222 | A | — |
| 223 | A | — |
| 224 | A | — |
| 225 | A | — |
| 226 | A | — |
| 227 | A | — |
| 228 | A | — |
| 229 | A | — |
| 230 | A | — |
| 231 | A | — |
| 232 | A | — |
| 233 | A | — |

Note:
Biochemical assay IC₅₀ data are designated within the following ranges:
A: ≤0.010 μM
B: >0.010 μM to ≤0.10 μM
C: >0.10 μM to ≤1.0 μM
D: >1.0 μM to ≤10 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

We claim:

1. A compound, or pharmaceutically acceptable salt or solvate thereof, having the structure of Formula (I): 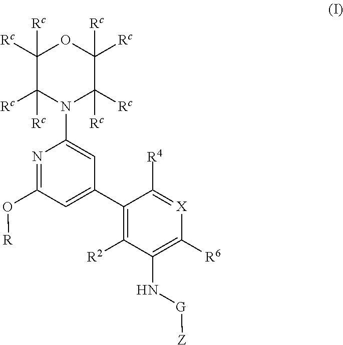
wherein,
G is C═O or SO₂;
R is C₁-C₈ optionally substituted alkyl, —(C1-C8 optionally substituted alkylene)-OPO(OH)₂, —(C1-C8 optionally substituted alkylene)-S(O)NHMe, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2; or optionally, if q is 2, then two R¹ groups join to form a fused ring;
R² is H, D or F;
R⁴ is halogen, optionally substituted C1-C3 alkyl, —CD₃, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b) 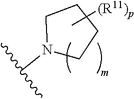
wherein m is 0, 1, 2, or 3; p is 0, 1, 2, 3, or 4; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl or two R¹¹ groups together form an oxo;
(c) 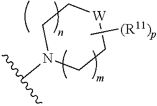
wherein m is 0, 1, 2, or 3; p is 0, 1, 2, 3, or 4;
W is O, S, S(O), SO₂, NH or N(optionally substituted C1-C6 alkyl); and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d) 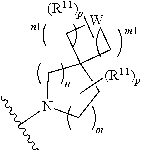
wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2;
W is O, S, S(O), SO₂, NH or N(optionally substituted C1-C6 alkyl), CH₂, CHR¹¹, or C(R¹¹)₂; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e) 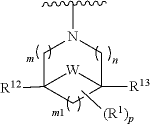
wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—, —O—CH₂—, or —CH₂—O—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f) 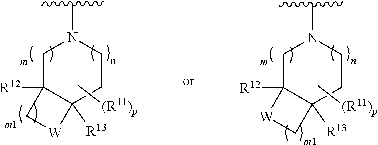
wherein m is 0, 1, or 2; n is 0, 1, or 2; m1 is 0, 1, or 2; p is 0, 1, or 2;
W is O, S, S(O), S02, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g) 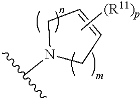
wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h) 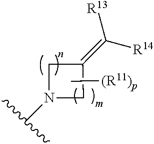
wherein m is 1, 2, or 3; n is 1, 2, or 3; p is 0, 1, or 2; and
each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl;
(i) 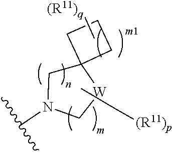
wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

2. A compound, or pharmaceutically acceptable salt or solvate thereof, having the structure of Formula (II): 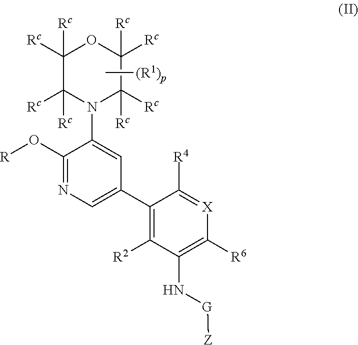
wherein
G is C═O or SO₂;
R is C1-C8 optionally substituted alkyl, —(C1-C8 optionally substituted alkylene)-OPO(OH)₂, —(C1-C8 optionally substituted alkylene)-S(O)NHMe, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
X is N, C—H, C-D, C—F, or C—CH₃;
R¹ is C1-C3 optionally substituted alkyl, and q is 0, 1, or 2; or optionally, if q is 2, then two R¹ groups join to form a fused ring;
R² is H, D or F;
R⁴ is halogen, optionally substituted C1-C3 alkyl, —CD3, or optionally substituted C1-C3 alkoxy;
R⁶ is H, D, Cl or F;
R^{c} is H or D;
Z is selected from:
(a) —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl;
(b) 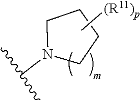
wherein m is 0, 1, 2, or 3; p is 0, 1, 2, 3, or 4; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo;
(c) 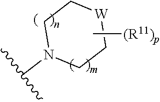
wherein m is 0, 1, 2, or 3; p is 0, 1, 2, 3, or 4;
W is O, S, S(O), SO₂, NH or N(optionally substituted C1-C6 alkyl); and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(d) 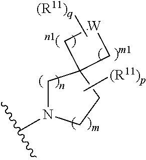
wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2;
W is O, S, S(O), SO₂, NH or N(optionally substituted C1-C6 alkyl), CH₂, CHR¹¹, or C(R¹¹)₂; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(e) 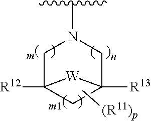
wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—, —O—CH₂—, or —CH₂—O—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl;
(f) 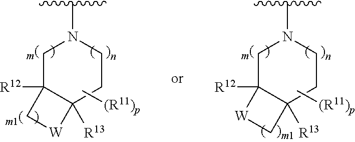
wherein m is 0, 1, or 2; n is 0, 1, or 2; m1 is 0, 1, or 2; p is 0, 1, or 2;
W is O, S, S(O), SO₂, NH or N(optionally substituted C1-C6 alkyl), CH₂, CHR¹¹, or C(R¹¹)₂ each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
and R¹² and R¹³ are each independently selected from H, or optionally substituted C1-C6 alkyl;
(g) 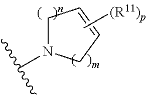
wherein m is 0, 1, 2, or 3; n is 0, 1, 2, or 3 provided both m and n are not both 0; p is 0, 1, 2, 3, or 4; and
each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo;
(h) 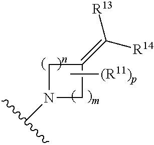
wherein m is 1, 2, or 3; n is 1, 2, or 3; p is 0, 1, or 2; and
each R¹³ or R¹⁴ is independently selected from hydrogen, halogen, —CN, optionally substituted C1-C6 alkyl, or optionally substituted C3-C6 cycloalkyl; each R¹¹ is independently selected from —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl;
(i) 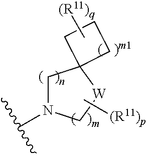
wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

3. The compound of claim 1 or 2, or pharmaceutically acceptable salt or solvate thereof, wherein G is C═O.

4. The compound of any one of the preceding claims, or pharmaceutically acceptable salt or solvate thereof, wherein R^{c} is hydrogen.

5. The compound of any one of claims 1-4, or pharmaceutically acceptable salt or solvate thereof, wherein R^{c} is deuterium.

6. The compound of any one of the preceding claims, or pharmaceutically acceptable salt or solvate thereof, wherein R² is hydrogen or deuterium.

7. The compound of any one of the preceding claims, or pharmaceutically acceptable salt or solvate thereof, wherein R⁶ is hydrogen or deuterium.

8. The compound of any one of the preceding claims, or pharmaceutically acceptable salt or solvate thereof, wherein R² is F.

9. The compound of any one of the preceding claims, or pharmaceutically acceptable salt or solvate thereof, wherein R⁶ is F.

10. The compound of any one of the preceding claims, or pharmaceutically acceptable salt or solvate thereof, wherein X is N.

11. The compound of any one of claims 1-9, or pharmaceutically acceptable salt or solvate thereof, wherein X is C—H or C-D.

12. The compound of any one of claims 1-9, or pharmaceutically acceptable salt or solvate thereof, wherein X is C—F.

13. The compound of any one of the preceding claims, or pharmaceutically acceptable salt or solvate thereof, wherein R¹ is optionally substituted C1 alkyl.

14. The compound of any one of the preceding claims, or pharmaceutically acceptable salt or solvate thereof, wherein q is 0.

15. The compound of any one of claims 1-13, or pharmaceutically acceptable salt or solvate thereof, wherein q is 1.

16. The compound of any one of claims 1-13 or 15, or pharmaceutically acceptable salt or solvate thereof, wherein R¹ is CH₃, q is 1, and R¹ is positioned to provide a 3-methylmorpholino.

17. The compound of any one of the preceding claims, or pharmaceutically acceptable salt or solvate thereof, wherein R is C1-C8 optionally substituted alkyl.

18. The compound of any one of claims 1-16, or pharmaceutically acceptable salt or solvate thereof, wherein R is —(C1-C8 optionally substituted alkylene)-OPO(OH)₂.

19. The compound of any one of claims 1-16, or pharmaceutically acceptable salt or solvate thereof, wherein R is C3-C6 optionally substituted cycloalkyl.

20. The compound of any one of claims 1-16, or pharmaceutically acceptable salt or solvate thereof, wherein R is C4-C6 optionally substituted cycloalkylalkyl.

21. The compound of any one of claims 1-16, or pharmaceutically acceptable salt or solvate thereof, wherein R is C3-C6 optionally substituted heterocyclyl.

22. The compound of any one of claims 1-16, or pharmaceutically acceptable salt or solvate thereof, wherein R is C3-C6 optionally substituted heterocyclylalkyl.

23. The compound of claim 17, or pharmaceutically acceptable salt or solvate thereof, wherein the C1-C8 optionally substituted alkyl is a C2 optionally substituted alkyl.

24. The compound of claim 18, or pharmaceutically acceptable salt or solvate thereof, wherein the —(C1-C8 optionally substituted alkylene)-OPO(OH)₂ is a C2 optionally substituted alkylene.

25. The compound of any one of the preceding claims, or pharmaceutically acceptable salt or solvate thereof, wherein R⁴ is halogen.

26. The compound of any one of the preceding claims, or pharmaceutically acceptable salt or solvate thereof, wherein R⁴ is optionally substituted C1-C3 alkyl.

27. The compound of any one of the preceding claims, or pharmaceutically acceptable salt or solvate thereof, wherein R⁴ is methyl.

28. The compound of any one of the preceding claims, or pharmaceutically acceptable salt or solvate thereof, wherein Z is —NR^{a}R^{b}, wherein R^{a} is selected from H, optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted heterocyclyl, or optionally substituted heterocyclylalkyl; and
R^{b} is selected from optionally substituted alkyl, optionally substituted C3-C6 alkenyl, optionally substituted C3-C6 alkynyl, optionally substituted C3-C6 cycloalkyl, optionally substituted C3-C6 cycloalkylalkyl, optionally substituted C4-C6 heterocyclyl, or optionally substituted heterocyclylalkyl.

29. The compound of claim 28, or pharmaceutically acceptable salt or solvate thereof, wherein R^{a} is H.

30. The compound of claim 28, or pharmaceutically acceptable salt or solvate thereof, wherein R^{a} is optionally substituted alkyl.

31. The compound of any one of claims 28-30, or pharmaceutically acceptable salt or solvate thereof, wherein R^{b} is optionally substituted alkyl.

32. The compound of any one of claims 1-27, or pharmaceutically acceptable salt or solvate thereof, wherein Z is 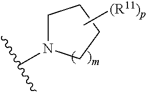
wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo.

33. The compound of claim 32, or pharmaceutically acceptable salt or solvate thereof, wherein m is 0.

34. The compound of claim 32, or pharmaceutically acceptable salt or solvate thereof, wherein m is 1.

35. The compound of claim 32, or pharmaceutically acceptable salt or solvate thereof, wherein m is 2.

36. The compound of claim 32, or pharmaceutically acceptable salt or solvate thereof, wherein m is 3.

37. The compound of any one of claims 32-36, or pharmaceutically acceptable salt or solvate thereof, wherein p is 0.

38. The compound of any one of claims 32-36, or pharmaceutically acceptable salt or solvate thereof, wherein p is 1.

39. The compound of any one of claims 32-36, or pharmaceutically acceptable salt or solvate thereof, wherein p is 2.

40. The compound of any one of claims 32-36, or pharmaceutically acceptable salt or solvate thereof, wherein p is 1.

41. The compound of any one of claims 32-40, or pharmaceutically acceptable salt or solvate thereof, wherein R¹¹ is optionally substituted C1-C6 alkyl, or optionally substituted C3-C6 cycloalkyl.

42. The compound of claim 41, or pharmaceutically acceptable salt or solvate thereof, wherein the optionally substituted C1-C6 alkyl, or optionally substituted C3-C6 cycloalkyl is substituted with at least a halogen.

43. The compound of any one of claims 1-27, or pharmaceutically acceptable salt or solvate thereof, wherein Z is 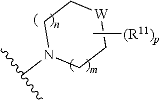
wherein m is 0, 1, 2, or 3; p is 0, 1, 2, 3, or 4; W is O, S, S(O), SO₂, NH or N(optionally substituted C1-C6 alkyl); and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo.

44. The compound of claim 43, or pharmaceutically acceptable salt or solvate thereof, wherein W is O.

45. The compound of claim 43, or pharmaceutically acceptable salt or solvate thereof, wherein W is S.

46. The compound of any one of claims 43-45, or pharmaceutically acceptable salt or solvate thereof, wherein m is 1, and n is 1.

47. The compound of any one of claims 43-45, or pharmaceutically acceptable salt or solvate thereof, wherein m is 1, and n is 2.

48. The compound of any one of claims 43-47, or pharmaceutically acceptable salt or solvate thereof, wherein R¹¹ is optionally substituted C1-C6 alkyl, or optionally substituted C3-C6 cycloalkyl.

49. The compound of claim 48, or pharmaceutically acceptable salt or solvate thereof, wherein the optionally substituted C1-C6 alkyl, or optionally substituted C3-C6 cycloalkyl is substituted with at least a halogen.

50. The compound of any one of claims 1-27, or pharmaceutically acceptable salt or solvate thereof, wherein Z is 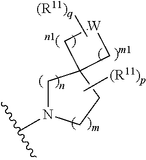
wherein m is 0, 1, or 2; n is 0, 1, or 2; m1 is 0, 1, or 2; n1 is 0, 1, or 2 provided both m1 and n1 are not both 0; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo.

51. The compound of claim 50, or pharmaceutically acceptable salt or solvate thereof, wherein m is 1, and n is 1.

52. The compound of any one of claims 50-51, or pharmaceutically acceptable salt or solvate thereof, wherein m is 0, and n is 2.

53. The compound of any one of claims 50-52, or pharmaceutically acceptable salt or solvate thereof, wherein m1 is 0, and n1 is 2.

54. The compound of any one of claims 50-53, or pharmaceutically acceptable salt or solvate thereof, wherein m1 is 1, and n1 is 1.

55. The compound of any one of claims 50-54, or pharmaceutically acceptable salt or solvate thereof, wherein W is O.

56. The compound of any one of claims 50-54, or pharmaceutically acceptable salt or solvate thereof, wherein W is CH₂.

57. The compound of any one of claims 50-54, or pharmaceutically acceptable salt or solvate thereof, wherein W is CHR¹¹.

58. The compound of any one of claims 50-54, or pharmaceutically acceptable salt or solvate thereof, wherein W is C(R¹¹)₂.

59. The compound of any one of claims 57-58, or pharmaceutically acceptable salt or solvate thereof, wherein R¹¹ is halogen and q is 1.

60. The compound of any one of claims 1-27, or pharmaceutically acceptable salt or solvate thereof, wherein Z is 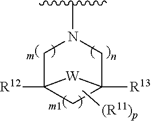
wherein m is 0, 1, or 2; n is 0, 1, or 2; m1 is 1, or 2; p is 0, 1, 2, or 3; W is O, S, S(O), SO₂, NH or N(optionally substituted C1-C6 alkyl), CH₂, CHR¹¹, —CH₂—CH₂—, —CH₂—CHR¹¹—, —CH₂—C(R¹¹)₂—, —CHR¹¹—CH₂—, —C(R¹¹)₂—CH₂—, —NH—CH₂—, —NH—CHR¹¹—, —NH—C(R¹¹)₂—, —CH₂—NH—, —CHR¹¹—NH—, —C(R¹¹)₂—NH—, —N(R¹¹)—CH₂—, —N(R¹¹)—CHR¹¹—, —N(R¹¹)—C(R¹¹)₂—, —CH₂—N(R¹¹)—, —CHR¹¹—N(R¹¹)—, —C(R¹¹)₂—N(R¹¹)—; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl.

61. The compound of claim 60, or pharmaceutically acceptable salt or solvate thereof, wherein m is 0, n is 1, and m1 is 1; and W is —O—CH₂—, or —CH₂—O—.

62. The compound of any one of claims 1-27, or pharmaceutically acceptable salt or solvate thereof, wherein Z is 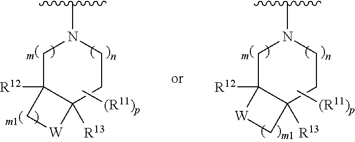
wherein m is 0, 1, or 2; n is 0, 1, or 2; m1 is 0, 1, or 2; p is 0, 1, or 2; W is O, S, S(O), SO₂, NH or N(optionally substituted C1-C6 alkyl), CH₂, CHR¹¹, or C(R¹¹)₂; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two R¹¹ groups together form an oxo; and R¹² and R¹³ are each independently selected from H, or optionally substituted C1-C6 alkyl.

63. The compound of claim 62, or pharmaceutically acceptable salt or solvate thereof, wherein W is O.

64. The compound of any one of claims 62-63, or pharmaceutically acceptable salt or solvate thereof, wherein W is CH₂, or CHR¹¹.

65. The compound of any one of claims 62-64, or pharmaceutically acceptable salt or solvate thereof, wherein m1 is 0.

66. The compound of any one of claims 62-64, or pharmaceutically acceptable salt or solvate thereof, wherein m1 is 1.

67. The compound of any one of claims 62-66, or pharmaceutically acceptable salt or solvate thereof, wherein m is 1 and n is 1.

68. The compound of any one of claims 62-66, or pharmaceutically acceptable salt or solvate thereof, wherein m is 1 and n is 0.

69. The compound of any one of claims 62-66, or pharmaceutically acceptable salt or solvate thereof, wherein m is 0 and n is 1.

70. The compound of any one of claims 1-27, or pharmaceutically acceptable salt or solvate thereof, wherein Z is 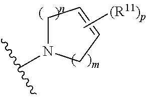
wherein m is 0, 1, 2, or 3; n is 0, 1, 2, or 3 provided both m and n are not both 0; p is 0, 1, 2, 3, or 4; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl.

71. The compound of claim 70, or pharmaceutically acceptable salt or solvate thereof, wherein m is 1, and n is 1.

72. The compound of claim 70, or pharmaceutically acceptable salt or solvate thereof, wherein m is 1, and n is 2.

73. The compound of any one of claims 70-72, or pharmaceutically acceptable salt or solvate thereof, wherein p is 1.

74. The compound of any one of claims 70-72, or pharmaceutically acceptable salt or solvate thereof, wherein p is 2.

75. The compound of any one of claims 70-74, or pharmaceutically acceptable salt or solvate thereof, wherein at least one R¹¹ is attached to an alkene carbon.

76. The compound of any one of claims 70-74, or pharmaceutically acceptable salt or solvate thereof, wherein at least one R¹¹ is not attached to an alkene carbon.

77. The compound of any one of claims 70-76, or pharmaceutically acceptable salt or solvate thereof, wherein R¹¹ is optionally substituted C1-C6 alkyl, or optionally substituted C3-C6 cycloalkyl.

78. The compound of any one of claims 70-72, or pharmaceutically acceptable salt or solvate thereof, wherein p is 0.

79. The compound of any one of claims 1-27, or pharmaceutically acceptable salt or solvate thereof, wherein Z is 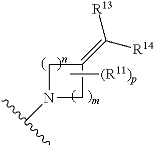
wherein m is 1, 2, or 3; n is 1, 2, or 3; p is 0, 1, or 2; and each R¹³ or R¹⁴ is independently selected from hydrogen, halogen, —CN, optionally substituted C1-C6 alkyl, or optionally substituted C3-C6 cycloalkyl; each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl.

80. The compound of claim 79, or pharmaceutically acceptable salt or solvate thereof, wherein m is 2, and n is 1.

81. The compound of claim 79 or 80, or pharmaceutically acceptable salt or solvate thereof, wherein p is 0.

82. The compound of claim 79 or 80, or pharmaceutically acceptable salt or solvate thereof, wherein p is 1.

83. The compound of any one of claims 79-82, or pharmaceutically acceptable salt or solvate thereof, wherein one of R¹³ or R¹⁴ is not hydrogen.

84. The compound of any one of claims 79-83, or pharmaceutically acceptable salt or solvate thereof, wherein one of R¹³ or R¹⁴ is optionally substituted C1-C6 alkyl.

85. The compound of claim 84, or pharmaceutically acceptable salt or solvate thereof, wherein R¹³ is optionally substituted C1-C6 alkyl.

86. The compound of claim 84, or pharmaceutically acceptable salt or solvate thereof, wherein R¹⁴ is optionally substituted C1-C6 alkyl.

87. The compound of any one of claims 1-27, or pharmaceutically acceptable salt or solvate thereof, wherein Z is 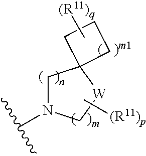
wherein m is 0, 1, or 2; n is 0, 1, or 2; m1 is 0, 1, or 2; p is 0, 1, or 2; and q is 0, 1 or 2; W is O, S, S(O), SO₂, NH or N(optionally substituted C1-C6 alkyl), CH₂, CHR¹¹, or C(R¹¹)₂; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —SO₂alkyl, optionally substituted C3-C6 cycloalkylalkyl, optionally substituted heterocyclyl, or optionally substituted heterocyclylalkyl, or two geminal R¹¹ groups together form an oxo.

88. The compound of claim 87, or pharmaceutically acceptable salt or solvate thereof, wherein W is O.

89. The compound of claim 87 or 88, or pharmaceutically acceptable salt or solvate thereof, wherein m is 2, and n is 1.

90. The compound of any one of claims 87-89, or pharmaceutically acceptable salt or solvate thereof, wherein m1 is 1 or 2.

91. The compound of any one of claims 87-90, or pharmaceutically acceptable salt or solvate thereof, wherein p is 0 or 1, and q is 0 or 1.

92. A compound, or pharmaceutically acceptable salt or solvate thereof, selected from a compound described in Table 1.

93. A pharmaceutical composition comprising a compound of Formula (I), or pharmaceutically acceptable salt or solvate thereof, as described in any one of claim 1 or 3-91.

94. A pharmaceutical composition comprising a compound of Formula (II), or pharmaceutically acceptable salt or solvate thereof, as described in any one of claims 2-91.

95. A pharmaceutical composition comprising a compound as described in claim 92, or pharmaceutically acceptable salt or solvate thereof, and a pharmaceutically acceptable excipient.

96. A pharmaceutical composition comprising a compound of Formula (III)-(VI), or pharmaceutically acceptable salt or solvate thereof, and a pharmaceutically acceptable excipient.

97. A method of preparing a pharmaceutical composition comprising mixing a compound, or pharmaceutically acceptable salt or solvate thereof, of any one of claims 1-92, and a pharmaceutically acceptable carrier.

98. A compound of any one of claims 1-92 or pharmaceutically acceptable salt or solvate thereof, for use in a method of treatment of the human or animal body.

99. A compound of any one of claims 1-92, or pharmaceutically acceptable salt or solvate thereof, for use in a method of treatment of cancer or neoplastic disease.

100. Use of a compound of any one of claims 1-92, or pharmaceutically acceptable salt or solvate thereof, in the manufacture of a medicament for the treatment of cancer or neoplastic disease.

101. A method of treating cancer in a patient in need thereof comprising administering to the patient a compound of Formula (I) as described in any one of claim 1 or 3-91, or pharmaceutically acceptable salt or solvate thereof.

102. A method of treating cancer in a patient in need thereof comprising administering to the patient a pharmaceutical composition comprising a compound of Formula (I) as described in any one of claim 1 or 3-91, or pharmaceutically acceptable salt or solvate thereof, and a pharmaceutically acceptable excipient.

103. A method of treating cancer in a patient in need thereof comprising administering to the patient a compound of Formula (II) as described in any one of claims 2-91, or pharmaceutically acceptable salt or solvate thereof.

104. A method of treating cancer in a patient in need thereof comprising administering to the patient a pharmaceutical composition comprising a compound of Formula (II) as described in any one of claims 2-91, or pharmaceutically acceptable salt or solvate thereof, and a pharmaceutically acceptable excipient.

105. A method of treating cancer in a patient in need thereof comprising administering to the patient a compound as described in claim 92, or pharmaceutically acceptable salt or solvate thereof.

106. A method of treating cancer in a patient in need thereof comprising administering to the patient a pharmaceutical composition comprising a compound as described in claim 92, or pharmaceutically acceptable salt or solvate thereof, and a pharmaceutically acceptable excipient.

107. A method of treating cancer in a patient in need thereof comprising administering to the patient a compound of Formula (III)-(VI), or pharmaceutically acceptable salt or solvate thereof.

108. A method of treating cancer in a patient in need thereof comprising administering to the patient a pharmaceutical composition comprising a compound of Formula (III)-(VI), or pharmaceutically acceptable salt or solvate thereof, and a pharmaceutically acceptable excipient.

109. The method of any one of claims 101-108 wherein the cancer is breast cancer, colorectal cancer, ovarian cancer, pancreatic cancer, prostate cancer, or lung cancer.
